(19) United States
(12) Patent Application Publication
(10) Pub. No.: US 2009/0325959 A1
(43) Pub. Date: Dec. 31, 2009

(54) METHOD FOR TREATING OPHTHALMIC DISEASES USING RHO KINASE INHIBITOR COMPOUNDS

(76) Inventors: Jason L. Vittitow, Durham, NC (US); Ward M. Peterson, Morrisville, NC (US); John W. Lampe, Cary, NC (US); Tomas Navratil, Carrboro, NC (US); Emilee H. Fulcher, Cary, NC (US)

Correspondence Address: HOWERY LLP, C/O IP DOCKETING DEPARTMENT, 2941 FAIRVIEW PARK DRIVE SUITE 200, FALLS CHURCH, VA, 22042, US

(21) Appl. No.: 12/492,603

(22) Filed: Jun. 26, 2009

Related U.S. Application Data
(60) Provisional application No. 61/075,861, filed on Jun. 26, 2008.
(60) Provisional application No. 61/169,239, filed on Apr. 14, 2009.
(60) Provisional application No. 61/169,639, filed on Apr. 15, 2009.
(60) Provisional application No. 61/169,635, filed on Apr. 15, 2009.

(51) Int. Cl. A61K 31/5377; A61K 31/454; A61K 31/416; A61K 31/496; A61K 31/4725; A61P 27/02; A61P 27/14

(52) U.S. Cl. 514/236.5; 514/322; 514/406; 514/253.09; 514/310

(57) ABSTRACT

This invention is directed to methods of preventing or treating ocular diseases with inflammation, excessive cell proliferation, remodeling, neurite retraction, corneal neurodegeneration, excessive vaso-permeability and edema. Particularly, this invention relates to methods treating ocular diseases such as allergic conjunctivitis, corneal hyposensitivity, neurotrophic keratopathy, dry eye disease, proliferative vitreal retinopathy, macular edema, macular degeneration, and blepharitis, using novel Rho kinase inhibitor compounds. The method comprises identifying a subject in need of the treatment, and administering to the subject an effective amount of a novel Rho kinase inhibitor compound to treat the disease.

This application claims the benefit of U.S. Provisional Application Nos. 61/075,861, filed Jun. 26, 2008; 61/169,239, filed Apr. 14, 2009; 61/169,639, filed Apr. 15, 2009; and 61/169,635, filed Apr. 15, 2009; which are incorporated herein by reference in their entirety.

### TECHNICAL FIELD

This invention relates to methods of preventing or treating diseases or conditions associated with inflammation, excessive cell proliferation, remodeling, neurite retraction, corneal neurodegeneration, excessive vaso-permeability and edema. Particularly, this invention relates to methods treating ophthalmic diseases such as allergic conjunctivitis, corneal hyposensitivity, neurotrophic keratopathy, dry eye disease, proliferative vitreal retinopathy, macular edema, macular degeneration, and blepharitis, using novel Rho kinase inhibitor compounds.

### BACKGROUND OF THE INVENTION

### Rho Kinase as a Target

The Rho family of small GTP binding proteins can be activated by several extracellular stimuli such as growth factors, hormones and mechanic stress and function as a molecular signaling switch by cycling between an inactive GDP-bound form and an active GTP-bound form to elicit cellular responses. Rho kinase (ROCK) functions as a key downstream mediator of Rho and exists as two isoforms (ROCK1 and ROCK2) that are ubiquitously expressed. ROCKs are serine/threonine kinases that regulate the function of a number of substrates including cytoskeletal proteins such as adducin, moesin, Na⁺—H⁺ exchanger 1 (NHE1), LIM-kinase and vimentin, contractile proteins such as the myosin light chain phosphatase binding subunit (MYPT-1), CPI-17, myosin light chain and calponin, microtubule associated proteins such as Tau and MAP-2, neuronal growth cone associate proteins such as CRMP-2, signaling proteins such as PTEN and transcription factors such as serum response factor (Loirand et al, Circ Res 98:322-334 (2006)). ROCK is also required for cellular transformation induced by RhoA. As a key intermediary of multiple signaling pathways, ROCK regulates a diverse array of cellular phenomena including cytoskeletal rearrangement, actin stress fiber formation, proliferation, chemotaxis, cytokinesis, cytokine and chemokine secretion, endothelial or epithelial cell junction integrity, apoptosis, transcriptional activation and smooth muscle contraction. In neurons ROCK plays a critical role in the inhibition of axonal growth by myelin-associated inhibitory factors such as myelin-associated glycoprotein (MAG). ROCK activity also mediates the collapse of growth cones in developing neurons. Both processes are thought to be mediated by ROCK-induced phosphorylation of substrates such as LIM kinase and myosin light chain phosphatase, resulting in increased contractility of the neuronal actin-myosin system. Mature neurons preferentially express one of the ROCK isoforms (ROCK2), which phosphorylates collapsin response mediator protein 2 (CRMP2) that disintegrate growth cones involved in axon branching and elongation in response to stimuli. Inhibiting ROCK2 prevents expression of CRMP2 that allows growth-cone collapse (Dergham P et al. The Journal of Neuroscience, 22(15):6570-6577, 2002). By not expressing CRMP2, ROCK2 inhibitors promote neurite expansion, axon elongation, axonal rewiring across lesions within the CNS, and neural regeneration. As a result of these cellular actions, ROCK regulates physiologic processes such as vasoconstriction, tissue remodeling, inflammation, edema, proliferative disorders, neurite extension/retraction, and neurodegeneration.

### Allergic Conjunctivitis

Allergic eye disease primarily affects the conjunctiva. The signs and symptoms include itching, tearing, conjunctival edema, hyperemia, watery discharge, burning, and photophobia. Symptoms are usually bilateral; however, one eye can be affected more than the other. The most common allergic eye disease, allergic conjunctivitis (AC) can be subdivided into acute, seasonal and perennial. All three types result from classic Type I IgE-mediated hypersensitivity (Abelson, M B., et. al. Surv Opthalmol; 38(S):115, 1993).

Two phases of the ocular allergic response have been identified. The immediate response to allergens is mediated predominantly by mast cells, which are present in high concentrations in the normal conjunctiva, and increase further in patients with AC (Tsubota, K, et al., Cornea, 10:525, 1991). Mast cells become activated when allergen-IgE cross linking occurs, and chemical mediators are released by exocytosis. Histamine, the main mediator of the early response, causes vasodilatation, vasopermeability, and itching. Mast cells also release a variety of cytokines and chemokines, resulting in the influx of other inflammatory cells and continued inflammation, representing the late phase of the allergic reaction. Eosinophils, basophils, and neutrophils appear 6 to 10 hours after allergen challenge, followed by lymphocytes and monocytes.

Allergic conjunctivitis is a relatively benign ocular disease of young adults (average age of onset of 20 years of age) that causes significant suffering and use of healthcare resources, although it does not threaten vision. Ocular allergy is estimated to affect 20 percent of the population on an annual basis, and the incidence is increasing (Abelson, M B et. al., Surv Opthalmol., 38(S): 115, 1993). AC impacts productivity and while there are a variety of agents available for the treatment of AC, numerous patients still lack good control of symptoms and some are tolerating undesired side effects. Surveys have shown 20% of patients with AC are not fully satisfied with their AC medications and almost 50% feel they receive insufficient attention from their physicians (Mahr, et al., Allergy Asthma Proc, 28(4):404-9, 2007).

### Corneal Hyposensitivity and Neurodegeneration

An undesirable effect following laser photorefractive keratectomy (PRK), laser-assisted-in-situ keratomileusis (LASIK), and keratoplasty, is a functional reduction of corneal sensitivity, which occurs from approximately 3 weeks to one year and is due to severing of the corneal nerves during surgery. For example, it has been reported that the corneal nerve is apparently severed after LASIK (Tuuli U, et al., Experimental Eye Research 66: 755-763, 1998), and the corneal sensitivity decreases in a corneal region where, after LASIK, neurogram is not observed or the nerve bundle is too short to create connection (Tuuli U, et al., Investigative Opthalmology & Visual Sciences, 41: 393-397, 2000). It has been demonstrated that the corneal hyposensitivity after PRK and LASIK causes lower lacrimal gland response and decreased lacrimal fluid (Ang R T, et al., Current Opinion in Opthalmology 12: 318-322, 2001). As a result of the hypofunction of corneal sensitivity, patients after a corneal surgery blink less number of times, problematically showing the symptoms of dry eye. Additionally, in the patients with dry eye, lacrimal hypofunction gives rise to corneal hyposensitivity, which, upon combination with further lacrimal hypofunction, problematically aggravates the sensory component of the corneal surface. At present, recovery of corneal hyposensitivity following corneal surgery is left to spontaneous recovery, and in the treatment of dry eye, no active treatment is provided to recover corneal sensitivity. Moreover, while corneal hyposensitivity is caused by the diseases accompanying corneal neurodegeneration, such as neuroparalytic keratopathy, corneal ulcer, diabetic keratopathy and the like, no appropriate treatment is available at present.

Corneal hyposensitivity is caused by the diseases accompanying corneal neurodegeneration, such as neuroparalytic keratopathy, corneal ulcer, diabetic keratopathy and the like. Rho is a low molecular weight G protein included in the Rho family (containing Rho, Rac, Cdc42, etc.), and is known to be involved in actin cytoskeleton organization and neurite retraction reaction. C3 enzyme, a Rho protein inhibitor, is known to extend cell protrusion of 3T3 fibroblast (Hirose, M. et al., The Journal of Cell Biology, 141: 1625-1636, 1998), and a method of promoting the growth of central nerve axon by the administration of an effective amount of Rho protein inhibitor to patients is disclosed (JP-T-2001-515018 and EP-1,011,330-A). In addition, a Rho kinase inhibitor, which is among the effector molecules of Rho protein, is known to have an axon extension action of retinal ganglion cells, and exhibit a regeneration promoting action on the optic nerve cell (WO 02/83175 and EP-1,142,585-A). WO 03/020281 teaches that a compound capable of promoting nerve regeneration or neurite extension can be used for the treatment of a disease state caused by a corneal nerve disorder after surgery such as LASIK and the like. As to the trigeminal nerve, it has been reported that, in a rat trigeminal nerve tissue culture (trigeminal tract in whole mount cultures) system, extension of neurotrophin-induced nerve axon of nerve growth factor (NGF) and the like is inhibited by a Rho activator (lysophosphatidic acid), and facilitated by introduction of dominant negative Rho into a cell (Ozdinler, P. Hande et al., The Journal of Comparative Neurology, 438:377-387, 2001).

### Dry Eye

There are many ocular conditions where it is therapeutically desirable to correct improper tear fluid production. Dry eye is the general term for disease abnormalities that impact the pre-corneal tear film leading to a loss of mucous-containing goblet cells of the conjunctiva and eventually desquamation of the corneal epithelium that leads to destabilization of the cornea-tear interface (Gilbard J et al. CLAO Journal 22(2), 141-45 (1996)). There are several main structures responsible for maintaining the properties of the tear film such as the glands and ducts surrounding the eye and the ocular surface. These structures maintain the tear film via regulation of water and electrolyte transport and via mucin release by goblet cells. Among the ocular conditions where disruption of one of these structures can cause or lead to “dry eye disease” are: keratoconjunctivitis sicca (KCS), age-related dry eye, Stevens-Johnson syndrome, Sjogren's syndrome, ocular cicatrical pemphigoid, blepharitis, corneal injury, infection, Riley-Day syndrome, congenital alacrima, nutritional disorders or deficiencies, pharmacologic side effects, eye stress and glandular and tissue destruction, environmental exposure to smog, smoke, excessively dry air, airborne particulates, autoimmune and other immunodeficient disorders, and comatose patients rendered unable to blink. This is not to be considered an exhaustive list but is used to describe some of the diseases that can lead to dry eye disease.

Treatment for dry eye disease is effective regulation of the tear film. This can be accomplished by enhancing natural production or improving flow from the glands surrounding the eye or applying artificial tears to the ocular surface. The glands can be blocked due to inflammation of the surrounding tissue or the duct and gland itself. Blockage due to inflammation can be seen by increases in pro-inflammatory cytokines, redness and puffiness on and surrounding the ocular surface. Reduction of this inflammation can help return tear production to normal function and improve corneal health. (Wilson S et al. American Academy of Opthalmology 114(1), 76-79 (2007)).

Currently, the pharmaceutical treatment of dry eye disease is mostly limited to administration of artificial tears (saline solution) to temporarily rehydrate the eyes and to reduction of inflammation ((Riento K et al. Nat Rev Mol Cell Biol, 4:446-456, 2003)). However, artificial tears, the most widely used group of products, often have contraindications and incompatibility with soft contact lenses (Lemp M et al. Cornea 9(1), S48-550 (1990)).

Rho kinase signaling pathways have been implicated in the down regulation of pro-inflammatory pathways (Riento K et al. Nat Rev Mol Cell Biol, 4:446-456 (2003)). Rho kinase inhibition by Y-27632 and fasudil in a murine model of airway hyper-reactivity has been shown to reduce the mediators of inflammation (Taki F et al. Clinical and Experimental Allergy, 37:599-607, (2007)).

### Macular Edema and Macular Degeneration

Macular edema is a condition that occurs when damaged (or newly formed) blood vessels leak fluid onto the macula, a critical part of the retina for visual acuity, causing it to swell and blur vision. Macular edema is a common problem in diabetic retinopathy, where retinal vessel injury causes edema. Edema also occurs in the proliferative phase of diabetic retinopathy, when newly formed vessels leak fluid into either, or both, the macula and/or vitreous. Macular edema is commonly problematic in age-related macular degeneration (wet form) as well, where newly formed capillaries (angiogenesis) leak fluid into the macula.

Age related macular degeneration (AMD) is a progressive eye condition affecting as many as 10 million Americans. AMD is the number one cause of vision loss and legal blindness in adults over 60 in the U.S. As the population ages, and the “baby boomers” advance into their 60's and 70's, a virtual epidemic of AMD will be prevalent. The disease affects the macula of the eye, where the sharpest central vision occurs. Although it rarely results in complete blindness, it robs the individual of all but the outermost, peripheral vision, leaving only dim images or black holes at the center of vision.

Macular degeneration is categorized as either dry (atrophic) or wet (neovascular). The dry form is more common than the wet, with about 90% of AMD patients diagnosed with dry AMD. The wet form of the disease usually leads to more serious vision loss. In the dry form, there is a breakdown or thinning of the retinal pigment epithelial cells (RPE) in the macula, hence the term “atrophy”. These RPE cells are important to the function of the retina, as they metabolically support the overlying photoreceptors.

The clinical hallmark of atrophic AMD is accumulation of macular drusen, yellowish deposits just deep to the retinal pigment epithelium (“RPE”). Histopathologic examination of eyes with atrophic AMD reveals deposition of lipid and proteinaceous material deep to the RPE in Bruch's membrane. Drusen formation occurs naturally with age, with ocular exposure to visible light and UV light, metabolic changes of ocular cells related to age, and the formation of lipofuscin. Genetic predisposition can also factor into drusen formation. The formation of drusen can result in local inflammation as extracellular debris forms around the RPE, photoreceptors, and other ocular structures. The immune response which results brings about a number of components, one of which is membrane attack complex. The membrane attack complex can cause the death of host cells, which would include the RPE and photoreceptors. As a consequence, more cellular debris and drusen form as a result of the local inflammatory response, perpetuating the cycle (Nowak J Z Pharmacol Rep. 58(3): 353-363, 2006).

In aged eyes with AMD, Bruch's membrane is often about 3 times thicker than normal. This thickening is thought to be comprised of lipid as well as modified and cross-linked protein, which impedes transport of nutrients across Bruch's membrane from the choriocapillaries to the outer retina. This thickened barrier comprised of lipid and cross-linked protein impedes transport of nutrients across Bruch's membrane from the choriocapillaries to the outer retina. At present, there is no proven effective treatment for dry AMD other than the use of multivitamins and micronutrients.

Wet AMD occurs when new vessels form and grow through Bruch's membrane into the sub-RPE and subretinal space. This neovascular tissue is very fragile and hyperpermeable. Frequently, it bleeds causing damage to the overlying retina. As the blood organizes, functional macular tissue is replaced by scar tissue. To prevent visual loss, it would be desirable to intervene therapeutically prior to the development of neovascularization.

AMD is a challenging disease for both patient and doctor, because there are very few treatment options and, with the exception of anti-oxidants, no proven preventative therapy. While some individuals experience only minor inconvenience from macular degeneration, many others with more severe forms of macular degeneration are incapacitated. Patients may experience a loss of central vision accompanied by metamorphopsia, central scotomas, increased glare sensitivity, decreased contrast sensitivity, and decreased color vision (Rosenburg et al. American Family Physician, 77(10): 1431-1436, 2008). Current therapies, including laser photocoagulation, photodynamic therapy, and anti-angiogenic therapeutics have had mixed results, and, in certain instances, have caused deleterious side effects. A need exists for additional treatments that reduce the effects of macular degeneration and edema.

### Proliferative Vitreal Retinopathy

One of the most common causes of retinal detachment is proliferative vitreoretinopathy (PVR), an intraocular, non-malignant cellular proliferation. This process results ultimately in a separation of the retina from the retinal pigment epithelium, or RPE, because of tractional forces applied directly to the inner and outer retinal surfaces. This is the major cause for failure of retinal re-attachment surgery. (Ryan et al. Am J Opthalmol, 100:188-193, 1985). PVR is characterized by the formation of contractile cellular epiretinal membranes (ERMs) on both sides of the retina. (Clarkson, et al. Am. J. Opthalmol., 84:1-17, 1977). While the pathobiology of PVR is not clear, it appears that RPE cells are key to the development of these ERM. (Laqua, et al. Am. J. Opthalmol., 80:602-618, 1975). A large body of evidence supports the concept that previously quiescent RPE cells, when displaced into the vitreous cavity and exposed to the appropriate combination of cytokines, will divide and differentiate. This differentiation results in cells having myofibroblastic characteristics including adhesiveness and contractility. As these membranes form tight adhesions with the retinal surfaces, tractional forces are generated and detachment ensues. (Hiscott, et al. Br. J. Opthalmol., 68:708-715, 1984). Most evidence indicates retinal tears as the pathway through which RPE cells move in order to enter the vitreous cavity (Hiscott, et al. Br. J. Opthalmol., 68:708-715, 1984), and there is a clear association between the size of a retinal tear and the incidence of PVR. (Ryan et al. Am. J. Opthalmol., 100:188-193, 1985). Viable retinal pigment epithelial cells, displaced into the vitreous cavity, are exposed to a wide variety of proteins, cytokines, and chemoattractants. Extracellular matrix proteins have profound effects on cell morphology and behavior (Glaser, et al. Opthalmology, 100:466-470, 1993). RPE cells, when exposed in vitro to the extracellular matrix proteins and collagens found in the vitreous, change from their typical epithelial cell morphology to a mesenchymal or fibroblast-like morphology (Hay, et al. Cell Biology of Extracellular Matrix, New York, Plenum Press, 1982). The pathobiology of PVR, while not understood completely, involves the exposure of previously quiescent cells to factors which promote abnormal differentiation and cell division. This differentiation results in adhesive cells which contract in an unregulated, disorganized fashion and produce the tractional forces which detach the retina. (Mandelcorn, et al. Am J Opthalmol, 80:227-237, 1975).

The small GTPase, Rho, regulates the organization of the actin cytoskeleton by promoting the assembly of focal adhesions and actin stress fibers. A family of Rho-associated serine/threonine kinase isozymes named p160ROCK and ROK_{α}/Rho-kinase/ROCK 2 has been identified as a new class of Rho effectors that can induce focal adhesions and stress fibers in cultured fibroblasts and epithelial cells in vitro. (Amano M, Chihara K, Kimura K, et al. Science, 275:1308-1311, 1997). In patients with PVR, ERMs are characterized by the diffuse presence of α-smooth muscle actin (α-SMA)-positive myofibroblasts, which is presumed to be dedifferentiated RPE cells. (Casaroli-Marano R P et al. Invest Opthalmol Vis Sci, 40:2062-2072, 1999). Dense bundles of α-SMA microfilaments forming stress fibers within the myofibroblast were observed by electron microscopy in the ERM of patients with PVR, which strongly suggests that α-SMA substantially contributes to PVR development. (Casaroli-Marano R P, et al. Invest Opthalmol V is Sci., 40:2062-2072, 1999). A previous study has shown that the Rho kinase inhibitor Y-27632 suppresses type I collagen gel contraction in RPE cells, probably by suppressing expression of α-SMA, which led to attenuation of PVR in an animal model. (Zheng Y. et al. Invest Opthalmol Vis Sci., 45(2):668-74, 2004).

The current treatment for PVR is vitreoretinal surgery. Although such treatment often is successful, recurrent vitreoretinal traction may result in redetachment. The resulting retinal detachment sometimes causes permanent impairment of visual function. Pharmacologic and other forms of therapy to inhibit recurrent membrane formation are needed.

### Blepharitis

Blepharitis, also known as Lid Margin Disease (LMD), is a non-contagious inflammation of the eyelids that manifests itself through scaling and flaking around the eyelashes, excess sebum production and oily scaly discharge, mucopurulent discharge, and matted, hard crusts around the lashes. Accumulation of crust, discharge or debris on the eyelashes and lid margins creates an ideal environment for overgrowth of the staphylococcal bacteria naturally found on the skin of the eyelids and increases the chance of infection, allergic reaction and tear break down. Blepharitis disturbs the production of the critical, outer lipid layer of the tear film which causes the entire tear to evaporate, resulting in dry eye. A reduced tear quantity doesn't properly dilute bacteria and irritants, nor wash inflammatory products away from the lashes and lid margin, so they accumulate and lead to further inflammation worsening the cycle of disease, with blepharitis, meibomian gland dysfunction and dry eye perpetuating each other.

Routine examination of the eyelids of blepharitis patients shows redness caused by capillary congestion (erythema) as well as crusting of the lashes and lid margins. More detailed inspection using a high magnification slit lamp microscope reveals additional features, including loss of lashes (madarosis), whitening of the lashes (poliosis), scarring and misdirection of lashes (trichiasis), crusting of the lashes and meibomian orifices, eyelid margin ulcers, plugging of the meibomian orifices, and lid irregularity (tylosis).

Blepharitis is a common eye disorder throughout the Unites States and the world. There is an apparently high incidence in the general population based on the frequency of diagnoses in ophthalmologists' offices. It affects people of all ages; however blepharitis caused by seborrhea is seen more often in older patients around the age of fifty. Chronic blepharitis has been associated with occupations in which the hands are dirty for much of the day, since poor hygiene is a risk factor. Acute blepharitis results most commonly from an allergic reaction to a drug or chemical substance. Likewise, exposure to irritants such as chemical fumes, smoke, and environmental pollutants can exacerbate the condition of chronic blepharitis. The use of certain drugs can also cause blepharitis. It has been documented that some patients on cancer chemotherapeutic agents such as 5-fluorouracil develop ocular surface and lacrimal complications, including blepharitis, conjunctivitis, keratitis, and eyelid dermatitis (Eiseman A S et al. Ophthal Plast Reconstr Surg, 19:3:216-224, 2003).

Designing an effective treatment plan for blepharitis can be challenging. Treatment includes good hygiene and relies heavily on the patient as a partner in achieving disease management. Since lid scrubs and hot compresses are required multiple times daily, long-term compliance to produce positive results can be an issue. If left untreated, blepharitis can lead to a more serious condition called ulcerative blepharitis accompanied by eyelid scarring, scarring of the cornea, and eventually loss of visual function.

It is well known that during acute and chronic inflammation various putative mediators of inflammation are released by the inflamed tissues and by leukocytes. The concentrations of these mediators and leukocytes are indicative of the level or degree of inflammation. Likewise, a reduction in concentration of these mediators and leukocytes is an indication of the effectiveness of a drug in treating inflammation. Anti-inflammatory steroidal preparations (e.g., corticosteroids) are currently the drug of choice in the treatment of ocular inflammatory conditions. The use of a topical ophthalmic steroid can be helpful in reducing acute inflammation, however extended use is complicated by severe and numerous side effects. It would be highly desirable to develop new nonsteroidal drugs which have a high therapeutic effectiveness but which do not exhibit steroid-like side effects.

Rho kinase signaling pathways have been implicated in the down regulation of pro-inflammatory pathways (Riento K et al. Nat Rev Mol Cell Biol, 4:446-456, 2003). For example, Rho kinase inhibition by Y-27632 and fasudil in a murine model of airway hyper-reactivity has been shown to reduce the mediators of inflammation (Taki F et al. Clinical and Experimental Allergy, 37:599-607, 2007).

There is a need for an effective or improved method for treating ophthalmic disease such as allergic conjunctivitis, corneal hyposensitivity and kerotopathy, dry eye disease, proliferative vitreal retinopathy, macular edema and degeneration, and blepharitis.

### SUMMARY OF THE INVENTION

The present invention is directed to methods of preventing or treating ocular diseases associated with excessive inflammation, proliferation, remodeling, neurite retraction, corneal neurodegeneration, vaso-permeability and edema. Particularly, this invention relates to methods treating ocular diseases such as allergic conjunctivitis, corneal hyposensitivity, neurotrophic keratopathy, dry eye disease, proliferative vitreal retinopathy, macular edema, macular degeneration, and blepharitis, using novel Rho kinase inhibitor compounds. The method comprises identifying a subject in need of the treatment, and administering to the subject an effective amount of a novel Rho kinase inhibitor compound of Formula I or II to treat the disease.

The active compound is delivered to a subject by systemic administration or local administration.

### BRIEF DESCRIPTION OF THE DRAWING

FIG. 1 shows the murine eosinophil chemotaxis. The data reported are mean number of migrated eosinophils per high power view field±SEM. Average of at least 2 view fields per well, each treatment ran in triplicate.

FIG. 2 shows the human eosinophil chemotaxis. The data reported are mean number of migrated eosinophils per high power view field±SEM. Average of at least 3 view fields per well, each treatment ran in duplicate.

FIG. 3 shows the anti-inflammatory dosing paradigm.

FIG. 4 shows the eosinophils per mL in ova-sensitized, ova-challenged, mice treated with Compound 2.038, mice treated with Compound 1.131 and normal mice.

FIG. 5 shows the dose response effect of Compound 1.091 on eosinophil influx when dosed to ova-sensitized, ova-challenged mice, *, p<0.05 when compared to ova-sensitized, ova-challenged mice using Student's t-test.

FIG. 6 shows the concentration of IL-5 (pg/mL) in BALF of (1) ova-sensitized, ova-challenged mice, (2) ova-sensitized, ova-challenged mice treated with Compound 2.038 (15 μmol/kg/oral), and (3) normal, saline-sensitized mice. Dashed line indicates the lower limit of detection for the cytokine of interest. Data represent mean±SEM, n=10 for ova-sensitized, ova-challenged mice, treated or untreated; n=5 for normal mice.

FIG. 7 shows the concentration of Eotaxin (pg/mL) in BALF of (1) ova-sensitized, ova-challenged, (2) ova-sensitized, ova-challenged mice treated with Compound 2.038 (15 μmol/kg/oral), and (3) normal, saline-sensitized mice. Dashed line indicates the lower limit of detection for the cytokine of interest. Data represent mean±SEM, n—10 for ova-sensitized, ova-challenged mice, treated or untreated; n=5 for normal mice.

FIG. 8 shows the concentration of IL-13 (pg/mL) in BALF of (1) ova-sensitized, ova-challenged, (2) ova-sensitized, ova-challenged mice treated with Compound 2.038 (15 μmol/kg/oral), and (3) normal, saline-sensitized mice. Dashed line indicates the lower limit of detection for the cytokine of interest, Data represent mean±SEM, n=10 for ova-sensitized, ova-challenged mice, treated or untreated; n=5 for normal mice.

FIG. 9 shows the dose response effect of Compound 1.091 on airway hyperreactivity when dosed using the anti-inflammatory dosing paradigm on Days 27 to 30. *, p<0.05 using statistical analysis described in Example 14.

FIG. 10 shows the % inhibition of ATP-stimulated IL-1β Secretion in Human Monocytes by Rho Kinase Inhibitors. Data represent the mean±SD of at least n=2 experiments.

FIG. 11A shows the dose-dependent inhibition of LPS-induced neutrophilia by Compound 1.091 when dosed intratracheally to mice. Data are reported as cells/ml and are mean±SEM. *, p<0.05 when compared to LPS-treated mice using Student's t-test.

FIG. 11B shows the reduction of IL-1β levels in BALF from LPS-challenged mice upon intratracheal administration of Compound 1.091 or Compound 2.059. Data are reported as pg/mL of IL-1β and are mean±SEM.

FIG. 12 shows percent of FBS induced proliferation. Each compound was tested at 30 μM and challenged with 10% FBS with an n—3. * indicates n=5.

FIGS. 13A and 13B show [³H]-thymidine incorporation in primary human LAM-derived cells. Cells were treated with vehicle alone (control) or with 10 μM of Compound 1.132, Compound 2.066 or Compound 1.161. Experiments were performed on two separate cell lines, LAM1 cells (FIG. 13A) and LAM2 cells (FIG. 13B). Data are reported as counts per minute (CPM) of incorporated [³H]-thymidine are mean±SEM.

### DETAILED DESCRIPTION OF THE INVENTION

### Definitions

When present, unless otherwise specified, the following terms are generally defined as, but are not limited to, the following:

Halo substituents are taken from fluorine, chlorine, bromine, and iodine.

“Alkyl” refers to groups of from 1 to 12 carbon atoms inclusively, either straight chained or branched, more preferably from 1 to 8 carbon atoms inclusively, and most preferably 1 to 6 carbon atoms inclusively.

“Alkenyl” refers to groups of from 2 to 12 carbon atoms inclusively, either straight or branched containing at least one double bond but optionally containing more than one double bond.

“Alkynyl” refers to groups of from 2 to 12 carbon atoms inclusively, either straight or branched containing at least one triple bond but optionally containing more than one triple bond, and additionally optionally containing one or more double bonded moieties.

“Alkoxy” refers to the group alkyl-O— wherein the alkyl group is as defined above including optionally substituted alkyl groups as also defined above.

“Alkenoxy” refers to the group alkenyl-O— wherein the alkenyl group is as defined above including optionally substituted alkenyl groups as also defined above.

“Alkynoxy” refers to the group alkynyl-O— wherein the alkynyl group is as defined above including optionally substituted alkynyl groups as also defined above.

“Aryl” refers to an unsaturated aromatic carbocyclic group of from 6 to 14 carbon atoms inclusively having a single ring (e.g., phenyl) or multiple condensed rings (e.g., naphthyl or anthryl). Preferred aryls include phenyl, naphthyl and the like.

“Arylalkyl” refers to aryl-alkyl-groups preferably having from 1 to 6 carbon atoms inclusively in the alkyl moiety and from 6 to 10 carbon atoms inclusively in the aryl moiety. Such arylalkyl groups are exemplified by benzyl, phenethyl and the like.

“Arylalkenyl” refers to aryl-alkenyl-groups preferably having from 2 to 6 carbon atoms in the alkenyl moiety and from 6 to 10 carbon atoms inclusively in the aryl moiety.

“Arylalkynyl” refers to aryl-alkynyl-groups preferably having from 2 to 6 carbon atoms inclusively in the alkynyl moiety and from 6 to 10 carbon atoms inclusively in the aryl moiety.

“Cycloalkyl” refers to cyclic alkyl groups of from 3 to 12 carbon atoms inclusively having a single cyclic ring or multiple condensed rings which can be optionally substituted with from 1 to 3 allyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple ring structures such as adamantyl, and the like.

“Cycloalkenyl” refers to cyclic alkenyl groups of from 4 to 12 carbon atoms inclusively having a single cyclic ring or multiple condensed rings and at least one point of internal unsaturation, which can be optionally substituted with from 1 to 3 alkyl groups. Examples of suitable cycloalkenyl groups include, for instance, cyclobut-2-enyl, cyclopent-3-enyl, cyclooct-3-enyl and the like.

“Cycloalkylalkyl” refers to cycloalkyl-alkyl-groups preferably having from 1 to 6 carbon atoms inclusively in the alkyl moiety and from 6 to 10 carbon atoms inclusively in the cycloalkyl moiety. Such cycloalkylalkyl groups are exemplified by cyclopropylmethyl, cyclohexylethyl and the like.

“Cycloalkylalkenyl” refers to cycloalkyl-alkenyl-groups preferably having from 2 to 6 carbon atoms inclusively in the alkenyl moiety and from 6 to 10 carbon atoms inclusively in the cycloalkyl moiety. Such cycloalkylalkenyl groups are exemplified by cyclohexylethenyl and the like.

“Cycloalkylalkynyl” refers to cycloalkyl-alkynyl-groups preferably having from 2 to 6 carbon atoms inclusively in the alkynyl moiety and from 6 to 10 carbon atoms inclusively in the cycloalkyl moiety. Such cycloalkylalkynyl groups are exemplified by cyclopropylethynyl and the like.

“Heteroaryl” refers to a monovalent aromatic heterocyclic group of from 1 to 10 carbon atoms inclusively and 1 to 4 heteroatoms inclusively selected from oxygen, nitrogen and sulfur within the ring. Such heteroaryl groups can have a single ring (e.g., pyridyl or furyl) or multiple condensed rings (e.g., indolizinyl or benzothienyl).

“Heteroarylalkyl” refers to heteroaryl-alkyl-groups preferably having from 1 to 6 carbon atoms inclusively in the alkyl moiety and from 6 to 10 atoms inclusively in the heteroaryl moiety. Such heteroarylalkyl groups are exemplified by pyridylmethyl and the like.

“Heteroarylalkenyl” refers to heteroaryl-alkenyl-groups preferably having from 2 to 6 carbon atoms inclusively in the alkenyl moiety and from 6 to 10 atoms inclusively in the heteroaryl moiety.

“Heteroarylalkynyl” refers to heteroaryl-alkynyl-groups preferably having from 2 to 6 carbon atoms inclusively in the alkynyl moiety and from 6 to 10 atoms inclusively in the heteroaryl moiety.

“Heterocycle” refers to a saturated or unsaturated group having a single ring or multiple condensed rings, from 1 to 8 carbon atoms inclusively and from 1 to 4 hetero atoms inclusively selected from nitrogen, sulfur or oxygen within the ring. Such heterocyclic groups can have a single ring (e.g., piperidinyl or tetrahydrofuryl) or multiple condensed rings (e.g., indolinyl, dihydrobenzofuran or quinuclidinyl). Preferred heterocycles include piperidinyl, pyrrolidinyl and tetrahydrofuryl.

“Heterocycle-alkyl” refers to heterocycle-alkyl-groups preferably having from 1 to 6 carbon atoms inclusively in the alkyl moiety and from 6 to 10 atoms inclusively in the heterocycle moiety. Such heterocycle-alkyl groups are exemplified by morpholino-ethyl, pyrrolidinylmethyl, and the like.

“Heterocycle-alkenyl” refers to heterocycle-alkenyl-groups preferably having from 2 to 6 carbon atoms inclusively in the alkenyl moiety and from 6 to 10 atoms inclusively in the heterocycle moiety.

“Heterocycle-alkynyl” refers to heterocycle-alkynyl-groups preferably having from 2 to 6 carbon atoms inclusively in the alkynyl moiety and from 6 to 10 atoms inclusively in the heterocycle moiety.

Examples of heterocycles and heteroaryls include, but are not limited to, furan, thiophene, thiazole, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, pyrrolidine, indoline and the like.

Unless otherwise specified, positions occupied by hydrogen in the foregoing groups can be further substituted with substituents exemplified by, but not limited to, hydroxy, oxo, nitro, methoxy, ethoxy, alkoxy, substituted alkoxy, trifluoromethoxy, haloalkoxy, fluoro, chloro, bromo, iodo, halo, methyl, ethyl, propyl, butyl, alkyl, alkenyl, alkynyl, substituted alkyl, trifluoromethyl, haloalkyl, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, phenyl, aryl, substituted aryl, aryloxy, arylalkyl, arylalkenyl, arylalkynyl, pyridyl, imidazolyl, heteroaryl, substituted heteroaryl, heteroaryloxy, heteroarylalkyl, heteroarylalkenyl, heteroarylalkynyl, cyclopropyl, cyclobutyl, cyclopentyl, cyclohexyl, cycloalkyl, cycloalkenyl, cycloalkylalkyl, substituted cycloalkyl, cycloalkyloxy, pyrrolidinyl, piperidinyl, morpholino, heterocycle, (heterocycle)oxy, and (heterocycle)alkyl; and preferred heteroatoms are oxygen, nitrogen, and sulfur. It is understood that where open valences exist on these substituents they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further understood that the above substitutions can be made provided that replacing the hydrogen with the substituent does not introduce unacceptable instability to the molecules of the present invention, and is otherwise chemically reasonable.

The term “heteroatom-containing substituent” refers to substituents containing at least one non-halogen heteroatom. Examples of such substituents include, but are not limited to, hydroxy, oxo, nitro, methoxy, ethoxy, alkoxy, substituted alkoxy, trifluoromethoxy, haloalkoxy, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, aryloxy, pyridyl, imidazolyl, heteroaryl, substituted heteroaryl, heteroaryloxy, heteroarylalkyl, heteroarylalkenyl, heteroarylalkynyl, cycloalkyloxy, pyrrolidinyl, piperidinyl, morpholino, heterocycle, (heterocycle)oxy, and (heterocycle)alkyl; and preferred heteroatoms are oxygen, nitrogen, and sulfur. It is understood that where open valences exist on these substituents they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further understood that the above substitutions can be made provided that replacing the hydrogen with the substituent does not introduce unacceptable instability to the molecules of the present invention, and is otherwise chemically reasonable.

“Pharmaceutically acceptable salts” are salts that retain the desired biological activity of the parent compound and do not impart undesired toxicological effects. Pharmaceutically acceptable salt forms include various polymorphs as well as the amorphous form of the different salts derived from acid or base additions. The acid addition salts can be formed with inorganic or organic acids. Illustrative but not restrictive examples of such acids include hydrochloric, hydrobromic, sulfuric, phosphoric, citric, acetic, propionic, benzoic, napthoic, oxalic, succinic, maleic, fumaric, malic, adipic, lactic, tartaric, salicylic, methanesulfonic, 2-hydroxyethanesulfonic, toluenesulfonic, benzenesulfonic, camphorsulfonic, and ethanesulfonic acids. The pharmaceutically acceptable base addition salts can be formed with metal or organic counterions and include, but are not limited to, alkali metal salts such as sodium or potassium; alkaline earth metal salts such as magnesium or calcium; and ammonium or tetraalkyl ammonium salts, i.e., NX₄⁺ (wherein X is C₁₋₄).

“Tautomers” are compounds that can exist in one or more forms, called tautomeric forms, which can interconvert by way of a migration of one or more hydrogen atoms in the compound accompanied by a rearrangement in the position of adjacent double bonds. These tautomeric forms are in equilibrium with each other, and the position of this equilibrium will depend on the exact nature of the physical state of the compound. It is understood that where tautomeric forms are possible, the current invention relates to all possible tautomeric forms.

“Solvates” are addition complexes in which a compound of Formula I or Formula II is combined with a pharmaceutically acceptable cosolvent in some fixed proportion. Cosolvents include, but are not limited to, water, methanol, ethanol, 1-propanol, isopropanol, 1-butanol, isobutanol, tert-butanol, acetone, methyl ethyl ketone, acetonitrile, ethyl acetate, benzene, toulene, xylene(s), ethylene glycol, dichloromethane, 1,2-dichloroethane, N-methylformamide, N,N-dimethylformamide, N-methylacetamide, pyridine, dioxane, and diethyl ether. Hydrates are solvates in which the cosolvent is water. It is to be understood that the definitions of compounds in Formula I and Formula II encompass all possible hydrates and solvates, in any proportion, which possess the stated activity.

“An effective amount” is the amount effective to treat a disease by ameliorating the pathological condition or reducing the symptoms of the disease. “An effective amount” is the amount effective to improve at least one of the parameters relevant to measurement of the disease.

The inventors of the present invention have discovered compounds of Formula I or II, which are Rho kinase inhibitors, are effective in reducing cell proliferation, decreasing remodeling that is defined by cell migration and/or proliferation, reducing inflammation via the inhibition of leukocytes chemotaxis and the inhibition of cytokine and chemokine secretion, lowering or preventing tissue or organ edema via the increase of endothelial cell junction integrity, and reducing neurite retraction and promoting neuro-regeneration via the disruption of acto-myosin-based cytoskeleton within sensory neurons. By having the above properties, compounds of Formula I or II are useful in a method of preventing or treating diseases or conditions associated with excessive cell proliferation, remodeling, inflammation, vasoconstriction, neural densitization/degeneration and vascular edema.

By resolving one or more of the above-described pathophysiologies, the present invention provides a method of treating ocular diseases, particularly allergic conjunctivitis, corneal neuritogenesis, dry eye, proliferative vitreal retinopathy, macular edema and degeneration, and blepharitis.

The present method comprises the steps of identifying a subject in need of treatment, and administering to the subject an effective amount of Rho kinase inhibitor compound of Formula I or II.

### Rho Kinase Inhibitor Compounds

The rho kinase inhibitor compounds useful for this invention include compounds of general Formula I and Formula II, and/or tautomers thereof, and/or pharmaceutically-acceptable salts, and/or solvates, and/or hydrates thereof. Compounds of general Formula I and Formula II can be prepared according to the methods disclosed in co-pending application US2008/0214614, which is incorporated herein by reference.

A compound according to Formula I or Formula II can exist in several diastereomeric forms. The general structures of Formula I and Formula II include all diastereomeric forms of such materials, when not specified otherwise. Formula I and Formula II also include mixtures of compounds of these Formulae, including mixtures of enantiomers, diastereomers and/or other isomers in any proportion.

### A. Formula I

Compounds of Formula I are as follows:

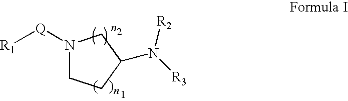

wherein: R₁ is aryl or heteroaryl, optionally substituted;
Q is C═O, SO₂, or (CR₄R₅)ₙ₃;
n₁ is 1, 2, or 3;
n₂ is 1 or 2;
n₃ is 0, 1, 2, or 3;
wherein the ring represented by

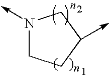

is optionally substituted by alkyl, halo, oxo, OR₆, NR₆R₇, or SR₆;
R₂ is selected from the following heteroaryl systems, optionally substituted:

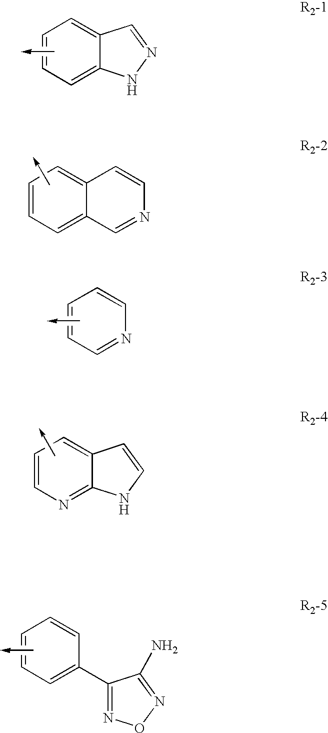

R₃-R₇ are independently H, alkyl, alkenyl, alkynyl, cycloalkyl, cycloalkenyl, cycloalkylalkyl, cycloalkylalkenyl, or cycloalkylalkynyl optionally substituted.

In Formula I, a preferred R₁ is substituted aryl, a more preferred R₁ is substituted phenyl, the preferred Q is (CR₄R₅)ₙ₃, the more preferred Q is CH₂, the preferred n₁ is 1 or 2, the preferred n₂ is 1, the preferred n₃ is 1 or 2, and the preferred R₃-R₇ are H.

In Formula I, a preferred R₂ substituent is halo, alkyl, cycloalkyl, hydroxyl, alkoxy, cycloalkyloxy, amino, alkylamino, or R₂ is unsubstituted. A more preferred R₂ substituent is halo, methyl, ethyl, isopropyl, cyclopropyl, hydroxyl, methoxy, ethoxy, amino, methylamino, dimethylamino, or R₂ is unsubstituted.

[1] One embodiment of the invention is represented by Formula I, in which R₂ is 5-indazolyl or 6-indazolyl (R₂-1), optionally substituted.

[1a] In embodiment 1, R₂-1 is substituted by one or more alkyl or halo substituents.

[1b] In embodiment 1, R₂-1 is substituted by one or more amino, alkylamino, hydroxyl, or alkoxy substituents.

[1c] In embodiment 1, R₂-1 is unsubstituted.

[2] In another embodiment, the invention is represented by Formula I in which R₂ is 5-isoquinolinyl or 6-isoquinolinyl (R₂-2), optionally substituted.

[2a] In embodiment 2, R₂-2 is substituted by one or more alkyl or halo substituents.

[2b] In embodiment 2, R₂-2 is substituted by one or more amino, alkylamino, hydroxyl, or alkoxy substituents.

[2c] In embodiment 2, R₂-2 is unsubstituted.

[3] In another embodiment, the invention is represented by Formula I in which R₂ is 4-pyridyl or 3-pyridyl (R₂-3), optionally substituted.

[3a] In embodiment 3, R₂-3 is substituted by one or more alkyl or halo substituents.

[3b] In embodiment 3, R₂-3 is substituted by one or more amino, alkylamino, hydroxyl, or alkoxy substituents.

[3c] In embodiment 3, R₂-3 is unsubstituted.

[4] In another embodiment, the invention is represented by Formula I in which R₂ is 7-azaindol-4-yl or 7-azaindol-5-yl (R₂-4), optionally substituted.

[4a] In embodiment 4, R₂-4 is substituted by one or more alkyl or halo substituents.

[4b] In embodiment 4, R₂-4 is substituted by one or more amino, alkylamino, hydroxyl, or alkoxy substituents.

[4c] In embodiment 4, R₂-4 is unsubstituted.

[5] In another embodiment, the invention is represented by Formula I in which R₂ is 4-(3-amino-1,2,5-oxadiazol-4-yl)phenyl or 3-(3-amino-1,2,5-oxadiazol-4-yl)phenyl (R₂-5), optionally substituted.

[5a] In embodiment 5, R₂-5 is unsubstituted.

[6] In another embodiment, the invention is represented by Formula I in which R₂ is one of the groups R₂-1-R₂-5, substituted by one or more alkyl, halo, amino, alkylamino, hydroxyl, or alkoxy substituents.

[6a] In embodiment 6, R₂ is substituted by one or more alkyl or halo substituents.

[6b] In embodiment 6, R₂ is substituted by one or more amino, alkylamino, hydroxyl, or alkoxy substituents.

[7] In another embodiment, the invention is represented by Formula I in which R₂ is one of the groups R₂-1-R₂-5, and is unsubstituted.

[8] In another embodiment, the invention is represented by Formula I in which R₃ is H.

[9] In another embodiment, the invention is represented by Formula I in which Q is (CR₄R₅)ₙ₃, and n₃ is 1 or 2.

[10] In another embodiment, the invention is represented by Formula I in which Q is (CH₂)ₙ₃, and n3 is 1.

[11] In another embodiment, the invention is represented by Formula I in which R₁ is aryl or heteroaryl substituted with one or more alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, optionally further substituted.

Compounds exemplifying embodiment 11 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table A.

[12] In another embodiment, the invention is represented by Formula I in which R₁ is aryl or heteroaryl substituted with one or more heteroatom-containing substituents, with the proviso that if the R₁ substituent is acyclic and is connected to R₁ by a carbon atom, then this substituent contains at least one nitrogen or sulfur atom, with the second proviso that if the substituent is acyclic and is connected to R₁ by an oxygen or nitrogen atom, then this substituent contains at least one additional oxygen, nitrogen or sulfur atom, and with the third proviso that if the substituent is connected to R₁ by a sulfone linkage “—SO₂—”, then R₂ is not nitrogen- or oxygen-substituted R₂-2.

[12a] In embodiment 12, the heteroatom-containing substituent is connected to R₁ by an oxygen or nitrogen atom.

[12b] In embodiment 12, the heteroatom-containing substituent is connected to R₁ by a sulfide linkage, “—S—”.

Compounds exemplifying embodiment 12 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table A.

[13] In another embodiment, the invention is represented by Formula I in which R₁ is aryl or heteroaryl substituted with one or more alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, which are further substituted with one or more heteroatom-containing substituents, with the proviso that if the R₁ substituent is acyclic and its heteroatom-containing substituent falls on the carbon by which it is attached to R₁, then the heteroatom-containing substituent contains at least one nitrogen or sulfur atom.

Compounds exemplifying embodiment 13 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, 1.122, and 1.123, shown below in Table A.

[14] In another embodiment, the invention is represented by Formula I in which R₁ is aryl or heteroaryl substituted with one or more alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, optionally further substituted, and R₂ is 5-indazolyl (R₂-1) or 5-isoquinolinyl (R₂-2), optionally substituted.

[14a] In embodiment 14, R₂ is 5-indazolyl (R₂-1), optionally substituted by one or more alkyl, halo, amino, alkylamino, hydroxyl, or alkoxy substituents.

[14b] In embodiment 14, R₂ is 5-isoquinolinyl (R₂-2), optionally substituted by one or more alkyl, halo, amino, alkylamino, hydroxyl, or alkoxy substituents.

[14c] In embodiment 14, R₂ is unsubstituted.

Compounds exemplifying embodiment 14 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table A.

[15] In another embodiment, the invention is represented by Formula I in which R₁ is aryl or heteroaryl substituted with one or more heteroatom-containing substituents, and R₂ is 5-indazolyl (R₂-1) or 5-isoquinolinyl (R₂-2), optionally substituted, with the proviso that if the R₁ substituent is acyclic and is connected to R₁ by a carbon atom, then this substituent contains at least one nitrogen or sulfur atom, with the second proviso that if the substituent is acyclic and is connected to R₁ by an oxygen or nitrogen atom, then this substituent contains at least one additional oxygen, nitrogen or sulfur atom, and with the third proviso that if the substituent is connected to R₁ by a sulfone linkage “—SO₂—”, then R₂ is not nitrogen- or oxygen-substituted R₂-2.

[15a] In embodiment 15, R₂ is 5-indazolyl (R₂-1), optionally substituted by one or more alkyl, halo, amino, alkylamino, hydroxyl, or alkoxy substituents.

[15b] In embodiment 15, R₂ is 5-isoquinolinyl (R₂-2), optionally substituted by one or more alkyl, halo, amino, alkylamino, hydroxyl, or alkoxy substituents.

[15c] In embodiment 15, R₂ is unsubstituted.

[15d] In embodiment 15, the heteroatom-containing substituent is connected to R₁ by an oxygen or nitrogen atom.

[15e] In embodiment 15, the heteroatom-containing substituent is connected to R₁ by a sulfide linkage, “—S—”.

Compounds exemplifying embodiment 15 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table A.

[16] In another embodiment, the invention is represented by Formula I in which R₁ is aryl or heteroaryl substituted with one or more alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, at least one of which is further substituted with one or more heteroatom-containing substituents, and R₂ is 5-indazolyl (R₂-1) or 5-isoquinolinyl (R₂-2), optionally substituted, with the proviso that if the R₁ substituent is acyclic and its heteroatom-containing substituent falls on the carbon by which it is attached to R₁, then the heteroatom-containing substituent contains at least one nitrogen or sulfur atom.

[16a] In embodiment 16, R₂ is 5-indazolyl (R₂-1), optionally substituted by one or more alkyl, halo, amino, alkylamino, hydroxyl, or alkoxy substituents.

[16b] In embodiment 16, R₂ is 5-isoquinolinyl (R₂-2), optionally substituted by one or more alkyl, halo, amino, alkylamino, hydroxyl, or alkoxy substituents.

[16c] In embodiment 16, R₂ is unsubstituted.

Compounds exemplifying embodiment 16 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, 1.122, and 1.123, shown below in Table A.

The inventors have discovered certain compounds of Formula I that have properties that render them particularly useful for treating the conditions addressed by the invention. In particular, these preferred compounds can be described as compounds of Formula I in which R₂, R₃, n₁, and n₂ are limited to the combinations shown in Formulae Ia, Ib, and Ic:

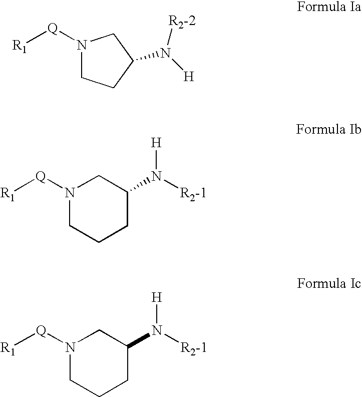

In Formulae Ia, Ib, and Ic, the stereochemistry of the central pyrrolidine or piperidine ring is limited to the R, R, and S configurations respectively, as drawn. Further, the group R₁ in these Formulae is limited to phenyl, thiophene, and 6,5- or 6,6-fused bicyclic heteroaryl rings. The group R₁ is either unsubstituted or is optionally substituted with 1, 2 or 3 substituents independently selected from halogen, methyl, ethyl, hydroxyl, methoxy, or ethoxy.

In Formula Ia, Ib, and Ic, Q is C═O, SO₂, or (CR₄R₅)ₙ₃; where R₄ and R₅ are independently H, alkyl, cycloalkyl, optionally substituted. The preferred R₄ and R₅ are H or unsubstituted alkyl. The preferred Q is CH₂.

In Formula Ia, Ib, and Ic, a preferred R₂ substituent is halo, alkyl, cycloalkyl, hydroxyl, alkoxy, cycloalkyloxy, amino, alkylamino, or R₂ is unsubstituted. A more preferred R₂ substituent is halo, methyl, ethyl, isopropyl, cyclopropyl, hydroxyl, methoxy, ethoxy, amino, methylamino, dimethylamino, or R₂ is unsubstituted.

In a more preferred form of Formulae Ia, Ib, and Ic, R₁ is phenyl or a 6,5-fused bicyclic heteroaryl ring, optionally substituted by 1 or 2 substituents, Q is CH₂, and the group R₂ is unsubstituted. The most preferred 6,5-fused bicyclic heteroaryl rings are benzofuran, benzothiophene, indole, and benzimidazole.

In another more preferred form, R₁ of Formulae Ia, Ib, and Ic is mono- or disubstituted when R₁ is phenyl, with 3-substituted, 4-substituted, 2,3-disubstituted, and 3,4-disubstituted being most preferred. When R₁ is bicyclic heteroaryl, an unsubstituted or monosubstituted R₁ is most preferred.

The inventors have found that certain members of Formulae Ia, Ib, and Ic, as defined above, are particularly useful in treating the conditions addressed in this invention. The compounds of the invention are multikinase inhibitors, with inhibitory activity against ROCK1 and ROCK2, in addition to several other kinases in individual compound cases. These kinase inhibitory properties endow the compounds of the invention not only with smooth muscle relaxant properties, but additionally with antiproliferative, antichemotactic, and cytokine secretion inhibitory properties that render them particularly useful in treating conditions with proliferative or inflammatory components as described in the invention.

[17] In particular, we have found that compounds in which R₂ is R₂-2 are particularly potent inhibitors of both ROCK1 and ROCK2, and that these agents inhibit the migration of neutrophils toward multiple chemotactic stimuli and inhibit the secretion of the cytokines IL-1β, TNF-α and IL-9 from LPS-stimulated human monocytes. Compounds in which R₁ is heteroaryl, particularly 6,5-fused bicyclic heteroaryl, are especially preferred. These compounds are of particular value in addressing conditions with an inflammatory component.

Compounds exemplifying embodiment 17 include compounds 2.025, 2.027, 2.046, 2.047, 2.048, 2.055, 2.056, 2.057, 2.061, 2.062, 2.065, 2.074, 2.075, 2.088, and 2.090.

[18] In another embodiment, we have found that compounds of Formula Ic are potent and selective inhibitors of ROCK2, with comparatively lower inhibitory potency against ROCK1. We have demonstrated that compounds of this class typically show good smooth muscle relaxation properties and that smooth muscle relaxation effects in this class are generally correlated with ROCK2 potency. Compounds in which R₁ is phenyl are particularly preferred. Compounds of this embodiment are of particular value in addressing conditions where relaxation of smooth muscle, in particular vascular and bronchial smooth muscle, is of highest importance.

Compounds exemplifying embodiment 18 include compounds 1.072, 1.078, 1.079, 1.080, 1.141, 1.142, 1.148, 1.149, 1.150, 1.151, 1.154, 1.155, 1.156, 1.163, 1.164, 1.166, 1.170, 1.171, 1.175, 1.179, 1.183, 1.227, 1.277, and 1.278.

[19] In another embodiment, the inventors have found that compounds of Formula Ib are potent mixed inhibitors of ROCK1 and ROCK2, display additional inhibitory activity against the kinases Akt3 and p70S6K, and that these compounds generally display potent antiproliferative activity in models of smooth muscle cell proliferation. Compounds of this class are of particular value in addressing conditions in which an antiproliferative component is desired in combination with a smooth muscle relaxing effect.

Compounds exemplifying embodiment 19 include compounds 1.073, 1.110, 1.131, 1.132, 1.133, 1.134, 1.135, 1.136, 1.137, 1.138, 1.143, 1.144, 1.145, 1.146, 1.172, 1.173, 1.177, 1.191, 1.192, 1.203, 1.210, 1.226, 1.241, 1.242, 1.245, 1.246, 1.252, and 1.254.

[20] In another embodiment, the inventors have found that certain compounds of Formulae Ia, Ib, and Ic distribute preferentially to the lung on oral dosing. In particular, compounds in which R₁ is a lipophilic bicyclic heteroaryl group are preferred for this dosing behavior. Compounds of this type are especially useful for treating diseases of the lung by oral dosing while minimizing impact on other tissues.

Compounds exemplifying embodiment 20 include compounds 1.131, 1.137, 1.138, 1.143, 1.148, 1.149, 1.150, 1.166, 1.175, 1.177, 1.246, 1.252, 2.055, 2.056, 2.057, 2.065, 2.074, and 2.075.

[21] In another embodiment, the inventors have found that certain compounds of Formulae Ia, Ib, and Ic produce low plasma concentrations of the compound when dosed by the oral route. Compounds in which one substituent on R₁ is selected from the group methyl, ethyl, or hydroxyl are preferred for typically exhibiting this pharmacokinetic behavior. Compounds displaying this property are particularly useful for inhalation dosing, since a large portion of the material dosed in this way is typically swallowed, and it is advantageous for this swallowed portion to remain unabsorbed or to be cleared rapidly so as to minimize the impact of the compound on other tissues.

Compounds exemplifying embodiment 21 include compounds 1.078, 1.133, 1.135, 1.136, 1.145, 1.151, 1.154, 1.155, 1.156, 1.163, 1.171, 1.172, 1.173, 1.192, 1.242, 2.025, and 2.061.

Preparation of compounds of Formulae Ia, Ib, and Ic can be problematic using methods commonly known in the art. In particular, syntheses of compounds of Formulae Ib and Ic using transition metal mediated coupling reactions to form the critical bond between R₂-1 and the nitrogen atom are hampered by low yields when the indazole ring is not protected properly to allow a successful reaction. Specifically, the methods disclosed in UA2006/0167043 fail to provide the desired amino indazole products when the indazole is unprotected or is protected with a standard acyl protecting group such as pivalate or alkoxycarbonyl protecting groups. The inventors prepare compounds of Formulae Ia, Ib, and Ic according to the methods disclosed in the co-pending application US2008/0214614, which allows the successful protection, coupling, and deprotection of the indazole ring, thereby allowing the successful preparation of the compounds of Formulae Ib and Ic and the demonstration of their useful biological properties.

### B. Formula II

A preferred compound of Formula I is where R₁=Ar—X, shown below as Formula II:

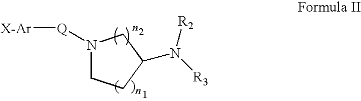

wherein:
Ar is a monocyclic or bicyclic aryl or heteroaryl ring, such as phenyl;
X is from 1 to 3 substituents on Ar, each independently in the form Y-Z, in which Z is attached to Ar;
Y is one or more substituents on Z, and each is chosen independently from H, halogen, or the heteroatom-containing substituents, including but not limited to OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀;
Each instance of Z is chosen independently from alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, heterocycle, (heterocycle)alkyl, (heterocycle)alkenyl, (heterocycle)alkynyl, or is absent;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents, including but not limited to OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, or NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents, including but not limited to OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅, NR₁₄C(═O)R₁₅, NR₁₄C(═O)OR₁₅, OC(═O)NR₁₄R₁₅, or NR₁₄C(═O)NR₁₅R₁₆;
any two of the groups R₈, R₉ and R₁₀ are optionally joined with a link selected from the group consisting of bond, —O—, —S—, —SO—, —SO₂—, and —NR₁₇— to form a ring;
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle.

In Formula II, the preferred Y is H, halogen, OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀, the more preferred Y is H, halogen, OR₈, SR₈, SOR₈, SO₂R₈, SO₂NR₈R₉, NR₈SO₂R₉, CONR₈R₉, or NR₈C(═O)NR₉R₁₀ the preferred Z is alkyl, alkenyl, alkynyl, cycloalkyl, cycloalkenyl, cycloalkylalkyl, or is absent; the more preferred Z is alkyl, alkenyl, alkynyl, cycloalkyl, or is absent, the preferred Q is (CR₄R₅)ₙ₃, the more preferred Q is CH₂, the preferred n1 is 1 or 2, the preferred n₂ is 1, the preferred n3 is 1 or 2, the preferred R₃-R₇ are H, the preferred R₈ is H, alkyl, arylalkyl, cycloalkyl, cycloalkylalkyl, or heterocycle, the preferred R₈ substituents are H, halogen, OR₁₁, NR₁₁R₁₂, SR₁₁, SOR₁₁, SO₂R₁₁SO₂NR₁₁R₁₂, NR₁₁SO₂R₁₂, CONR₁₁R₁₂, NR₁₁C(═O)R₁₂, and the preferred R₉-R₁₇ are H or alkyl.

In Formula II, a preferred R₂ substituent is halo, alkyl, cycloalkyl, hydroxyl, alkoxy, cycloalkyloxy, amino, alkylamino, or R₂ is unsubstituted. A more preferred R₂ substituent is halo, methyl, ethyl, isopropyl, cyclopropyl, hydroxyl, methoxy, ethoxy, amino, methylamino, dimethylamino, or R₂ is unsubstituted.

[1] One embodiment of the invention is represented by Formula II in which R₂ is 5-indazolyl or 6-indazolyl (R₂-1), optionally substituted.

[1a] In embodiment 1, R₂-1 is substituted by one or more alkyl or halo substituents.

[1b] In embodiment 1, R₂-1 is substituted by one or more amino, alkylamino, hydroxyl, or alkoxy substituents.

[1c] In embodiment 1, R₂-1 is unsubstituted.

[2] In another embodiment, the invention is represented by Formula II in which R₂ is 5-isoquinolinyl or 6-isoquinolinyl (R₂-2), optionally substituted.

[2a] In embodiment 2, R₂-2 is substituted by one or more alkyl or halo substituents.

[2b] In embodiment 2, R₂-2 is substituted by one or more amino, alkylamino, hydroxyl, or alkoxy substituents.

[2c] In embodiment 2, R₂-2 is unsubstituted.

[3] In another embodiment, the invention is represented by Formula II in which R₂ is 4-pyridyl or 3-pyridyl (R₂-3), optionally substituted.

[3a] In embodiment 3, R₂-3 is substituted by one or more alkyl or halo substituents.

[3b] In embodiment 3, R₂-3 is substituted by one or more amino, alkylamino, hydroxyl, or alkoxy substituents.

[3c] In embodiment 3, R₂-3 is unsubstituted.

[4] In another embodiment, the invention is represented by Formula II in which R₂ is 7-azaindol-4-yl or 7-azaindol-5-yl (R₂-4), optionally substituted.

[4a] In embodiment 4, R₂-4 is substituted by one or more alkyl or halo substituents.

[4b] In embodiment 4, R₂-4 is substituted by one or more amino, alkylamino, hydroxyl, or alkoxy substituents.

[4c] In embodiment 4, R₂-4 is unsubstituted.

[5] In another embodiment, the invention is represented by Formula II in which R₂ is 4-(3-amino-1,2,5-oxadiazol-4-yl)phenyl or 3-(3-amino-1,2,5-oxadiazol-4-yl)phenyl (R₂-5), optionally substituted.

[5a] In embodiment 5, R₂-5 is unsubstituted.

[6] In another embodiment, the invention is represented by Formula II in which R₂ is one of the groups R₂-1-R₂-5, substituted by one or more alkyl, halo, amino, alkylamino, hydroxyl, or alkoxy substituents.

[6a] In embodiment 6, R₂ is substituted by one or more alkyl or halo substituents.

[6b] In embodiment 6, R₂ is substituted by one or more amino, alkylamino, hydroxyl, or alkoxy substituents.

[7] In another embodiment, the invention is represented by Formula II in which R₂ is one of the groups R₂-1-R₂-5, and is unsubstituted.

[8] In another embodiment, the invention is represented by Formula II in which R₃ is H.

[9] In another embodiment, the invention is represented by Formula II in which Q is (CR₄R₅)ₙ₃, and n₃ is 1 or 2.

[10] In another embodiment, the invention is represented by Formula II in which Q is (CH₂)ₙ₃, and n₃ is 1.

[11] In another embodiment, the invention is represented by Formula II in which for at least one substituent X, Z is alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkylalkenyl, cycloalkylalkynyl, cycloalkenyl, cycloalkylalkyl, heterocycle, (heterocycle)alkyl, (heterocycle)alkenyl, or (heterocycle)alkynyl.

Compounds exemplifying embodiment 11 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table A.

[12] In another embodiment, the invention is represented by Formula II in which for at least one substituent X, Z is absent, and Y is a heteroatom-containing substituent, including but not limited to OR₈, NR₈R₉, SR₈, SOR₈, SO₂R₈, SO₂NR₈R₉, NR₈SO₂R₉, CONR₈R₉, NR₈C(═O)R₉, NR₈C(═O)OR₉, OC(═O)NR₈R₉, or NR₈C(═O)NR₉R₁₀, with the proviso that if the substituent Y is acyclic and is connected to Ar by a carbon atom, then this substituent contains at least one nitrogen or sulfur atom, with the second proviso that if the substituent Y is acyclic and is connected to Ar by an oxygen or nitrogen atom, then this substituent contains at least one additional oxygen, nitrogen or sulfur atom, and with the third proviso that if the substituent Y is connected to Ar by a sulfone linkage “—SO₂—”, then R₂ is not nitrogen- or oxygen-substituted R₂-2.

[12a] In embodiment 12, the heteroatom-containing substituent is connected to R₁ by an oxygen or nitrogen atom.

[12b] In embodiment 12, the heteroatom-containing substituent is connected to R₁ by a sulfide linkage, “—S—”.

Compounds exemplifying embodiment 12 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table A.

[13] In another embodiment, the invention is represented by Formula II in which for at least one substituent X, Z is alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl, and Y is a heteroatom-containing substituent, including but not limited to OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀, with the proviso that if Z is acyclic and Y falls on the carbon by which Z is attached to Ar, then Y contains at least one nitrogen or sulfur atom.

Compounds exemplifying embodiment 13 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, 1.122, and 1.123, shown below in Table A.

[14] In another embodiment, the invention is represented by Formula II in which for at least one substituent X, Z is alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl, and R₂ is 5-indazolyl (R₂-1) or 5-isoquinolinyl (R₂-2), optionally substituted.

[14a] In embodiment 14, R₂ is 5-indazolyl (R₂-1), optionally substituted by one or more alkyl, halo, amino, alkylamino, hydroxyl, or alkoxy substituents.

[14b] In embodiment 14, R₂ is 5-isoquinolinyl (R₂-2), optionally substituted by one or more alkyl, halo, amino, alkylamino, hydroxyl, or alkoxy substituents.

[14c] In embodiment 14, R₂ is unsubstituted.

Compounds exemplifying embodiment 14 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table A.

[15] In another embodiment, the invention is represented by Formula II in which for at least one substituent X, Z is absent, and Y is a heteroatom-containing substituent, including but not limited to OR₈, NR₈R₉, SR₈, SOR₈, SO₂R₈, SO₂NR₈R₉, NR₈SO₂R₉, CONR₈R₉, NR₈C(═O)R₉, NR₈C(═O)OR₉, OC(═O)NR₈R₉, or NR₈C(═O)NR₉R₁₀, and R₂ is 5-indazolyl (R₂-1) or 5-isoquinolinyl (R₂-2), optionally substituted, with the proviso that if the substituent Y is acyclic and is connected to Ar by a carbon atom, then this substituent contains at least one nitrogen or sulfur atom, with the second proviso that if the substituent Y is acyclic and is connected to Ar by an oxygen or nitrogen atom, then this substituent contains at least one additional oxygen, nitrogen or sulfur atom, and with the third proviso that if the substituent Y is connected to Ar by a sulfone linkage “—SO₂—”, then R₂ is not nitrogen- or oxygen-substituted R₂-2.

[15a] In embodiment 15, R₂ is 5-indazolyl (R₂-1), optionally substituted by one or more alkyl, halo, amino, alkylamino, hydroxyl, or alkoxy substituents.

[15b] In embodiment 15, R₂ is 5-isoquinolinyl (R₂-2), optionally substituted by one or more alkyl, halo, amino, alkylamino, hydroxyl, or alkoxy substituents.

[15c] In embodiment 15, R₂ is unsubstituted.

[15d] In embodiment 15, the heteroatom-containing substituent is connected to R₁ by an oxygen or nitrogen atom.

[15e] In embodiment 15, the heteroatom-containing substituent is connected to R₁ by a sulfide linkage, “—S—”.

Compounds exemplifying embodiment 15 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table A.

[16] In another embodiment, the invention is represented by Formula II in which for at least one substituent X, Z is alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl, and Y is a heteroatom-containing substituent, including but not limited to OR₈, NR₈R₉, NO₂, SR₈, SOR₈, SO₂R₈, SO₂NR₈R₉, NR₉SO₂R₉, OCF₃, CONR₈R₉, NR₈C(═O)R₉, NR₈C(═O)OR₉, OC(═O)NR₉R₉, or NR₈C(═O)NR₉R₁₀, and R₂ is 5-indazolyl (R₂-1) or 5-isoquinolinyl (R₂-2), optionally substituted, with the proviso that if Z is acyclic and Y falls on the carbon by which Z is attached to Ar, then Y contains at least one nitrogen or sulfur atom.

[16a] In embodiment 16, R₂ is 5-indazolyl (R₂-1), optionally substituted by one or more alkyl, halo, amino, alkylamino, hydroxyl, or alkoxy substituents.

[16b] In embodiment 16, R₂ is 5-isoquinolinyl (R₂-2), optionally substituted by one or more alkyl, halo, amino, alkylamino, hydroxyl, or alkoxy substituents.

[16c] In embodiment 16, R₂ is unsubstituted.

[16d] In embodiment 16, Ar is heteroaryl.

Compounds exemplifying embodiment 16 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, 1.122, and 1.123, shown below in Table A.

In Embodiments 11-16 of Formula II, the preferred Q is (CR₄R₅)ₙ₃, the more preferred Q is CH₂, the preferred n₁ is 1 or 2, the preferred n₂ is 1, the preferred n3 is 1 or 2, and the preferred R₃ is H.

The inventors have discovered certain compounds of Formula II that have properties that render them particularly useful for treating the conditions addressed by the invention. In particular, these preferred compounds of Embodiments 14, 15 and 16 can be described as compounds of Formula II in which R₂, R₃, n₁, and n₂ are limited to the combinations shown in Formulae Ia, IIb, and IIc:

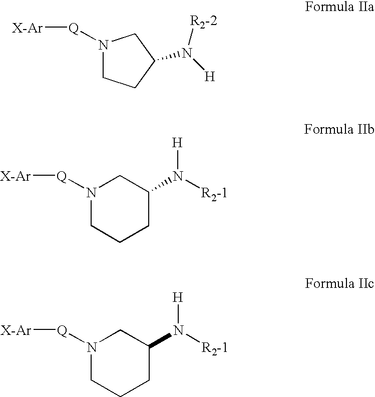

In Formulae Ia, IIb, and IIc, the stereochemistry of the central pyrrolidine or piperidine ring is limited to the R, R, and S configurations respectively, as drawn.

In Formula IIa, IIb, and IIc, Q is C═O, SO₂, or (CR₄R₅)ₙ₃; where R₄ and R₅ are independently H, alkyl, cycloalkyl, optionally substituted. The preferred R₄ and R₅ are H or unsubstituted alkyl. The preferred Q is CH₂.

In Formula Ia, IIb, and IIc, a preferred R₂ substituent is halo, alkyl, cycloalkyl, hydroxyl, alkoxy, cycloalkyloxy, amino, alkylamino, or R₂ is unsubstituted. A more preferred R₂ substituent is halo, methyl, ethyl, isopropyl, cyclopropyl, hydroxyl, methoxy, ethoxy, amino, methylamino, dimethylamino, or R₂ is unsubstituted.

In a more preferred form of Formulae IIa, IIb, and IIc, Ar is phenyl or a 6,5- or 6,6-fused bicyclic heteroaryl ring, substituted by 1 or 2 substituents X, and Q is CH₂. The most preferred 6,5-fused bicyclic heteroaryl rings are benzofuran, benzothiophene, indole, and benzimidazole.

In its more preferred form, Ar of Formulae Ia, IIb, and IIc is mono- or disubstituted when Ar is phenyl, with 3-substituted, 4-substituted, 2,3-disubstituted, and 3,4-disubstituted being most preferred. When Ar is bicyclic heteroaryl, a monosubstituted Ar is most preferred.

The inventors have found that certain members of Formulae IIa, IIb, and IIc, as defined above, are particularly useful in treating the conditions addressed in this invention. The compounds of the invention are multikinase inhibitors, with inhibitory activity against ROCK1 and ROCK2, in addition to several other kinases in individual compound cases. These kinase inhibitory properties endow the compounds of the invention not only with smooth muscle relaxant properties, but additionally with antiproliferative, antichemotactic, and cytokine secretion inhibitory properties that render them particularly useful in treating conditions with proliferative or inflammatory components as described in the invention.

[17] In particular, we have found that compounds in which R₂ is R₂-2 are particularly potent inhibitors of both ROCK1 and ROCK2, and that these agents inhibit the migration of neutrophils toward multiple chemotactic stimuli and inhibit the secretion of the cytokines IL-1β, TNF-α and IL-9 from LPS-stimulated human monocytes. Compounds in which Ar is heteroaryl, particularly 6,5-fused bicyclic heteroaryl, are especially preferred. These compounds are of particular value in addressing conditions with an inflammatory component.

Compounds exemplifying embodiment 17 include compounds 2.020, 2.021, 2.022, 2.026, 2.031, 2.033, 2.034, 2.038, 2.039, 2.040, 2.041, 2.043, 2.044, 2.054, 2.058, 2.059, 2.060, 2.063, 2.064, 2.066, 2.067, 2.068, 2.069, 2.070, 2.071, 2.072, 2.073, 2.076, 2.077, 2.078, 2.079, 2.080, 2.081, 2.082, 2.087, 2.092, 2.093, 2.094, 2.095, 2.096, 2.097, 2.098, 2.099, and 2.100.

[18] In another embodiment, we have found that compounds of Formula IIc are potent and selective inhibitors of ROCK2, with comparatively lower inhibitory potency against ROCK1. We have demonstrated that compounds of this class typically show good smooth muscle relaxation properties and that smooth muscle relaxation effects in this class are generally correlated with ROCK2 potency. Compounds in which Ar is phenyl are particularly preferred, and compounds bearing one polar group XI in the 3-position and a second group X2 in the 4-position are most preferred. Compounds of this embodiment are of particular value in addressing conditions where relaxation of smooth muscle, in particular vascular and bronchial smooth muscle, is of highest importance.

Compounds exemplifying embodiment 18 include compounds 1.075, 1.077, 1.090, 1.091, 1.094, 1.095, 1.107, 1.109, 1.117, 1.118, 1.124, 1.152, 1.153, 1.157, 1.158, 1.165, 1.168, 1.176, 1.181, 1.182, 1.184, 1.185, 1.186, 1.187, 1.195, 1.196, 1.197, 1.198, 1.199, 1.200, 1.201, 1.213, 1.214, 1.215, 1.217, 1.218, 1.219, 1.223, 1.224, 1.228, 1.229, 1.230, 1.233, 1.234, 1.236, 1.237, 1.238, 1.239, 1.240, 1.253, 1.255, 1.261, 1.269, 1.270, 1.272, 1.274, 1.275, 1.280, and 1.282.

[19] In another embodiment, the inventors have found that compounds of Formula IIb are potent mixed inhibitors of ROCK1 and ROCK2, display additional inhibitory activity against the kinases Akt3 and p70S6K, and that these compounds generally display potent antiproliferative activity in models of smooth muscle cell proliferation. Compounds of this class are of particular value in addressing conditions in which an antiproliferative component is desired in combination with a smooth muscle relaxing effect.

Compounds exemplifying embodiment 19 include compounds 1.074, 1.076, 1.092, 1.093, 1.096, 1.097, 1.106, 1.108, 1.113, 1.115, 1.116, 1.123, 1.125, 1.126, 1.127, 1.128, 1.129, 1.139, 1.140, 1.147, 1.159, 1.160, 1.161, 1.162, 1.174, 1.188, 1.189, 1.193, 1.194, 1.202, 1.205, 1.206, 1.207, 1.208, 1.211, 1.212, 1.221, 1.222, 1.225, 1.231, 1.232, 1.235, 1.244, 1.248, 1.249, 1.258, 1.259, 1.260, 1.262, 1.263, 1.264, 1.265, 1.266, 1.267, 1.268, 1.271, 1.273, 1.276, and 1.281.

[20] In another embodiment, the inventors have found that certain compounds of Formulae IIa, IIb, and IIc distribute preferentially to the lung on oral dosing. In particular, compounds in which Ar is a lipophilic bicyclic heteroaryl group are preferred for this dosing behavior. Compounds of this type are especially useful for treating diseases of the lung by oral dosing while minimizing impact on other tissues.

Compounds exemplifying embodiment 20 include compounds 1.107, 1.109, 1.165, 1.106, 1.108, 2.058, 1.162, 1.264, 1.268, 1.271, 1.273, 1.217, 1.269, 2.059, 2.060, 2.066, and 2.072.

As discussed above for the compounds of Formulae Ia, Ib, and Ic, preparation of compounds of Formulae IIa, IIb, and IIc can be problematic using methods commonly known in the art. The inventors have disclosed and exemplified in US2008/0214614A1 methods to allow successful protection, coupling, and deprotection sequence that allows the successful preparation of the compounds of Formulae IIb and IIc and the demonstration of their useful biological properties.

The present compounds are useful for both oral and topical use, including use by the inhalation route. To be therapeutically effective in this way, the compounds must have both adequate potency and proper pharmacokinetic properties such as good permeability across the biological surface relevant to the delivery route. In general, compounds of Formulae I and II bearing polar functionality, particularly on Ar, have preferred absorption properties and are particularly suitable for topical use. In general, compounds bearing small lipophilic functional groups have good ROCK inhibitory potency.

R₁ substitution in Formula I and X in Formula II are important factors for pharmacokinetic properties and ROCK inhibitory potency. Specifically, compounds bearing polar functionality, especially those specified in the embodiments 11, 12, 13, 14, 15, and 16 in Formulae I and II, above, are particularly suitable for topical use with adequate ROCK inhibiting activity. Compounds bearing small lipophilic functional groups, as specified in the embodiments 11, 12, 13, 14, 15, and 16 in Formulae I and II, above, display ROCK inhibition with adequate permeability across biological surfaces. Compounds bearing substituents of both types are particularly preferred, and when R₁ (Formula I) or Ar (Formula II) is a phenyl ring, compounds with small lipophilic groups in the 4-position and polar functionality in the 3-position are most preferred.

Specific compounds illustrative of Formula I and Formula II are shown in the following Table A. The example compounds have been numbered in such a way that numbers of the form 1.nnn indicate compounds in which R₂ is R₂-1, numbers of the form 2.nnn indicate compounds in which R₂ is R₂-2, and so on in a similar fashion for the remaining compound numbers and groups R₂. In the following structures, hydrogens are omitted from the drawings for the sake of simplicity. Tautomers drawn represent all tautomers possible. Structures are drawn to indicate the preferred stereochemistry; where stereoisomers may be generated in these compounds, structures are taken to mean any of the possible stereoisomers alone or a mixture of stereoisomers in any ratio.

TABLE A
Exemplified Compounds.

| | | Select |
|---|---|---|
| Compound | Structure | Embodiments 1-16 |
| 1.001 | 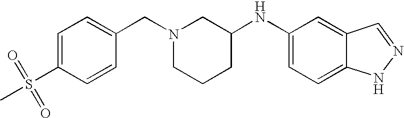 | 1c, 7, 8, 9, 10, 12, 15c |
| | N-(1-(4-(methylsulfonyl)benzyl)piperidin-3-yl)-1H- | |
| | indazol-5-amine | |
| 1.002 | 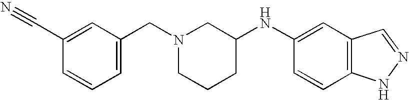 | 1c, 7, 8, 9, 10, 12, 15c |
| | 3-((3-(1H-indazol-5-ylamino)piperidin-1- | |
| | yl)methyl)benzonitrile | |
| 1.003 | 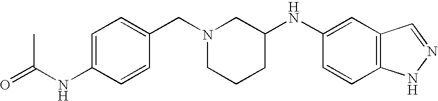 | 1c, 7, 8, 9, 10, 12a, 15c, 15d |
| | N-(4-((3-(1H-indazol-5-ylamino)piperidin-1- | |
| | yl)methyl)phenyl)acetamide | |
| 1.004 | 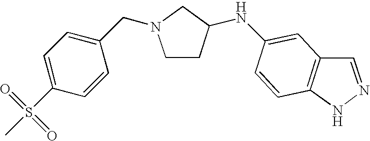 | 1c, 7, 8, 9, 10, 12, 15c |
| | N-(1-(4-(methylsulfonyl)benzyl)pyrrolidin-3-yl)-1H- | |
| | indazol-5-amine | |
| 1.005 | 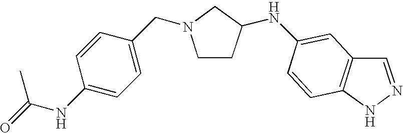 | 1c, 7, 8, 9, 10, 12, 15c |
| | 3-((3-(1H-indazol-5-ylamino)pyrrolidin-1- | |
| | yl)methyl)benzonitrile | |
| 1.006 | 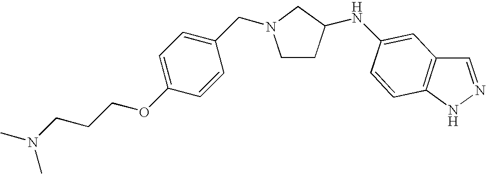 | 1c, 7, 8, 9, 10, 12a, 15c, 15d |
| | N-(4-((3-(1H-indazol-5-ylamino)pyrrolidin-1- | |
| | yl)methyl)phenyl)acetamide | |
| 1.007 | 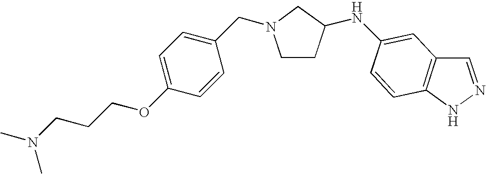 | 1c, 7, 8, 9, 10, 12a, 15c, 15d |
| | N-(1-(4-(3-(dimethylamino)propoxy)benzyl)pyrrolidin- | |
| | 3-yl)-1H-indazol-5-amine | |
| 1.008 | 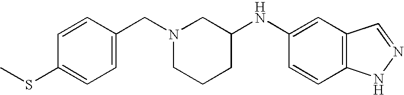 | 1c, 7, 8, 9, 10, 12b, 15c, 15e |
| | N-(1-(4-(methylthio)benzyl)piperidin-3-yl)-1H-indazol- | |
| | 5-amine | |
| 1.009 | 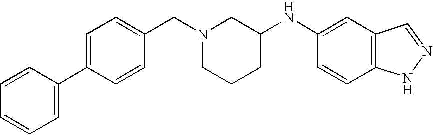 | 1c, 7, 8, 9, 10, 11, 14c |
| | N-(1-(biphenyl-4-ylmethyl)piperidin-3-yl)-1H-indazol-5- | |
| | amine | |
| 1.010 | 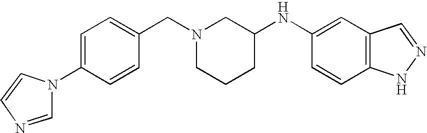 | 1c, 7, 8, 9, 10, 11, 14c |
| | N-(1-(1H-imidazol-1-yl)benzyl)piperidin-3-yl)-1H- | |
| | indazol-5-amine | |
| 1.011 | 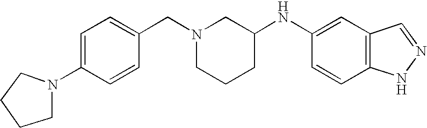 | 1c, 7, 8, 9, 10 11, 14c |
| | N-(1-(4-(pyrrolidin-1-yl)benzyl)piperidin-3-yl)-1H- | |
| | indazol-5-amine | |
| 1.012 | 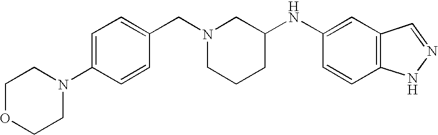 | 1c, 7, 8, 9, 10, 11, 14c |
| | N-(1-(4-morpholinobenzyl)piperidin-3-yl)-1H-indazol-5- | |
| | amine | |
| 1.013 | 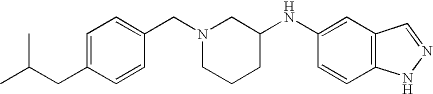 | 1c, 7, 8, 9, 10 |
| | N-(1-(4-isobutylbenzyl)piperidin-3-yl)-1H-indazol-5- | |
| | amine | |
| 1.014 | 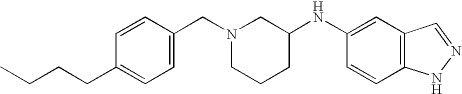 | 1c, 7, 8, 9, 10 |
| | N-(1-(4-butylbenzyl)piperidin-3-yl)-1H-indazol-5-amine | |
| 1.015 | 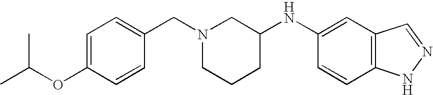 | 1c, 7, 8, 9, 10 |
| | N-(1-(4-isopropoxybenzyl)piperidin-3-yl)-1H-indazol-5- | |
| | amine | |
| 1.016 | 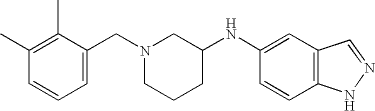 | 1c, 7, 8, 9, 10 |
| | N-(1-(2,3-dimethylbenzyl)piperidin-3-yl)-1H-indazol-5- | |
| | amine | |
| 1.017 | 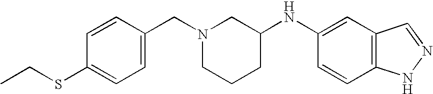 | 1c, 7, 8, 9, 10, 12b, 15c, 15e |
| | N-(1-(4-(ethylthio)benzyl)piperidin-3-yl)-1H-indazol-5- | |
| | amine | |
| 1.018 | 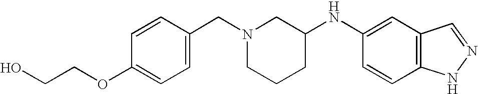 | 1c, 7, 8, 9, 10, 12a, 15c, 15d |
| | 2-(4-((3-(1H-indazol-5-ylamino)piperidin-1-yl)methyl) | |
| | phenoxy)ethanol | |
| 1.019 | 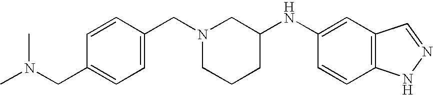 | 1c, 7, 8, 9, 10, 13, 16c |
| | N-(1-(4-((dimethylamino)methyl)benzyl)piperidin-3-yl)- | |
| | 1H-indazol-5-amine | |
| 1.020 | 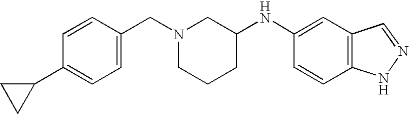 | 1c, 7, 8, 9, 10, 11, 14c |
| | N-(1-(4-cyclopropylbenzyl)piperidin-3-yl)-1H-indazol-5- | |
| | amine | |
| 1.021 | 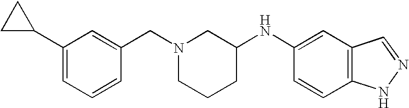 | 1c, 7, 8, 9, 10, 11, 14c |
| | N-(1-(3-cyclopropylbenzyl)piperidin-3-yl)-1H-indazol-5- | |
| | amine | |
| 1.022 | 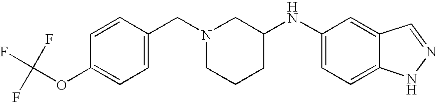 | 1c, 7, 8, 9, 10 |
| | N-(1-(4-(trifluoromethoxy)benzyl)piperidin-3-yl)-1H- | |
| | indazol-5-amine | |
| 1.023 | 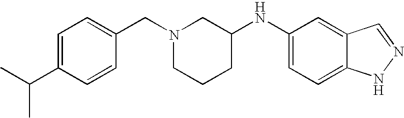 | 1c, 7, 8, 9, 10 |
| | N-(1-(4-isopropylbenzyl)piperidin-3-yl)-1H-indazol-5- | |
| | amine | |
| 1.024 | 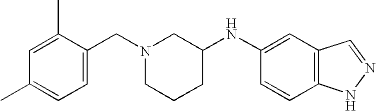 | 1c, 7, 8, 9, 10 |
| | N-(1-(2,4-dimethylbenzyl)piperidin-3-yl)-1H-indazol-5- | |
| | amine | |
| 1.025 | 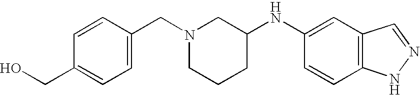 | 1c, 7, 8, 9, 10 |
| | (4-((3-(1H-indazol-5-ylamino)piperidin-1- | |
| | yl)methyl)phenyl)methanol | |
| 1.026 | 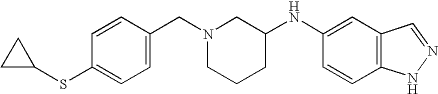 | 1c, 7, 8, 9, 10, 12b, 15c, 15e |
| | N-(1-(4-(cyclopropylthio)benzyl)piperidin-3-yl)-1H- | |
| | indazol-5-amine | |
| 1.027 | 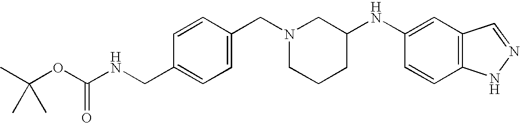 | 1c, 7, 8, 9, 10, 13 16c |
| | tert-butyl 4-((3-(1H-indazol-5-ylamino)piperidin-1- | |
| | yl)methyl)benzylcarbamate | |
| 1.028 | 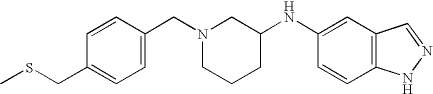 | 1c, 7, 8, 9, 10, 13, 16c |
| | N-(1-(4-(methylthiomethyl)benzyl)piperidin-3-yl)-1H- | |
| | indazol-5-amine | |
| 1.029 | 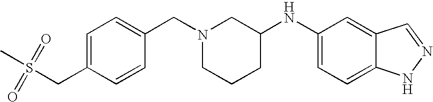 | 1c, 7, 8, 9, 10, 13, 16c |
| | N-(1-(4-(methylsulfonylmethyl)benzyl)piperidin-3-yl)- | |
| | 1H-indazol-5-amine | |
| 1.030 | 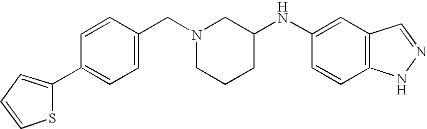 | 1c, 7, 8, 9, l0, 11, |
| | N-(1-(4-(thiophen-2-yl)benzyl)piperidin-3-yl)-1H- | |
| | indazol-5-amine | |
| 1.031 | 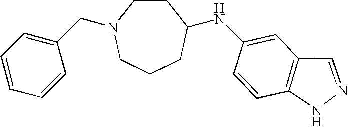 | 1c, 7, 8, 9, 10 |
| | N-(1-benzylazepan-4-yl)-1H-indazol-5-amine | |
| 1.032 | 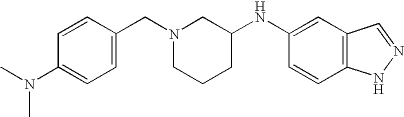 | 1c, 7, 8, 9, 10 |
| | N-(1-(4-(dimethylamino)benzyl)piperidin-3-yl)-1H- | |
| | indazol-5-amine | |
| 1.033 | 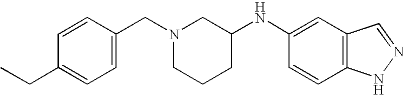 | 1c, 7, 8, 9, 10 |
| | N-(1-(4-ethylbenzyl)piperidin-3-yl)-1H-indazol-5-amine | |
| 1.034 | 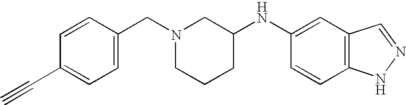 | 1c, 7, 8, 9, 10, 11, 14c |
| | N-(1-(4-ethynylbenzyl)piperidin-3-yl)-1H-indazol-5- | |
| | amine | |
| 1.035 | 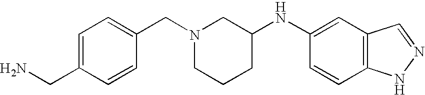 | 1c, 7, 8, 9, 10, 13, 16c |
| | N-(1-(4-(aminomethyl)benzyl)piperidin-3-yl)-1H- | |
| | indazol-5-amine | |
| 1.036 | 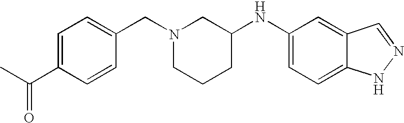 | 1c, 7, 8, 9, 10 |
| | 1-(4-((3-(1H-indazol-5-ylamino)piperidin-1- | |
| | yl)methyl)phenyl)ethanone | |
| 1.037 | 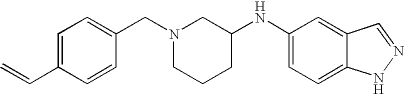 | 1c, 7, 8, 9, 10, 11, 14c |
| | N-(1-(4-vinylbenzyl)piperidin-3-yl)-1H-indazol-5-amine | |
| 1.038 | 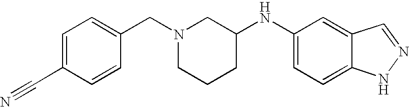 | 1c, 7, 8, 9, 10, 12, 15c |
| | 4-((3-(1H-indazol-5-ylamino)piperidin-1- | |
| | yl)methyl)benzonitrile | |
| 1.039 | 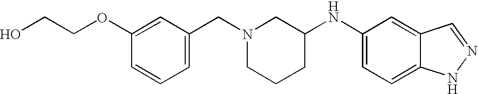 | 1c, 7, 8, 9, 10, 12a, 15c, 15d |
| | 2-(3-((3-(1H-indazol-5-ylamino)piperidin-1-yl)methyl) | |
| | phenoxy)ethanol | |
| 1.040 | 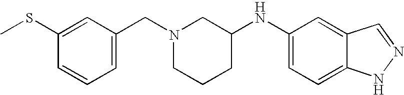 | 1c, 7, 8, 9, 10, 12b, 15c, 15e |
| | N-(1-(3-(methylthio)benzyl)piperidin-3-yl)-1H-indazol- | |
| | 5-amine | |
| 1.041 | 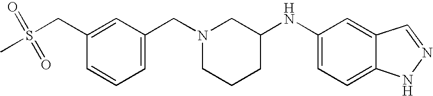 | 1c, 7, 8, 9, 10, 13, 16c |
| | N-(1-(3-(methylsulfonylmethyl)benzyl)piperidin-3-yl)- | |
| | 1H-indazol-5-amine | |
| 1.042 | 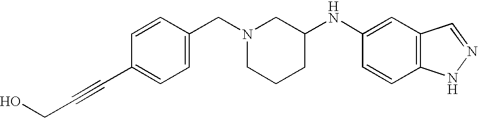 | 1c, 7, 8, 9, 10, 13, 16c |
| | 3-(4-((3-(1H-indazol-5-ylamino)piperidin-1- | |
| | yl)methyl)phenyl)prop-2-yn-1-ol | |
| 1.043 | 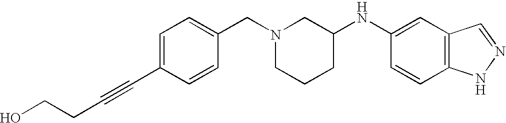 | 1c, 7, 8, 9, 10, 13, 16c |
| | 4-(4-((3-(1H-indazol-5-ylamino)piperidin-1- | |
| | yl)methyl)phenyl)but-3-yn-1-ol | |
| 1.044 | 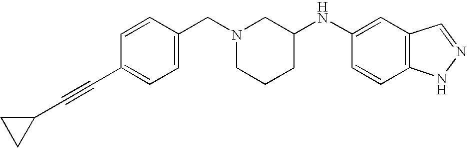 | 1c, 7, 8, 9, 10, 11, 14c |
| | N-(1-(4-(cyclopropylethynyl)benzyl)piperidin-3-yl)-1H- | |
| | indazol-5-amine | |
| 1.045 | 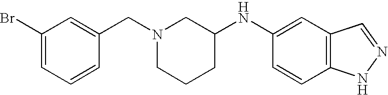 | 1c, 7, 8, 9, 10 |
| | N-(1-(3-bromobenzyl)piperidin-3-yl)-1H-indazol-5- | |
| | amine | |
| 1.046 | 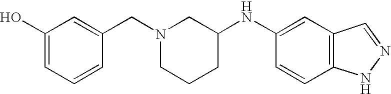 | 1c, 7, 8, 9, 10 |
| | 3-((3-(1H-indazol-5-ylamino)piperidin-1- | |
| | yl)methyl)phenol | |
| 1.047 | 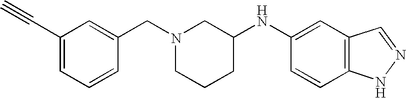 | 1c, 7, 8, 9, 10, 11, 14c |
| | N-(1-(3-ethynylbenzyl)piperidin-3-yl)-1H-indazol-5- | |
| | amine | |
| 1.048 | 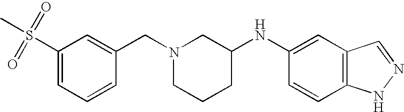 | 1c, 7, 8, 9, 10, 12, 15c |
| | N-(1-(3-(methylsulfonyl)benzyl)piperidin-3-yl)-1H- | |
| | indazol-5-amine | |
| 1.049 | 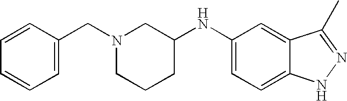 | 1a, 6a, 8, 9, 10 |
| | N-(1-benzylpiperidin-3-yl)-3-methyl-1H-indazol-5- | |
| | amine | |
| 1.050 | 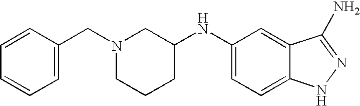 | 1b, 6b, 8, 9, 10 |
| | N-(1-benzylpiperidin-3-yl)-1H-indazole-3,5-diamine | |
| 1.051 | 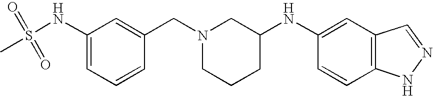 | 1c, 7, 8, 9, 10, 12a, 15c, 15d |
| | N-(3-((3-(1H-indazol-5-ylamino)piperidin-1- | |
| | yl)methyl)phenyl)methanesulfonamide | |
| 1.052 | 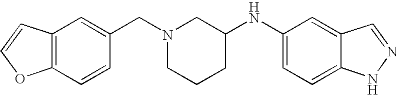 | 1c, 7, 8, 9, 10 |
| | N-(1-(benzofuran-5-ylmethyl)piperidin-3-yl)-1H- | |
| | indazol-5-amine | |
| 1.053 | 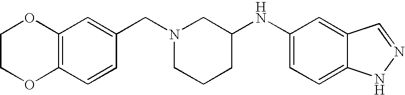 | 1c, 7, 8, 9, 10 |
| | N-(1-((2,3-dihydrobenzo[b][1,4]dioxin-6- | |
| | yl)methyl)piperidin-3-yl)-1H-indazol-5-amine | |
| 1.054 | 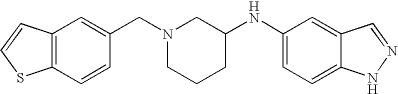 | 1c, 7, 8, 9, 10 |
| | N-(1-(benzo[b]thiophen-5-ylmethyl)piperidin-3-yl)-1H- | |
| | indazol-5-amine | |
| 1.055 | 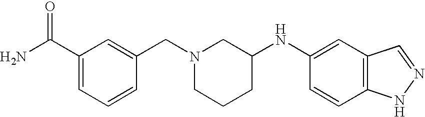 | 1c, 7, 8, 9, 10, 12, 15c |
| | 3-((3-(1H-indazol-5-ylamino)piperidin-1- | |
| | yl)methyl)benzamide | |
| 1.056 | 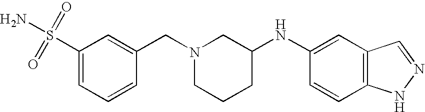 | 1c, 7, 8, 9, 10, 12, 15c |
| | 3-((3-(1H-indazol-5-ylamino)piperidin-1- | |
| | yl)methyl)benzenesulfonamide | |
| 1.057 | 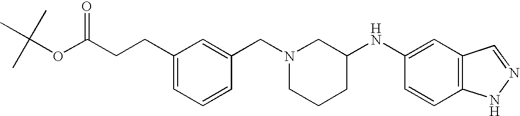 | 1c, 7, 8, 9, 10, 13, 16c |
| | tert-butyl 3-((3-(1H-indazol-5-ylamino)piperidin-1- | |
| | yl)methyl)benzylcarbamate | |
| 1.058 | 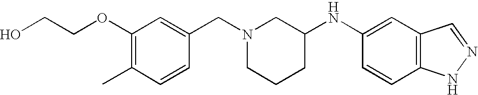 | 1c, 7, 8, 9, 10, 12a, 15c, 15d |
| | 2-(5-((3-(1H-indazol-5-ylamino)piperidin-1-yl)methyl)- | |
| | 2-methylphenoxy)ethanol | |
| 1.059 | 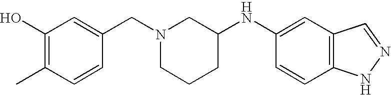 | 1c, 7, 8, 9, 10 |
| | 5-((3-(1H-indazol-5-ylamino)piperidin-1-yl)methyl)-2- | |
| | methylphenol | |
| 1.060 | 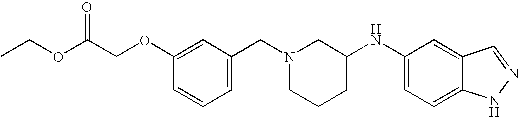 | 1c, 7, 8, 9, 10, 12a 15c, 15d |
| | ethyl 2-(3-((3-(1H-indazol-5-ylamino)piperidin-1- | |
| | yl)methyl)phenoxy)acetate | |
| 1.061 | 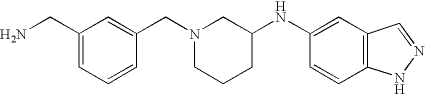 | 1c, 7, 8, 9, 10, 13 16c |
| | N-(1-(3-(aminomethyl)benzyl)piperidin-3-yl)-1H- | |
| | indazol-5-amine | |
| 1.062 | 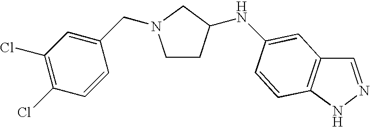 | 1c, 7, 8, 9, 10 |
| | N-(1-(3,4-dichlorobenzyl)pyrrolidin-3-yl)-1H-indazol-5- | |
| | amine | |
| 1.063 | 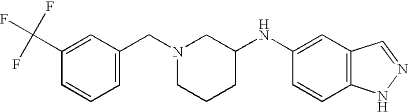 | 1c, 7, 8, 9, 10 |
| | N-(1-(3-(trifluoromethyl)benzyl)piperidin-3-yl)-1H- | |
| | indazol-5-amine | |
| 1.064 | 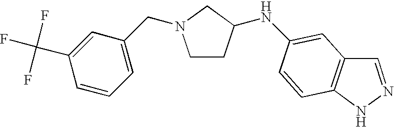 | 1c, 7, 8, 9, 10 |
| | N-(1-(3-(trifluoromethyl)benzyl)pyrrolidin-3-yl)-1H- | |
| | indazol-5-amine | |
| 1.065 | 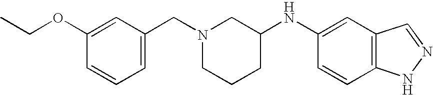 | 1c, 7, 8, 9, 10 |
| | N-(1-(3-ethoxybenzyl)piperidin-3-yl)-1H-indazol-5- | |
| | amine | |
| 1.066 | 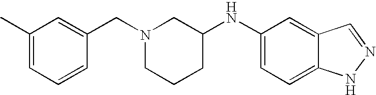 | 1c, 7, 8, 9, 10 |
| | N-(1-(3-methylbenzyl)piperidin-3-yl)-1H-indazol-5- | |
| | amine | |
| 1.067 | 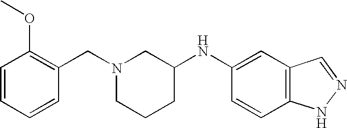 | 1c, 7, 8, 9, 10 |
| | N-(1-(2-methoxybenzyl)piperidin-3-yl)-1H-indazol-5- | |
| | amine | |
| 1.068 | 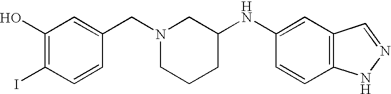 | 1c, 7, 8, 9, 10 |
| | 5-((3-(1H-indazol-5-ylamino)piperidin-1-yl)methyl)-2- | |
| | iodophenol | |
| 1.069 | 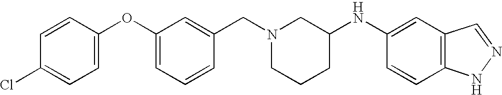 | 1c, 7, 8, 9, 10 |
| | N-(1-(3-(4-chlorophenoxy)benzyl)piperidin-3-yl)-1H- | |
| | indazol-5-amine | |
| 1.070 | 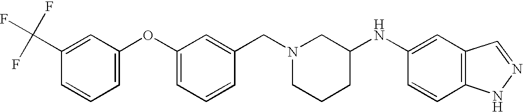 | 1c, 7, 8, 9, 10 |
| | N-(1-(3-(3-(trifluoromethyl)phenoxy)benzyl)piperidin-3- | |
| | yl)-1H-indazol-5-amine | |
| 1.071 | 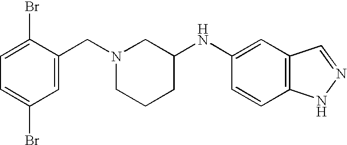 | 1c, 7, 8, 9, 10 |
| | N-(1-(2,5-dibromobenzyl)piperidin-3-yl)-1H-indazol-5- | |
| | amine | |
| 1.072 | 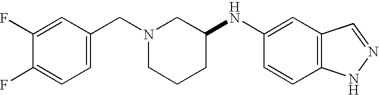 | 1c, 7, 8, 9, 10 |
| | (S)-N-(1-(3,4-difluorobenzyl)piperidin-3-yl)-1H-indazol- | |
| | 5-amine | |
| 1.073 | 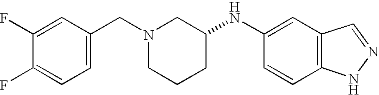 | 1c, 7, 8, 9, 10 |
| | (R)-N-(1-(3,4-difluorobenzyl)piperidin-3-yl)-1H-indazol- | |
| | 5-amine | |
| 1.074 | 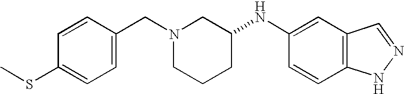 | 1c, 7, 8, 9, 10, 12b, 15c, 15d |
| | (R)-N-(1-(4-(methylthio)benzyl)piperidin-3-yl)-1H- | |
| | indazol-5-amine | |
| 1.075 | 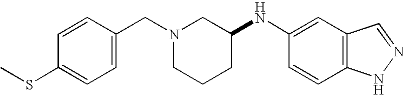 | 1c, 7, 8, 9, 10. 12b, 15c, 15e |
| | (S)-N-(1-(4-(methylthio)benzyl)piperidin-3-yl)-1H- | |
| | indazol-5-amine | |
| 1.076 | 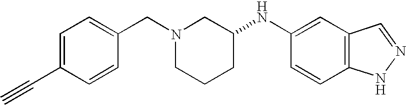 | 1c, 7, 8, 9, 10, 11, 14c |
| | (R)-N-(1-(4-ethynylbenzyl)piperidin-3-yl)-1H-indazol-5- | |
| | amine | |
| 1.077 | 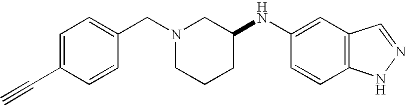 | 1c, 7, 8, 9, 10, 11, 14c |
| | (S)-N-(1-(4-ethynylbenzyl)piperidin-3-yl)-1H-indazol-5- | |
| | amine | |
| 1.078 | 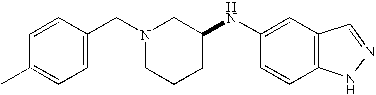 | 1c, 7, 8, 9, 10 |
| | (S)-N-(1-(4-methylbenzyl)piperidin-3-yl)-1H-indazol-5- | |
| | amine | |
| 1.079 | 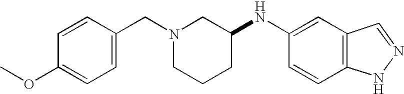 | 1c, 7, 8, 9, 10 |
| | (S)-N-(1-(4-methoxybenzyl)piperidin-3-yl)-1H-indazol- | |
| | 5-amine | |
| 1.080 | 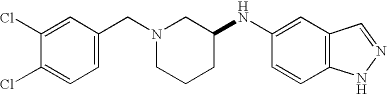 | 1c, 7, 8, 9, 10 |
| | (S)-N-(1-(3,4-dichlorobenzyl)piperidin-3-yl)-1H- | |
| | indazol-5-amine | |
| 1.082 | 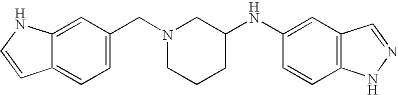 | 1c, 7, 8, 9, 10 |
| | N-(1-((1H-indol-6-yl)methyl)piperidin-3-yl)-1H-indazol- | |
| | 5-amine | |
| 1.083 | 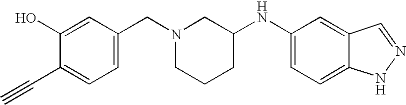 | 1c, 7, 8, 9, 10, 11, 14c |
| | 5-((3-(1H-indazol-5-ylamino)piperidin-1-yl)methyl)-2- | |
| | ethynylphenol | |
| 1.084 | 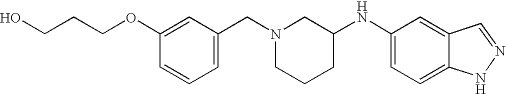 | 1c, 7, 8, 9, 10, 12a, 15c, 15d |
| | 3-(3-((3-(1H-indazol-5-ylamino)piperidin-1-yl)methyl) | |
| | phenoxy)propan-1-ol | |
| 1.085 | 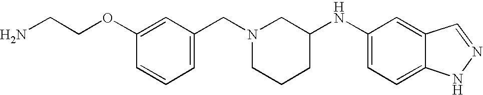 | 1c, 7, 8, 9, 10, 12a, 15c, 15d |
| | N-(1-(3-(2-aminoethoxy)benzyl)piperidin-3-yl)-1H- | |
| | indazol-5-amine | |
| 1.086 | 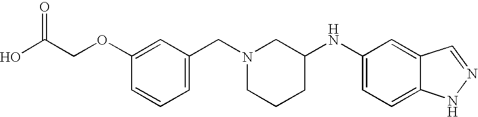 | 1c, 7, 8, 9, 10, 12a, 15c, 15d |
| | 2-(3-((3-(1H-indazol-5-ylamino)piperidin-1-yl)methyl) | |
| | phenoxy)acetic acid | |
| 1.087 | 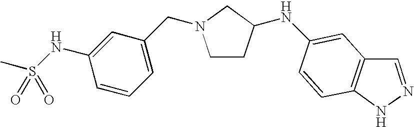 | 1c, 7, 8, 9, 10, 12a, 15c, 15d |
| | N-(3-((3-(1H-indazol-5-ylamino)pyrrolidin-1- | |
| | yl)methyl)phenyl)methanesulfonamide | |
| 1.088 | 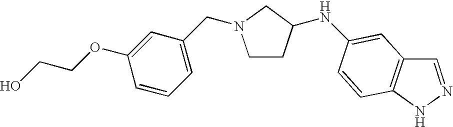 | 1c, 7, 8, 9, 10, 12a, 15c, 15d |
| | 2-(3-((3-(1H-indazol-5-ylamino)pyrrolidin-1-yl)methyl) | |
| | phenoxy)ethanol | |
| 1.089 | 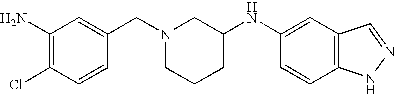 | 1c, 7, 8, 9, 10 |
| | N-(1-(3-amino-4-chlorobenzyl)piperidin-3-yl)-1H- | |
| | indazol-5-amine | |
| 1.090 | 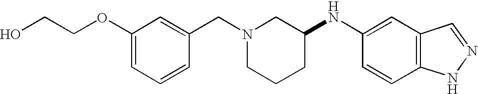 | 1c, 7, 8, 9, 10, 12a, 15c, 15d |
| | (S)-2-(3-((3-(1H-indazol-5-ylamino)piperidin-1- | |
| | yl)methyl)phenoxy)ethanol | |
| 1.091 | 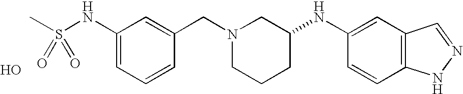 | 1c, 7, 8, 9, 10, 12a, 15c, 15d |
| | (S)-N-(3-((3-(1H-indazol-5-ylamino)piperidin-1- | |
| | yl)methyl)phenyl)methanesulfonamide | |
| 1.092 | 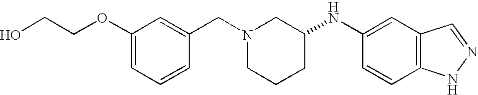 | 1c, 7, 8, 9, 10, 12a, 15c, 15d |
| | (R)-2-(3-((3-(1H-indazol-5-ylamino)piperidin-1- | |
| | yl)methyl)phenoxy)ethanol | |
| 1.093 | 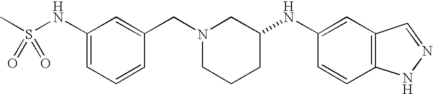 | 1c, 7, 8, 9, 10, 12a, 15c, 15d |
| | (R)-N-(3-((3-(1H-indazol-5-ylamino)piperidin-1- | |
| | yl)methyl)phenyl)methanesulfonamide | |
| 1.094 | 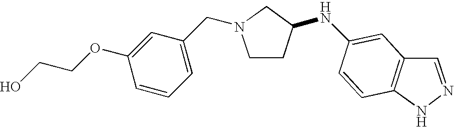 | 1c, 7, 8, 9, 10, 12a, 15c, 15d |
| | (S)-2-(3-((3-(1H-indazol-5-ylamino)pyrrolidin-1- | |
| | yl)methyl)phenoxy)ethanol | |
| 1.095 | 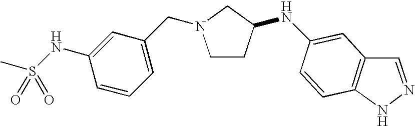 | 1c, 7, 8, 9, 10, 12a, 15c, 15d |
| | (S)-N-(3-((3-(1H-indazol-5-ylamino)pyrrolidin-1- | |
| | yl)methyl)phenyl)methanesulfonamide | |
| 1.096 | 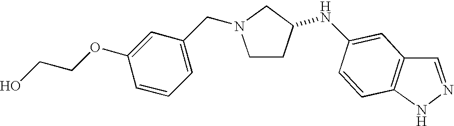 | 1c, 7, 8, 9, 10, 12a, 15c, 15d |
| | (R)-2-(3-((3-(1H-indazol-5-ylamino)pyrrolidin-1- | |
| | yl)methyl)phenoxy)ethanol | |
| 1.097 | 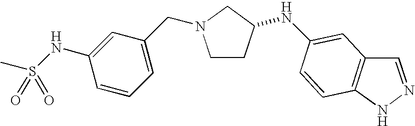 | 1c, 7, 8, 9, 10, 12a, 15c, 15d |
| | (R)-N-(3-((3-(1H-indazol-5-ylamino)pyrrolidin-1- | |
| | yl)methyl)phenyl)methanesulfonamide | |
| 1.098 | 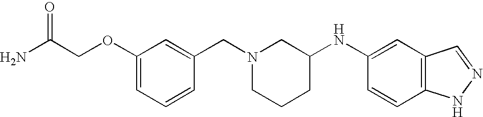 | 1c, 7, 8, 9, 10, 12a, 15c, 15d |
| | 2-(3-((3-(1H-indazol-5-ylamino)piperidin-1- | |
| | yl)methyl)phenoxy)acetamide | |
| 1.099 | 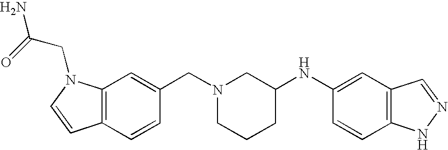 | 1c, 7, 8, 9, 10, 13, 16c |
| | 2-(6-((3-(1H-indazol-5-ylamino)piperidin-1-yl)methyl)- | |
| | 1H-indol-1-yl)acetamide | |
| 1.100 | 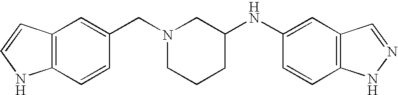 | 1c, 7, 8, 9, 10, 13, 16c |
| | N-(1-((1H-indol-5-yl)methyl)piperidin-3-yl)-1H-indazol- | |
| | 5-amine | |
| 1.101 | 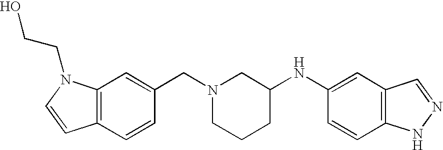 | 1c, 7, 8, 9, 10, 13, 16c |
| | 2-(6-((3-(1H-indazol-5-ylamino)piperidin-1-yl)methyl)- | |
| | 1H-indol-1-yl)ethanol | |
| 1.102 | 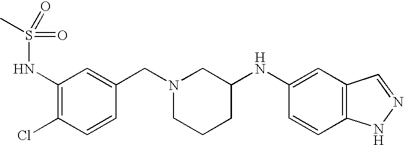 | 1c, 7, 8, 9, 10, 12a, 15c, 15d |
| | N-(5-((3-(1H-indazol-5-ylamino)piperidin-1-yl)methyl)- | |
| | 2-chlorophenyl)methanesulfonamide | |
| 1.103 | 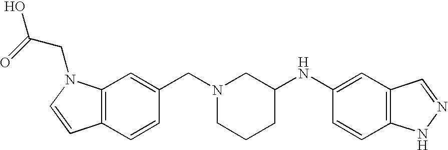 | 1c, 7, 8, 9, 10, 13, 16c |
| | 2-(6-((3-(1H-indazol-5-ylamino)piperidin-1-yl)methyl)- | |
| | 1H-indol-1-yl)acetic acid | |
| 1.104 | 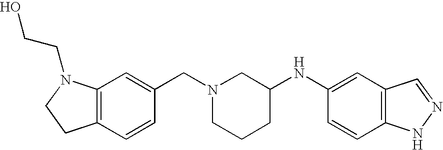 | 1c, 7, 8, 9, 10, 13, 16c |
| | 2-(6-((3-(1H-indazol-5-ylamino)piperidin-1- | |
| | yl)methyl)indolin-1-yl)ethanol | |
| 1.105 | 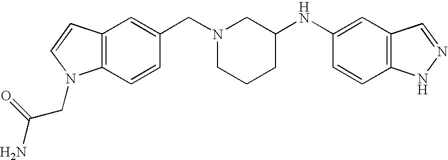 | 1c, 7, 8, 9, 10, 13, 16c |
| | 2-(5-((3-(1H-indazol-5-ylamino)piperidin-1-yl)methyl)- | |
| | 1H-indol-1-yl)-acetamide | |
| 1.106 | 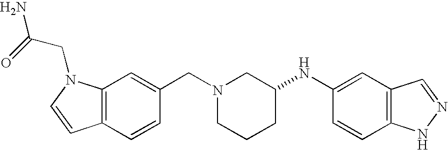 | 1c, 7, 8, 9, 10, 13, 16c |
| | (R)-2-(6-((3-(1H-indazol-5-ylamino)piperidin-1- | |
| | yl)methyl)-1H-indol-1-yl)acetamide | |
| 1.107 | 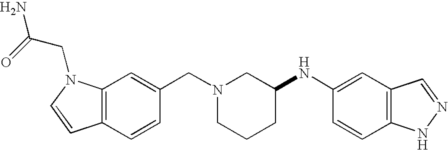 | 1c, 7, 8, 9, 10, 13, 16c |
| | (S)-2-(6-((3-(1H-indazol-5-ylamino)piperidin-1- | |
| | yl)methyl)-1H-indol-1-yl)acetamide | |
| 1.108 | 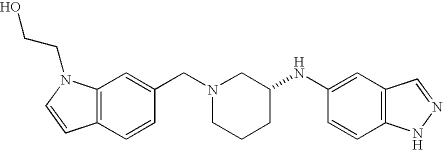 | 1c, 7, 8, 9, 10, 13, 16c |
| | (R)-2-(6-((3-(1H-indazol-5-ylamino)piperidin-1- | |
| | yl)methyl)-1H-indol-1-yl)ethanol | |
| 1.109 | 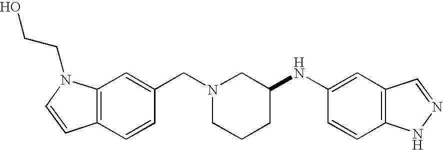 | 1c, 7, 8, 9, 10, 13, 16c |
| | (S)-2-(6-((3-(1H-indazol-5-ylamino)piperidin-1- | |
| | yl)methyl)-1H-indol-1-yl)ethanol | |
| 1.110 | 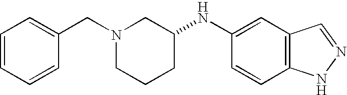 | 1c, 7, 8, 9, 10 |
| | (R)-N-(1-benzylpiperidin-3-yl)-1H-indazol-5-amine | |
| 1.111 | 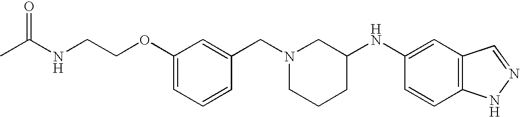 | 1c, 7, 8, 9, 10, 12a, 15c, 15d |
| | N-(2-(3-((3-(1H-indazol-5-ylamino)piperidin-1- | |
| | yl)methyl)phenoxy)ethyl)acetamide | |
| 1.112 | 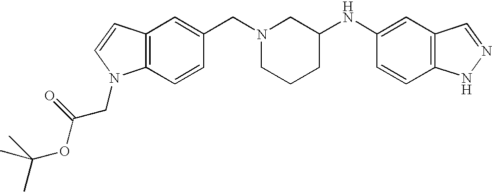 | 1c, 7, 8, 9, 10, 13, 16c |
| | tert-butyl 2-(5-((3-(1H-indazol-5-ylamino)piperidin-1- | |
| | yl)methyl)-1H-indol-1-yl)acetate | |
| 1.113 | 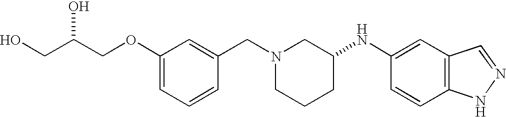 | 1c, 7, 8, 9, 10, 12a, 15c, 15d |
| | (S)-3-(3-(((R)-3-(1H-indazol-5-ylamino)piperidin-1- | |
| | yl)methyl)phenoxy)propane-1,2-diol | |
| 1.114 | 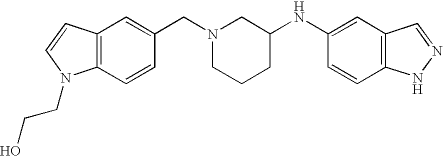 | 1c, 7, 8, 9, 10, 13, 16c |
| | 2-(5-((3-(1H-indazol-5-ylamino)piperidin-1-yl)methyl)- | |
| | 1H-indol-1-yl)ethanol | |
| 1.115 | 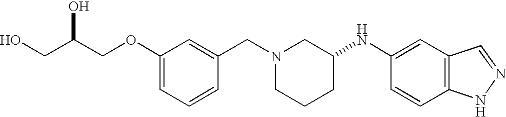 | 1c, 7, 8, 9, 10, 12a, 15c, 15d |
| | (R)-3-(3-(((R)-3-(1H-indazol-5-ylamino)piperidin-1- | |
| | yl)methyl)phenoxy)propane-1,2-diol | |
| 1.116 | 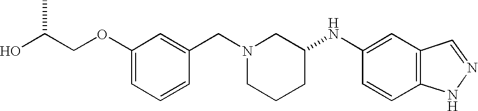 | 1c, 7, 8, 9, 10, 12a, 15c, 15d |
| | (R)-1-(3-(((R)-3-(1H-indazol-5-ylamino)piperidin-1- | |
| | yl)methyl)phenoxy)propan-2-ol | |
| 1.117 | 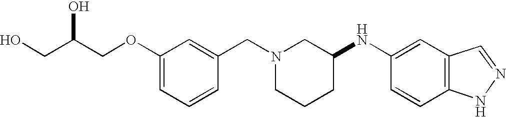 | 1c, 7, 8, 9, 10, 12a, 15c, 15d |
| | (R)-3-(3-(((S)-3-(1H-indazol-5-ylamino)piperidin-1- | |
| | yl)methyl)phenoxy)propane-1,2-diol | |
| 1.118 | 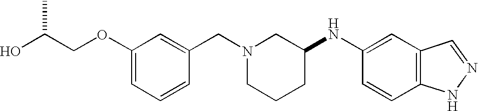 | 1c, 7, 8, 9, 10, 12a 15c, 15d |
| | (R)-1-(3-(((S)-3-(1H-indazol-5-ylamino)piperidin-1- | |
| | yl)methyl)phenoxy)propan-2-ol | |
| 1.119 | 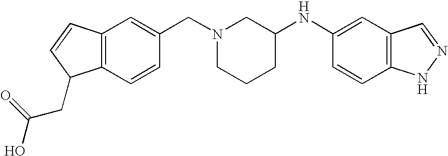 | 1c, 7, 8, 9, 10, 13, 16c |
| | 2-(5-((3-(1H-indazol-5-ylamino)piperidin-1-yl)methyl)- | |
| | 1H-indol-1-yl)acetic acid | |
| 1.120 | 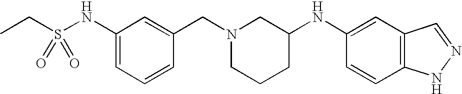 | 1c, 7, 8, 9, 10, 12a, 15c, 15d |
| | N-(3-((3-(1H-indazol-5-yJamino)piperidin-1- | |
| | yl)methyl)phenyl)ethanesulfonamide | |
| 1.121 | 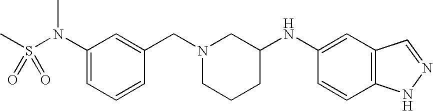 | 1c, 7, 8, 9, 10, 12a, 15c, 15d |
| | N-(3-((3-(1H-indazol-5-ylamino)piperidin-1- | |
| | yl)methyl)phenyl)-N-methylmethanesulfonamide | |
| 1.122 | 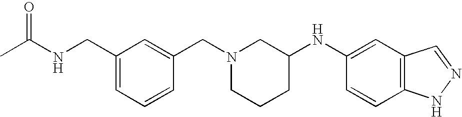 | 1c, 7, 8, 9, 10, 13, 16c |
| | N-(3-((3-(1H-indazol-5-ylamino)piperidin-1- | |
| | yl)methyl)benzyl)acetamide | |
| 1.123 | 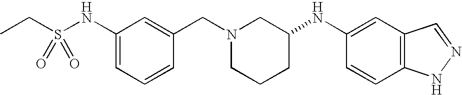 | 1c, 7, 8, 9, 10, 12a, 15c, 15d |
| | (R)-N-(3-((3-(1H-indazol-5-ylamino)piperidin-1- | |
| | yl)methyl)phenyl)ethanesulfonamide | |
| 1.124 | 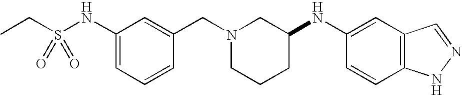 | 1c, 7, 8, 9, 10, 12a, 15c, 15d |
| | (S)-N-(3-((3-(1H-indazol-5-ylamino)piperidin-1- | |
| | yl)methyl)phenyl)ethanesulfonamide | |
| 1.125 | 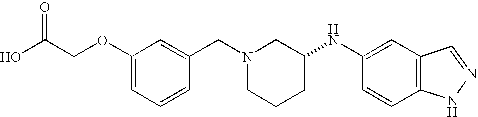 | 1c, 7, 8, 9, 10, 12a, 15c, 15d |
| | (R)-2-(3-((3-(1H-indazol-5-ylamino)piperidin-1- | |
| | yl)methyl)phenoxy)acetic acid | |
| 1.126 | 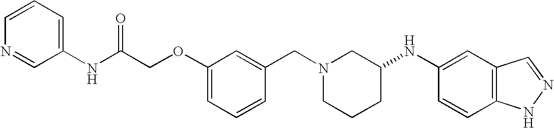 | 1c, 7, 8, 9, 10, 12a, 15c, 15d |
| | (R)-2-(3-((3-(1H-indazol-5-ylamino)piperidin-1- | |
| | yl)methyl)phenoxy)-N-(pyridin-3-yl)acetamide | |
| 1.127 | 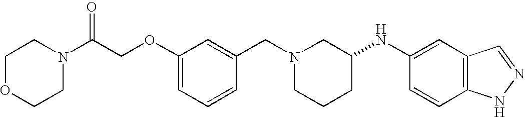 | 1c, 7, 8, 9, 10, 12a, 15c, 15d |
| | (R)-2-(3-((3-(1H-indazol-5-ylamino)piperidin-1- | |
| | yl)methyl)phenoxy)-1-morpholinoethanone | |
| 1.128 | 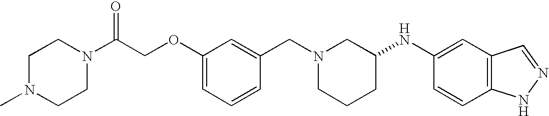 | 1c, 7, 8, 9, 10, 12a, 15c, 15d |
| | (R)-2-(3-((3-(1H-indazol-5-ylamino)piperidin-1- | |
| | yl)methyl)phenoxy)-1-(4-methylpiperazin-1- | |
| | yl)ethanone | |
| 1.129 | 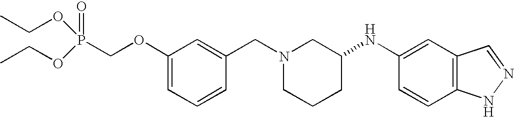 | 1c, 7, 8, 9, 10, 12a, 15c, 15d |
| | (R)-diethyl(3-((3-(1H-indazol-5-ylamino)piperidin-1- | |
| | yl)methyl)phenoxy)methylphosphonate | |
| 1.130 | 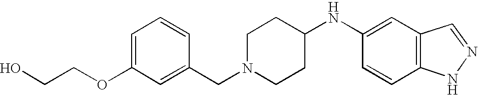 | 1c, 7, 8, 9, 10, 12a, 15c, 15d |
| | 2-(3-((4-(1H-indazol-5-ylamino)piperidin-1- | |
| | yl)methyl)phenoxy)ethanol | |
| 1.131 | 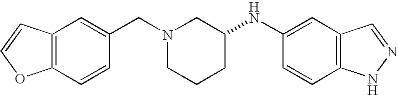 | 1c, 7, 8, 9, 10 |
| | (R)-N-(1-(benzofuran-5-ylmethyl)piperidin-3-yl)-1H- | |
| | indazol-5-amine | |
| 1.132 | 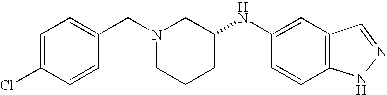 | 1c, 7, 8, 9, 10 |
| | (R)-N-(1-(4-chlorobenzyl)piperidin-3-yl)-1H-indazol-5- | |
| | amine | |
| 1.133 | 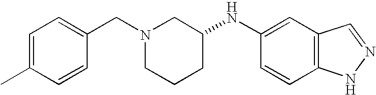 | 1c, 7, 8, 9, 10 |
| | (R)-N-(1-(4-methylbenzyl)piperidin-3-yl)-1H-indazol-5- | |
| | amine | |
| 1.134 | 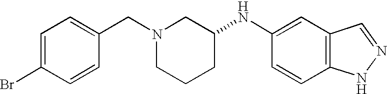 | 1c, 7, 8, 9, 10 |
| | (R)-N-(1-(4-bromobenzyl)piperidin-3-yl)-1H-indazol-5- | |
| | amine | |
| 1.136 | 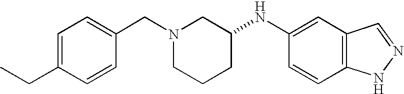 | 1c, 7, 8, 9, 10 |
| | (R)-N-(1-(4-ethylbenzyl)piperidin-3-yl)-1H-indazol-5- | |
| | amine | |
| 1.137 | 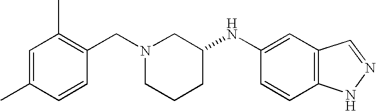 | 1c, 7, 8, 9, 10 |
| | (R)-N-(1-(2,4-dimethylbenzyl)piperidin-3-yl)-1H- | |
| | indazol-5-amine | |
| 1.138 | 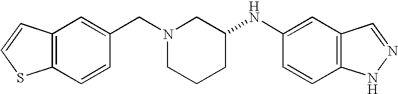 | 1c, 7, 8, 9, 10 |
| | (R)-N-(1-(benzo[b]thiophen-5-ylmethyl)piperidin-3-yl)- | |
| | 1H-indazol-5-amine | |
| 1.139 | 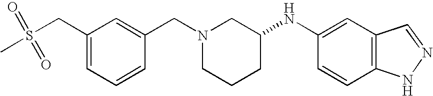 | 1c, 7, 8, 9, 10, 12, 15c |
| | (R)-N-(1-(3-(methylsuifonylmethyl)benzyl)piperidin-3- | |
| | yl)-1H-indazol-5-amine | |
| 1.140 | 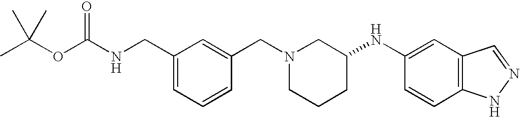 | 1c, 7, 8, 9, 10, 13, 16c |
| | (R)-tert-butyl 3-((3-(1H-indazol-5-ylamino)piperidin-1- | |
| | yl)methyl)benzylcarbamate | |
| 1.141 | 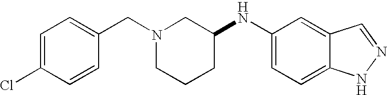 | 1c, 7, 8, 9, 10 |
| | (S)-N-(1-(4-chlorobenzyl)piperidin-3-yl)-1H-indazol-5- | |
| | amine | |
| 1.142 | 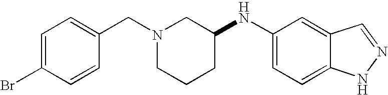 | 1c, 7, 8, 9, 10 |
| | (S)-N-(1-(4-bromobenzyl)piperidin-3-yl)-1H-indazoi-5- | |
| | amine | |
| 1.143 | 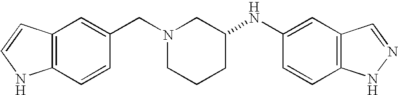 | 1c, 7, 8, 9, 10, 13, 16c |
| | (R)-N-(1-((1H-indol-5-yl)methyl)piperidin-3-yl)-1H- | |
| | indazol-5-amine | |
| 1.144 | 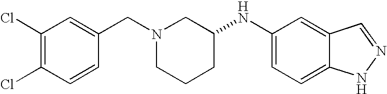 | 1c, 7, 8, 9, 10 |
| | (R)-N-(1-(3,4-dichlorobenzyl)piperidin-3-yl)-1H- | |
| | indazol-5-amine | |
| 1.145 | 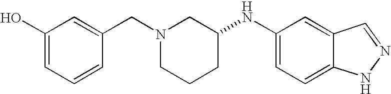 | 1c, 7, 8, 9, 10 |
| | (R)-3-((3-(1H-indazol-5-ylamino)piperidin-1- | |
| | yl)methyl)phenol | |
| 1.146 | 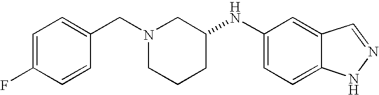 | 1c, 7, 8, 9, 10 |
| | (R)-N-(1-(4-fluorobenzyl)piperidin-3-yl)-1H-indazol-5- | |
| | amine | |
| 1.147 | 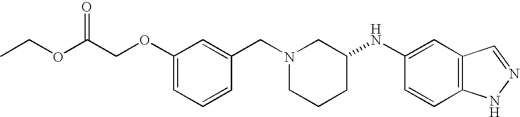 | 1c, 7, 8, 9, 10, 12a, 15c, 15d |
| | (R)-ethyl 2-(3-((3-(1H-indazol-5-ylamino)piperidin-1- | |
| | yl)methyl)phenoxy)acetate | |
| 1.148 | 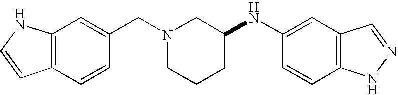 | 1c, 7, 8, 9, 10 |
| | (S)-N-(1-((1H-indol-6-yl)methyl)piperidin-3-yl)-1H- | |
| | indazol-5-amine | |
| 1.149 | 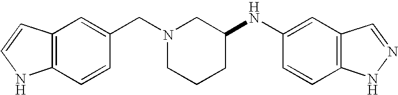 | 1c, 7, 8, 9, 10 |
| | (S)-N-(1-((1H-indol-5-yl)methyl)piperidin-3-yl)-1H- | |
| | indazol-5-amine | |
| 1.150 | 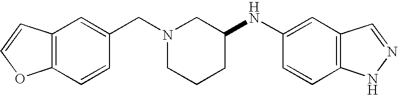 | 1c, 7, 8, 9, 10 |
| | (S)-N-(1-(benzofuran-5-ylmethyl)piperidin-3-yl)-1H- | |
| | indazol-5-amine | |
| 1.151 | 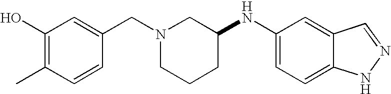 | 1c, 7, 8, 9, 10 |
| | (S)-5-((3-(1H-indazol-5-ylamino)piperidin-1-yl)methyl) | |
| | 2-methylphenol | |
| 1.152 | 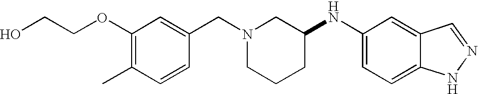 | 1c, 7, 8, 9, 10, 12a, 15c, 15d |
| | (S)-2-(5-((3-(1H-indazol-5-ylamino)piperidin-1- | |
| | yl)methyl)-2-methylphenoxy)ethanol | |
| 1.153 | 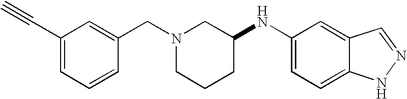 | 1c, 7, 8, 9, 10, 11, 14c |
| | (S)-N-(1-(3-ethynylbenzyl)piperidin-3-yl)-1H-indazol-5- | |
| | amine | |
| 1.154 | 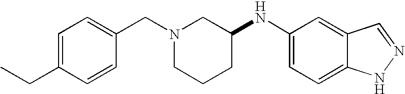 | 1c, 7, 8, 9, 10 |
| | (S)-N-(1-(4-ethylbenzyl)piperidin-3-yl)-1H-indazol-5- | |
| | amine | |
| 1.155 | 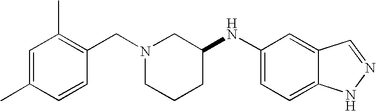 | 1c, 7, 8, 9, 10 |
| | (S)-N-(1-(2,4-dimethylbenzyl)piperidin-3-yl)-1H | |
| | indazol-5-amine | |
| 1.156 | 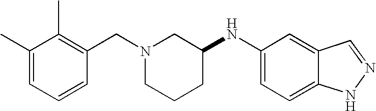 | 1c, 7, 8, 9, 10 |
| | (S)-N-(1-(2,3-dimethylbenzyl)piperidin-3-yl)-1H- | |
| | indazol-5-amine | |
| 1.157 | 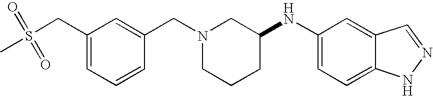 | 1c, 7, 8, 9, 10, 12, 15c |
| | (S)-N-(1-(3-(methylsulfonylmethyl)benzyl)piperidin-3- | |
| | yl)-1H-indazol-5-amine | |
| 1.158 | 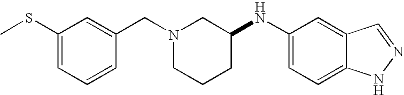 | 1c, 7, 8, 9, 10, 12b, 15c, 15e |
| | (S)-N-(1-(3-(methylthio)benzyl)piperidin-3-yl)-1H- | |
| | indazol-5-amine | |
| 1.159 | 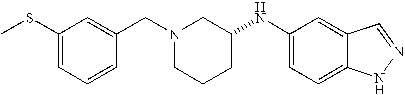 | 1c, 7, 8, 9, 10, 12b, 15c, 15e |
| | (R)-N-(1-(3-(methylthio)benzyl)piperidin-3-yl)-1H- | |
| | indazol-5-amine | |
| 1.160 | 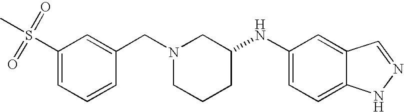 | 1c, 7, 8, 9, 10, 12, 15c |
| | (R)-N-(1-(3-(methylsulfonyl)benzyl)piperidin-3-yl)-1H- | |
| | indazol-5-amine | |
| 1.161 | 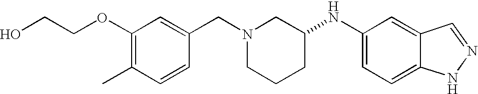 | 1c, 7, 8, 9, 10, 12a, 15c, 15d |
| | (R)-2-(5-((3-(1H-indazol-5-ylamino)piperidin-1- | |
| | yl)methyl)-2-methylphenoxy)ethanol | |
| 1.162 | 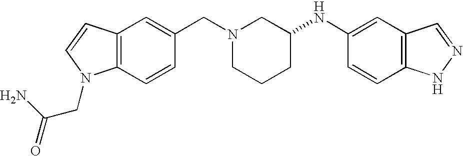 | 1c, 7, 8, 9, 10, 13, 16c |
| | (R)-2-(5-((3-(1H-indazol-5-ylamino)piperidin-1- | |
| | yl)methyl)-1H-indol-1-yl)acetamide | |
| 1.163 | 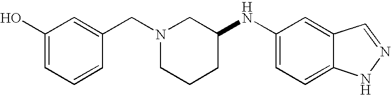 | 1c, 7, 8, 9, 10 |
| | (S)-3-((3-(1H-indazol-5-ylamino)piperidin-1- | |
| | yl)methyl)phenol | |
| 1.164 | 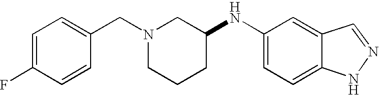 | 1c, 7, 8, 9, 10 |
| | (S)-N-(1-(4-fluorobenzyl)piperidin-3-yl)-1H-indazol-5- | |
| | amine | |
| 1.165 | 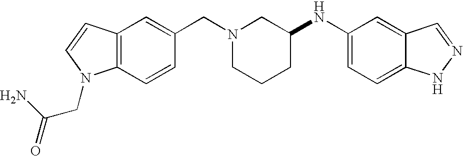 | 1c, 7, 8, 9, 10, 13, 16c |
| | (S)-2-(5-((3-(1H-indazol-5-ylamino)piperidin-1- | |
| | yl)methyl)-1H-indol-1-yl)acetamide | |
| 1.166 | 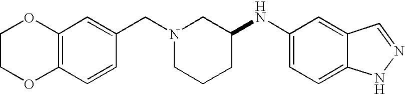 | 1c, 7, 8, 9, 10 |
| | (S)-N-(1-((2,3-dihydrobenzo[b][1,4]dioxin-6- | |
| | yl)methyl)piperidin-3-yl)-1H-indazol-5-amine | |
| 1.167 | 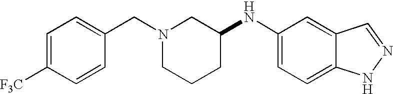 | 1c, 7, 8, 9, 10 |
| | (S)-N-(1-(4-(trifluoromethyl)benzyl)piperidin-3-yl)-1H- | |
| | indazol-5-amine | |
| 1.168 | 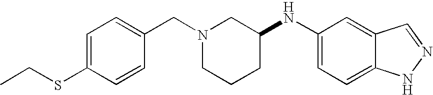 | 1c, 7, 8, 9, 10, 12b, 15c, 15e |
| | (S)-N-(1-(4-(ethylthio)benzyl)piperidin-3-yl)-1H- | |
| | indazol-5-amine | |
| 1.169 | 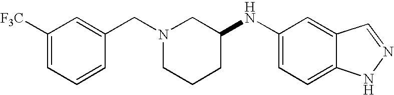 | 1c, 7, 8, 9, 10 |
| | (S)-N-(1-(3-(trifluoromethyl)benzyl)piperidin-3-yl)-1H- | |
| | indazol-5-amine | |
| 1.170 | 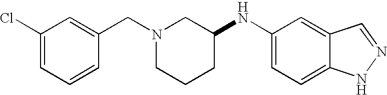 | 1c, 7, 8, 9, 10 |
| | (S)-N-(1-(3-chlorobenzyl)piperidin-3-yl)-1H-indazol-5- | |
| | amine | |
| 1.171 | 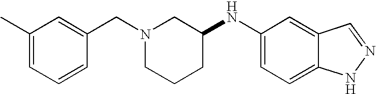 | 1.171 |
| | (S)-N-(1-(3-methylbenzyl)piperidin-3-yl)-1H-indazol-5- | |
| | amine | |
| 1.172 | 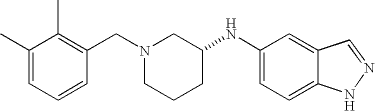 | 1.172 |
| | (R)-N-(1-(2,3-dimethylbenzyl)piperidin-3-yl)-1H- | |
| | indazol-5-amine | |
| 1.173 | 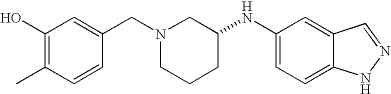 | 1.173 |
| | (R)-5-((3-(1H-indazol-5-ylamino)piperidin-1-yl)methyl)- | |
| | 2-methylphenol | |
| 1.174 | 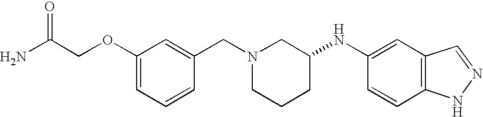 | 1.174 |
| | (R)-2-(3-((3-(1H-indazol-5-ylamino)piperidin-1- | |
| | yl)methyl)phenoxy)acetamide | |
| 1.175 | 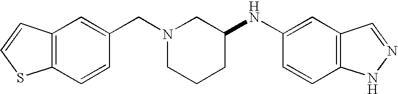 | 1.175 |
| | (S)-N-(1-(benzo[b]thiophen-5-ylmethyl)piperidin-3-yl)- | |
| | 1H-indazol-5-amine | |
| 1.176 | 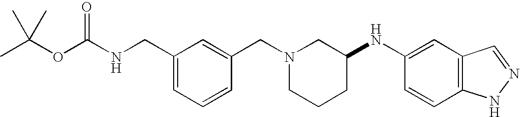 | 1.176 |
| | (S)-tert-butyl 3-((3-(1H-indazol-5-ylamino)piperidin-1- | |
| | yl)methyl)benzylcarbamate | |
| 1.177 | 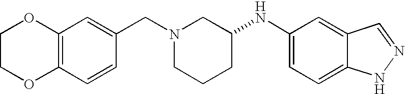 | 1.177 |
| | (R)-N-(1-((2,3-dihydrobenzo[b][1,4]dioxin-6- | |
| | yl)methyl)piperidin-3-yl)-1H-indazol-5-amine | |
| 1.178 | 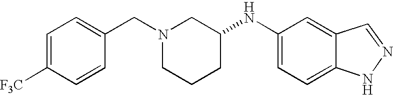 | 1.178 |
| | (R)-N-(1-(4-(trifluoromethyl)benzyl)piperidin-3-yl)-1H- | |
| | indazol-5-amine | |
| 1.179 | 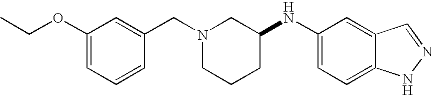 | 1.179 |
| | (S)-N-(1-(3-ethoxybenzyl)piperidin-3-yl)-1H-indazol-5- | |
| | amine | |
| 1.180 | 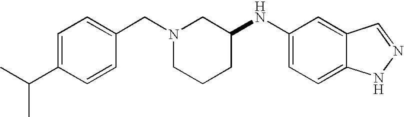 | 1.180 |
| | (S)-N-(1-(4-isopropylbenzyl)piperidin-3-yl)-1H-indazol- | |
| | 5-amine | |
| 1.181 | 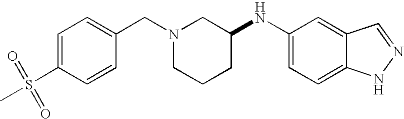 | 1.181 |
| | (S)-N-(1-(4-(methylsulfonyl)benzyl)piperidin-3-yl)-1H- | |
| | indazol-5-amine | |
| 1.182 | 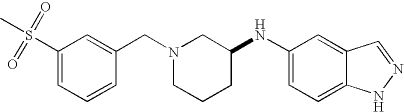 | 1.182 |
| | (S)-N-(1-(3-(methylsulfonyl)benzyl)piperidin-3-yl)-1H- | |
| | indazol-5-amine | |
| 1.183 | 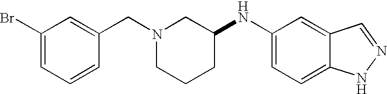 | 1.183 |
| | (S)-N-(1-(3-bromobenzyl)piperidin-3-yl)-1H-indazol-5- | |
| | amine | |
| 1.184 | 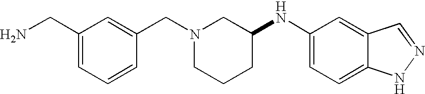 | 1.184 |
| | (S)-N-(1-(3-(aminomethyl)benzyl)piperidin-3-yl)-1H- | |
| | indazol-5-amine | |
| 1.185 | 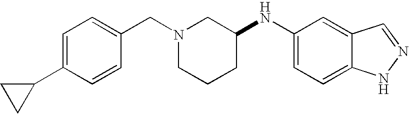 | 1.185 |
| | (S)-N-(1-(4-cyclopropylbenzyl)piperidin-3-yl)-1H- | |
| | indazol-5-amine | |
| 1.186 | 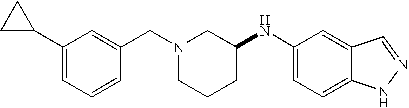 | 1.186 |
| | (S)-N-(1-(3-cyclopropylbenzyl)piperidin-3-yl)-1H- | |
| | indazol-5-amine | |
| 1.187 | 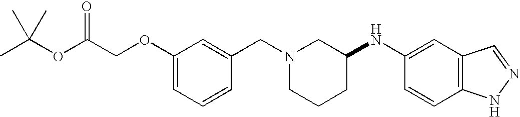 | 1.187 |
| | (S)-tert-butyl 2-(3-((3-(1H-indazol-5-ylamino)piperidin- | |
| | 1-yl)methyl)phenoxy)acetate | |
| 1.188 | 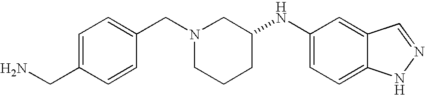 | 1.188 |
| | (R)-N-(1-(4-(aminomethyl)benzyl)piperidin-3-yl)-1H- | |
| | indazol-5-amine | |
| 1.189 | 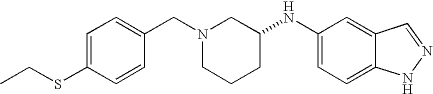 | 1.189 |
| | (R)-N-(1-(4-(ethylthio)benzyl)piperidin-3-yl)-1H- | |
| | indazol-5-amine | |
| 1.190 | 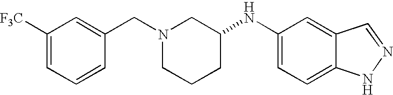 | 1.190 |
| | (R)-N-(1-(3-(trifluoromethyl)benzyl)piperidin-3-yl)-1H- | |
| | indazol-5-amine | |
| 1.191 | 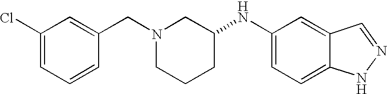 | 1c, 7, 8, 9, 10 |
| | (R)-N-(1-(3-chlorobenzyl)piperidin-3-yl)-1H-indazol-5- | |
| | amine | |
| 1.192 | 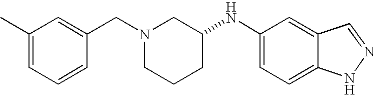 | 1c, 7, 8, 9, 10 |
| | (R)-N-(1-(3-methylbenzyl)piperidin-3-yl)-1H-indazol-5- | |
| | amine | |
| 1.193 | 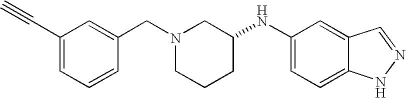 | 1c, 7, 8, 9, 10, 11, 14c |
| | (R)-N-(1-(3-ethynylbenzyl)piperidin-3-yl)-1H-indazol-5- | |
| | amine | |
| 1.194 | 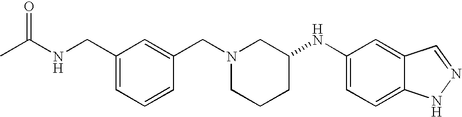 | 1c, 7, 8, 9, 10, 13, 16c |
| | (R)-N-(1H-indazol-5-ylamino)piperidin-1- | |
| | yl)methyl)benzyl)acetamide | |
| 1.195 | 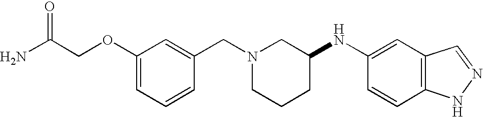 | 1c, 7, 8, 9, 10, 12a, 15c, 15d |
| | (S)-2-(3-((3-(1H-indazol-5-ylamino)piperidin-1- | |
| | yl)methyl)phenyl-1H-indazol-5-ylamino)piperidin-1- | |
| | yl)methyl)phenoxy)acetamide | |
| 1.196 | 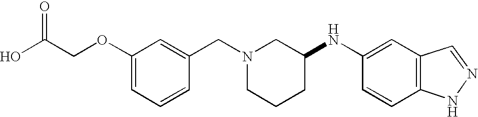 | 1c, 7, 8, 9, 10, 12a, 15c, 15s |
| | (S)-2-(3-((3-(1H-indazol-5-ylamino)piperidin-1- | |
| | yl)methyl)phenoxy)acetic acid | |
| 1.197 | 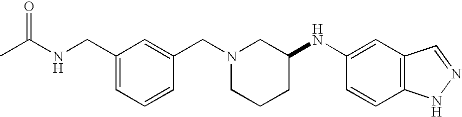 | 11c, 7, 8, 9, 10, 13, 16c |
| | (S)-N-(1H-indazol-5-ylamino)piperidin-1- | |
| | yl)methyl)benzyl)acetamide | |
| 1.198 | 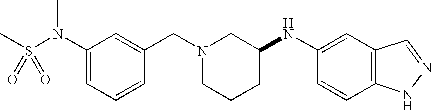 | 1c, 7, 8, 9, 10, 12a, 15c, 15d |
| | (S)-N-(3-((3-(1H-indazol-5-ylamino)piperidin-1- | |
| | yl)methyl)phenyl)-N-methylmethanesulfonamide | |
| 1.199 | 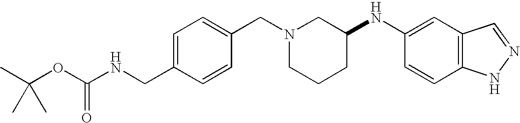 | 1c, 7, 8, 9, 10, 13, 16c |
| | (S)-tert-butyl 4-((3-(1H-indazol-5-ylamino)piperidin-1- | |
| | yl)methyl)benzylcarbamate | |
| 1.200 | 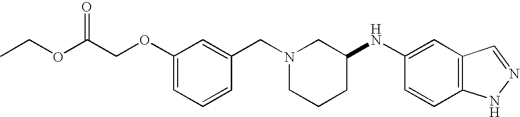 | 1c, 7, 8, 9, 10, 12a, 15c, 15d |
| | (S)-ethyl 2-(3-((3-(1H-indazol-5-ylamino)piperidin-1- | |
| | yl)methyl)phenoxy)acetate | |
| 1.201 | 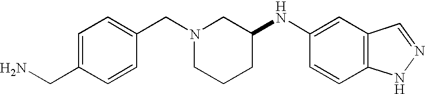 | 1c, 7, 8, 9, 10, 13, 16c |
| | (S)-N-(1-(4-(aminomethyl)benzyl)piperidin-3-yl)-1H- | |
| | indazol-5-amine | |
| 1.202 | 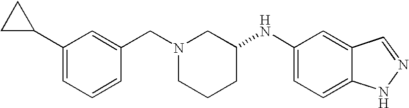 | 1c, 7, 8, 9, 10, 11, 14c |
| | (R)-N-(1-(3-cyclopropylbenzyl)piperidin-3-yl)-1H- | |
| | indazol-5-amine | |
| 1.203 | 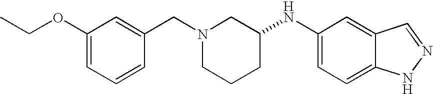 | 1c, 7, 8, 9, 10 |
| | (R)-N-(1-(3-ethoxybenzyl)piperidin-3-yl)-1H-indazol-5- | |
| | amine | |
| 1.204 | 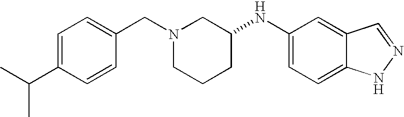 | 1c, 7, 8, 9, 10 |
| | (R)-N-(1-(4-isopropylbenzyl)piperidin-3-yl)-1H-indazol- | |
| | 5-amine | |
| 1.205 | 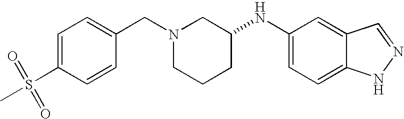 | 1c, 7, 8, 9, 10, 12, 15c |
| | (R)-N-(1-(4-(methylsulfonyl)benzyl)piperidin-3-yl)-1H- | |
| | indazol-5-amine | |
| 1.206 | 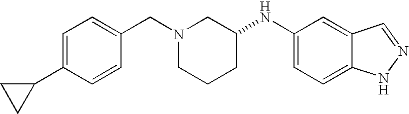 | 1c, 7, 8, 9, 10, 11, 14c |
| | (R)-N-(1-(4-cyclopropylbenzyl)piperidin-3-yl)-1H- | |
| | indazol-5-amine | |
| 1.207 | 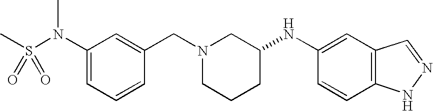 | 1c, 7, 8, 9, 10, 12a, 15c, 15d |
| | (R)-N-(3-((3-(1H-indazol-5-ylamino)piperidin-1- | |
| | yl)methyl)phenyl)-N-methylmethanesulfonamide | |
| 1.208 | 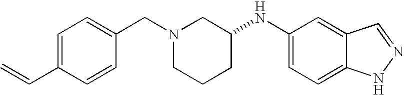 | 1c, 7, 8, 9 10, 11, 14c |
| | (R)-N-(1-(4-vinylbenzyl)piperidin-3-yl)-1H-indazol-5- | |
| | amine | |
| 1.209 | 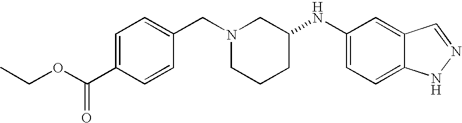 | 1c, 7, 8, 9, 10 |
| | (R)-ethyl 4-((3-(1H-indazol-5-ylamino)piperidin-1- | |
| | yl)methyl)benzoate | |
| 1.210 | 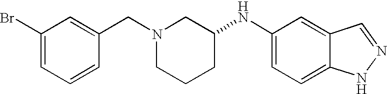 | 1c, 7, 8, 9, 10 |
| | (R)-N-(1-(3-bromobenzyl)piperidin-3-yl)-1H-indazol-5- | |
| | amine | |
| 1.211 | 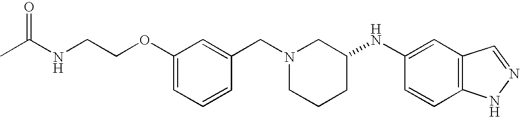 | 1c, 7, 8, 9, 10, 12a, 15c, 15d |
| | (R)-N-(2-(3-((3-(1H-indazol-5-ylamino)piperidin-1- | |
| | yl)methyl)phenoxy)ethyl)acetamide | |
| 1.212 | 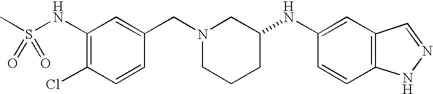 | 1c, 7, 8, 9, 10, 12a, 15c, 15d |
| | (R)-N-(5-((3-(1H-indazol-5-ylamino)piperidin-1- | |
| | yl)methyl)-2-chlorophenyl)methanesulfonamide | |
| 1.213 | 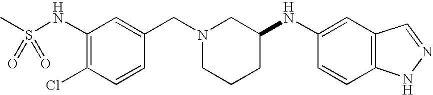 | 1c, 7, 8, 9, 10, 12a, 15c, 15d |
| | (S)-N-(5-((3-(1H-indazol-5-ylamino)piperidin-1- | |
| | yl)methyl)-2-chlorophenyl)methanesulfonamide | |
| 1.214 | 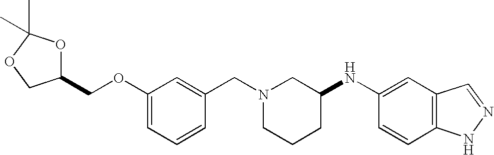 | 1c, 7, 8, 9, 10, 12a, 15c, 15d |
| | N-((S)-1-(3-(((S)-2,2-dimethyl-1,3-dioxolan-4- | |
| | yl)methoxy)benzyl)piperidin-3-yl)-1H-indazol-5-amine | |
| 1.215 | 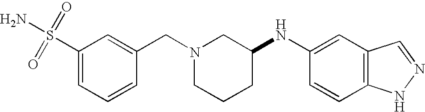 | 1c, 7, 8, 9, 10, 12, 15c |
| | (S)-3-((3-(1H-indazol-5-ylamino)piperidin-1- | |
| | yl)methyl)benzenesulfonamide | |
| 1.216 | 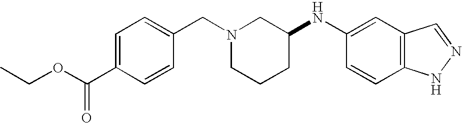 | 1c, 7, 8, 9, 10 |
| | (S)-ethyl 4-((3-(1H-indazol-5-ylamino)piperidin-1- | |
| | yl)methyl)benzoate | |
| 1.217 | 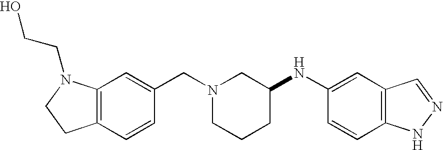 | 1c, 7, 8, 9, 10, 13, 16c |
| | (S)-2-(6-((3-(1H-indazol-5-ylamino)piperidin-1- | |
| | yl)methyl)indolin-1-yl)ethanol | |
| 1.218 | 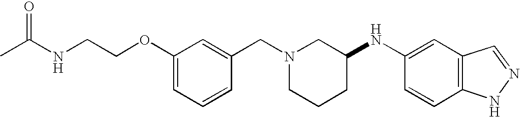 | 1c, 7, 8, 9, 10, 12a, 15c, 15d |
| | (S)-N-(2-(3-((3-(1H-indazol-5-ylamino)piperidin-1- | |
| | yl)methyl)phenoxy)ethyl)acetamide | |
| 1.219 | 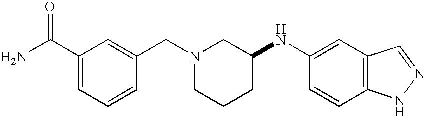 | 1c, 7, 8, 9, 10, 12, 15c |
| | (8)-3-((3-(1H-indazol-5-ylamino)piperidin-1- | |
| | yl)methyl)benzamide | |
| 1.221 | 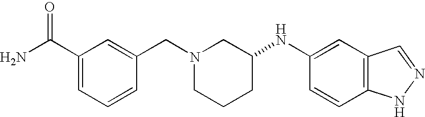 | 1c, 7, 8, 9, 10, 12, 15c |
| | (R)-3-((3-(1H-indazol-5-ylamino)piperidin-1- | |
| | yl)methyl)benzamide | |
| 1.222 | 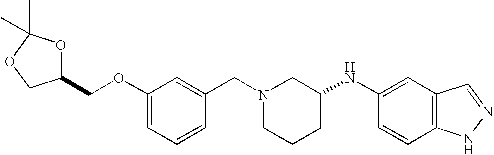 | 1c, 7, 8, 9, 10, 12a, 15c, 15d |
| | N-((R)-1-(3-(((S)-2,2-dimethyl-1,3-dioxolan-4- | |
| | yl)methoxy)benzyl)piperidin-3-yl)-1H-indazol-5-amine | |
| 1.223 | 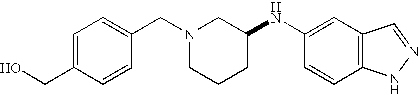 | 1c, 7, 8, 9, 10, 13, 16c |
| | (S)-(4-((3-(1H-indazol-5-ylamino)piperidin-1- | |
| | yl)methyl)phenyl)methanol | |
| 1.224 | 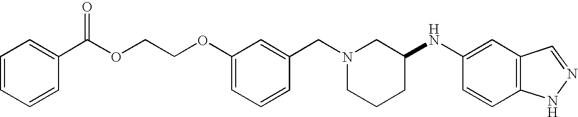 | 1c, 7, 8, 9, 10, 12a, 15c, 15d |
| | (S)-2-(3-((3-(1H-indazol-5-ylamino)piperidin-1- | |
| | yl)methyl)phenoxy)ethyl benzoate | |
| 1.225 | 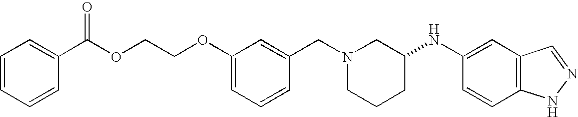 | 1c, 7, 8, 9, 10, 12a, 15c, 15d |
| | (R)-2-(3-((3-(1H-indazol-5-ylamino)piperidin-1- | |
| | yl)methyl)phenoxy)ethyl benzoate | |
| 1.226 | 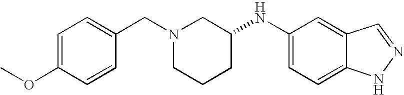 | 1c, 7, 8, 9, 10 |
| | (R)-N-(1-(4-methoxybenzyl)piperidin-3-yl)-1H-indazol- | |
| | 5-amine | |
| 1.227 | 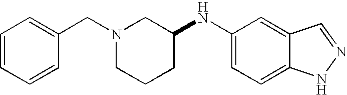 | 1c, 7, 8, 9, 10 |
| | (S)-N-(1-benzylpiperidin-3-yl)-1H-indazol-5-amine | |
| 1.228 | 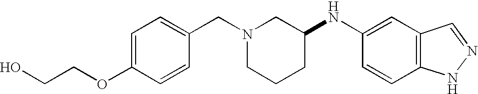 | 1c, 7, 8, 9, 10, 12a, 15c, 15d |
| | (S)-2-(4-((3-(1H-indazol-5-ylamino)piperidin-1- | |
| | yl)methyDphenoxy)ethanol | |
| 1.229 | 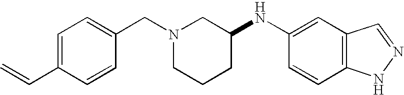 | 1c, 7, 8, 9, 10, 11, 14c |
| | (S)-N-(1-(4-vinylbenzyl)piperidin-3-yl)-1H-indazol-5- | |
| | amine | |
| 1.230 | 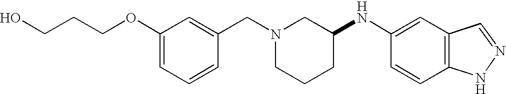 | 1c, 7, 8, 9, 10, 12a, 15c, 15d |
| | (S)-3-(3-((3-(1H-indazol-5-ylamino)piperidin-1- | |
| | yl)methyl)phenoxy)propan-1-ol | |
| 1.231 | 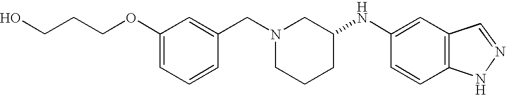 | 1c, 7, 8, 9, 10, 12a, 15c, 15d |
| | (R)-3-(3-((3-(1H-indazol-5-ylamino)piperidin-1- | |
| | yl)methyl)phenoxy)propan-1-ol | |
| 1.232 | 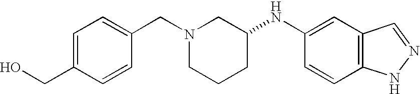 | 1c, 7, 8, 9, 10 |
| | (R)-(4-((3-(1H-indazol-5-ylamino)piperidin-1- | |
| | yl)methyl)phenyl)methanol | |
| 1.233 | 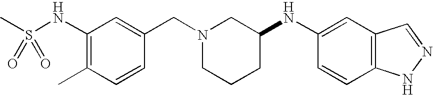 | 1c, 7, 8, 9, 10, 12a, 15c, 15d |
| | (S)-N-(5-((3-(1H-indazol-5-ylamino)piperidin-1- | |
| | yl)methyl)-2-methylphenyl)methanesulfonamide | |
| 1.234 | 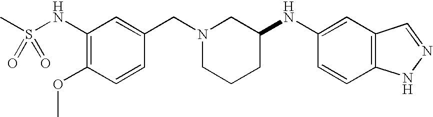 | 1c, 7, 8, 9, 10, 12a, 15c, 15d |
| | (S)-N-(5-((3-(1H-indazol-5-ylamino)piperidin-1- | |
| | yl)methyl)-2-methoxyphenyl)methanesulfonamide | |
| 1.235 | 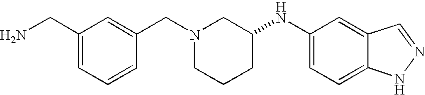 | 1c, 7, 8, 9, 10, 13, 16c |
| | (R)-N-(1-(3-(aminomethyl)benzyl)piperidin-3-yl)-1H- | |
| | indazol-5-amine | |
| 1.236 | 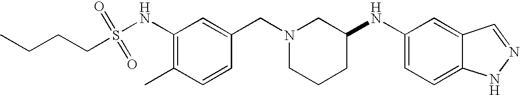 | 1c, 7, 8, 9, 10, 12a, 15c,15d |
| | (S)-N-(5-((3-(1H-indazol-5-ylamino)piperidin-1- | |
| | yl)methyl)-2-methylphenyl)butane-1-sulfonamide | |
| 1.237 | 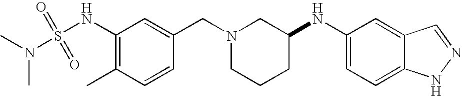 | 1c, 7, 8, 9, 10, 12a, 15c, 15d |
| | (S)-N-(2-((3-(1H-indazol-5-ylamino)piperidin-1- | |
| | yl)methyl)-5-methylphenyl)-N′,N′ | |
| | dimethylaminosulfamide | |
| 1.238 | 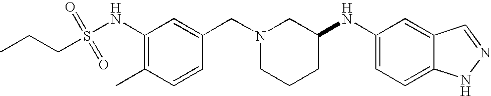 | 1c, 7, 8, 9, 10, 12a, 15c, 15d |
| | (S)-N-(5-((3-(1H-indazol-5-ylamino)piperidin-1- | |
| | yl)methyl)-2-methylphenyl)propane-1-sulfonamide | |
| 1.239 | 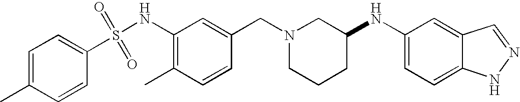 | 1c, 7, 8, 9, 10, 12a, 15c, 15d |
| | (S)-N-(5-((3-(1H-indazol-5-ylamino)piperidin-1- | |
| | yl)methyl)-2-methylphenyl)-4- | |
| | methylbenzenesulfonamide | |
| 1.240 | 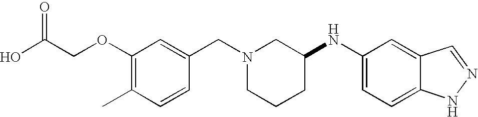 | 1c, 7, 8, 9, 10, 12a, 15c, 15d |
| | (S)-2-(5-((3-(1H-indazol-5-ylamino)piperidin-1- | |
| | yl)methyl)-2-methylphenyl-1H-indazol-5- | |
| | ylamino)piperidin-1-yl)methyl)-2-methylphenoxy)acetic | |
| | acid | |
| 1.241 | 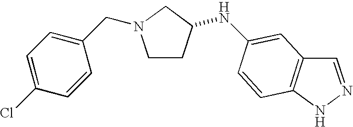 | 1c, 7, 8, 9, 10 |
| | (R)-N-(1-(4-chlorobenzyl)pyrrolidin-3-yl)-1H-indazol-5- | |
| | amine | |
| 1.242 | 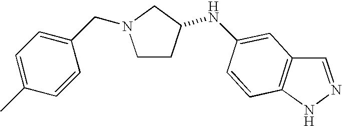 | 1c, 7, 8, 9, 10 |
| | (R)-N-(1-(4-methylbenzyl)pyrrolidin-3-yl)-1H-indazol-5- | |
| | amine | |
| 1.243 | 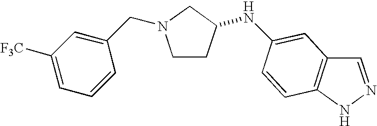 | 1c, 7, 8, 9, 10 |
| | (R)-N-(1-(3-(trifluoromethyl)benzyl)pyrrolidin-3-yl)-1H- | |
| | indazol-5-amine | |
| 1.244 | 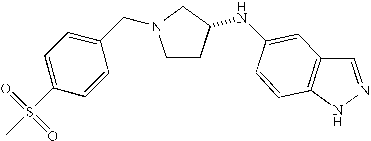 | 1c, 7, 8, 9, 10, 12b, 15c, 15e |
| | (R)-N-(1-(4-(methylsulfonyl)benzyl)pyrrolidin-3-yl)-1H- | |
| | indazol-5-amine | |
| 1.245 | 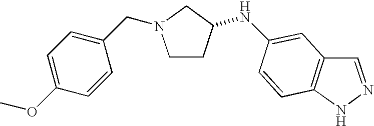 | 1c, 7, 8, 9, 10 |
| | (R)-N-(1-(4-methoxybenzyl)pyrrolidin-3-yl)-1H-indazol- | |
| | 5-amine | |
| 1.246 | 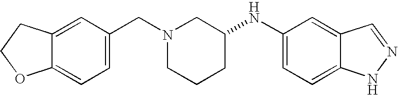 | 1c, 7, 8, 9, 10 |
| | (R)-N-(1-((2,3-dihydrobenzofuran-5- | |
| | yl)methyl)piperidin-3-yl)-1H-indazol-5-amine | |
| 1.247 | 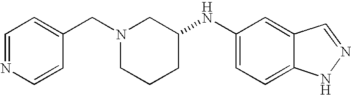 | 1c, 7, 8, 9, 10 |
| | (R)-N-(1-(pyridin-4-ylmethyl)piperidin-3-yl)-1H-indazol | |
| | 5-amine | |
| 1.248 | 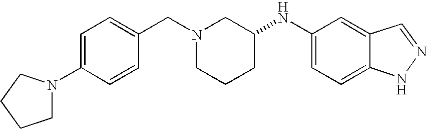 | 1c, 7, 8, 9, 10, 11, 14c |
| | (R)-N-(1-(4-(pyrrolidin-1-yl)benzyl)piperidin-3-yl)-1H- | |
| | indazol-5-amine | |
| 1.249 | 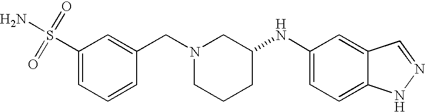 | 1c, 7, 8, 9, 10, 12b, 15c, 15e |
| | (R)-3-((3-(1H-indazol-5-ylamino)piperidin-1- | |
| | yl)methyl)benzenesulfonamide | |
| 1.250 | 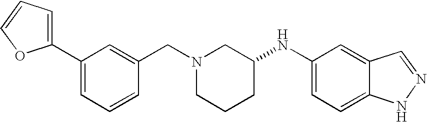 | 1c, 7, 8, 9, 10, 11, 14c |
| | (R)-N-(1-(3-(furan-2-yl)benzyl)piperidin-3-yl)-1H- | |
| | indazol-5-amine | |
| 1.251 | 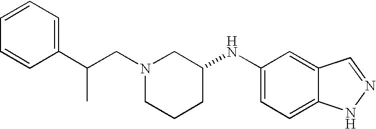 | 1c, 7, 8, 9 |
| | N-((3R)-1-(2-phenylpropyl)piperidin-3-yl)-1H-indazol- | |
| | 5-amine | |
| 1.252 | 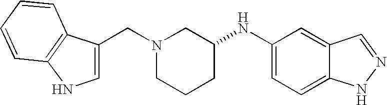 | 1c, 7, 8, 9, 10 |
| | (R)-N-(1-((1H-indol-3-yl)methyl)piperidin-3-yl)-1H- | |
| | indazol-5-amine | |
| 1.253 | 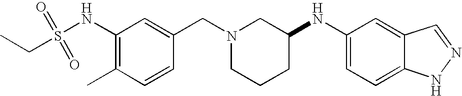 | 1c, 7, 8, 9, 10, 12a, 15c, 15d |
| | (S)-N-(5-((3-(1H-indazol-5-ylamino)piperidin-1- | |
| | yl)methyl)-2-methylphenyl)ethanesulfonamide | |
| 1.254 | 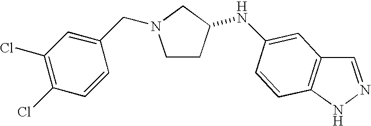 | 1c, 7, 8, 9, 10 |
| | (R)-N-(1-(3,4-dichlorobenzyl)pyrrolidin-3-yl)-1H- | |
| | indazol-5-amine | |
| 1.255 | 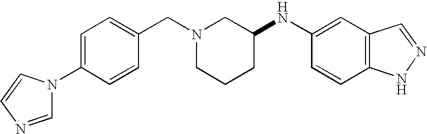 | 1c, 7, 8, 9, 10, 11, 14c |
| | (S)-N-(1-(1H-imidazol-1-yl)benzyl)piperidin-3-yl)-1H- | |
| | indazol-5-amine | |
| 1.256 | 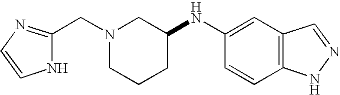 | 1c, 7, 8, 9, 10 |
| | (S)-N-( 1-((1H-imidazol-2-yl)methyl)piperidin-3-yl)-1H- | |
| | indazol-5-amine | |
| 1.257 | 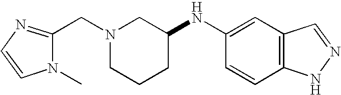 | 1c, 7, 8, 9, 10 |
| | (S)-N-(1-((1-methyl-1H-imidazol-2-yl)methyl)piperidin- | |
| | 3-yl)-1H-indazol-5-amine | |
| 1.258 | 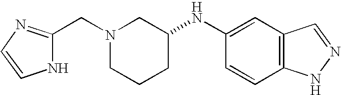 | 1c, 7, 8, 9, 10, 12a, 15c, 15d |
| | (R)-N-(5-((3-(1H-indazol-5-ylamino)piperidin-1- | |
| | yl)methyl)-2-methylphenyl)methanesulfonamide | |
| 1.259 | 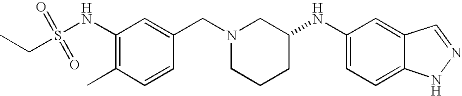 | 1c, 7, 8, 9, 10, 12a, 15c, 15d |
| | (R)-N-(5-((3-(1H-indazol-5-ylamino)piperidin-1- | |
| | yl)methyl)-2-methylphenyl)ethanesulfonamide | |
| 1.260 | 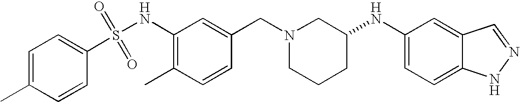 | 1c, 7, 8, 9, 10, 12a, 15c, 15d |
| | (R)-N-(5-((3-(1H-indazol-5-ylamino)piperidin-1- | |
| | yl)methyl)-2-methylphenyl)-4- | |
| | methylbenzenesulfonamide | |
| 1.261 | 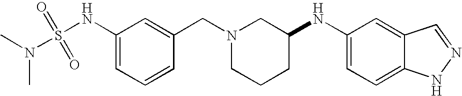 | 1c, 7, 8, 9, 10, 12a, 15c, 15d |
| | (S)-N-(3-((3-(1H-indazol-5-ylamino)piperidin-1- | |
| | yl)methyl)phenyl)-N′,N′-dimethylaminosulfamide | |
| 1.262 | 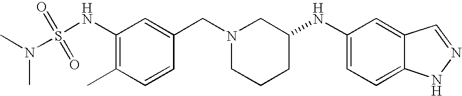 | 1c, 7, 8, 9, 10, 12a, 15c, 15d |
| | (R)-N-(2-((3-(1H-indazol-5-ylamino)piperidin-1- | |
| | yl)methyl)-5-methylphenyl)-N′,N′ | |
| | dimethylaminosulfamide | |
| 1.263 | 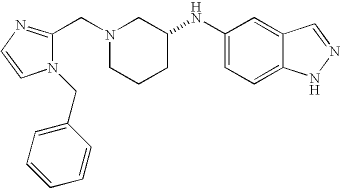 | 1c, 7, 8, 9, 10, 11, 14c |
| | (R)-N-(1-((1-benzyl-1H-imidazol-2-yl)methyl)piperidin- | |
| | 3-yl)-1H-indazol-5-amine | |
| 1.264 | 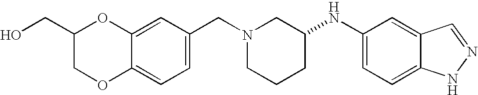 | 1c, 7, 8, 9, 10, 13, 16c |
| | (7-(((R)-3-(1H-indazol-5-ylamino)piperidin-1- | |
| | yl)methyl)-2,3-dihydrobenzo[b][1,4]dioxin-2- | |
| | yl)methanol | |
| 1.265 | 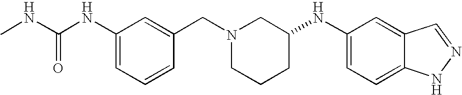 | 1c, 7, 8, 9, 10, 12a, 15c, 15d |
| | (R)-1-(3-((3-(1H-indazol-5-ylamino)piperidin-1- | |
| | yl)methyl)phenyl)-3-methylurea | |
| 1.266 | 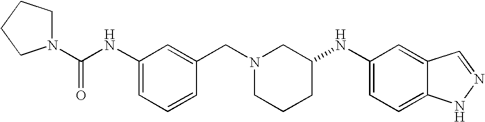 | 1c, 7, 8, 9, 10, 12a, 15c, 15d |
| | (R)-N-(3-((3-(1H-indazol-5-ylamino)piperidin-1- | |
| | yl)methyl)phenyl)pyrrolidine-1-carboxamide | |
| 1.267 | 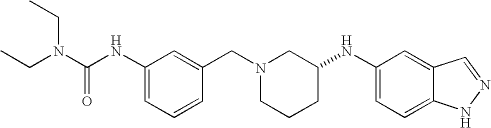 | 1c, 7, 8, 9, 10, 12a, 15c, 15d |
| | (R)-3-(3-((3-(1H-indazol-5-ylamino)piperidin-1- | |
| | yl)methyl)phenyl)-1,1-diethylurea | |
| 1.268 | 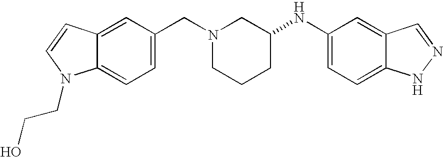 | 1c, 7, 8, 9, 10, 13, 16c |
| | (R)-2-(5-((3-(1H-indazol-5-ylamino)piperidin-1- | |
| | yl)methyl)-1H-indol-1-yl)ethanol | |
| 1.269 | 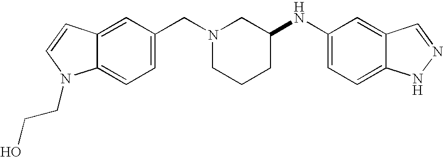 | 1c, 7, 8, 9, 10, 13, 16c |
| | (S)-2-(5-((3-(1H-indazol-5-ylamino)piperidin-1- | |
| | yl)methyl)-1H-indol-1-yl)ethanol | |
| 1.270 | 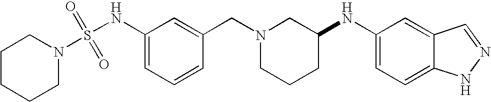 | 1c, 7, 8, 9, 10, 12a, 15c, 15d |
| | (S)-N-(3-((3-(1H-indazol-5-ylamino)piperidin-1- | |
| | yl)methyl)phenyl)piperidine-1-sulfonamide | |
| 1.271 | 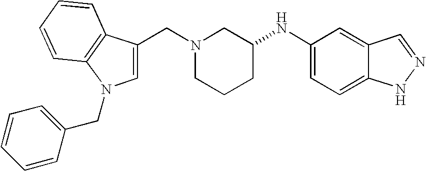 | 1c, 7, 8, 9, 10, 11, 14c |
| | (R)-N-(11-((1-benzyl-1H-indol-3-yl)methyl)piperidin-3- | |
| | yl)-1H-indazol-5-amine | |
| 1.272 | 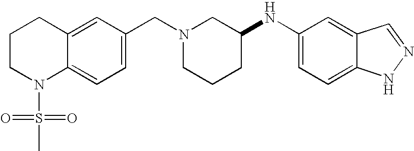 | 1c, 7, 8, 9, 10, 12b, 15c, 15e |
| | (S)-N-(1-((1-(methylsulfonyl)-1,2,3,4- | |
| | tetrahydroquinolin-6-yl)methyl)piperidin-3-yl)-1H- | |
| | indazol-5-amine | |
| 1.273 | 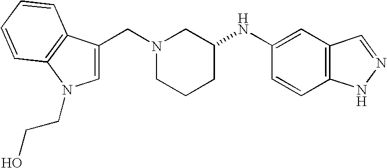 | 1c, 7, 8, 9, 10, 13, 16c |
| | (R)-2-(3-((3-(1H-indazol-5-ylamino)piperidin-1- | |
| | yl)methyl)-1H-indol-1-yl)ethanol | |
| 1.274 | 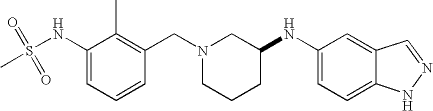 | 1c, 7, 8, 9, 10, 12a, 15c, 15d |
| | (S)-N-(3-((3-(1H-indazol-5-ylamino)piperidin-1- | |
| | yl)methyl)-2-methylphenyl)methanesulfonamide | |
| 1.275 | 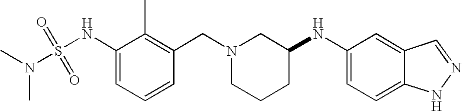 | 1c, 7, 8, 9, 10, 12a, 15c, 15d |
| | (S)-N-(3-((3-(1H-indazol-5-ylamino)piperidin-1- | |
| | yl)methyl)-2-methylphenyl)-N′,N′ | |
| | dimethylaminosulfamide | |
| 1.276 | 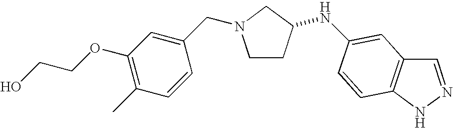 | 1c, 7, 8, 9, 10, 12a, 15c, 15d |
| | (R)-2-(5-((3-(1H-indazol-5-ylamino)pyrrolidin-1- | |
| | yl)methyl)-2-methylphenyl-1H-indazol-5- | |
| | ylamino)pyrrolidin-1-yl)methyl)-2- | |
| | methylphenoxy)ethanol | |
| 1.277 | 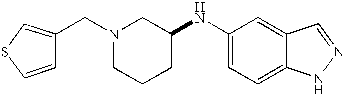 | 1c, 7, 8, 9, 10 |
| | (S)-N-(1-(thiophen-3-ylmethyl)piperidin-3-yl)-1H- | |
| | indazol-5-amine | |
| 1.278 | 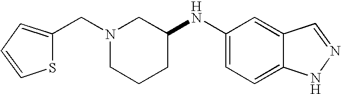 | 1c, 7, 8, 9, 10 |
| | (S)-N-(1-(thiophen-2-ylmethyl)piperidin-3-yl)-1H- | |
| | indazol-5-amine | |
| 1.279 | 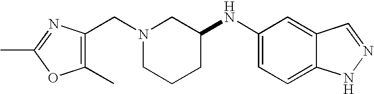 | 1c, 7, 8, 9, 10 |
| | (S)-N-(1-((2,5-dimethyloxazol-4-yl)methyl)piperidin-3- | |
| | yl)-1H-indazol-5-amine | |
| 1.280 | 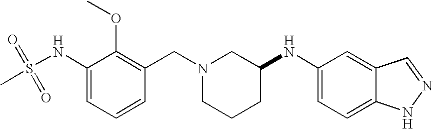 | 1c, 7, 8, 9, 10, 12a, 15c, 15d |
| | (S)-N-(3-((3-(1H-indazol-5-ylamino)piperidin-1- | |
| | yl)methyl)-2-methoxyphenyl)methanesulfonamide | |
| 1.281 | 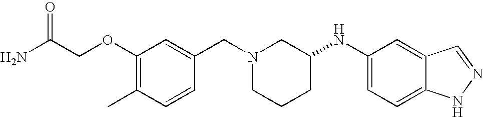 | 1c, 7, 8, 9, 10, 12a, 15c, 15d |
| | (R)-2-(5-((3-(1H-indazol-5-ylamino)piperidin-1- | |
| | yl)methyl)-2-methylphenyl-1H-indazol-5- | |
| | ylamino)piperidin-1-yl)methyl)-2- | |
| | methylphenoxy)acetamide | |
| 1.282 | 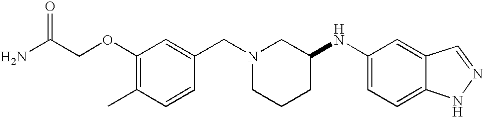 | 1c, 7, 8, 9, 10, 12a, 15c, 15d |
| | (S)-2-(5-((3-(1H-indazol-5-ylamino)piperidin-1- | |
| | yl)methyl)-2-methylphenyl-1H-indazol-5- | |
| | ylamino)piperidin-1-yl)methyl)-2- | |
| | methylphenoxy)acetamide | |
| 2.001 | 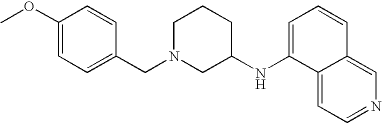 | 2c, 7, 8, 9, 10 |
| | N-(1-(4-methoxybenzyl)piperidin-3-yl)isoquinolin-5- | |
| | amine | |
| 2.002 | 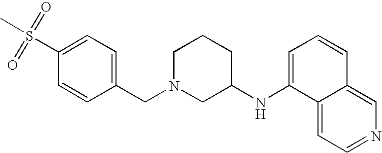 | 2c, 7, 8, 9, 10, 12, 15c |
| | N-(1-(4-(methylsulfonyl)benzyl)piperidin-3- | |
| | yl)isoquinolin-5-amine | |
| 2.003 | 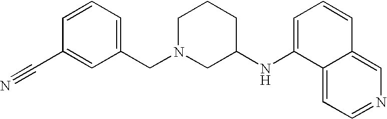 | 2c, 7, 8, 9, 10, 12, 15c |
| | 3-((3-(isoquinolin-5-ylamino)piperidin-1- | |
| | yl)methyl)benzonitrile | |
| 2.004 | 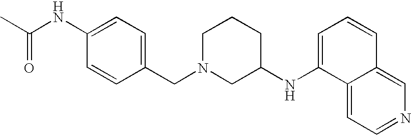 | 2c, 7, 8, 9, 10, 12a, 15c, 15d |
| | N-(4-((3-(isoquinolin-5-ylamino)piperidin-1- | |
| | yl)methyl)phenyl)acetamide | |
| 2.005 | 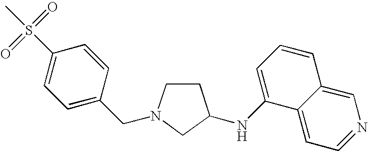 | 2c, 7, 8, 9, 10, 12, 15c |
| | N-(1-(4-(methylsulfonyl)benzyl)pyrrolidin-3- | |
| | yl)isoquinolin-5-amine | |
| 2.006 | 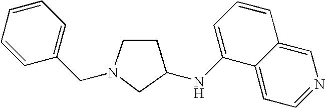 | 2c, 7, 8, 9, 10 |
| | N-(1-benzylpyrrolidin-3-yl)isoguinolin-5-amine | |
| 2.007 | 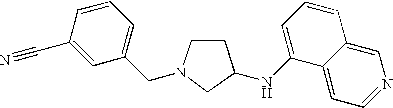 | 2c, 7, 8, 9, 10, 12, 15c |
| | 3-((3-(isoquinolin-5-ylamino)pyrrolidin-1- | |
| | yl)methyl)benzonitrile | |
| 2.008 | 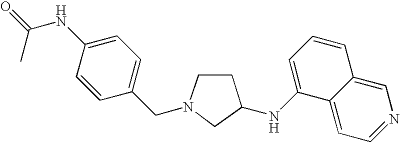 | 2c, 7, 8, 9, 10, 12a, 15c, 15d |
| | N-(4-((3-(isoquinolin-5-ylamino)pyrrolidin-1- | |
| | yl)methyl)phenyl)acetamide | |
| 2.009 | 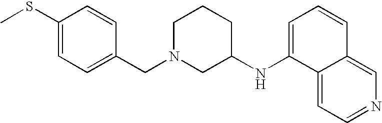 | 2c, 7, 8, 9, 10, 12b, 15c, 15e |
| | N-(1-(4-(methylthio)benzyl)piperidin-3-yl)isoquinolin-5- | |
| | amine | |
| 2.010 | 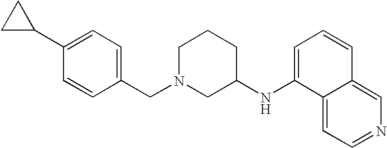 | 2c, 7, 8, 9, 10, 11, 14c |
| | N-(1-(4-cyclopropylbenzyl)piperidin-3-yl)isoquinolin-5- | |
| | amine | |
| 2.011 | 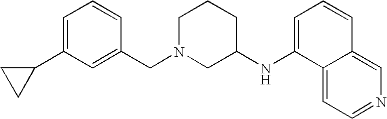 | 2c, 7, 8, 9, 10, 11, 14c |
| | N-(1-(3-cyclopropylbenzyl)piperidin-3-yl)isoquinolin-5- | |
| | amine | |
| 2.012 | 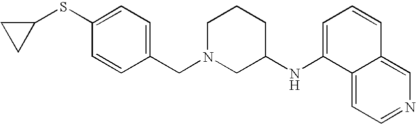 | 2c, 7, 8, 9, 10, 12b, 15c, 15e |
| | N-(1-(4-(cyclopropylthio)benzyl)piperidin-3- | |
| | yl)isoquinolin-5-amine | |
| 2.013 | 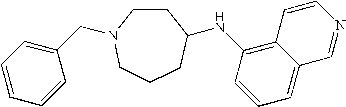 | 2c, 7, 8, 9, 10 |
| | N-(1-benzylazepan-4-yl)isoquinolin-5-amine | |
| 2.014 | 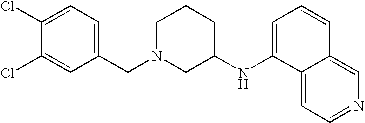 | 2c, 7, 8, 9, 10 |
| | N-(1-(3, 4-dichlorobenzyl)piperidin-3-yl)isoquinolin-5- | |
| | amine | |
| 2.015 | 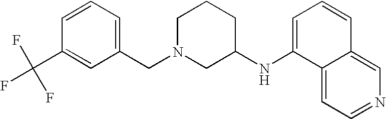 | 2c, 7, 8, 9, 10 |
| | N-(1-(3-(trifluoromethyl)benzyl)piperidin-3- | |
| | yl)isoquinolin-5-amine | |
| 2.016 | 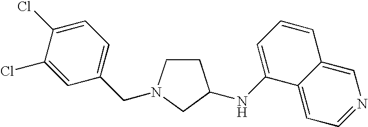 | 2c, 7, 8, 9, 10 |
| | N-(1-(3,4-dichlorobenzyl)pyrrolidin-3-yl)isoquinolin-5- | |
| | amine | |
| 2.017 | 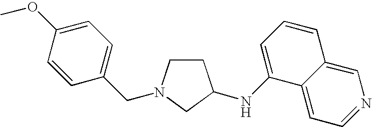 | 2c, 7, 8, 9, 10 |
| | N-(1-(4-methoxybenzyl)pyrrolidin-3-yl)isoquinolin-5- | |
| | amine | |
| 2.018 | 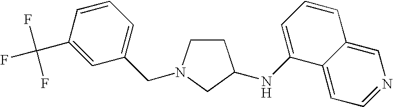 | 2c, 7, 8, 9, 10 |
| | N-(1-(3-(trifluoromethyl)benzyl)pyrrolidin-3- | |
| | yl)isoquinolin-5-amine | |
| 2.019 | 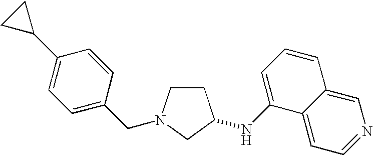 | 2c, 7, 8, 9, 10, 11, 14c |
| | (S)-N-(1-(4-cyclopropylbenzyl)pyrrolidin-3- | |
| | yl)isoquinolin-5-amine | |
| 2.020 | 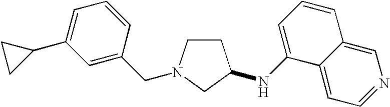 | 2c, 7, 8, 9, 10, 11, 14c |
| | (R)-N-(1-(3-cyclopropylbenzyl)pyrrolidin-3- | |
| | yl)isoquinolin-5-amine | |
| 2.021 | 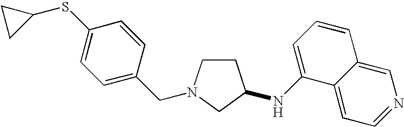 | 2c, 7, 8, 9, 10, 12b, 15c, 15e |
| | (R)-N-(1-(4-(cyclopropylthio)benzyl)pyrrolidin-3- | |
| | yl)isoquinolin-5-amine | |
| 2.022 | 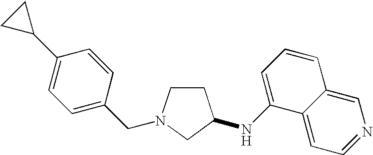 | 2c, 7, 8, 9, 10, 11, 14c |
| | (R)-N-(1-(4-cyclopropylbenzyl)pyrrolidin-3- | |
| | yl)isoquinolin-5-amine | |
| 2.023 | 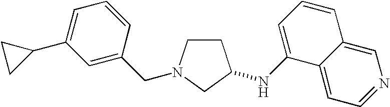 | 2c, 7, 8, 9, 10, 11, 14c |
| | (S)-N-(1-(3-cyclopropylbenzyl)pyrrolidin-3- | |
| | yl)isoquinolin-5-amine | |
| 2.024 | 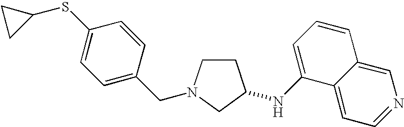 | 2c, 7, 8, 9, 10, 12b, 15c, 15e |
| | (S)-N-(1-(4-(cyclopropylthio)benzyl)pyrrolidin-3- | |
| | yl)isoquinolin-5-amine | |
| 2.025 | 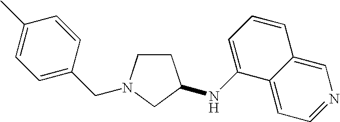 | 2c, 7, 8, 9, 10 |
| | (R)-N-(1-(4-methylbenzyl)pyrrolidin-3-yl)isoquinolin-5- | |
| | amine | |
| 2.026 | 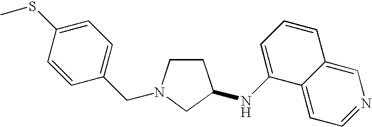 | 2c, 7, 8, 9, 10, 12b, 15c, 15e |
| | (R)-N-(1-(4-(methylthio)benzyl)pyrrolidin-3- | |
| | yl)isoquinolin-5-amine | |
| 2.027 | 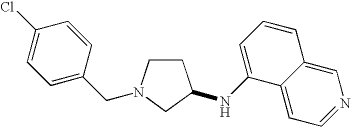 | 2c, 7, 8, 9, 10 |
| | (R)-N-(1-(4-chlorobenzyl)pyrrolidin-3-yl)isoquinolin-5- | |
| | amine | |
| 2.028 | 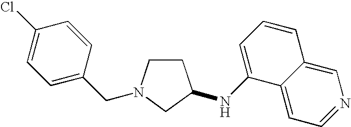 | 2c, 7, 8, 9, 10 |
| | (S)-N-(1-(4-methylbenzyl)pyrrolidin-3-yl)isoquinolin-5- | |
| | amine | |
| 2.029 | 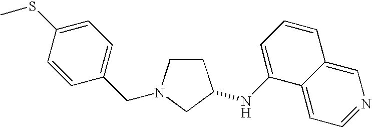 | 2c, 7, 8, 9, 10, 12b, 15c, 15e |
| | (S)-N-(1-(4-(methylthio)benzyl)pyrrolidin-3- | |
| | yl)isoquinolin-5-amine | |
| 2.030 | 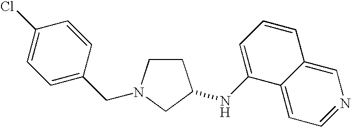 | 2c, 7, 8, 9, 10 |
| | (S)-N-(1-(4-chlorobenzyl)pyrrolidin-3-yl)isoquinolin-5- | |
| | amine | |
| 2.031 | 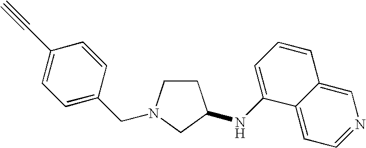 | 2c, 7, 8, 9, 10, 11, 14c |
| | (R)-N-(1-(4-ethynylbenzyl)pyrrolidin-3-yl)isoquinolin-5- | |
| | amine | |
| 2.032 | 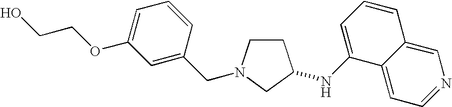 | 2c, 7, 8, 9, 10, 12a, 15c, 15d |
| | (S)-2-(3-((3-(isoquinolin-5-ylamino)pyrrolidin-1- | |
| | yl)methyl)phenoxy)ethanol | |
| 2.033 | 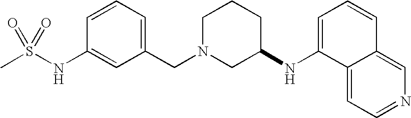 | 2c, 7, 8, 9, 10, 12a, 15c, 15d |
| | (R)-N-(3-((3-(isoquinolin-5-ylamino)piperidin-1- | |
| | yl)methyl)phenyl)methanesulfonamide | |
| 2.034 | 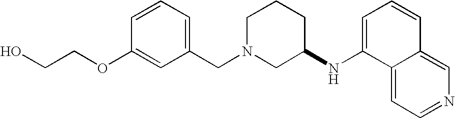 | 2c, 7, 8, 9, 10, 12a, 15c, 15d |
| | (R)-2-(3-((3-(isoquinolin-5-ylamino)piperidin-1- | |
| | yl)methyl)phenoxy)ethanol | |
| 2.035 | 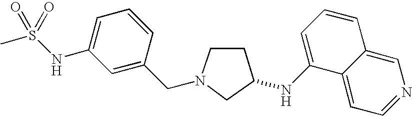 | 2c, 7, 8, 9, 10, 12a, 15c, 15d |
| | (S)-N-(3-((3-(isoquinolin-5-ylamino)pyrrolidin-1- | |
| | yl)methyl)phenyl)methanesulfonamide | |
| 2.036 | 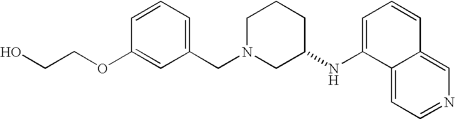 | 2c, 7, 8, 9, 10, 12a, 15c, 15d |
| | (S)-2-(3-((3-(isoquinolin-5-ylamino)piperidin-1- | |
| | yl)methyl)phenoxy)ethanol | |
| 2.037 | 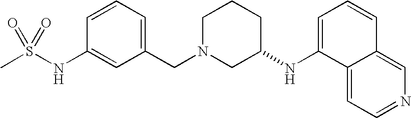 | 2c, 7, 8, 9, 10, 12a, 15c, 15d |
| | (S)-N-(3-((3-(isoquinolin-5-ylamino)piperidin-1- | |
| | yl)methyl)phenyl)methanesulfonamide | |
| 2.038 | 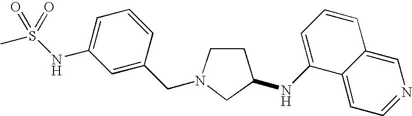 | 2c, 7, 8, 9, 10, 12a, 15c, 15d |
| | (R)-N-(3-((3-(isoquinolin-5-ylamino)pyrrolidin-1- | |
| | yl)methyl)phenyl)methanesulfonamide | |
| 2.039 | 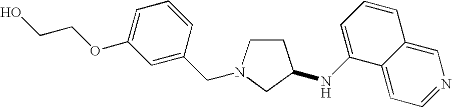 | 2c, 7, 8, 9, 10, 12a, 15c, 15d |
| | (R)-2-(3-((3-(isoquinolin-5-ylamino)pyrrolidin-1- | |
| | yl)methyl)phenoxy)ethanol | |
| 2.040 | 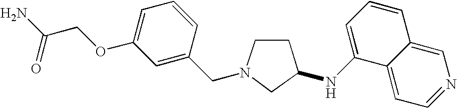 | 2c, 7, 8, 9, 10, 12a, 15c, 15d |
| | (R)-2-(3-((3-(isoquinolin-5-ylamino)pyrrolidin-1- | |
| | yl)methyl)phenoxy)acetamide | |
| 2.041 | 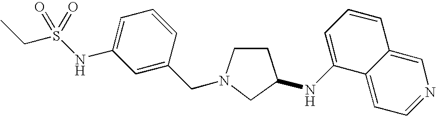 | 2c, 7, 8, 9, 10, 12a, 15c, 15d |
| | (R)-N-(3-((3-(isoquinolin-5-ylamino)pyrrolidin-1- | |
| | yl)methyl)phenyl)ethanesulfonamide | |
| 2.042 | 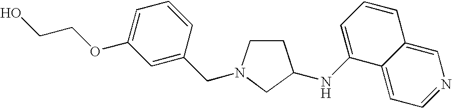 | 2c, 7, 8, 9, 10, 12a, 15c, 15d |
| | 2-(3-((3-(isoquinolin-5-ylamino)pyrrolidin-1- | |
| | yl)methyl)phenoxy)ethanol | |
| 2.043 | 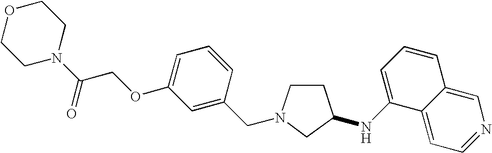 | 2c, 7, 8, 9, 10, 12a, 15c, 15d |
| | (R)-2-(3-((3-(isoquinolin-5-ylamino)pyrrolidin-1- | |
| | yl)methyl)phenoxy)-1-morpholinoethanone | |
| 2.044 | 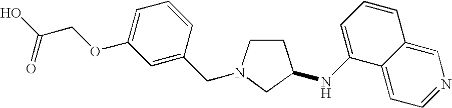 | 2c, 7, 8, 9, 10 12a, 15c, 15d |
| | (R)-2-(3-((3-(isoquinolin-5-ylamino)pyrrolidin-1- | |
| | yl)methyl)phenoxy)acetic acid | |
| 2.045 | 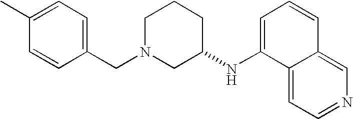 | 2c, 7, 8, 9, 10 |
| | (S)-N-(1-(4-methylbenzyl)piperidin-3-yl)isoquinolin-5- | |
| | amine | |
| 2.046 | 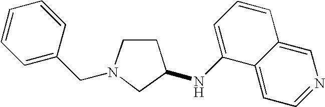 | 2c, 7, 8, 9, 10 |
| | (R)-N-(1-benzylpyrrolidin-3-yl)isoquinolin-5-amine | |
| 2.047 | 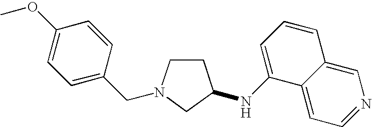 | 2c, 7, 8, 9, 10 |
| | (R)-N-(1-(4-methoxybenzyl)pyrrolidin-3-yl)isoquinolin- | |
| | 5-amine | |
| 2.048 | 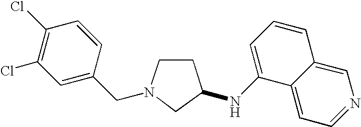 | 2c, 7, 8, 9, 10 |
| | (R)-N-(1-(3,4-dichlorobenzyl)pyrrolidin-3- | |
| | yl)isoquinolin-5-amine | |
| 2.049 | 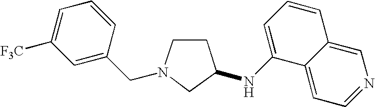 | 2c, 7, 8, 9, 10 |
| | (R)-N-(1-(3-(trifluoromethyl)benzyl)pyrrolidin-3- | |
| | yl)isoquinolin-5-amlne | |
| 2.050 | 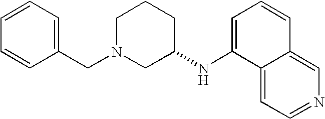 | 2c, 7, 8, 9, 10 |
| | (S)-N-(1-benzylpiperidin-3-yl)isoquinolin-5-amine | |
| 2.051 | 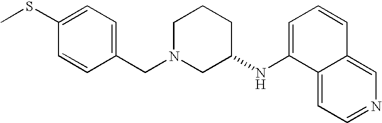 | 2c, 7, 8, 9, 10, 12b, 15c, 15e |
| | (S)-N-(1-(4-(methylthio)benzyl)piperidin-3- | |
| | yl)isoquinolin-5-amine | |
| 2.052 | 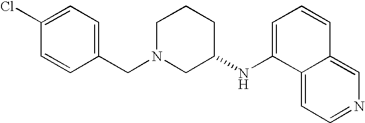 | 2c, 7, 8, 9, 10 |
| | (S)-N-(1-(4-chlorobenzyl)piperidin-3-yl)isoquinolin-5- | |
| | amine | |
| 2.053 | 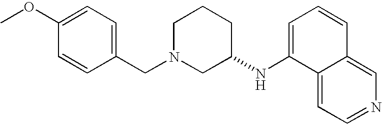 | 2c, 7, 8, 9, 10 |
| | (S)-N-(1-(4-methoxybenzyl)piperidin-3-yl)isoquinolin- | |
| | 5-amine | |
| 2.054 | 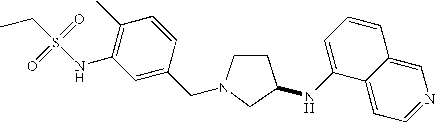 | 2c, 7, 8, 9, 10, 12a, 15c, 15d |
| | (R)-N-(5-((3-(isoquinolin-5-ylamino)pyrrolidin-1- | |
| | yl)methyl)-2-methylphenyl)ethanesulfonamide | |
| 2.055 | 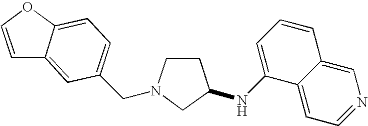 | 2c, 7, 8, 9, 10 |
| | (R)-N-(1-(benzofuran-5-ylmethyl)pyrrolidin-3- | |
| | yl)isoquinolin-5-amine | |
| 2.056 | 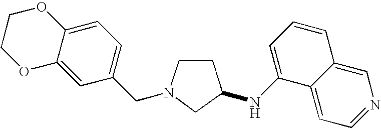 | 2c, 7, 8, 9, 10 |
| | (R)-N-(1-((2,3-dihydrobenzo[b][1,4]dioxin-6- | |
| | yl)methyl)pyrrolidin-3-yl)isoquinolin-5-amine | |
| 2.057 | 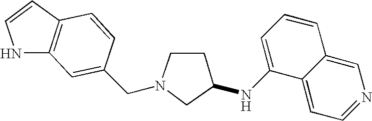 | 2c, 7, 8, 9, 10 |
| | (R)-N-(1-((1H-indol-6-yl)methyl)pyrrolidin-3- | |
| | yl)isoquinolin-5-amine | |
| 2.058 | 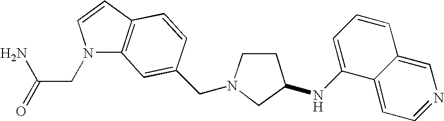 | 2c, 7, 8, 9, 10, 13, 16c |
| | (R)-2-(6-((3-(isoquinolin-5-ylamino)pyrrolidin-1- | |
| | yl)methyl)-1H-indol-1-yl)acetamide | |
| 2.059 | 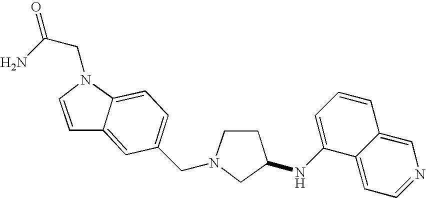 | 2c, 7, 8, 9, 10, 13, 16c |
| | (R)-2-(5-((3-(isoquinolin-5-ylamino)pyrrolidin-1- | |
| | yl)methyl)-1H-indol-1-yl)acetamide | |
| 2.060 | 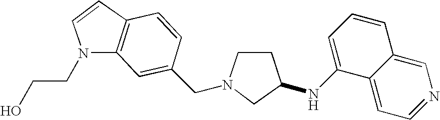 | 2c, 7, 8, 9, 10, 13, 16c |
| | (R)-2-(6-((3-(isoquinolin-5-ylamino)pyrrolidin-1- | |
| | yl)methyl)-1H-indol-1-yl)ethanol | |
| 2.061 | 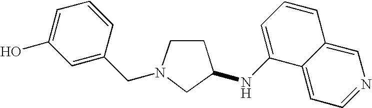 | 2c, 7, 8, 9, 10 |
| | (R)-3-((3-(isoquinolin-5-ylamino)pyrrolidin-1- | |
| | yl)methyl)phenol | |
| 2.062 | 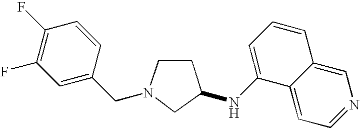 | 2c, 7, 8, 9, 10 |
| | (R)-N-(1-(3,4-difluorobenzyl)pyrrolidin-3-yl)isoquinolin- | |
| | 5-amine | |
| 2.063 | 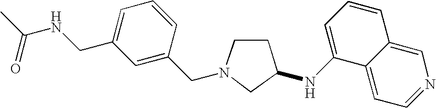 | 2c, 7, 8, 9, 10, 13, |
| | (R)-N-(3-((3-(isoquinolin-5-ylamino)pyrrolidin-1- | |
| | yl)methyl)benzyl)acetamide | |
| 2.064 | 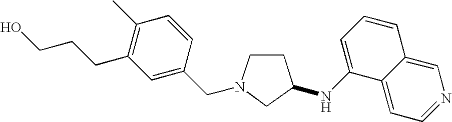 | 2c, 7, 8, 9, 10, 12a, |
| | (R)-2-(5-((3-(isoquinolin-5-ylamino)pyrrolidin-1- | |
| | yl)methyl)-2-methylphenoxy)ethanol | |
| 2.065 | 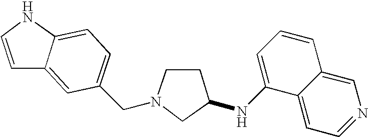 | 2c, 7, 8, 9, 10 |
| | (R)-N-( 1-((1H-indol-5-yl)methyl)pyrrolidin-3- | |
| | yl)isoquinolin-5-amine | |
| 2.066 | 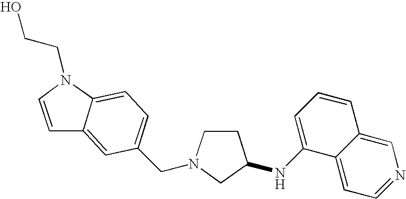 | 2c, 7, 8, 9, 10, 13, 16c |
| | (R)-2-(5-((3-(isoquinolin-5-ylamino)pyrrolidin-1- | |
| | yl)methyl)-1H-indol-1-yl)ethanol | |
| 2.067 | 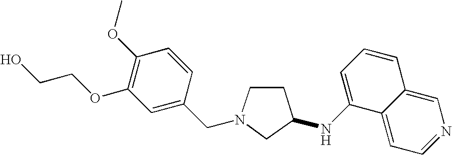 | 2c, 7, 8, 9, 10, 12a, 15c, 15d |
| | (R)-2-(5-((3-(isoquinolin-5-ylamino)pyrrolidin-1- | |
| | yl)methyl)-2-methoxyphenoxy)ethanol | |
| 2.068 | 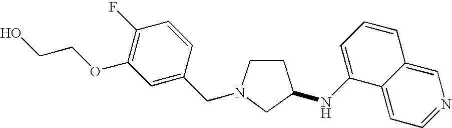 | 2c, 7, 8, 9, 10, 12a, 15c, 15d |
| | (R)-2-(2-fluoro-5-((3-(isoquinolin-5-ylamino)pyrrolidin- | |
| | 1-yl)methyl)phenoxy)ethanol | |
| 2.069 | 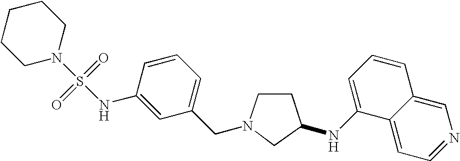 | 2c, 7, 8, 9, 10, 12a, 15c, 15d |
| | (R)-N-(3-((3-(isoquinolin-5-ylamino)pyrrolidin-1- | |
| | yl)methyl)phenyl)piperidine-1-sulfonamide | |
| 2.070 | 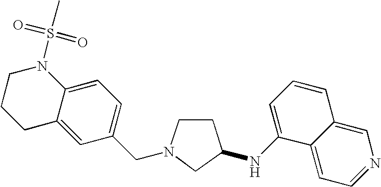 | 2c, 7, 8, 9, 10, 12b, 15c, 15e |
| | (R)-N-(1-((1-(methylsulfonyl)-1,2,3,4- | |
| | tetrahydroquinolin-6-yl)methyl)pyrrolidin-3- | |
| | yl)isoquinolin-5-amine | |
| 2.071 | 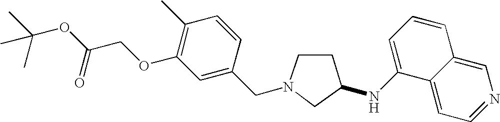 | 2c, 7, 8, 9, 10, I2a, 15c, 15d |
| | (R)-tert-butyl 2-(5-((3-(isoquinolin-5-ylamino)pyrrolidin- | |
| | 1-yl)methyl)-2-methylphenoxy)acetate | |
| 2.072 | 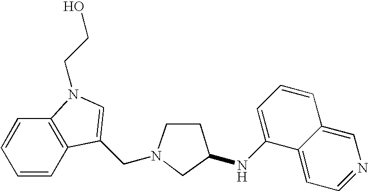 | 2c, 7, 8, 9, 10, 13, 16c |
| | (R)-2-(3-((3-(isoquinolin-5-ylamino)pyrrolidin-1- | |
| | yl)methyl)-1H-indol-1-yl)ethanol | |
| 2.073 | 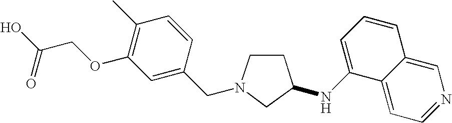 | 2c, 7, 8, 9, 10, 12a, 15c, 15d |
| | (R)-2-(5-((3-(isoquinolin-5-ylamino)pyrrolidin-1- | |
| | yl)methyl)-2-methylphenoxy)acetic acid | |
| 2.074 | 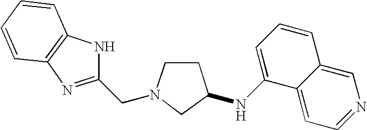 | 2c, 7, 8, 9, 10 |
| | (R)-N-(1-((1H-benzo[d]imidazol-2-yl)methyl)pyrrolidin- | |
| | 3-yl)isoquinolin-5-amine | |
| 2.075 | 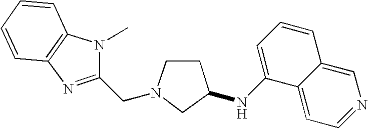 | 2c, 7, 8, 9, 10 |
| | (R)-N-(1-((1-methyl-1H-benzo[d]imidazol-2- | |
| | yl)methyl)pyrrolidin-3-yl)isoquinolin-5-amine | |
| 2.076 | 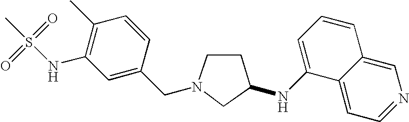 | 2c, 7, 8, 9, 10, 12a, 15c, 15d |
| | (R)-N-(5-((3-(isoquinolin-5-ylamino)pyrrolidin-1- | |
| | yl)methyl)-2-methylphenyDmethanesulfonamide | |
| 2.077 | 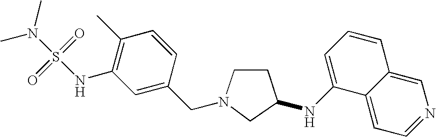 | 2c, 7, 8, 9, 10, 12a, 15c, 15d |
| | (R)-N-(5-((3-(isoquinolin-5-ylamino)pyrrolidin-1- | |
| | yl)methyl)-2-methylphenyl)-N′, N′ | |
| | dimethylaminosulfamide | |
| 2.078 | 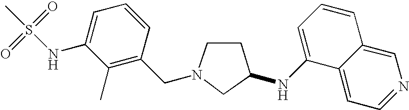 | 2c, 7, 8, 9, 10, 12a, 15c, 15d |
| | (R)-N-(3-((3-(isoquinolin-5-ylamino)pyrrolidin-1- | |
| | yl)methyl)-2-methylphenyl)methanesulfonamide | |
| 2.079 | 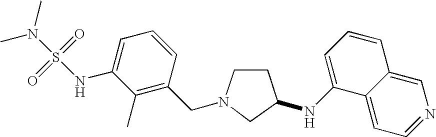 | 2c, 7, 8, 9, 10, 12a, 15c, 15d |
| | (R)-N-(3-((3-(isoquinolin-5-ylamino)pyrrolidin-1- | |
| | yl)methyl)-2-methylphenyl)-N′,N′ | |
| | dimethylaminosulfamide | |
| 2.080 | 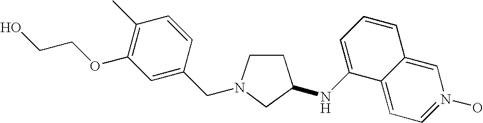 | 2b, 6b, 8, 9, 10, 12a, 15b, 15d |
| | (R)-5-(1-(3-(2-hydroxyethoxy)-4- | |
| | methylbenzyl)pyrrolidin-3-ylamino)isoquinoline 2-oxide | |
| 2.081 | 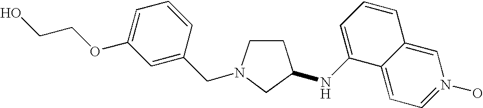 | 2b, 6b, 8, 9, 10, 12a, 15b, 15d |
| | (R)-5-(1-(3-(2-hydroxyethoxy)benzyl)pyrrolidin-3- | |
| | ylamino)isoquinoline 2-oxide | |
| 2.082 | 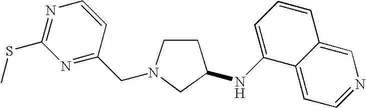 | 2c, 7, 8, 9, 10, 12b, 15c, 15e |
| | (R)-N-(1-((2-(methylthio)pyrimidin-4- | |
| | yl)methyl)pyrrolidin-3-yl)isoquinolin-5-amine | |
| 2.083 | 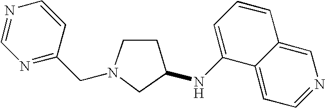 | 2c, 7, 8, 9, 10 |
| | (R)-N-(1-(pyrimidin-4-ylmethyl)pyrrolidin-3- | |
| | yl)isoquinolin-5-amine | |
| 2.084 | 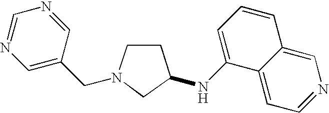 | 2c, 7, 8, 9, 10 |
| | (R)-N-(1-(pyrimidin-5-ylmethyl)pyrrolidin-3- | |
| | yl)isoquinolin-5-amine | |
| 2.085 | 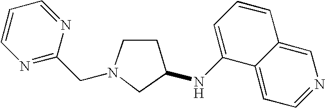 | 2c, 7, 8, 9, 10 |
| | (R)-N-(1-(pyrimidin-2-ylmethyl)pyrrolidin-3- | |
| | yl)isoquinolin-5-amine | |
| 2.086 | 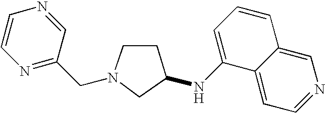 | 2c, 7, 8, 9, 10 |
| | (R)-N-(1-(pyrazin-2-ylmethyl)pyrrolidin-3- | |
| | yl)isoquinolin-5-amine | |
| 2.087 | 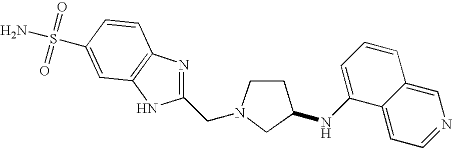 | 2c, 7, 8, 9, 10, 12b, 15c, 15e |
| | (R)-2-((3-(isoquinolin-5-ylamino)pyrrolidin-1- | |
| | yl)methyl)-1H-benzo[d]imidazole-6-sulfonamide | |
| 2.088 | 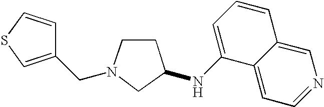 | 2c, 7, 8, 9, 10 |
| | (R)-N-(1-(thiophen-3-ylmethyl)pyrrolidin-3- | |
| | yl)isoquinolin-5-amine | |
| 2.089 | 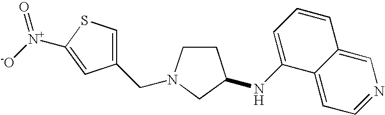 | 2c, 7, 8, 9, 10 |
| | (R)-N-(1-((5-nitrothiophen-3-yl)methyl)pyrrolidin-3- | |
| | yl)isoquinolin-5-amine | |
| 2.090 | 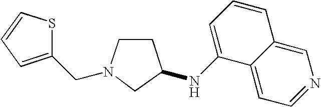 | 2c, 7, 8, 9, 10 |
| | (R)-N-(1-(thiophen-2-ylmethyl)pyrrolidin-3- | |
| | yl)isoquinolin-5-amine | |
| 2.091 | 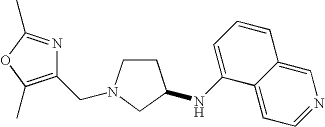 | 2c, 7, 8, 9, 10 |
| | (R)-N-(1-((2,5-dimethyloxazol-4-yl)methyl)pyrrolidin-3- | |
| | yl)isoquinolin-5-amine | |
| 2.092 | 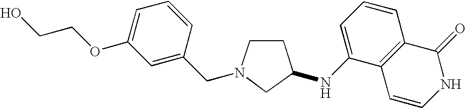 | 2b, 6b, 8, 9, 10, 12a, 15b, 15d |
| | (R)-5-(1-(3-(2-hydroxyethoxy)benzyl)pyrrolidin-3- | |
| | ylamino)isoquinolin-1(2H)-one | |
| 2.093 | 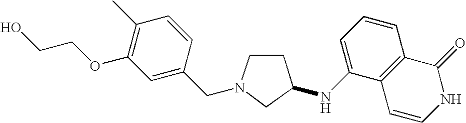 | 2b, 6b, 8, 9, 10, 12a, 16b, 15d |
| | (R)-5-(1-(3-(2-hydroxyethoxy)-4- | |
| | methylbenzyl)pyrrolidin-3-ylamino)isoquinolin-1(2H)- | |
| | one | |
| 2.094 | 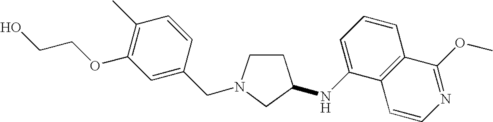 | 2b, 6b, 8, 9, 10, 12a, 15b, 15d |
| | (R)-2-(5-((3-(1-methoxyisoquinolin-5- | |
| | ylamino)pyrrolidin-1-yl)methyl)-2- | |
| | methylphenoxy)ethanol | |
| 2.095 | 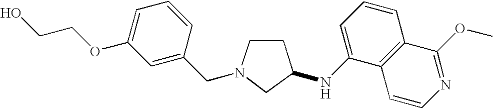 | 2b, 6b, 8, 9, 10, 12a, 15b, 15d |
| | (R)-2-(3-((3-(1-methoxyisoquinolin-5- | |
| | ylamino)pyrrolidin-1-yl)methyl)phenoxy)ethanol | |
| 2.096 | 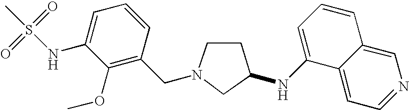 | 2c, 7, 8, 9, 10, 12a, 15c, 15d |
| | (R)-N-(3-((3-(isoquinolin-5-ylamino)pyrrolidin-1- | |
| | yl)methyl)-2-methoxyphenyl)methanesuifonamide | |
| 2.097 | 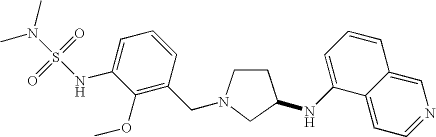 | 2c, 7, 8, 9, 10, 12a, 15c, 15d |
| | (R)-N-(3-((3-(isoquinolin-5-ylamino)pyrrolidin-1- | |
| | yl)methyl)-2-methoxyphenyl)-N′,N′ | |
| | dimethylaminosulfamide | |
| 2.098 | 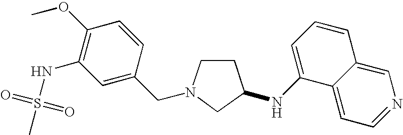 | 2c, 7, 8, 9, 10, 12a, 15c, 15d |
| | (R)-N-(5-((3-(isoquinolin-5-ylamino)pyrrolidin-1- | |
| | yl)methyl)-2-methoxyphenyl)methanesulfonamide | |
| 2.099 | 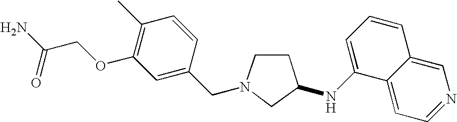 | 2c, 7, 8, 9, 10, 12a, 15c, 15d |
| | (R)-2-(5-((3-(isoquinolin-5-ylamino)pyrrolidin-1- | |
| | yl)methyl)-2-methylphenoxy)acetamide | |
| 2.100 | 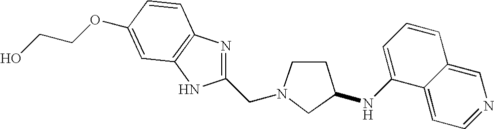 | 2c, 7, 8, 9, 10, 12a, 15c, 15d |
| | (R)-2-(2-((3-(isoquinolin-5-ylamino)pyrrolidin-1- | |
| | yl)methyl)-1H-benzo[d]imidazol-6-yloxy)ethanol | |
| 3.001 | 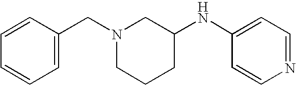 | 3c, 7, 8, 9, 10 |
| | N-(1-benzylpiperidin-3-yl)pyridin-4-amine | |
| 3.002 | 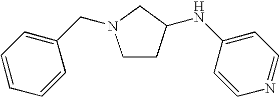 | 3c, 7, 8, 9, 10 |
| | N-(1-benzylpyrrolidin-3-yl)pyridin-4-amine | |
| 4.001 | 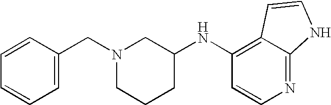 | 4c, 7, 8, 9, 10 |
| | N-(1-benzylpiperidin-3-yl)-1H-pyrrolo[2,3-b]pyrldln-4- | |
| | amine | |
| 4.002 | 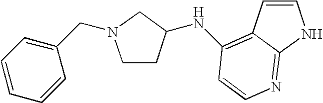 | 4c, 7, 8, 9, 10 |
| | N-(1-benzylpyrrolidin-3-yl)-1H-pyrrolo[2,3-b]pyridln-4- | |
| | amine | |
| 5.001 | 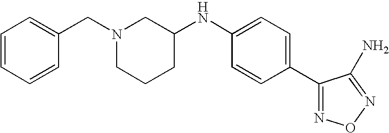 | 5a, 7, 8, 9, 10 |
| | 4-(4-(1-benzylpiperidin-3-ylamino)phenyl)-1,2,5- | |
| | oxadiazol-3-amine | |
| 5.002 | 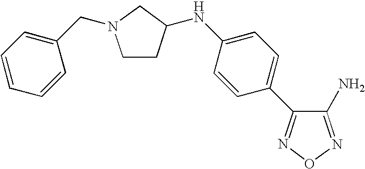 | 5a, 7, 8, 9, 10 |
| | 4-(4-(1-benzylpyrrolidin-3-ylamino)phenyl)-1,2,5- | |
| | oxadiazol-3-amine | |

Preferred ROCK inhibitor compounds of this invention include, but are not limited to the ROCK inhibitor compounds of embodiments 5, 14, 15, 16, 17, 18, 19, 20, and 21 as described above, and their associated salts, tautomers, solvates, or hydrates. In particular, preferred Compounds include 1.074, 1.075, 1.076, 1.077, 1.079, 1.091, 1.093, 1.108, 1.109, 1.123, 1.124, 1.126, 1.131, 1.132, 1.133, 1.134, 1.135, 1.136, 1.137, 1.138, 1.141, 1.148, 1.149, 1.150, 1.152, 1.153, 1.155, 1.156, 1.157, 1.158, 1.161, 1.162, 1.163, 1.164, 1.165, 1.166, 1.171, 1.173, 1.175, 1.176, 1.186, 1.193, 1.195, 1.197, 1.200, 1.206, 1.212, 1.213, 1.215, 1.217, 1.219, 1.223, 1.233, 1.236, 1.237, 1.238, 1.239, 1.249, 1.252, 1.253, 1.258, 1.259, 1.260, 1.261, 1.262, 1.270, 1.273, 1.275, 1.277, 1.281, 2.025, 2.026, 2.031, 2.038, 2.039, 2.041, 2.046, 2.047, 2.054, 2.055, 2.057, 2.058, 2.059, 2.060, 2.061, 2.064, 2.065, 2.066, 2.067, 2.068, 2.069, 2.072, 2.073, 2.076, 2.077, 2.078, 2.079, 2.082, 2.096, 2.097, and 2.099.

### Pharmaceutical Formulations

The present invention provides a pharmaceutical formulation comprising compounds of Formula I or II and a pharmaceutically acceptable carrier. Pharmaceutically acceptable carriers can be selected by those skilled in the art using conventional criteria. Pharmaceutically acceptable carriers include, but are not limited to, saline solution, aqueous electrolyte solutions, isotonicity modifiers, water polyethers such as polyethylene glycol, polyvinyls such as polyvinyl alcohol and povidone, cellulose derivatives such as methylcellulose and hydroxypropyl methylcellulose, polymers of acrylic acid such as carboxypolymethylene gel, polysaccharides such as dextrans, and glycosaminoglycans such as sodium hyaluronate and salts such as sodium chloride and potassium chloride.

The pharmaceutical formulation useful for the present invention in general is an aqueous solution comprising water, suitable ionic or non-ionic tonicity modifiers, suitable buffering agents, and a compound of Formula I or II. In one embodiment, the compound is at 0.005 to 3% w/v, and the aqueous solution has a tonicity of 200-400 mOsm/kG and a pH of 4-9.

In one embodiment, the tonicity modifier is ionic such as NaCl, for example, in the amount of 0.5-0.9% w/v, preferably 0.6-0.9% w/v.

In another embodiment, the tonicity modifier is non-ionic, such as mannitol, dextrose, in the amount of at least 2%, or at least 2.5%, or at least 3%, and no more than 7.5%; for example, in the range of 3-5%, preferably 4-5% w/v.

The pharmaceutical formulation can be sterilized by filtering the formulation through a sterilizing grade filter, preferably of a 0.22-micron nominal pore size. The pharmaceutical formulation can also be sterilized by terminal sterilization using one or more sterilization techniques including but not limited to a thermal process, such as an autoclaving process, or a radiation sterilization process, or using pulsed light to produce a sterile formulation. In one embodiment, the pharmaceutical formulation is a concentrated solution of the active ingredient; the formulation can be serially diluted using appropriate acceptable sterile diluents prior to systemic administration.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or acetyl alcohol. Sweetening agents such as those set forth above, and flavoring agents can be added to provide palatable oral preparations. These compositions can be preserved by the addition of an anti-oxidant such as ascorbic acid.

Pharmaceutical compositions of the invention can be in the form of oil-in-water emulsions. The oily phase can be a vegetable oil, for example olive oil or arachis oil, or a mineral oil, for example liquid paraffin or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions can also contain sweetening and flavoring agents.

Pharmaceutical compositions of the invention can be in the form of an aerosol suspension of respirable particles comprising the active compound, which the subject inhales. The respirable particles can be liquid or solid, with a particle size sufficiently small to pass through the mouth and larynx upon inhalation. In general, particles having a size of about 1 to 10 microns, preferably 1-5 microns, are considered respirable.

The pharmaceutical formulation for systemic administration such as injection and infusion is prepared in a sterile medium. The active ingredient, depending on the vehicle and concentration used, can either be suspended or dissolved in the vehicle. Adjuvants such as local anesthetics, preservatives and buffering agents can also be dissolved in the vehicle. The sterile injectable preparation can be a sterile injectable solution or suspension in a non-toxic acceptable diluent or solvent. Among the acceptable vehicles and solvents that can be employed are sterile water, saline solution, or Ringer's solution.

The pharmaceutical compositions for oral administration contain active compounds in the form of tablets, lozenges, aqueous or oily suspensions, viscous gels, chewable gums, dispersible powders or granules, emulsion, hard or soft capsules, or syrups or elixirs.

For oral use, an aqueous suspension is prepared by addition of water to dispersible powders and granules with a dispersing or wetting agent, suspending agent one or more preservatives, and other excipients. Suspending agents include, for example, sodium carboxymethylcellulose, methylcellulose and sodium alginate. Dispersing or wetting agents include naturally-occurring phosphatides, condensation products of an allylene oxide with fatty acids, condensation products of ethylene oxide with long chain aliphatic alcohols, condensation products of ethylene oxide with partial esters from fatty acids and a hexitol, and condensation products of ethylene oxide with partial esters derived from fatty acids and hexitol anydrides. Preservatives include, for example, ethyl, and n-propyl p-hydroxybenzoate. Other excipients include sweetening agents (e.g., sucrose, saccharin), flavoring agents and coloring agents. Those skilled in the art will recognize the many specific excipients and wetting agents encompassed by the general description above.

For oral application, tablets are prepared by mixing the active compound with nontoxic pharmaceutically acceptable excipients suitable for the manufacture of tablets. These excipients can be, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets can be uncoated or they can be coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil. Formulation for oral use can also be presented as chewable gums by embedding the active ingredient in gums so that the active ingredient is slowly released upon chewing.

The pharmaceutical compositions can be in the form of suppositories, which are prepared by mixing the active ingredient with a suitable non-irritating excipient that is solid at ordinary temperatures but liquid at the rectal temperature and will thus melt in the rectum to release the compound. Such excipients include cocoa butter and polyethylene glycols.

### Method of Treating Ocular Diseases Using Rho Kinase Inhibitor Compounds

The present invention is useful in treating ocular diseases associated with excessive cell proliferation, tissue remodeling, inflammation, neurite retraction/degeneration, and vascular permeability and edema. The present invention is particularly effective in treating ocular diseases such as allergic conjunctivitis, corneal hyposensitivity, dry eye, proliferative vitreal retinopathy, macular edema and degeneration, and blepharitis.

### Allergic Conjunctivitis

The inventors have discovered that compounds of Formula I or II are useful in treating the defects in inflammation seen in allergic conjunctivitis.

The present invention is directed to a method of treating allergic conjunctivitis. The method comprises the steps of first identifying a subject suffering from allergic conjunctivitis, then administering to the subject an effective amount of a compound of Formula I or II to treat allergic conjunctivitis.

Indicia of efficacy for allergic conjunctivitis include demonstrable improvement in measurable signs, symptoms and other variables clinically relevant to this condition. Specifically, an improving effect on the signs and symptoms of allergic conjunctivitis include itching, tearing, conjunctival edema, hyperemia, watery discharge, burning, and photophobia and eyelid edema. Restoration of normal blink frequency, improved tear film stability, improvement in corneal staining, improvement in tear volume as determined by Schirmer scores, improvement in ocular surface discomfort, increased visual acuity, restoration of normal corneal function (corneal fluid transport and corneal thickness), increased success in maintaining refractive index of cornea following refractive procedure, decreased conjunctival hyperemia, decreased reliance on ocular palliative treatments (artificial tears), decreases need for topical/systemic analgesics, decreased incidence of dry eye disease, decreased ocular surface inflammation (cytokines and pro-inflammatory mediators) and decreased doctor visits are expected.

### Corneal Hyposensitivity and Neurotrophic Keratopathy

The inventors have discovered that compounds of Formula I or II are useful in treating neurite retraction and neurodegeneration seen in corneal hyposensitivity following PRK and LASIK procedures and neurotrophic keratopathy.

The present invention is directed to a method of treating corneal hyposensitivity or neurotrophic keratopathy. The method comprises the steps of first identifying a subject suffering from corneal hyposensitivity or neurotrophic keratopathy, then administering to the subject an effective amount of a compound of Formula I or II to treat corneal hyposensitivity or neurotrophic keratopathy.

Indicia of efficacy for corneal hyposensitivity or neurotrophic keratopathy include demonstrable improvement in measurable signs, symptoms and other variables clinically relevant to corneal hyposensitivity. Since the pharmaceutical agent of the present invention has a corneal neuritogenesis promoting effect, it is useful for improvement of hypofunction of corneal sensitivity due to damaged corneal nerve and the like, as well as improvement of dry eye associated with hypofunction of corneal sensitivity. Improvements include increased corneal sensitivity, increased corneal epithelial wound healing rate, restoration of normal blink frequency, improved tear film stability, improvement in corneal staining, improvement in tear volume as determined by Schirmer scores, improvement in ocular surface discomfort, improved quality of life, increased visual acuity, restoration of normal corneal function (corneal fluid transport and corneal thickness), increased success in maintaining refractive index of cornea following refractive procedure, decreased conjunctival hyperemia, decreased reliance on ocular palliative treatments (artificial tears), decreases need for topical/systemic analgesics, decreased incidence of dry eye disease, decreased ocular surface inflammation (cytokines and proinflammatory mediators) and decreased doctor visits.

### Dry Eye

The inventors have discovered that compounds of Formula I or II inhibit the ROCK-mediated regulation of chemotaxis, cytokinesis, cytokine and chemokine secretion. Furthermore, the inventors have discovered that compounds of Formula I or II are useful in treating the defects in inflammation as seen in dry eye disease.

The present invention is directed to a method of treating dry eye. The method comprises the steps of first identifying a subject suffering from dry eye, then administering to the subject an effective amount of a compound of Formula I or II to treat dry eye.

A method for treating dry eye is based on the properties of the Formula I or II compounds to reduce inflammation that accompany this disorder.

Indicia of efficacy for treating dry eye by the present method include demonstrable improvement in measurable signs, symptoms and other variables clinically relevant to dry eye. Such improvements include reducing the evaporation rate of normal or artificial tears, minimizing the loss of tears, maximizing the preservation of tears, increasing tear film stability, decreasing tear film osmolarity, increasing tear volume, increasing tear secretion, decreasing tear break-up time, decreasing immune-mediated inflammation, increasing gland function, decreasing irritation and itching, decreasing grittiness, decreasing foreign body sensation, increasing aqueous component of tears, decreasing photophobia, decreasing accumulation of mucus filaments, decreasing punctate conjunctival and corneal damage, inducing contraction of the bulbar conjunctival vessels, decreasing dullness of the conjunctiva and cornea, decreasing corneal punctate fluorescein staining, reducing symptoms of blurred vision, increasing secretion of natural anti-inflammatory factors and decreasing production of pro-inflammatory cytokines and proteolytic enzymes. Ophthalmic formulations containing compounds of Formula I or II, that inhibit ROCK-mediated regulation of certain secreted pro-inflammatory factors and thus improve tear production and tear break up time by reducing immune-mediated inflammation, would clinically lead to decreased irritation and itching, decreased grittiness and foreign body sensation, decreased photophobia, a measurable decrease in corneal damage, contraction of the bulbar conjunctival vessels, decrease in corneal punctate fluorescein staining and reduced symptoms of blurred vision. Inspire's Rho kinase inhibitor compounds have the potential to provide a novel mechanism for the treatment of Dry Eye.

### Macular Edema and Macular Degeneration

The inventors have discovered that compounds of Formula I or II inhibit the ROCK-mediated regulation of chemotaxis, cytokinesis, cytokine and chemokine secretion, proliferation, cell motility and endothelial integrity. Furthermore, the inventors have discovered that compounds of Formula I or II are useful in treating the defects in inflammation, excessive cell proliferation, remodeling, tissue edema, angiogenesis, vascular permeability, endothelial cell invasion and remodeling seen in macular edema and macular degeneration.

The present invention is directed to a method of treating macular edema or macular degeneration. The method comprises the steps of first identifying a subject suffering from macular edema or macular degeneration, then administering to the subject an effective amount of a compound of Formula I or II to treat macular edema or macular degeneration.

A method for treating macular edema and macular degeneration is based on the properties of the Formula I or II compounds to reduce at least one of the following processes contributing to pathophysiologies that accompany this disorder: inflammation, excessive cell proliferation, remodeling, tissue edema, angiogenesis, vascular permeability, endothelial cell invasion and remodeling.

Indicia of efficacy for treating macular edema or macular degeneration by the present method include demonstrable improvement in measurable signs, symptoms and other variables clinically relevant to macular edema and degeneration. Indicia of efficacy for macular edema and degeneration include increased or maintained central vision, reduction of blurred vision, enhanced visual acuity, decreased metamorphopsia, reduced or absent central scotomas, reduced sensitivity to glare, increased contrast sensitivity, increased color vision, decreased macular inflammation, decreased fluid retention in the macula, decreased macular swelling, decreased onset or prevention of retinal neovasculature, decreased macular drusen formation, maintenance or decrease in Bruch's membrane thickness.

### Proliferative Vitreal Retinopathy

The inventors have discovered that compounds of Formula I or II inhibit the ROCK-mediated regulation of focal adhesions, remodeling, proliferation, and contractility. Furthermore, the inventors have discovered that compounds of Formula I or II are useful in treating the defects in excessive cell proliferation, adhesion and cellular contractility seen in PVR.

The present invention is directed to a method of treating PVR. The method comprises the steps of first identifying a subject suffering from PVR, then administering to the subject an effective amount of a compound of Formula I or II to treat PVR.

A method for treating PVR is based on the properties of the Formula I or II compounds to reduce at least one of the following processes contributing to pathophysiologies that accompany this disorder: excessive cell proliferation, remodeling, adhesion and contractility. Indicia of efficacy of proliferative vitreoretinopathy include: reduction in the frequency of failed surgical outcomes to repair rhegmatogenous retinal detachment; reduction in vitreous flare and pigment clumps in vitreous; ability to correct PVR through pharmacological, non-surgical intervention; improvement in vision central and peripheral vision following RRD surgery; reduction in ocular hypotony; and reduction in macular pucker following retinal detachment surgery.

### Blepharitis

The inventors have discovered that compounds of Formula I or II inhibit the ROCK-mediated regulation of chemotaxis, cytokinesis, cytokine and chemokine secretion, proliferation, cell motility and endothelial integrity. Furthermore, the inventors have discovered that compounds of Formula I or II are useful in treating the defects in inflammation, excessive cell proliferation, remodeling and tissue edema seen in blepharitis.

The present invention is directed to a method of treating blepharitis. The method comprises the steps of first identifying a subject suffering from blepharitis, then administering to the subject an effective amount of a compound of Formula I or II to treat blepharitis.

A method for treating blepharitis is based on the properties of the Formula I or II compounds to reduce at least one of the following processes contributing to pathophysiologies that accompany this disorder: inflammation, excessive cell proliferation, remodeling and tissue edema.

Indicia of efficacy for treating blepharitis by the present method include demonstrable improvement in measurable signs, symptoms and other variables clinically relevant to blepharitis. Such improvements include elimination of redness, swelling, burning, watering, and itching of the eyelids; decrease in flaking and debris accumulation on the eyelashes; decrease in a foreign body sensation; crusting and closure of eyelids upon waking; attenuation of abnormal growth or loss of lashes; decrease in pain sensation and sensitivity to light; a decrease in the incidence of associated complications such as styes, chalzions, dry eye, meibomitis, keratitis, and recurrent conjunctivitis; and heightened sense of well being and self-confidence along with an enhanced ability to carry out daily life activities.

### Methods of Administration

The present invention is particularly effective in treating ophthalmic diseases such as allergic conjunctivitis, corneal hyposensitivity and kerotopathy, dry eye disease, proliferative vitreal retinopathy, macular edema and degeneration, and blepharitis. Any method of delivering the compound to the relevant tissues of the eye, including local administration and systemic administration, is suitable for the present invention.

The active compounds disclosed herein may be administered to the eyes of a patient by any suitable means, but are preferably administered by administering a liquid or gel suspension of the active compound in the form of drops, spray or gel. Alternatively, the active compounds may be applied to the eye via liposomes. Further, the active compounds may be infused into the tear film via a pump-catheter system. Another embodiment of the present invention involves the active compound contained within a continuous or selective-release device. As an additional embodiment, the active compounds can be contained within, carried by, or attached to contact lenses that are placed on the eye. Another embodiment of the present invention involves the active compound contained within a swab or sponge that can be applied to the ocular surface. Another embodiment of the present invention involves the active compound contained within a liquid spray that can be applied to the ocular surface. Another embodiment of the present invention involves an injection of the active compound directly into the lachrymal tissues or onto the eye surface.

The active compounds disclosed herein are preferably administered by administering an aqueous suspension into the vitreous. Intravitreal administration comprising: single or multiple intravitreal injections; administration directly into the vitreal chamber during surgery separately or in conjunction with intraocular irrigation solutions, or other similar solutions or devices, routinely used during vitreoretinal surgery; administration via liposomes or other suitable pharmaceutical carriers; administration via continuous or selective-release intravitreal-implantable devices. The intravitreal solution containing the active compound may contain a physiologically compatible vehicle, as those skilled in the ophthalmic art can select using conventional criteria. The vehicles may be selected from the known ophthalmic vehicles which include, but are not limited to, saline solution, water polyethers such as polyethylene glycol, polyvinyls such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate and salts such as sodium chloride and potassium chloride. The preferred embodiment is an intravitreal solution comprising active compound and saline at neutral pH and physiological osmolarity.

The topical solution containing the active compound may also contain a physiologically compatible vehicle, as those skilled in the ophthalmic art can select using conventional criteria. The vehicles may be selected from the known ophthalmic vehicles which include, but are not limited to, saline solution, water polyethers such as polyethylene glycol, polyvinyls such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate and salts such as sodium chloride and potassium chloride.

In addition to the topical method of administration described above, there are various methods of administering the active compounds of the present invention systemically. One such means would involve an aerosol suspension of respirable particles comprised of the active compound, which the subject inhales. The active compound would be absorbed into the bloodstream via the lungs or contact the ocular tissues via the nasolacrimal ducts, and subsequently contact the retinal pigment epithelial cells in a pharmaceutically effective amount. The respirable particles may be liquid or solid, with a particle size sufficiently small to pass through the mouth and larynx upon inhalation; in general, particles ranging from about 1 to 10 microns, but more preferably 1-5 microns, in size are considered respirable.

Another means of systemically administering the active compounds to the eyes of the subject would involve administering a liquid/liquid suspension in the form of eye drops or eye wash or nasal drops of a liquid formulation, or a nasal spray of respirable particles that the subject inhales. Liquid pharmaceutical compositions of the active compound for producing a nasal spray or nasal or eye drops may be prepared by combining the active compound with a suitable vehicle, such as sterile pyrogen free water or sterile saline by techniques known to those skilled in the art.

Other means of systemic administration of the active compound would involve oral administration, in which pharmaceutical compositions containing compounds of Formula I are in the form of tablets, lozenges, aqueous or oily suspensions, dispersible powders or granules, emulsion, hard or soft capsules, or syrups or elixirs. Compositions intended for oral use may be prepared according to any method known to the art for the manufacture of pharmaceutical compositions and such compositions may contain one or more agents selected from the group consisting of: of sweetening agents, flavoring agents, coloring agents and preserving agents in order to provide pharmaceutically elegant and palatable preparations. Tablets contain the active ingredient in admixture with nontoxic pharmaceutically acceptable excipients that are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Additional means of systemic administration of the active compound to the eyes of the subject would involve a suppository form of the active compound, such that a therapeutically effective amount of the compound reaches the eyes via systemic absorption and circulation.

Further means of systemic administration of the active compound would involve direct intra-operative instillation of a gel, cream, or liquid suspension form of a therapeutically effective amount of the active compound.

High doses may be required for some therapeutic agents to achieve levels to effectuate the target response, but may often be associated with a greater frequency of dose-related adverse effects. Combined use of the compounds of the present invention with agents commonly used to treat ocular diseases permits relatively lower doses of such agents resulting in a lower frequency of adverse side effects associated with long-term administration of such therapeutic agents.

For systemic administration, plasma concentrations of active compounds delivered can vary according to compounds; but are generally 1×10⁻¹⁰-1×10⁻⁴ moles/liter, and preferably 1×10⁻⁸-1×10⁻⁵ moles/liter.

Preferred dosage levels are about 0.05-100, 0.1-100, or 1-100 mg/kg body weight per day. The amount of active ingredient that can be combined with the carrier materials to produce a single dosage form will vary depending upon the host treated and the particular mode of administration. Dosage unit forms will generally contain between from about 0.01-10 mg for topical administration and about 1.0-100 mg for systemic or oral administration.

The invention is illustrated further by the following examples that are not to be construed as limiting the invention in scope to the specific procedures described in them.

### EXAMPLES

### Example 1

### Rho Kinase Inhibition Assay

### Relevance:

This assay demonstrates a compound's ability to inhibit ROCK2 and ROCK1 in an in vitro setting using the isolated enzyme. Compounds having ROCK2 IC₅₀ values on the order of 2 μM or below have been shown to possess efficacy in many studies using in vivo models of the disease processes described in this application.

### Protocol

Inhibition of ROCK2 and ROCK1 activity was determined using the IMAP™ Screening Express Kit (Molecular Devices product number #8073). ROCK2 enzyme (Upstate/Chemicon #14-451), ROCK1 (Upstate/Chemicon #14-601) and Flourescein tagged substrate peptide Fl-AKRRRLSSLRA (Molecular Devices product number R7184) was pre-incubated with a test compound (a Formula II compound or other Rho kinase compound such as fasudil, H-1152, H7, Y-27632, Y-39983) for 5 minutes in buffer containing 10 mM Tris-HCl pH 7.2, 10 mM MgCl₂, and 0.1% BSA. Following the pre-incubation, 10 μM ATP was added to initiate the reaction. After 60 minutes at room temperature, Molecular Devices IMAP™ binding solution was added to bind phosphorylated substrate. After 30 minutes of incubation in the presence of the IMAP™ beads, the fluorescence polarization was read and the ratio was reported as mP. IC₅₀ values for compounds and EC₅₀ values for ATP were calculated using the Prism software from Graphpad.

### Results:

TABLE 1
Rho Kinase I and II Potency Data

| | ROCK1 Ki, | ROCK1 Ki, | ROCK2 Ki, | ROCK2 Ki, |
|---|---|---|---|---|
| Compound | Avg, nM | StdDev, nM | Avg, nM | StdDev, nM |
| 1.008 | 30.5 | 0.8 | 3.9 | 0.1 |
| 1.034 | 36.0 | 22.2 | 5.3 | 2.6 |
| 1.039 | 208.6 | 109.0 | 24.7 | 8.4 |
| 1.051 | 37.2 | 4.0 | 3.8 | 0.0 |
| 1.072 | 33.7 | 22.1 | 5.6 | 3.1 |
| 1.074 | 40.1 | 3.3 | 4.1 | 1.5 |
| 1.075 | 48.7 | 2.8 | 4.4 | 0.3 |
| 1.076 | 14.3 | 5.4 | 2.6 | 0.6 |
| 1.077 | 76.1 | 30.9 | 11.1 | 5.8 |
| 1.078 | 36.3 | 10.1 | 3.6 | 0.9 |
| 1.079 | 71.5 | 9.1 | 4.7 | 1.1 |
| 1.080 | 130.8 | 42.6 | 15.2 | 4.4 |
| 1.087 | 84.1 | 11.1 | 15.4 | 1.4 |
| 1.090 | 281.0 | 103.7 | 24.9 | 7.9 |
| 1.091 | 71.4 | 22.0 | 3.3 | 1.0 |
| 1.092 | 190.5 | 42.2 | 28.4 | 10.6 |
| 1.093 | 64.5 | 21.9 | 7.7 | 5.2 |
| 1.095 | 274.8 | 88.0 | 49.5 | 35.9 |
| 1.098 | 205.6 | 69.4 | 25.0 | 6.4 |
| 1.106 | 223.4 | 82.0 | 15.1 | 4.9 |
| 1.107 | 233.7 | 137.2 | 14.0 | 8.5 |
| 1.108 | 25.6 | 3.2 | 6.5 | 0.3 |
| 1.109 | 58.8 | 25.8 | 9.6 | 2.5 |
| 1.110 | 59.0 | 4.1 | 11.2 | 0.3 |
| 1.115 | 89.7 | 17.5 | 20.6 | 1.7 |
| 1.116 | 257.8 | 45.6 | 48.9 | 5.5 |
| 1.117 | 208.0 | 1.9 | 35.8 | 2.3 |
| 1.118 | 461.7 | 28.3 | 81.7 | 52.7 |
| 1.123 | 82.3 | 11.0 | 9.6 | 4.3 |
| 1.124 | 64.5 | 7.9 | 3.3 | 0.8 |
| 1.125 | 557.1 | 1.7 | 50.9 | 16.8 |
| 1.126 | 76.2 | 16.7 | 17.2 | 3.9 |
| 1.127 | 96.6 | 11.6 | 11.2 | 0.4 |
| 1.130 | 577.1 | 340.0 | 142.0 | 38.1 |
| 1.131 | 19.7 | 5.9 | 3.8 | 0.9 |
| 1.132 | 22.5 | 6.5 | 3.5 | 0.4 |
| 1.133 | 25.0 | 7.2 | 4.3 | 1.1 |
| 1.134 | 22.4 | 6.0 | 4.4 | 0.6 |
| 1.136 | 40.3 | 15.3 | 5.4 | 0.4 |
| 1.137 | 25.8 | 10.7 | 5.1 | 1.2 |
| 1.138 | 36.3 | 12.2 | 7.2 | 1.1 |
| 1.139 | 200.3 | 26.3 | 23.2 | 9.6 |
| 1.140 | 236.1 | 199.3 | 32.9 | 24.9 |
| 1.141 | 28.5 | 11.1 | 3.8 | 1.1 |
| 1.142 | 104.2 | 26.6 | 12.0 | 4.4 |
| 1.143 | 49.7 | 30.8 | 12.6 | 11.9 |
| 1.144 | 97.6 | 65.0 | 19.5 | 13.0 |
| 1.145 | 35.0 | 13.5 | 6.4 | 0.9 |
| 1.146 | 39.8 | 10.9 | 10.7 | 1.5 |
| 1.147 | 58.3 | 15.6 | 45.7 | 52.0 |
| 1.148 | 24.3 | 13.7 | 3.6 | 0.9 |
| 1.149 | 46.8 | 21.3 | 4.2 | 2.2 |
| 1.150 | 33.2 | 17.5 | 3.2 | 1.2 |
| 1.151 | 22.8 | 6.0 | 2.9 | 0.5 |
| 1.152 | 19.8 | 13.3 | 3.3 | 0.9 |
| 1.153 | 62.8 | 8.7 | 4.2 | 0.8 |
| 1.154 | 52.7 | 9.5 | 6.6 | 1.0 |
| 1.155 | 45.4 | 14.7 | 7.0 | 2.0 |
| 1.156 | 135.8 | 34.3 | 13.0 | 3.0 |
| 1.157 | 263.8 | 73.9 | 8.8 | 1.6 |
| 1.158 | 64.1 | 20.1 | 5.1 | 1.0 |
| 1.159 | 48.1 | 9.2 | 10.1 | 2.6 |
| 1.160 | 218.3 | 28.3 | 49.4 | 13.4 |
| 1.161 | 9.9 | 3.4 | 2.5 | 0.5 |
| 1.162 | 15.2 | 1.5 | 2.8 | 0.8 |
| 1.163 | 33.6 | 5.8 | 2.9 | 0.4 |
| 1.164 | 42.4 | 7.2 | 6.1 | 1.2 |
| 1.165 | 50.7 | 4.4 | 3.4 | 0.6 |
| 1.166 | 95.2 | 8.6 | 8.0 | 0.8 |
| 1.167 | 118.6 | 17.1 | 18.5 | 1.7 |
| 1.168 | 162.2 | 68.3 | 22.9 | 10.4 |
| 1.169 | 256.2 | 132.7 | 33.8 | 20.0 |
| 1.170 | 80.0 | 25.9 | 12.5 | 6.1 |
| 1.171 | 109.2 | 60.1 | 16.0 | 8.4 |
| 1.172 | 103.0 | 40.6 | 20.5 | 7.3 |
| 1.173 | 15.1 | 6.8 | 3.6 | 1.0 |
| 1.175 | 65.9 | 28.3 | 7.6 | 1.5 |
| 1.176 | 314.3 | 77.6 | 11.2 | 3.2 |
| 1.177 | 156.1 | 55.0 | 18.2 | 5.5 |
| 1.178 | 137.6 | 58.0 | 24.9 | 17.6 |
| 1.179 | 292.0 | 70.7 | 19.3 | 4.4 |
| 1.180 | 138.5 | 46.5 | 23.1 | 4.8 |
| 1.181 | 567.8 | 191.3 | 32.8 | 3.5 |
| 1.182 | 408.3 | 106.6 | 30.6 | 4.3 |
| 1.183 | 165.1 | 46.3 | 16.8 | 3.7 |
| 1.184 | 843.1 | 53.0 | 90.9 | 13.9 |
| 1.185 | 81.6 | 33.0 | 12.6 | 6.4 |
| 1.186 | 129.3 | 42.2 | 11.9 | 4.9 |
| 1.187 | 296.2 | 78.8 | 17.3 | 5.8 |
| 1.188 | 3468.8 | | 652.7 | |
| 1.189 | 187.9 | 62.0 | 34.3 | 5.1 |
| 1.190 | 325.6 | 38.9 | 71.8 | 9.0 |
| 1.191 | 147.3 | 24.7 | 33.4 | 2.0 |
| 1.192 | 158.4 | 33.5 | 37.7 | 4.7 |
| 1.193 | 64.9 | 4.2 | 14.8 | 1.2 |
| 1.194 | 175.7 | 6.3 | 20.2 | 2.4 |
| 1.195 | 196.2 | 58.0 | 10.3 | 3.6 |
| 1.196 | 710.7 | 191.7 | 39.8 | 15.0 |
| 1.197 | 120.2 | 36.0 | 5.0 | 1.4 |
| 1.198 | 584.5 | 139.5 | 24.7 | 9.9 |
| 1.199 | 1856.6 | | 213.0 | 34.4 |
| 1.200 | 76.5 | 17.9 | 5.9 | 0.9 |
| 1.201 | 1585.4 | | 229.5 | |
| 1.202 | 203.5 | 40.9 | 33.0 | 2.1 |
| 1.203 | 329.4 | 67.4 | 41.6 | 6.4 |
| 1.204 | 196.1 | 42.0 | 31.9 | 2.2 |
| 1.205 | 498.1 | 95.2 | 46.4 | 3.7 |
| 1.206 | 64.4 | 15.1 | 9.1 | 3.8 |
| 1.207 | 516.3 | 27.5 | 43.7 | 1.1 |
| 1.208 | 54.2 | 25.0 | 12.9 | 2.8 |
| 1.209 | 4591.0 | | 469.6 | 58.3 |
| 1.210 | 95.1 | 18.2 | 25.5 | 3.8 |
| 1.211 | 395.5 | 58.5 | 57.6 | 0.6 |
| 1.212 | 44.2 | 11.2 | 3.9 | 0.2 |
| 1.213 | 106.3 | 10.9 | 3.0 | 0.5 |
| 1.214 | 546.5 | 10.9 | 143.0 | 7.0 |
| 1.215 | 102.8 | 5.8 | 3.5 | 0.3 |
| 1.216 | 1885.4 | | 402.9 | 79.5 |
| 1.217 | 70.1 | 9.5 | 12.1 | 1.1 |
| 1.218 | 401.8 | 34.4 | 30.7 | 3.0 |
| 1.219 | 343.6 | 37.6 | 15.4 | 2.3 |
| 1.221 | 264.4 | 41.6 | 30.0 | 2.6 |
| 1.222 | 228.8 | 41.9 | 75.5 | 1.2 |
| 1.223 | 239.5 | 21.5 | 15.7 | 1.9 |
| 1.224 | 487.0 | 151.5 | 77.5 | 23.0 |
| 1.225 | 605.0 | 133.2 | 189.4 | 48.9 |
| 1.226 | 91.7 | 31.5 | 8.8 | 2.6 |
| 1.227 | 47.5 | 2.8 | 5.3 | 0.4 |
| 1.228 | 1883.4 | 681.9 | 139.6 | 28.2 |
| 1.229 | 121.4 | 86.2 | 18.4 | 5.8 |
| 1.230 | 345.9 | 85.2 | 35.3 | 9.8 |
| 1.231 | 305.1 | 62.8 | 60.3 | 18.2 |
| 1.232 | 136.6 | 41.1 | 20.8 | 8.8 |
| 1.233 | 47.2 | 7.2 | 1.3 | 0.1 |
| 1.234 | 1735.2 | 179.0 | 166.4 | 11.6 |
| 1.235 | 1386.4 | 173.1 | 335.4 | 29.4 |
| 1.236 | 49.3 | 7.1 | 2.1 | 0.1 |
| 1.237 | 286.7 | 55.0 | 4.0 | 0.4 |
| 1.238 | 61.2 | 22.1 | 1.5 | 0.3 |
| 1.239 | 282.6 | 36.2 | 6.3 | 0.6 |
| 1.240 | 624.8 | 74.2 | 60.1 | 9.3 |
| 1.241 | 65.1 | 11.8 | 21.0 | 6.4 |
| 1.242 | 71.4 | 14.1 | 17.5 | 1.8 |
| 1.243 | 219.3 | 29.7 | 84.3 | 17.2 |
| 1.244 | 683.1 | 80.9 | 138.7 | 25.4 |
| 1.245 | 199.0 | 27.7 | 49.5 | 7.9 |
| 1.246 | 92.1 | 6.3 | 11.2 | 0.8 |
| 1.247 | 1312.4 | 268.7 | 242.6 | 53.1 |
| 1.248 | 2349.7 | | 890.6 | 509.8 |
| 1.249 | 91.7 | 25.0 | 8.6 | 3.8 |
| 1.250 | 247.0 | 63.7 | 45.8 | 13.8 |
| 1.251 | 206.8 | 44.0 | 49.2 | 10.5 |
| 1.252 | 30.5 | 1.5 | 4.5 | 0.4 |
| 1.253 | 59.9 | 7.4 | 1.7 | 0.2 |
| 1.254 | 116.0 | 19.4 | 39.0 | 8.7 |
| 1.255 | 3559.3 | 1202.9 | 358.9 | 99.3 |
| 1.256 | 700.1 | 179.5 | 85.5 | 18.8 |
| 1.257 | 1273.7 | 237.3 | 168.0 | 35.4 |
| 1.258 | 9.5 | 3.5 | 1.3 | 0.4 |
| 1.259 | 19.5 | 11.6 | 2.1 | 0.3 |
| 1.260 | 70.9 | 48.0 | 7.1 | 1.9 |
| 1.261 | 307.4 | 139.0 | 14.8 | 6.5 |
| 1.262 | 54.9 | 13.3 | 4.0 | 0.7 |
| 1.263 | 2130.5 | 673.5 | 453.4 | 105.3 |
| 1.264 | 494.5 | 1.1 | 59.4 | 9.5 |
| 1.265 | 161.7 | 25.9 | 21.6 | 0.8 |
| 1.266 | 53.8 | 15.1 | 17.1 | 2.8 |
| 1.267 | 98.8 | 21.6 | 23.9 | 6.2 |
| 1.268 | 403.6 | 78.8 | 40.7 | 7.5 |
| 1.269 | 239.1 | 62.6 | 22.8 | 9.0 |
| 1.270 | 130.5 | 45.0 | 9.9 | 0.6 |
| 1.271 | 332.1 | 99.9 | 77.7 | 5.8 |
| 1.272 | 1823.7 | 1294.6 | 194.3 | 17.0 |
| 1.273 | 31.3 | 8.3 | 8.2 | 1.0 |
| 1.274 | 223.4 | 46.3 | 10.7 | 1.1 |
| 1.275 | 401.7 | 44.9 | 14.1 | 2.0 |
| 1.276 | 64.2 | 5.2 | 12.3 | 2.5 |
| 1.277 | 42.3 | 10.4 | 4.6 | 1.3 |
| 1.278 | 80.2 | 10.5 | 10.2 | 1.8 |
| 1.279 | 455.9 | 20.3 | 34.2 | 1.6 |
| 1.280 | 746.0 | 58.3 | 38.0 | 4.0 |
| 1.281 | 71.8 | | 7.4 | |
| 2.007 | 390.4 | | 179.1 | |
| 2.016 | 100.5 | 14.8 | 42.4 | 10.2 |
| 2.020 | 100.5 | 13.1 | 36.5 | 4.7 |
| 2.022 | 44.8 | 6.9 | 15.3 | 1.1 |
| 2.025 | 6.9 | 1.3 | 2.9 | 0.5 |
| 2.026 | 38.0 | 15.2 | 13.0 | 4.1 |
| 2.027 | 15.7 | 3.8 | 7.4 | 2.3 |
| 2.031 | 14.6 | 4.9 | 5.3 | 1.2 |
| 2.034 | 1002.6 | 392.4 | 221.1 | 312.7 |
| 2.035 | 601.0 | | 201.9 | |
| 2.036 | 579.5 | 139.9 | 232.8 | |
| 2.037 | 920.8 | | 182.2 | |
| 2.038 | 28.9 | 4.5 | 6.3 | 1.0 |
| 2.039 | 18.8 | 9.6 | 6.7 | 1.9 |
| 2.040 | 59.6 | 10.7 | 25.4 | 5.0 |
| 2.041 | 30.8 | 2.6 | 9.6 | 2.6 |
| 2.043 | 49.4 | 9.5 | 21.5 | 2.4 |
| 2.044 | 81.4 | 20.2 | 24.1 | 3.7 |
| 2.045 | 90.6 | 64.6 | 88.0 | 57.3 |
| 2.046 | 16.7 | 1.1 | 5.6 | 0.8 |
| 2.047 | 26.4 | 3.6 | 7.0 | 2.3 |
| 2.048 | 71.5 | 22.8 | 34.6 | 9.7 |
| 2.049 | 113.0 | 42.1 | 48.0 | 17.1 |
| 2.050 | 367.7 | 115.4 | 250.7 | |
| 2.051 | 1437.2 | 595.4 | 1179.8 | |
| 2.052 | 508.5 | 169.1 | 142.6 | |
| 2.053 | 951.6 | 157.1 | 182.4 | |
| 2.054 | 17.1 | 2.3 | 3.7 | 0.1 |
| 2.055 | 16.0 | 5.3 | 6.4 | 1.2 |
| 2.056 | 106.6 | 12.7 | 48.7 | 26.5 |
| 2.057 | 6.2 | 1.3 | 3.7 | 0.7 |
| 2.058 | 15.3 | 2.8 | 3.3 | 0.6 |
| 2.059 | 3.9 | 0.3 | 2.7 | 0.2 |
| 2.060 | 4.9 | 0.3 | 3.2 | 0.1 |
| 2.061 | 10.5 | 3.2 | 1.8 | 0.4 |
| 2.062 | 63.4 | 25.1 | 30.5 | 2.2 |
| 2.063 | 206.2 | 88.8 | 73.9 | 3.5 |
| 2.064 | 4.1 | 1.8 | 2.2 | 0.4 |
| 2.065 | 4.1 | 1.4 | 1.8 | 0.2 |
| 2.066 | 10.2 | 3.4 | 2.3 | 0.4 |
| 2.067 | 19.6 | 5.8 | 4.2 | 0.5 |
| 2.068 | 8.0 | 2.0 | 5.8 | 0.4 |
| 2.069 | 16.7 | 4.9 | 2.4 | 0.3 |
| 2.070 | 285.9 | 122.0 | 48.4 | 6.1 |
| 2.071 | 21.2 | 2.7 | 11.9 | 0.5 |
| 2.072 | 7.5 | 1.4 | 4.4 | 0.5 |
| 2.073 | 12.7 | 2.6 | 4.2 | 0.4 |
| 2.074 | 133.3 | 31.1 | 36.4 | 7.7 |
| 2.075 | 123.0 | 25.7 | 21.7 | 1.5 |
| 2.076 | 8.0 | 1.8 | 2.4 | 0.3 |
| 2.077 | 33.7 | 12.5 | 5.0 | 0.8 |
| 2.078 | 18.3 | 4.4 | 2.6 | 0.0 |
| 2.079 | 18.5 | 5.5 | 2.3 | 0.2 |
| 2.080 | 213.7 | 18.5 | 125.9 | 17.7 |
| 2.081 | 1446.1 | 317.4 | 1111.2 | 989.8 |
| 2.082 | 131.7 | 30.1 | 9.0 | 2.9 |
| 2.083 | 1882.9 | 380.5 | 857.6 | 706.9 |
| 2.084 | 1174.6 | 172.9 | 349.6 | 116.2 |
| 2.085 | 2391.7 | 219.6 | 812.0 | 417.7 |
| 2.086 | 1246.0 | 57.7 | 358.0 | 28.5 |
| 2.087 | 896.4 | 67.0 | 59.3 | 6.2 |
| 2.088 | 38.7 | 6.1 | 13.6 | 1.6 |
| 2.089 | 102.1 | 3.7 | 32.9 | 3.1 |
| 2.090 | 53.3 | 10.2 | 19.5 | 2.4 |
| 2.091 | 776.1 | 94.2 | 236.7 | 16.1 |
| 2.092 | 1132.5 | 128.2 | 458.0 | 73.1 |
| 2.093 | 576.3 | 99.5 | 127.7 | 19.5 |
| 2.094 | 16570.6 | 1465.6 | | |
| 2.096 | 70.2 | 9.7 | 9.6 | 1.5 |
| 2.097 | 35.4 | 2.1 | 2.8 | 0.8 |
| 2.098 | 382.5 | 13.6 | 73.5 | 3.6 |
| 2.099 | 15.0 | | 3.8 | |
| fasudil | 346.3 | 17.6 | 96.4 | 6.4 |
| H-1152 | 18.5 | 5.3 | 2.0 | 0.3 |
| H7 | | | 124.7 | 5.6 |
| Y-27632 | 197.2 | 50.6 | 60.9 | 16.9 |
| Y-39983 | 34.7 | 11.1 | 3.6 | 0.9 |

### Conclusion

Most of the compounds studied inhibited ROCK2 with a Kᵢ below 600 nM, many of these values below 60 nM. The most potent compounds in this assay showed Kᵢ values below 15 nM.

### Example 2

### Human Neutrophil Chemotaxis

### Relevance

This assay is an in vitro assay of neutrophil chemotaxis that can be used to evaluate the ability of Rho Kinase inhibitor compounds of Formula I or II to inhibit the migration of human neutrophils, an inflammatory cell that has been implicated in the pathophysiology of allergic conjunctivitis.

### Protocol

Peripheral blood from healthy human volunteers was collected and the neutrophils were isolated by Ficoll-paque density centrifugation followed by dextran sedimentation and hypotonic lysis of the red blood cells. Neutrophil chemotaxis was assessed using a modified Boyden Chamber (Neuroprobe, 96-well) with a 3 μm pore polycarbonate membrane. The ability of the tested compounds to block chemotaxis induced by a 1 μM fMLP challenge during a one hour incubation at 37° C. with 5% CO₂ was assessed in a dose response manner. The results are shown in Table 1.

### Results

The results demonstrate that Rho Kinase inhibition by Formula I or II compounds inhibited human neutrophil migration toward a chemotactic stimulant in vitro with IC₅₀ potencies ranging from less than 1 μM to nearly 24 μM (Table 2)

TABLE 2
Inhibition of fMLP-induced neutrophil chemotaxis by
Rho kinase inhibitors compounds of Formula I and/or II.

| | Chemotaxis | |
|---|---|---|
| Compound | Avg. IC₅₀ | Chemotaxis |
| Number | (nM) | SEM (nM) |
| 2.038 | 734 | 367 |
| Y-39983 | 1,390 | 803 |
| 1.131 | 1,587 | 916 |
| 2.039 | 1,643 | 949 |
| 2.025 | 1,650 | 636 |
| 1.138 | 1,850 | 212 |
| 1.091 | 2,332 | 2,077 |
| 1.136 | 2,600 | 424 |
| 1.092 | 2,747 | 1,586 |
| 2.036 | 2,767 | 1,597 |
| 1.123 | 3,050 | 778 |
| 1.124 | 3,402 | 1,964 |
| 2.026 | 3,800 | 2,970 |
| H-1152 | 4,350 | 1,202 |
| 1.087 | 4,500 | 2,598 |
| 2.034 | 4,733 | 2,733 |
| 1.034 | 5,601 | 3,234 |
| 2.035 | 6,600 | 3,811 |
| Y-27632 | 6,765 | 1,747 |
| Fasudil | 23,800 | 13,741 |

### Example 3

### Human and Murine Eosinophil Chemotaxis

Eosinophils are known to play a pivotal role in the pathogenesis of allergic conjunctivitis. Eosinophils are a major source of growth factors, lipids, basic granule proteins, cytokines and chemokines that contribute to the asthmatic disease state. Although infiltration and activation of other inflammatory cells actively contribute, it is the chemotaxis of eosinophils that is considered to be the single most important event in the pathogenesis of allergic inflammation. (See Adachi, T et. al., The Journal of Immunology. 167:4609-4615, 2001.)

### Human Eosinophil Isolation

Peripheral blood from healthy human volunteers was collected and the PMNs separated via Ficoll-paque density centrifugation followed by hypotonic lysis of the red blood cells. Subsequently, the human eosinophils were isolated from the cell suspension via StemCell Technologies Human Eosinophil Enrichment kit (Cat. No 19256) according to the manufacturer's recommendations. Briefly, unwanted cells were specifically labeled with dextran-coated magnetic nanoparticles using bispecific Tetrameric Antibody Complexes (TAC) directed against cell surface antigens on human blood cells: CD2, CD3, CD14, CD16, CD19, CD20, CD36, CD56, CD123, glycophorin A and dextran. The unwanted cells are then separated from the unlabelled eosinophils using the EasySep® magnetic isolation procedure.

### Mouse Eosinophil Isolation

Bronchoalveolar lavage was collected from ovalbumin sensitized and challenged mice in a volume of 2.5 mL lavage buffer. The lavage buffer was 0.9% saline with 10% fetal bovine serum. The pooled lavages were maintained on ice until use. The murine eosinophils were isolated using MACS cell separation (Miltenyi Biotech) by depletion of B cells and T cells by positive selection following incubation with antibody conjugated magnetic beads specific for CD45-R (B220) and CD90 (Thy 1.2), which bind B cells and T cells, respectively.

### In Vitro Chemotaxis

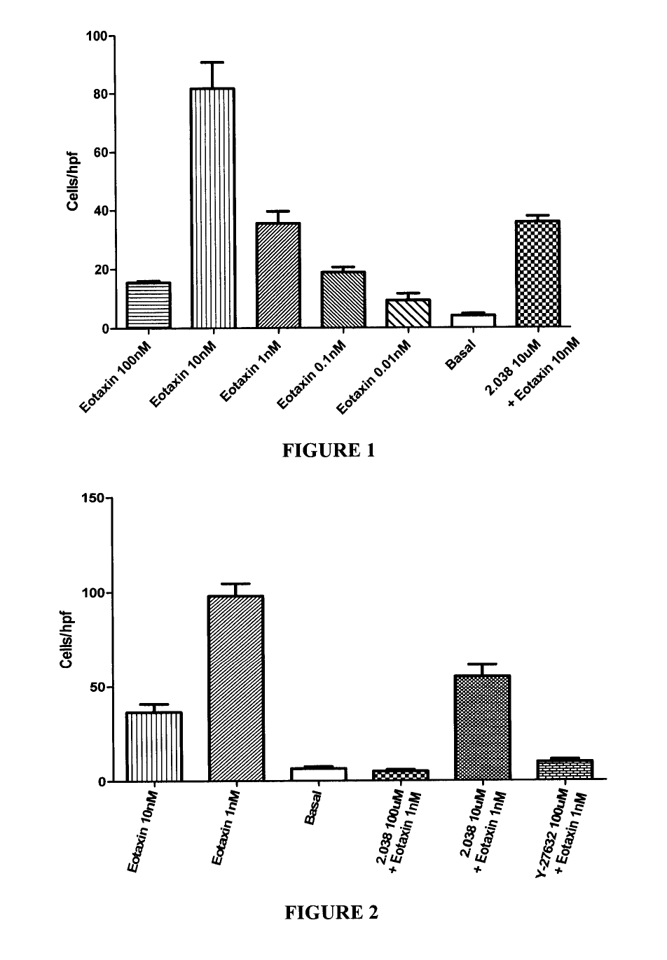

Eosinophil chemotaxis was assessed using a modified Boyden Chamber (Neuroprobe, 96-well) with a 5 μm pore membrane. The ability of the tested compounds to block chemotaxis induced by a 10 nM eotaxin challenge (mouse) or 1 nM eotaxin challenge (human) during one hour incubation at 37° C. with 5% CO₂ was assessed. Chemotaxis was quantified via microscopy by counting the number of migrated cells in at least 3 view fields per treatment. The results are shown in FIGS. 1 and 2, FIG. 1 demonstrates that chemotaxis was induced by eotaxin in murine eosinophils; the chemotactic response was subsequently inhibited by Rho kinase inhibitor Compound 2.038. FIG. 2 demonstrates that chemotaxis was induced by eotaxin in human eosinophils. The chemotactic response was subsequently inhibited by Rho kinase inhibitor Compound 2.038.

### Example 4

### Murine Model of Allergic Conjunctivitis

This example illustrates the efficacy of compounds of Formula I or II of this invention in treatment of allergic conjunctivitis (AC) in ragweed induced experimental allergic conjunctivitis. Model was prepared essentially as in Ozaki, A. et al. The J of Immunol. 175:5489-5497, 2005.

### Protocol

Induction of experimental AC by active immunization BALB/c or C57BL/6 mice (6-9 wk old) are systemically subcutaneously (s.c.) sensitized with ragweed (RW) emulsified in aluminum hydroxide hydrate gel on day 0. On days 7 and 14, mice are immunized i.p. with RW (100 μg/mouse) in PBS. Rho kinase inhibitors are instilled t.i.d. for three days prior to challenge via eye drops (˜2.5 μl) at concentrations ranging from 0.01-5%. A week after the second immunization, mice are challenged with RW (1 mg/5 μl PBS/eye) via eye drops. Clinical symptoms in the conjunctiva 15 and 30 min after administration of the challenge eye drop are evaluated as chemosis, redness, tearing, discharge, and scratching behavior, based on modified Draize criteria, Clinical appearance and photographs are evaluated by two masked observers. Scratching behavior is monitored for 30 seconds, and the frequency of scratching counted and evaluated as follows: one to three times, mild; four to six times, moderate; and more than seven times, severe. The final score is calculated as the sum of both eyes in each mouse. After 24 h, eyes are collected for histological analysis, and infiltrating cell number is counted in the conjunctiva. Vertical plane sections, including the optic nerve, are subjected to Giemsa and H&E staining.

### Results

Groups treated with Rho kinase inhibitor demonstrate improvements in at least one of the follow outcomes when compared with control animals: lid swelling, chemosis, redness, discharge, swelling, scratching as compared to control animals. Additionally histological assessments of eye vertical plane sections indicate attenuation of infiltration of inflammatory cells in Rho kinase inhibitor treatment groups as compared to controls.

### Example 5

### Increase of Endothelial Integrity and Decrease in Endothelial Permeability Following Treatment with Compounds of this Invention

Endothelial integrity is crucial in the regulation of movement of fluid and extravasation of leukocytes/inflammatory cells into tissue. Increased endothelial integrity leads to decreased fluid movement and decreased extravasation of leukocytes into tissue thus resulting in decreased tissue edema (Dudek S M et al., J Appl Physiol, 91:1487-1500, 2001 and Vandenbroucke E et al., Ann NY Acad Sci, 1123:134-145, 2008).

### Protocol

The assay is conducted essentially as in Tasaka S et al. Am J Resp Cell Mol Biol, 32:503-510, 2004. Pulmonary artery endothelial cells (PAECs) are collected and cultured in a humidified 5% CO₂ atmosphere in the medium provided by the manufacturer supplemented with 2% fetal calf serum. Endothelial cell monolayers are prepared on filters. In brief, tissue culture plate well inserts are incubated with bovine fibronectin at 37° C. for three hours to facilitate cell attachment. The fibronectin solution is aspirated, and the endothelial cells are suspended in the culture medium that is placed on a membrane filter at a density of 4×10⁵ cells per filter insert. The inserts are placed into a 6-well culture plate, where each individual well is filled with 2 ml of culture medium and incubated at 37° C. in a humidified 5% CO₂ atmosphere until PAECs reach confluence on the filter.

In order to measure permeability, the albumin that is transferred across a cultured endothelial cell monolayer grown on a porous filter is measured. PAECs on the filter are pretreated with 0.1 μM to 100 μM of a compound of Formula I or II for thirty minutes and then incubated with 10² U/ml of TNF-alpha for six or twenty-four hours. Following the incubation, the TNF-alpha containing supernatant is aspirated and 500 μl of phosphate buffered saline (PBS) containing 0.1% bovine albumin is added to the chamber located on the top of the filter insert. The insert is then placed into a culture plate well which is filled with 0.7 ml of PBS. This PBS solution is now surrounding the filter insert and occupies the lower chamber. After incubation for twenty minutes, the insert is removed from the well. The albumin concentration of the lower chamber is measured with a protein assay kit.

### Results

The TNF-alpha induced permeability of the endothelial monolayer to albumin is decreased following the treatment of the EC monolayer with the Formula I or II compounds.

### Example 6

### Promoting Effect on Neuritogenesis in Cultured Rabbit Trigeminal Nerve Cell

Restoration of corneal sensitivity in conditions leading to corneal hyposensitivity (such as following PRK and LASIK surgery and other corneal neuropathies) can be achieved by agents that induce neurotiogenesis. This example illustrates the efficacy of compounds of Formula I or II of this invention to induce neuritogenesis.

### Protocol

The trigeminal nerve cell is isolated from 2-3 day-old NZW rabbits according to the report of Chan et al. (Chan, Kuan Y. and Haschke, Richard H., Exp. Eye Res., 41: 687-699, 1985). Under ether anesthesia, after cardiac perfusion with saline, the trigeminal ganglia is removed, dispersed using a nerve dispersion solution to give a cell suspension. For the cell culture, Neurobasal medium supplemented with B27 Supplement (Invitrogen Corp., final concentration 2% v/v) and L-glutamine is used and the cultured conditions are 5% CO₂, 95% air at 37° C. The cells are seeded at about 3×10³ cells/well on a cover glass with a polylysine/laminin coating, which is immersed in a 24 well plate. As the test substance, a Rho kinase inhibitor compound of Formula I or II is added, and the control group is free of addition. After 48 hr of culture, the cells are fixed with 4% paraformaldehyde at room temperature for 2 hr, and nerve cell body and neurite are fluorescence stained using an anti-neurofilament 200 antibody that specifically recognizes neurofilaments which are intermediate filaments specific to a nerve cell and a fluorescent secondary antibody reactive therewith. The stained cells are imported as images from a fluorescence microscope into a computer and the cell body diameter and neurite length of the imported cell images are measured using an image analysis software. The cells of 3 wells are measured for each treatment group (kinase inhibitor and control). The cells having a neurite with a length of not less than twice the diameter of a cell body are taken as neuritogenetic cells, and the percentage (%) of the neuritogenetic cells in the total cells measured is calculated. Fluorescence microscope images of cultured rabbit trigeminal nerve cells demonstrate that not many cells in the non-treated control group had an extended neurite growth. However, in the kinase inhibitor-treated group, many cells have a long-extended neurite outgrowth and have a higher neuritogenetic cell percentage relative to the control group.

### Results

The results indicate that Compound of Formula I or II promotes neuritogenesis of cultured rabbit trigeminal nerve cells.

### Example 7

### Improving Effect on Rabbit Corneal Hyposensitivity Following Microkeratome Sectioning

### Protocol

New Zealand white rabbits are used. The animals are housed separately in cages in a room set to room temperature, 12 hr light cycle from arrival to the end of the test. Animals have a free access to pelletized food and tap water. Prior to the start of the test, the anterior segment of eye of the animal is visually observed and cornea stained marks by fluorescein observed, and the rabbits showing no abnormality are selected. Using Cochet-Bonnet corneal sensitivity meter, the initial value of corneal sensitivity is measured. Intramuscular injection ketamine and xylazine is given to the animals to perform systemic anesthesia, and the eyeball sufficiently exposed. Using a microkeratome, a corneal flap (diameter 8.5 mm) is prepared with a 130 μm thick blade (Arbelaez M C. et al., J. Refract Surg., May-Jun. 18, 2002 (3 Suppl): S357-60). The corneal flap is placed back into position under a microscope, and the animal woken from the anesthesia while observing the animal to prevent displacement of the flap. The next day, the condition of the animals is observed, and the animals having normally positioned corneal flap are selected.

The solution containing compounds of the Formula I or II and the control solution are administered by instillation for 1 week or 2 weeks from the next day of the corneal flap preparation. The instillation administration is performed to the surgery eye 4 times a day (30 μl installations) at 2 hr intervals using a micropipette. Concurrently, the test substance is instilled 4 times every day for one week after the surgery, 0.1% Bromfenac sodium ophthalmic solution is instilled as an anti-inflammatory agent at the first and the third instillations and 0.3% ophthalmic solution of Lomefloxacin hydrochloride are instilled as an antibacterial agent at the second and the fourth instillations

Corneal sensitivity is measured once every week from one to eight weeks after the surgery. The masked measurements are performed so that the operator would not know which administration group the subject rabbit belonged to. The corneal sensitivity is expressed by the maximal length of a nylon filament (diameter 0.12 mm) of Cochet-Bonnet corneal sensitivity meter, which induces a brink reflex upon contact of a tip of the filament with the center of the cornea.

### Results

The above test results indicate that Rho-kinase inhibitor of Formula I or II has an effect of promoting the recovery of corneal hyposensitivity due to corneal nerve section.

### Example 8

### Efficacy of a Compound of Formula I or II in Reducing Inflammation in Model of Lacrimal Gland Inflammation-Induced Dry Eye in Rabbits

### Protocol

The rabbit model of lacrimal gland inflammation-induced dry eye is used as an animal model of human dry eye disease. Rabbit lacrimal glands are injected with the T-cell mitogen Concanavalin A (Con A) to induce the conditions of dry eye. Measurements of inflammation, tear function, and corneal epithelial cell integrity are subsequently assessed as markers of efficacy. Matrix metalloproteinase-9 (MMP-9) and pro-inflammatory cytokines are quantified in tissue extracts. Tear function is monitored by measuring tear fluorescein clearance and tear breakup time (TBUT). Corneal epithelial cell integrity is determined by quantifying the uptake of methylene blue dye following the exposure of rabbits to a low humidity environment.

The compounds of Formula I or II in the concentration range 0.01-5% w/v or vehicle control is administered as a topical ophthalmic formulation with a positive displacement pipette in a volume of 30 μl to rabbits randomly assigned into treatment groups and dosed topically 4 times per day (QID) at various times during (prophylactic) or after (therapeutic) lacrimal gland injection.

### Results

Improvement in tear function and/or reduction of ocular surface injury or inflammation is observed in Compound-treated animals compared with vehicle-treated animals.

### Example 9

### Efficacy of Compounds of Formula I or II in Reducing Angiogenesis

Wet macular degeneration and edema is characterized by the accumulation of fluid in the macula as a result of leaky blood vessels. Angiogenesis, resulting in leaky blood vessels in the macula, can cause fluid retention leading to macular edema and wet macular degeneration. Reduction in angiogenesis or vascular permeability in the macula may help in the prevention of macular edema and wet macular degeneration.

### Protocol

### Directed In Vivo Angiogenesis Angioreactor

Sterile, surgical silicone tubing is cut to standard 1-cm lengths. These are plugged at one end, and are sterilized by steam autoclave. These are referred to as “angioreactors.” Using a Hamilton syringe, sterilized angioreactors are filled at 4° C. with 18 μl of Matrigel with or without angiogenic factors. These are incubated at 37° C. for 1 hour to allow gel formation, before subcutaneous implantation into the dorsal flank of C57/BL6, C57/BL6 MMP-2-deficient or athymic nude mice. Before collection of the angioreactors, mice receive a 100 μl injection of 25 mg/ml of FITC-dextran in phosphate-buffered saline (PBS) via tail vein. Quantification is performed by removal of the Matrigel and digestion in 200 μl of Dispase solution for 1 hour at 37° C. After digestion, the incubation mix is cleared by centrifugation in a benchtop centrifuge and fluorescence of the supernatant aliquots are measured in 96-well plates using an HP model spectrofluorimeter. The mean relative fluorescence±SD is determined.

### Characterization of Vascular Permeability During DIVAA

The contributions of vascular permeability to the FITC-dextran signal during quantification of angiogenic responses in the DIVAA assay are determined. The time course of FITC-dextran accumulation within the angioreactor in response to 500 ng/ml of either FGF-2 or VEGF is obtained at 9 days after implantation in angioreactors containing either FGF-2 or VEGF. Mice are injected intravenously with 100 μl of FITC-labeled dextran by tail vein, Angioreactors are then recovered at 10, 30, and 45 minutes and 1 hour after intravenous injection. FITC-dextran levels are assayed after Dispase digestion by fluorescence spectrometry as described (Guedez, et al. Am J Pathol. 162(5): 1431-1439).

### Endothelial Cell Invasion Assay

FITC-labeled Griffonia lectin (FITC-lectin), an endothelial cell selective reagent, is used to quantify invading endothelial cells into the Matrigel. Briefly, after recovery of DIVAA angioreactors and digestion with Dispase as described above, cell pellets and insoluble fractions are collected by centrifugation. The cell pellets are resuspended in 1 ml of phosphate buffered saline (PBS) and washed three times with PBS. After the final wash the cells are again collected by centrifugation and resuspended in 200 μl of 25 μg/ml of FITC-lectin and incubated at 4° C. overnight. The stained cell pellets are again centrifuged and washed three times with cold PBS. The final pellet is resuspended in 100 μl and relative fluorescence is determined for triplicate assays as described above. Mean relative fluorescence units±SD are determined as above (Guedez, et al. Am J Pathol. 162(5): 1431-1439).

### Histological Examination

Nine days after implantation, angioreactors together with the immediate surrounding tissue are dissected and fixed in 10% neutral buffered formalin. Histological sections of paraffin-embedded assays are prepared by 10-μm sectioning and stained by conventional hematoxylin and eosin methods. Sections are also stained using Griffonia lectin (FITC-lectin). Stained sections are examined and photographed using a Zeiss Axioscope fluorescent microscope with a digital camera attachment (Spot model 1.3.0; Diagnostic Instruments, Sterling Heights, Mich.). The FITC-dextran signals within whole implants are examined using an inverted fluorescent microscope (Olympus IX70) and photographed (Guedez, et al. Am J Pathol. 162(5): 1431-1439).

### Gelatinase Activity

Biochemical analysis of the gelatinase (MMP-2 and MMP-9) activity is performed by zymogram analysis. Matrigel is removed from recovered implants and resuspended in 200 μl of PBS. After mechanical disruption with a pipette tip samples are centrifuged. Aliquots of the supernatant are prepared with 2× Novex Tris-glycine sample buffer (Invitrogen, Carlsbad, Calif.) and applied to Novex 10% zymogram gels. Electrophoresis and zymogram analysis are performed as previously described (Guedez, et al. Am J Pathol. 162(5): 1431-1439).

Dosing of Compounds of this Invention

Compounds of this invention are dosed i.p. or p.o. at the dose of 1 mg/kg to 100 mg/kg of body weight one to five times per day.

### Results

Angiogenesis in this model examines the formation of neovasculature in the angioreactors of the test animals. Different contributing factors to angiogenesis are examined by DIVAA, characterization of vascular permeability, endothelial cell invasion, histological examination, and gelatinase activity. Improvement in at least one of the above-mentioned endpoints is observed in animals dosed with the compounds of Formula I or II.

### Example 10

### Efficacy of Compounds of Formula I or II in Treating Proliferative Vitreoretinopathy (PVR)

### Type I Collagen Gel Contraction Assay

The type I collagen gel contraction assay is used as an in vitro assay for studying the contractile properties of cells and is a surrogate assay for PVR. The contraction assay, previously described, (Ikuno Y, Kazlauskas A. et al. Invest Opthalmol Vis Sci., 43:41-46, 2002) is performed with slight modifications. Cells are suspended in 1.5 mg/mL neutralized collagen I at a density of 10⁶ cells/mL and transferred into a 24-well plate that has been preincubated with a solution of PBS and 5 mg/mL BSA overnight. The gel is solidified by incubating at 37° C. for 90 minutes, and then the well is flooded with EMEM and 5 mg/mL BSA. The cells are treated with 1 to 100 μM Rho kinase inhibitor compounds of Formula I or II or with control PBS. The gels are incubated at 37° C. with 5% CO₂. The initial gel diameter is 15 mm. The medium is replaced every 24 hours. The extent of contraction is calculated by subtracting the diameter of the well at a given time point from the initial diameter (15 mm).

### Effect of Compounds on PVR in a Rabbit Model

PVR is induced in the left eyes of pigmented rabbits by using a gas vitrectomy technique by injection of 0.4 mL of C₃F₈ into the vitreous cavity 4 mm posterior to the corneal limbus after anesthesia is induced (Ikuno Y, Leong F L, Kazlauskas et al. Invest Opthalmol Vis Sci., 43:483-489, 2002). Ten days later, 0.1 mL of RPE medium containing 1×10⁵ of retinal pigment epithelial (RPE) cells is injected into the vitreous cavity together with 0.1 mL of platelet-rich plasma (PRP), with a 30-gauge needle. The sixth-passage RPE cells are used in this model. Compounds of this invention were dosed by direct injection of 50 μl of formulated compound or vehicle directly into the mid-vitreous cavity. In the treated group, the experimental eye of each rabbit is injected with sufficient Rho kinase inhibitor compound of Formula I or II dissolved in 0.05 mL physiological saline immediately after RPE cell injection to achieve a final intraocular concentration of approximately of 50 μM to 10 mM. For the control group, 0.05 mL saline solution is injected. Rabbits are treated in a similar manner on days 7, 14, and 21.

Each eye is examined by indirect opthalmoscopy, and fundus video photographs are taken 3, 7, 14, 21, and 28 days after the RPE injection. The development of PVR is evaluated on videography in a masked fashion, and the PVR is graded according to the scale of Fastenberg et al. (Fastenberg D M, Diddie K R, Dorey K, Ryan S J. Am. J Opthalmol, 93:565-572, 1982).

### Results

Treatment with compound significantly inhibits RPE-induced gel contraction in a dose-dependent manner. Rabbits that receive RPE and PRP followed by either compound or the control saline solution injection every week show significant improvements in at least one of the following outcomes: (1) decreased percentage of total retinal detachment; (2) lower PVR score.

### Example 11

### Efficacy of a Compound of Formula I or II in Reducing Inflammation in Rabbit Model of Meibomianitis, Blepharitis, and Conjunctivitis

Blepharitis is accompanied by increased inflammation in the eye lid and the surrounding tissue. The following assays demonstrates efficacy of a Compound of Formula I or II in decreasing this inflammation.

New Zealand white rabbits are anesthetized with ketamine (100 mg/kg) and xylazine (10 mg/kg). Meibomian gland duct orifices are closed by cautery in the right eyes of all rabbits as previously described (Gilbard J P, et al. “Tear film and ocular surface changes after meibomian gland orifice closure in the rabbit.” Opthalmology, 96:1180-1186, 1989). Animals are divided into four treatment groups (designated groups I, II, III, and IV): group I receives no treatment; group II receives vehicle only four times a day for five days each week; group III receives tetracycline hydrochloride 1% (w/v) (Sigma Chemical, St. Louis, Mo.) four times a day for five days each week; and group IV receives a Compound of Formula I or II (between 0.01 and 5.0%, w/v) four times a day for five days each week. Treatments begin at 8 weeks post-op and continue until 20 weeks.

All rabbits are sacrificed at 20 weeks postoperatively by overdose with pentobarbital. At the time of death, corneal epithelium is removed for measurement of corneal epithelial glycogen level as previously described (Friend J et al. Invest Opthalmol Vis Sci, 24:203-207, 1983; Sherwood M B et al. Opthalmology, 96:327-335, 1989). Conjunctival biopsies are then taken for counting of goblet cell density as previously described (Gilbard J P et al. Invest Opthalmol Vis Sci, 28:225-228, 1987). Lower eyelids are then removed by sharp dissection and placed in one-half strength Karnovsky's fixative. The tissue is dehydrated through graded alcohols and embedded in methacrylate. Three micron sections are cut through the eyelids horizontally for light microscopy, and stained with alkaline giemsa.

Leukocytes are quantified in tissue sections using a method similar to that described by Sherwood et al. (Sherwood M B et al. Opthalmology, 96:327-335, 1989). For descriptive purposes, eyelid tissues are divided into three zones: 1) tarsal conjunctival epithelium, 2) underlying stroma, and 3) meibomian glands and adjacent tissue, including tarsal plate. Two separate sections, separated by a distance sufficient to provide two separate inflammatory cell populations, are examined for each eyelid. Leukocytes are identified as either neutrophils, eosinophils, basophils, or mast cells.

Twenty weeks after meibomian gland orifice closure, untreated rabbits have a significant increase in eyelid tissue mast cells, eosinophils, neutrophils and basophils relative to unoperated controls. Mast cells are not seen in the conjunctival epithelium of normal eyes nor after meibomian gland orifice closure. With this exception, all leukocyte types increase in all three tissue zones. Treatment with a Compound of Formula I or II decreases the number of leukocytes in the tissue when compared with vehicle-treated animals.

### Example 12

### NIH/3T3 Cell Morphology Assay

### Relevance

The assay demonstrates that a compound's in vitro ROCK inhibition activity manifests itself in morphology changes, such as actin stress fiber disassembly and alteration in focal adhesions in intact cells leading to inhibition of acto-myosin driven cellular contraction. These morphology changes provide the basis for the beneficial pharmacological effects sought in the setting of the disease processes described in this application, specifically the disruption of the actin stress fibers and regulation of focal adhesions and its impact on smooth muscle contractility, cell mobility, remodeling and neurite retraction (Howard et. al. The J of Cell Biology 98:1265-1271, 1984); and vasopermeability, endothelial and epithelial permeability and associated edema (Stephens et al., Am. Rev. Respir. Dis. 137:4220-5, 1988 and Vandenbroucke et al., Ann. N.Y. Acad. Sci. 1123: 134-145, 2008.)

### Protocol

NIH/3T3 cells were grown in DMEM-H containing glutamine and 10% Colorado Calf Serum. Cells were passaged regularly prior to reaching confluence. Eighteen to 24 hours prior to experimentation, the cells were plated onto Poly-L-Lysine-coated glass bottom 24-well plates. On the day of experimentation, the cell culture medium was removed and was replaced with the same medium containing from 10 nM to 25 μM of the test compound, and the cells were incubated for 60 minutes at 37° C. The culture medium was then removed and the cells were washed with warmed PBS and fixed for 10 minutes with warmed 4% paraformaldehyde. The cells were permeabilized with 0.5% Triton-X, stained with TRITC-conjugated phalloidin and imaged using a Nikon Eclipse E600 epifluorescent microscope to determine the degree of actin disruption. Results were expressed as a numerical score indicating the observed degree of disruption of the actin cytoskeleton at the test concentration, ranging from 0 (no effect) to 4 (complete disruption), and were the average of at least 2 determinations.

All compounds tested show measurable activity in the cell morphology assay, with most of the compounds providing substantial effects (score of >2 at 1 μM) on the actin cytoskeleton at the tested concentration (see Table 3).

TABLE 3
Cell Morphology Assay Data

| Compound | Cell score at 1 μM |
|---|---|
| 1.002 | 1.4 |
| 1.004 | 1.8 |
| 1.005 | 1.3 |
| 1.006 | 2 |
| 1.008 | 2 |
| 1.024 | 2.4 |
| 1.025 | 2 |
| 1.034 | 2 |
| 1.039 | 2 |
| 1.041 | 2.5 |
| 1.046 | 2.5 |
| 1.048 | 1.5 |
| 1.051 | 2.5 |
| 1.052 | 2.8 |
| 1.062 | 2.3 |
| 1.066 | 2 |
| 2.002 | 1.8 |
| 2.006 | 2.8 |
| 2.008 | 1 |
| 2.016 | 1.8 |
| 2.017 | 2 |
| 2.018 | 1.8 |
| 2.026 | 2 |

### Example 13

### In Vivo Anti-Inflammatory Activity

### Relevance

The mouse ovalbumin sensitization model has been developed by investigators to study malfunctioning of the immune system, cellular infiltration composed primarily of eosinophils and neutrophils, acute and chronic inflammation, and fluid accumulation (edema), especially in asthma. Although the model is primarily utilized in the context of asthma, this model can be utilized to demonstrate the in vivo anti-inflammatory properties of Compounds of Formula I or II.

### Protocol

Male BALB/c mice were ordered from Charles River Laboratories (Raleigh, N.C.). The animals were approximately 19 to 21 grams at time of receipt. Upon arrival, the animals were randomized into groups of five males per cage and assigned to a dosing group. Animals were quarantined for 7 days under test conditions. They were observed daily for general health status and ability to adapt to the water bottles. Animals were sensitized on day 0 and 14 of study by an intraperitoneal injection with 20 μg of ovalbumin (ova) and 2.0 mg aluminum hydroxide (alum) which initiates the development of a specific T-helper (Th) cells type 2 resulting in asthmatic animals (denoted as Ova in the figures). One group of animals received an injection of saline to use as control animals (denoted as normal in the figures). All animals were challenged with aerosolized 1% ova once daily for 25 minutes on days 28, 29, and 30 (Zosky, et al. Respiratory Research. 2004; 5:15). Aerosol challenge consists of using an Aerogen Aeroneb nebulizer and controller with a particle size of 4-6 μm mass median aerodynamic diameter (MMAD) with a distribution of 400 μl per minute. This aerosol challenge is necessary to target the Th2-driven allergic inflammation in the lower airways.

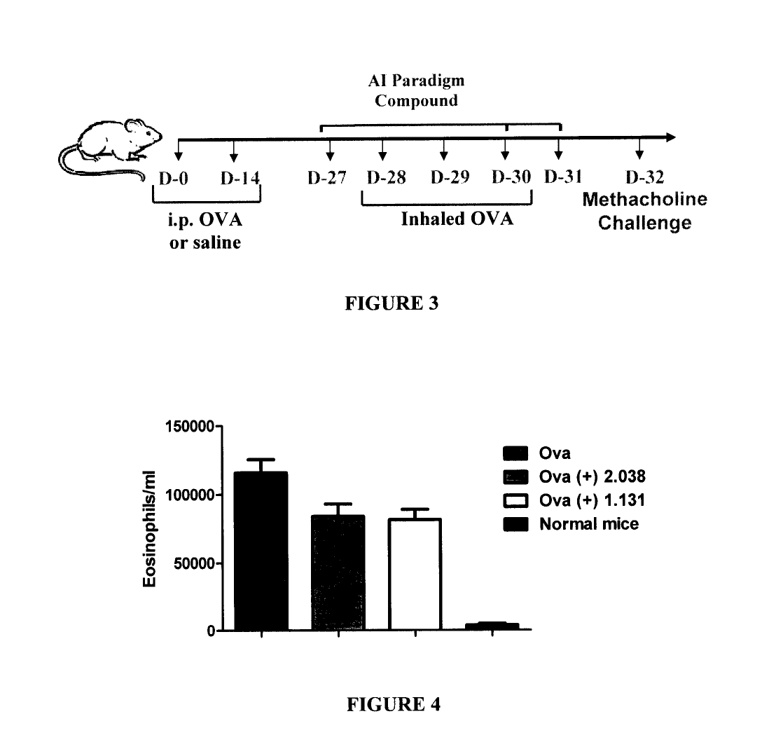

The anti-inflammatory dosing paradigm (FIG. 3) was utilized to evaluate the anti-inflammatory effects of experimental compounds. The anti-inflammatory dosing paradigm consists of dosing the animals once a day starting on day 27 and finishing on either day 30 or 31 (1 hr prior to the aerosolized ovalbumin challenges on days 28 to 30) but not on day 32 when hyperreactivity evaluation occurs (described in Example 14). On day 32 of the experiment, after measurement of airway hyperreactivity, BALF was collected and all animals were anesthetized, bled and euthanized.

Bronchoalveolar lavage fluid (BALF) was collected by infusing 3.0 ml of saline with 10% fetal calf serum into the lungs via the trachea and then withdrawing the fluid. The total amount of cells/ml of BALF fluid was determined via manual cell count on hemocytometer. The BALF was centrifuged, supernatant removed and analyzed for cytokine concentrations as described below, and cell pellet reconstituted in 500 μL of fluid. Cytospin slides were prepared from the cell pellet using 100 μL of fluid and spinning samples for 5 minutes at 5000 rpms in a cytospin centrifuge. Following Hema3 stain, relative percentages of individual leukocytes were determined on a 200 cell count for each sample. The final concentration of individual leukocyte cell types per ml of BALF was determined by multiplication of the relative percentage of individual leukocytes with the total amount of cells/ml of BALF fluid.

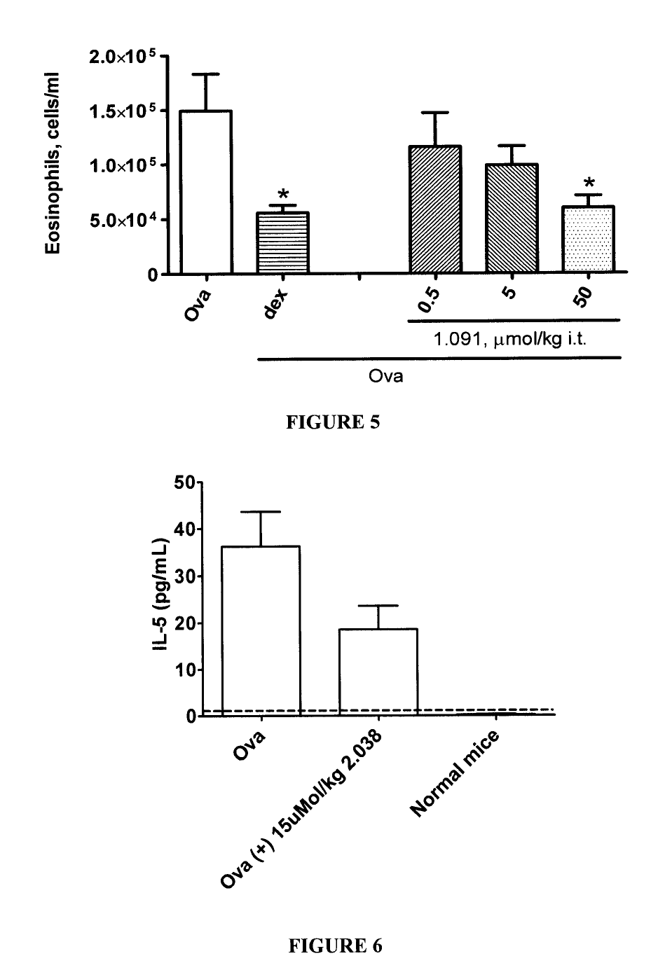

Evaluation of the differential counts performed on these samples showed an increased number of inflammatory cells in the ova-sensitized, ova-challenged animals. FIG. 4 shows the eosinophils per ml of BALF in ova-sensitized, ova-challenged mice, mice treated with Compound 2.038, mice treated with Compound 1.131 and normal mice. Compounds were dosed orally to day 31 according to the anti-inflammatory dosing paradigm shown in FIG. 3. Airway eosinophil infiltration was reduced in animals treated with the two tested compounds (FIG. 4). As shown in FIG. 5, Compound 1.091 generates a reduction of eosinophils when dosed i.t. to day 30 according to the anti-inflammatory dosing paradigm shown in FIG. 3.

The concentrations of cytokines in the BALF samples were determined using commercially available Bio-plex kits (Bio-Rad) for the detection of mouse IL-5, IL-13, and Eotaxin. The analysis of cytokine levels was measured using the Bio-Plex 200 (Bio-Rad) system according to the manufacturer's instructions. Substantial evidence suggests that cytokines play an important role in orchestrating and regulating inflammatory processes through the involvement of T-helper type 2 lymphocytes.

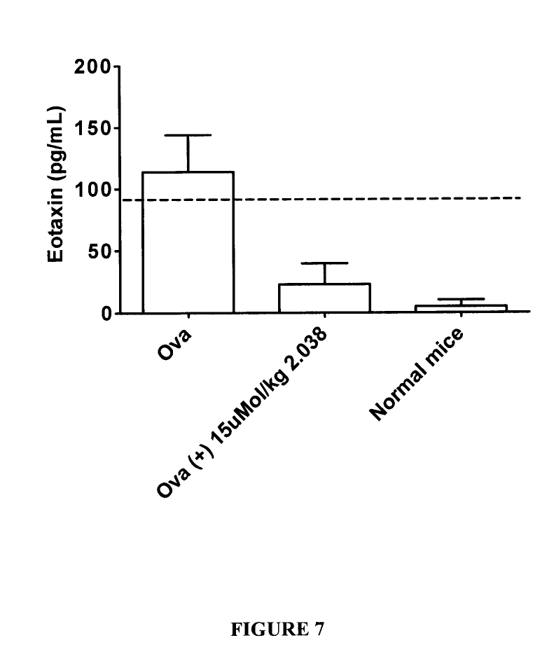

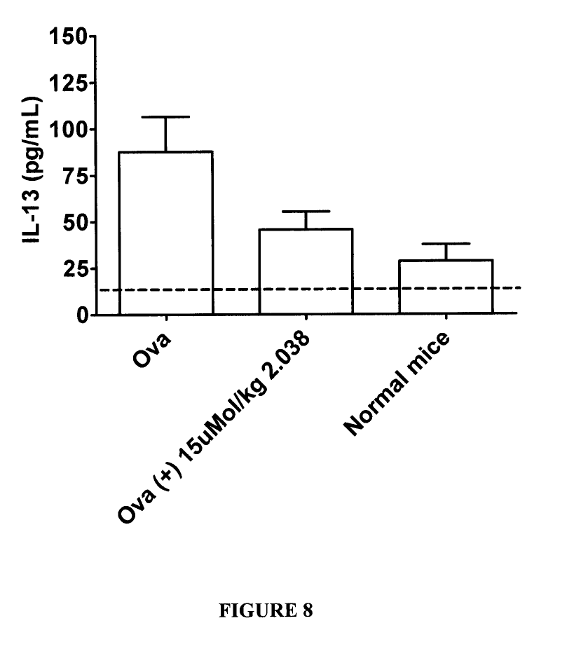

FIGS. 6-8 show the concentration of IL-5, Eotaxin, and IL-13 in (1) ova-sensitized, ova-challenged mice, (2) ova-sensitized, ova-challenged mice treated with Compound 2.038 (15 μmol/kg/oral on days 27 to 31), and (3) normal, saline-sensitized mice. The results showed that ova-sensitized, ova-challenged mice treated with Compound 2.038 had reduced levels of IL-5, Eotaxin, and IL-13.

### Example 14

### Prevention of Airway Hyperreactivity Development Via Decrease in Inflammation

### Relevance

Airway hyperreactivity is a downstream physiologic effect of inflammation in the mouse ovalbumin sensitization model. The objective of the experiment was to answer whether the decrease in inflammation due to ROCK inhibitor anti-inflammatory dosing results in the prevention of downstream physiological consequences as measured by Penh. Although this concept is demonstrated in a model of airway hyperreactivity due to pulmonary inflammation, these data support the general use of these compounds as anti-inflammatory agents to prevent the downstream physiological consequences of inflammation in an in vivo model.

### Protocol

Mouse model of ovalbumin sensitization was created as described in Example 13, The anti-inflammatory dosing paradigm (FIG. 3) was utilized to evaluate the prevention of airway hyperreactivity due to the anti-inflammatory effects of experimental compounds. The anti-inflammatory dosing paradigm consists of dosing the animals once a day starting on day 27 and finishing on either day 30 or 31 (1 hr prior to the aerosolized ovalbumin challenges on days 28 to 30) but not on day 32 when hyperreactivity evaluation occurs. On day 32 of the experiment, airway hyperreactivity was evaluated by placing conscious, unrestrained animals in a whole body plethysmometer (Buxco Wilmington, N.C.) and exposing them to escalating doses of nebulized methacholine, a known bronchial constrictor which acts through the muscarinic receptors of the lungs, (doses: 0.325-50 mg/ml). Exposure to the methacholine doses consisted of a 3 minute period during which a nebulizer was aerosolizing the methacholine and an additional 3 minute period following the cessation of nebulization. Over this 6 minute period, the plethysmometer monitors and generates numerical values for all parameters of the breath pattern. Enhanced pause (Penh), a unitless index of airway hyperreactivity, is derived from the expiratory side of the respiratory waveform measured via the plethysmograph and is used as an indirect measure of airway resistance and hyperreactivity. Penh is an indicator of changes in resistance within the airways and has been shown to be a valid marker for airway responsiveness to allergen challenge (Hamelmann, et al. Am J Respir Crit. Care Med. 1997; 156:768-775). Following the methacholine dose response, BALF was collected and all animals were anesthetized, bled and euthanized.

### Statistical Methods

Within each experiment, a mouse was given a single compound and exposed to increasing doses of methacholine [0 (baseline), 0.375, 0.75, 1.5, 3, 6, 12, 25, 50 mg/ml]. The Penh value at each of the dose levels of methacholine represents the 6-minute average response. Change from baseline (CFB) in Penh was calculated at each methacholine dose and the area under the curve (AUC) for these CFB values was calculated using the trapezoidal rule. This same approach was applied for each mouse across multiple experiments.

For statistical analyses, a linear mixed-effects model where the response was the log 10 transformed value of AUC described above was used. Data from equal experimental conditions across experiments performed on different days were pooled for statistical analysis and data reporting. The various compounds were compared adjusting for the log 10-transformed baseline value of Penh and the chamber (1 of 10) of the plethysmometer each mouse was contained in during an experiment. A random intercept for each experiment was assumed to account for possible similarities of the results obtained from a given experiment (i.e., as a “blocking effect”). Pairwise comparisons of the compounds were performed using approximate t-tests to test the null hypothesis of no compound difference of the least-squares means of log 10(AUC). p values of less than 0.05 were considered statistically significant Computations were performed using PROC MIXED (SAS Version 9.1).

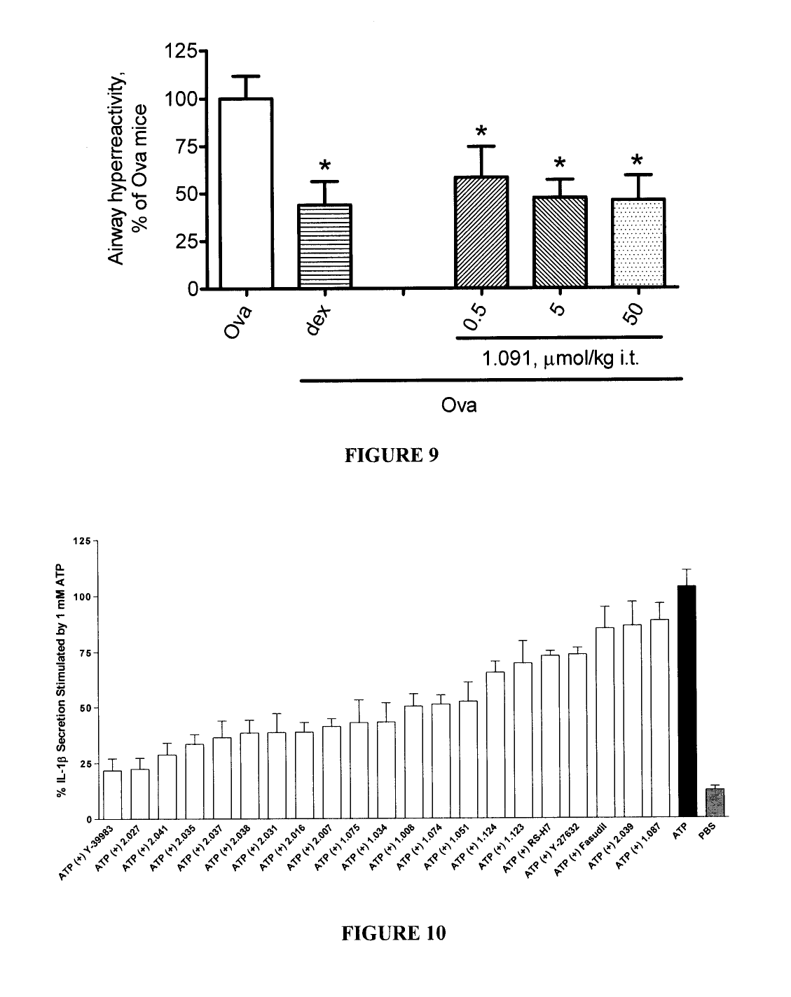

For Table 4, Penh values are reported as log 10 transformed AUC values. For FIG. 9, linear AUC values from compound treated mice were reported as a percent of linear AUC values from vehicle-treated ovalbumin-sensitized/ovalbumin-challenged (asthmatic) mice.

The oral administration of 15 μMol/kg of Compound 1.131 or 2.038 once a day during days 27 to 31 resulted in prevention of airway hyperreactivity to methacholine dosed on Day 32 (Table 4). As shown in FIG. 9 and Table 4, intratracheal administration of Compound 1.091 once a day during days 27 to 30 (FIG. 9) or Compounds 1.161, 2.066 or 2.059 once a day during days 27 to 31 (Table 4) according to the anti-inflammatory dosing paradigm shown in FIG. 4 resulted in prevention of airway hyperreactivity. Compound 1.091, 1.161, 2.066 or 2.059 had similar efficacy to dexamethasone, a corticosteroid anti-inflammatory control. These data support the use of these compounds to prevent the downstream physiologic consequences of inflammation.

TABLE 4
Anti-inflammatory dosing: Statistical Analysis of the AUC for
Average Penh Values Determined During Experiment Normalized
to Baseline for Each Animal

| | | Number | | | |
|---|---|---|---|---|---|
| | Dosing | of | | | |
| | concentration/ | animals | log10A | Stan- | Student |
| | route of | per | UC | dard | t-test |
| | administration | group | (Penh) | Error | p-value |
| asthmatic | Vehicle/oral | 70 | 2.3354 | 0.04751 | |
| 1.131 | 15 μmol/kg/oral | 10 | 2.0674 | 0.1061 | 0.0133 |
| 2.038 | 15 μmol/kg/oral | 20 | 1.8981 | 0.07966 | <0.0001 |
| 1.161 | 0.5 μmol/kg/ | 10 | 2.0405 | 0.1083 | 0.0077 |
| | intratracheal | | | | |
| 2.066 | 0.5 μmol/kg/ | 10 | 2.0248 | 0.1091 | 0.0055 |
| | intratracheal | | | | |
| 2.059 | 0.5 μmol/kg/ | 10 | 1.9979 | 0.1084 | 0.0024 |
| | intratracheal | | | | |
| Y-27632 | 30 μmol/kg/oral | 10 | 1.9942 | 0.1062 | 0.0017 |
| Dexamethasone | 1 mg/kg/oral | 30 | 2.0216 | 0.06546 | <0.0001 |
| non-asthmatic | Vehicle/oral | 20 | 1.7810 | 0.07973 | <0.0001 |

Compounds were administered on days 27 to 31 according to the anti-inflammatory dosing paradigm.
The t-test was conducted for the comparison of compound-treated to vehicle-treated “asthmatic groups” based on the vehicle which was run in every study.

### Example 15

### IL-1β Monocyte Secretion Assay

IL-1β plays a major role in a number of inflammatory diseases. In the presence of increased IL-1β levels, certain tissues show an up-regulation of adhesion molecules, increased vascular permeability, and increased extravasation of leukocytes including neutrophils, macrophages, and lymphocytes. In this assay, lipopolysaccharide (LPS) was used as the inflammatory stimulus to induce cytokine production in human monocytes, and ATP was used to stimulate release of the pro-inflammatory cytokine IL-1β. Monocytes are known to orchestrate the innate immunity response to LPS by expressing a variety of inflammatory cytokines including IL-1β, TNF-α, IL-6, and many others (Gua M, et al., Cellular Signalling. 13: 85-94, 2001).

Peripheral blood from healthy human volunteers was collected and the monocytes isolated via Ficoll-paque density centrifugation. The resultant pellet was re-suspended in media containing 1 ng/mL lipopolysaccharide (LPS) and plated at a density of 500,000 cells/mL. After 3 hours of incubation (37° C., 5% CO₂, humidified air), monocytes were selected by adherence to the tissue culture plastic by washing wells with media. Following the media wash, cells were incubated for 2 minutes with the Rho kinase inhibitors (10 μM) prior to the addition of 1 mM ATP. Cells were allowed to incubate with compounds for 30 minutes at 37° C. after which the supernatant was removed for immediate determination of IL-1β concentration. The concentration of IL-1β in cell supernatants was measured using the Human IL-1 kit and Bio-Plex system (Bio-Rad) according to manufacture's instructions.

FIG. 10 shows percent inhibition of IL-1β secretion in human monocytes by rho kinase inhibitors. The tested Rho kinase inhibitors of Formula I or II at a 10 μM concentration demonstrated a varying efficacy range. Many compounds effectively reduced IL-1β secretion to low level.

### Example 16

### Human Monocyte Cytokine Secretion Assay

### Relevance:

This assay demonstrates a compound's ability to inhibit the secretion of multiple pro-inflammatory cytokines from human monocytes. Reduction in the levels of pro-inflammatory cytokines is associated with improvement in disorders with an inflammatory component.

### Protocol

Peripheral blood from healthy human volunteers was collected and the monocytes isolated via Ficoll-paque density centrifugation. Monocytes were purified via an Easy Sep© Monocyte Enrichment Kit (Product number 19059) according to the manufacturer's instructions, The purified monocytes were then plated in 96-well plates at a density of 300,000 cells/mL in RPMI 1640+10% heat inactivated FBS media. The cells were allowed to pre-incubate with test compound at the indicated concentration for 30 minutes (37° C., 5% CO₂, humidified air); after which the supernatant was removed and media containing compound and 1 ng/mL LPS was added. Cells were allowed to incubate with compounds and LPS for 4 hours at 37° C. after which the supernatant was removed and stored at −80° C. Cytokine concentrations in the supernatant were determined using commercially available Bio-Rad Bio-plex™ kits according the manufacturer's instructions.

### Results:

Compounds of Formulae I and II inhibit the release of multiple cytokines from human monocytes when incubated at 10 μM concentration in vitro, as shown in Table 5. Shown further in Table 6, potency determinations on compounds 2.059 and 2.066, both potent inhibitors of ROCK1 and ROCK2 and both of the chemical class in which R₂ is R₂-2, dose-dependently reduced the secretion of IL-1β, TNF-α and IL-9 from LPS-stimulated human monocytes, with potencies ranging from approximately 170 nM to 1 μM.

TABLE 5
Percent inhibition values for inhibition of cytokine secretion
at 10 μM of test compound

| Compound | IL-1β % | IL-6 % | TNF-α % |
|---|---|---|---|
| 1.072 | 98.2 | 96.1 | 83.8 |
| 1.074 | 43.9 | 96.0 | 87.7 |
| 1.075 | 49.7 | 73.9 | 51.6 |
| 1.076 | 51.0 | 81.2 | 78.9 |
| 1.077 | 30.3 | 43.3 | 52.3 |
| 1.078 | 60.4 | 111.0 | 88.1 |
| 1.079 | 59.3 | 31.1 | 56.5 |
| 1.091 | 165.5 | 108.2 | 104.6 |
| 1.093 | 109.0 | 49.7 | 76.1 |
| 1.106 | 121.5 | 95.0 | 80.6 |
| 1.107 | 111.3 | 122.1 | 83.1 |
| 1.108 | 131.3 | 89.8 | 116.7 |
| 1.109 | 190.5 | 312.9 | 118.3 |
| 1.110 | 133.6 | 111.7 | 118.6 |
| 1.123 | 82.6 | 64.7 | 62.7 |
| 1.124 | 99.5 | 101.4 | 61.5 |
| 1.127 | 198.0 | 67.3 | 97.3 |
| 1.131 | 48.3 | 68.6 | 85.2 |
| 1.132 | 58.6 | 72.5 | 80.3 |
| 1.133 | 54.5 | 70.7 | 66.2 |
| 1.134 | 43.2 | 74.6 | 69.1 |
| 1.135 | 57.0 | 123.2 | 108.0 |
| 1.136 | 66.3 | 95.0 | 71.5 |
| 1.137 | 40.3 | 46.2 | 58.0 |
| 1.138 | 257.4 | 76.6 | 130.9 |
| 1.141 | 50.4 | 71.7 | 75.7 |
| 1.142 | 82.8 | 40.7 | 68.6 |
| 1.143 | 76.8 | 130.5 | 66.4 |
| 1.145 | 129.2 | 95.1 | 88.9 |
| 1.146 | 85.2 | 128.0 | 97.7 |
| 1.148 | 63.9 | 78.6 | 56.1 |
| 1.149 | 69.8 | 121.5 | 119.9 |
| 1.150 | 78.2 | 89.2 | 94.4 |
| 1.151 | 84.5 | 114.1 | 88.9 |
| 1.152 | 74.7 | 94.7 | 120.1 |
| 1.153 | 64.1 | 106.2 | 74.3 |
| 1.154 | 52.3 | 104.4 | 86.4 |
| 1.155 | 76.7 | 121.8 | 79.7 |
| 1.156 | 60.7 | 92.5 | 70.5 |
| 1.157 | 121.4 | 92.6 | 65.1 |
| 1.158 | 80.8 | 133.1 | 86.6 |
| 1.159 | 97.1 | 84.8 | 76.1 |
| 1.161 | 87.7 | 86.3 | 153.5 |
| 1.162 | 95.5 | 99.8 | 158.7 |
| 1.163 | 166.7 | 140.9 | 91.6 |
| 1.164 | 80.1 | 109.5 | 89.0 |
| 1.165 | 129.9 | 114.3 | 103.5 |
| 1.166 | 107.0 | 87.2 | 82.2 |
| 1.170 | 80.6 | 72.7 | 67.8 |
| 1.171 | 78.9 | 91.8 | 72.2 |
| 1.173 | 86.1 | 79.5 | 80.1 |
| 1.175 | 29.3 | 38.2 | 47.4 |
| 1.176 | 95.2 | 112.4 | 72.4 |
| 1.183 | 68.7 | 123.3 | 76.5 |
| 1.185 | 39.8 | 63.0 | 66.6 |
| 1.186 | 64.1 | 105.3 | 68.2 |
| 1.195 | 115.4 | 94.4 | 67.7 |
| 1.197 | 179.1 | 128.8 | 83.3 |
| 1.200 | 0.0 | 0.0 | 0.2 |
| 1.206 | 88.7 | 164.0 | 97.3 |
| 1.208 | 62.0 | 109.0 | 92.0 |
| 1.212 | 116.3 | 111.0 | 108.1 |
| 1.213 | 111.1 | 81.7 | 77.4 |
| 1.215 | 136.7 | 63.2 | 60.4 |
| 1.217 | 118.6 | 73.8 | 71.3 |
| 1.219 | 138.9 | 127.7 | 82.1 |
| 1.223 | 117.0 | 88.5 | 60.7 |
| 1.226 | 99.3 | 52.2 | 66.6 |
| 1.227 | 69.4 | 66.7 | 79.3 |
| 1.229 | 44.9 | 63.2 | 50.7 |
| 1.233 | 78.5 | 78.9 | 79.0 |
| 1.236 | 75.2 | 93.0 | 98.0 |
| 1.237 | 97.1 | 100.9 | 70.6 |
| 1.238 | 101.1 | 62.9 | 73.2 |
| 1.239 | 39.4 | 84.7 | 58.5 |
| 1.246 | 103.0 | 108.3 | 79.0 |
| 1.249 | 133.8 | 56.2 | 60.0 |
| 1.252 | 139.2 | 68.3 | 101.6 |
| 1.253 | 160.6 | 228.6 | 126.8 |
| 1.258 | 104.1 | 83.5 | 94.0 |
| 1.262 | 145.7 | 156.6 | 135.3 |
| 2.026 | 166.0 | 180.7 | 109.1 |
| 2.031 | 49.0 | 89.3 | 66.4 |
| 2.038 | 90.8 | 79.7 | 70.2 |
| 2.039 | 49.8 | 70.3 | 47.8 |
| 2.054 | 24.0 | 56.8 | 37.9 |
| 2.058 | 1.2 | 1.3 | 10.6 |
| 2.059 | 0.3 | 0.0 | 6.9 |
| 2.060 | 5.9 | 19.6 | 33.0 |
| 2.064 | 14.3 | 45.7 | 66.2 |
| 2.066 | 0.0 | 0.0 | 25.2 |

TABLE 6
IC₅₀ values for inhibition of cytokine secretion

| | IL-1β (nM) | TNF-α (nM) | IL-9 (nM) |
|---|---|---|---|
| Compound 2.059 | 169.4 ± 13.0 | 207.1 ± 17.0 | 268.6 ± 28.1 |
| Compound 2.066 | 346.2 ± 182.3 | 610.6 ± 154.1 | 934.9 ± 407.5 |

### Example 17

### LPS-Induced Neutrophilia and Cytokine Production Assay

### Relevance

Marked neutrophilia can occur upon tissue inflammation. The LPS-induced neutrophilia model is often used to determine the potential efficacy of therapeutic approaches to limit inflammatory responses. This assay is an in vivo assay of neutrophil accumulation and cytokine production that can be used to evaluate the activity of Rho Kinase inhibitor compounds of Formula I or II as anti-inflammatory agents in a whole animal model, Neutrophil accumulation and cytokine production are indicative of an inflammatory response and the activity of compounds to decrease neutrophil accumulation and cytokine production in this assay supports the use of these compounds to treat disorders with an inflammatory component

### Protocol

Male BALB/c mice, approximately 19 to 21 grams, were ordered from Charles River Laboratories (Raleigh, N.C.). All animals were challenged with aerosolized LPS (10 μg/ml) for 25 minutes on study day 0. LPS aerosol was generated using an Aerogen Aeroneb nebulizer and controller providing a flow of 400 μl/min and a particle size of 2-4 μm MMAD. Rolipram was administered i.p at 20 mg/kg. Compound 1.091 or Compound 2.059 was administered intratracheally (i.t.) at 0.5-50 μmol/kg body weight one hour prior to LPS challenge. Four hours following LPS challenge, BALF was collected using a total of 3 ml of 0.9% sodium chloride containing 10% fetal calf serum. Total cell counts were determined using the Coulter Counter. For differential evaluations, BALF was centrifuged and cytospin slides prepared and stained with Hema3 stain. Manual leukocyte counts were then completed on 200 cells. The final concentration of individual leukocyte cell types per ml of BALF was determined by multiplication of the relative percentage of individual leukocytes with the total amount of cells/ml of BALF fluid. The concentration of IL-1β in the BALF samples was determined using commercially available Bio-plex kits (Bio-Rad). The analysis of cytokine levels was measured using the Bio-Plex 200 (Bio-Rad) system according to the manufacturer's instructions.

### Results

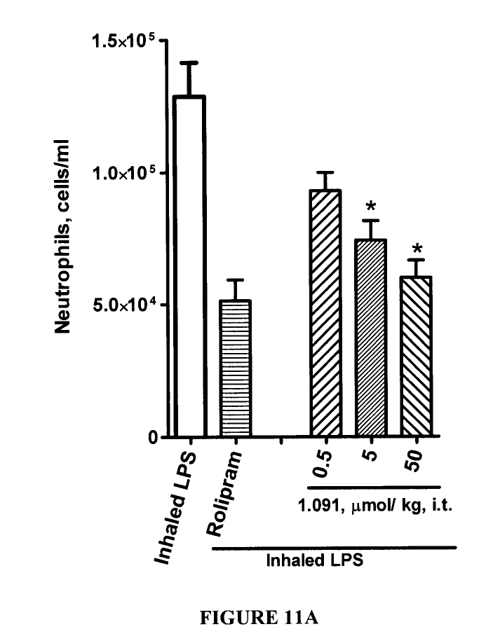

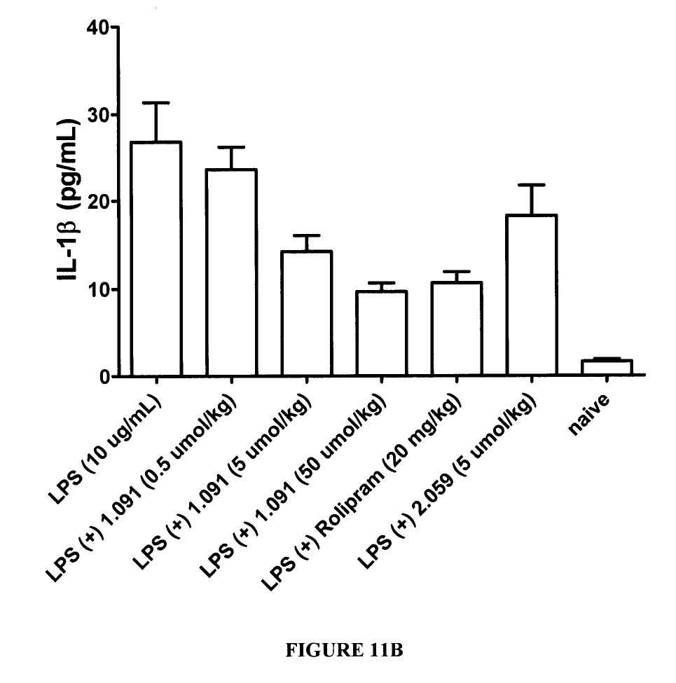

FIG. 11A shows a significant reduction in pulmonary neutrophilia influx after intratracheal dosing of Compound 1.091. The efficacy of Compound 1.091 when dosed intratracheally is similar to the efficacy of the control compound rolipram dosed i.p. FIG. 11B shows the reduction in IL-1β after intratracheal administration of Compound 1.091 or Compound 2.059. These data demonstrate the efficacy of Rho kinase inhibitors of Formula I or II to inhibit inflammatory responses in vivo.

### Example 18

### PDGF-Stimulated Smooth Muscle Cell Proliferation Assay

### Relevance:

This assay demonstrates a compound's ability to inhibit cellular proliferation induced by platelet derived growth factor (PDGF). Activity of compounds in the assay demonstrates the anti-proliferative properties of these compounds and supports the use of these compounds in the treatment of disorders associated with a proliferative component.

### Protocol

Effects on cell proliferation were measured using a bromodeoxyuridine (BrdU) incorporation assay. A-10 rat thoracic aorta cells (ATCC #CRL 1476) were plated at 11000 cells per well in 96-well plates in Dulbecco's Modified Eagles Medium-High Glucose (Gibco cat. # 11995-065) containing 10% Fetal Bovine Serum (Sigma EC# 232-690-6) and allowed to grow for 24 hrs in an incubator at 37° C. Growth media was then removed and the cells were washed with warmed PBS (Gibco cat# 14190-144). Serum free media containing 01% BSA was added to the cells. 24 hours later the media was removed and replaced with warmed serum free media. Cells were treated with either 1 μM or 10 μM of test compound and incubated for 60 min at 37° C. prior to the addition of 10 ng/mL PDGF (BD Biosciences cat. # 354051) and placed in an incubator at 37° C. for 18 hrs with both compound and stimulant present. Proliferation was then monitored using the BrdU Cell Proliferation Assay, HTS (Calbiochem cat. #t HTS01). BrdU was allowed to incorporate into cells for 24 hours prior to the addition of fixative/denaturing solution and the fluorometric detection of incorporated BrdU using a BrdU antibody as per manufacturer's directions. Data are reported as a percent of the PDGF-stimulated BrdU incorporation.

### Results:

As shown in Table 7, compounds of Formulae I and II reduced PDGF-stimulated proliferation of A10 cells with efficacy ranging from 10-80% inhibition when dosed in vitro at 1 μM.

TABLE 7
Reduction of PDGF-stimulated proliferation of A-10 cells as a
percent of the total challenge-stimulated proliferation.

| | Percent of | Percent of | Percent of | Percent of |
|---|---|---|---|---|
| | PDGF | PDGF | PDGF | PDGF |
| | Induced | Induced | Induced | Induced |
| | Proliferation | Proliferation | Proliferation | Proliferation |
| | at 10 μM | at 10 μM | at 1 μM | at 1 μM |
| Compound | Avg | SEM | Avg | SEM |
| 1.074 | 46.9 | 3.5 | 79.9 | 9.7 |
| 1.076 | 53.7 | 4.1 | 84.0 | 8.5 |
| 1.091 | 69.3 | 5.5 | 85.7 | 5.3 |
| 1.108 | 43.7 | 1.6 | 83.1 | 6.7 |
| 1.124 | 61.6 | 2.6 | 68.5 | 3.1 |
| 1.131 | 36.6 | 2.4 | 61.7 | 4.8 |
| 1.132 | 30.3 | 1.3 | 48.9 | 3.4 |
| 1.135 | 35.0 | 3.9 | 52.6 | 4.9 |
| 1.136 | 39.8 | 2.6 | 71.4 | 1.3 |
| 1.138 | 27.0 | 1.7 | 46.3 | 1.5 |
| 1.148 | 63.5 | 3.0 | 56.9 | 2.7 |
| 1.151 | 63.8 | 4.1 | 51.0 | 2.1 |
| 1.161 | 33.4 | 0.9 | 50.0 | 3.7 |
| 1.162 | 42.5 | 1.6 | 55.6 | 2.3 |
| 1.165 | 57.9 | 1.2 | 74.8 | 6.1 |
| 1.167 | 52.7 | 4.6 | 78.8 | 4.5 |
| 1.173 | 35.8 | 2.8 | 55.4 | 4.2 |
| 1.175 | 49.0 | 2.5 | 58.2 | 2.3 |
| 1.180 | 64.8 | 5.0 | 92.4 | 7.9 |
| 1.197 | 48.9 | 2.8 | 52.5 | 1.5 |
| 1.204 | 42.8 | 5.3 | 79.3 | 3.0 |
| 1.206 | 51.1 | 2.1 | 77.5 | 5.8 |
| 1.213 | 52.3 | 3.6 | 70.1 | 2.3 |
| 1.215 | 54.0 | 5.3 | 70.8 | 4.0 |
| 1.237 | 51.4 | 4.8 | 63.5 | 5.2 |
| 1.238 | 48.6 | 3.2 | 40.7 | 1.9 |
| 1.239 | 37.8 | 1.6 | 41.7 | 2.7 |
| 1.253 | 47.9 | 2.0 | 44.8 | 3.1 |
| 1.258 | 43.4 | 4.7 | 50.5 | 3.3 |
| 2.009 | 56.5 | 3.9 | 128.9 | 13.4 |
| 2.022 | 39.4 | 1.1 | 89.7 | 4.5 |
| 2.025 | 68.0 | 4.1 | 69.8 | 4.6 |
| 2.026 | 52.0 | 2.5 | 74.5 | 6.5 |
| 2.027 | 64.4 | 5.8 | 79.4 | 5.6 |
| 2.031 | 52.6 | 2.8 | 90.3 | 9.9 |
| 2.038 | 62.7 | 3.5 | 58.6 | 1.2 |
| 2.041 | 61.5 | 3.1 | 81.8 | 4.8 |
| 2.046 | 32.1 | 1.4 | 57.4 | 1.2 |
| 2.047 | 53.8 | 3.2 | 65.3 | 3.0 |
| 2.054 | 84.6 | 6.4 | 68.2 | 4.0 |
| 2.059 | 25.5 | 1.1 | 75.0 | 5.7 |
| 2.064 | 56.2 | 3.9 | 53.1 | 1.9 |
| 2.066 | 19.8 | 0.7 | 20.0 | 0.7 |

### Example 19

### Smooth Muscle Proliferation Assay

Cellular proliferation and remodeling play a role in the pathophysiology of multiple disease states. In this assay, inhibition of proliferation induced by fetal bovine serum is measured. Fetal bovine serum contains a complex mix of growth factors that contribute to the activation of multiple growth signals within the cells. Activity of compounds in this assay demonstrates a robust anti-proliferative effect of the compounds and is supportive of the use of these compounds to treat diseases associated with a proliferative component.

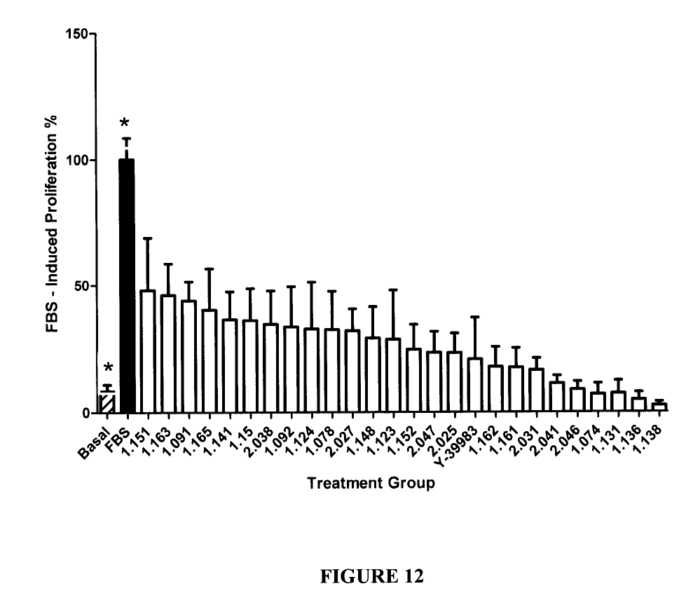

Effects on cell proliferation were measured using a radiographic technique know as [³H]thymidine incorporation. A-10 rat thoracic aorta cells (ATCC #CRL 1476) were grown on 24-well plates in Dulbecco's Modified Eagles Medium-High Glucose (Gibco cat. # 11995-065) containing 10% Fetal Bovine Serum (Sigma EC# 232-690-6) for 24 hrs in an incubator at 37° C. Growth media was then removed, the cells were washed with warmed PBS (Gibco cat# 14190-144) and warmed serum free media containing 0.1% BSA in order to force the cells into a quiescent state. 24 hours later the media was removed and replaced with warmed serum free media containing from 10 nM to 30 μM of test compound. The cells were incubated for 60 min at 37° C. The cells were then stimulated with either 10% FBS or 10 ng/mL PDGF (BD Biosciences cat# 354051) and placed in an incubator at 37° C. for 18 hrs. [³H] thymadine (Perkin Elmer NET027A001MC) was then added to the cells at a final concentration of 3 uCi/mL and placed in an incubator at 37° C. for 24 hrs. The media was removed and the cells were washed with warmed PBS twice. 500 μL of warmed trypsin (Gibco cat# 25300-054) was added to each well and they were place in an incubator at 37° C. for 15 min. To precipitate the DNA, 500 μL of ice cold 20% TCA (MP Biomedicals cat# 152592) was added to each well. The resulting suspension was filtered using a vacuum manifold and glass fiber filters (Whatman cat# 1827-025). The fiber filters were then counted using a liquid scintillation counter (Wallac 1409). Results were normalized to the total signal of the challenge, graphed using Graphpad Prism (Ver, 5.00) and reported as % of FBS stimulated proliferation. The results are shown in FIG. 12. The results demonstrate that the tested Rho kinase inhibitors of Formula I or II compounds reduced the smooth muscle cell proliferation in vitro. The majority of the tested compounds decreased the proliferation to less than 50% of the normal rate at a concentration of 30 μM.

### Example 20

### Akt3 and p70S6K Inhibition Assay

### Relevance:

This assay demonstrates a compound's ability to inhibit the kinases Akt3 and p70S6K in vitro. Both kinases are known to play a role in proliferation pathways.

### Protocol

Inhibition of Akt3 and p70S6K activity was determined using the IMAP™ FP Progressive Binding Kit (Molecular Devices product number R8127). Akt3 human enzyme (Upstate Chemicon #14-502), or p70S6K human enzyme (Upstate Chemicon #14-486), and Flourescein tagged substrate peptide (Molecular Devices product number R7110) or (Molecular Devices product number R7184), for Akt3 and p70S6K respectively, was pre-incubated with test compound for 5 minutes in buffer containing 10 mM Tris-HCL pH 7.2, 10 mM MgCl₂, 1 mM DTT and 0.1% BSA. Following the pre-incubation, 30 μM ATP was added to initiate the reaction. After 60 minutes at RT, Molecular Devices IMAP™ binding solution was added to bind phosphorylated substrate. After 30 minutes of incubation in the presence of the IMAP™ beads the fluorescence polarization was read and the ratio was reported as mP. IC₅₀ results were calculated using the Prism software from Graphpad. The Kᵢ values were determined according to the following formula: Kᵢ=IC₅₀/(1+([ATP Challenge]/EC₅₀ ATP)).

### Results:

As shown in Table 8, many compounds of Formulae I and II show sub-micromolar inhibitory potencies against both Akt3 and p70S6K.

TABLE 8
Akt3 and p70S6K potency data

| | | Akt3 | p70S6K Ki, | p70S6K |
|---|---|---|---|---|
| | Akt3 Ki, Avg, | Ki, StdDev, | Avg, | Ki, StdDev, |
| Compound | nM | nM | nM | nM |
| 1.072 | 4752.1 | 617.1 | 1130.3 | 263.7 |
| 1.074 | 437.4 | 13.2 | 548.3 | 170.9 |
| 1.075 | 5321.5 | 61.8 | 974.6 | 166.8 |
| 1.076 | 240.9 | 6.2 | 414.3 | 162.7 |
| 1.077 | 5253.2 | 1422.9 | 715.5 | 291.5 |
| 1.078 | 3267.4 | 150.9 | 1678.1 | 640.4 |
| 1.079 | 7191.7 | 445.6 | 3012.8 | 963.8 |
| 1.091 | 5388.5 | 171.6 | 1420.4 | 78.5 |
| 1.093 | 1824.9 | 27.9 | 2025.6 | 356.8 |
| 1.106 | 3914.9 | 257.1 | 1329.1 | 268.0 |
| 1.107 | 16304.0 | 1575.9 | 3356.5 | 701.7 |
| 1.108 | 205.0 | 2.2 | 510.6 | 106.0 |
| 1.109 | 5190.9 | 318.3 | 2495.5 | 314.8 |
| 1.110 | 462.6 | 2.3 | 1298.2 | 175.9 |
| 1.123 | 2406.9 | 287.1 | 2810.7 | 597.6 |
| 1.124 | 7868.0 | 909.4 | 3325.3 | 542.0 |
| 1.127 | 975.4 | 126.4 | 2065.5 | 54.3 |
| 1.131 | 282.6 | 2.0 | 502.8 | 112.4 |
| 1.132 | 81.8 | 8.2 | 514.6 | 111.1 |
| 1.133 | 148.3 | 3.7 | 531.8 | 45.6 |
| 1.134 | 150.7 | 22.1 | 519.7 | 81.1 |
| 1.135 | 444.2 | 32.9 | 588.6 | 142.4 |
| 1.136 | 289.7 | 12.5 | 1236.7 | 413.1 |
| 1.137 | 197.9 | 10.3 | 353.6 | 132.2 |
| 1.138 | 91.3 | 48.3 | 443.5 | 36.3 |
| 1.141 | 1263.0 | 133.1 | 387.5 | 5.8 |
| 1.142 | 8268.5 | 702.6 | 2524.8 | 882.2 |
| 1.143 | 706.5 | 130.5 | 538.2 | 173.7 |
| 1.145 | 1190.5 | 63.5 | 2296.4 | 602.2 |
| 1.146 | 204.9 | 24.7 | 741.5 | 272.3 |
| 1.148 | 1131.4 | 161.7 | 435.5 | 138.0 |
| 1.149 | 7395.9 | 410.0 | 1888.4 | 661.8 |
| 1.150 | 3183.1 | 98.7 | 1273.8 | 106.7 |
| 1.151 | 708.9 | 112.8 | 530.7 | 69.6 |
| 1.152 | 1976.2 | 155.8 | 523.5 | 295.5 |
| 1.153 | 9950.2 | 2150.4 | 2376.1 | 553.3 |
| 1.154 | 4947.5 | 541.2 | 1130.1 | 355.3 |
| 1.155 | 5680.5 | 644.8 | 1751.6 | 502.8 |
| 1.156 | 8772.6 | 427.6 | 3244.6 | 675.0 |
| 1.157 | 29192.3 | 10235.1 | 8693.4 | 2357.4 |
| 1.158 | 5905.2 | 343.4 | 1971.7 | 454.0 |
| 1.159 | 1232.9 | 459.5 | 2061.8 | 271.7 |
| 1.161 | 63.5 | 3.6 | 129.4 | 73.5 |
| 1.162 | 92.0 | 0.9 | 387.4 | 217.4 |
| 1.163 | 4423.8 | 182.3 | 1875.2 | 496.6 |
| 1.164 | 4306.8 | 26.6 | 1957.4 | 729.2 |
| 1.165 | 4140.0 | 293.7 | 1627.1 | 584.4 |
| 1.166 | 18132.9 | 4816.3 | 5163.5 | 1419.0 |
| 1.167 | 8247.3 | 802.7 | 1071.0 | 516.6 |
| 1.170 | 7814.3 | 82.1 | 2046.3 | 580.9 |
| 1.171 | 9326.9 | 448.0 | 3419.0 | 841.6 |
| 1.173 | 157.0 | 0.5 | 339.7 | 204.4 |
| 1.175 | 2820.2 | 294.6 | 853.0 | 92.0 |
| 1.176 | 20941.5 | 4664.9 | 8755.7 | 3209.3 |
| 1.178 | 711.4 | 5.8 | 1116.2 | 637.4 |
| 1.180 | 12022.9 | 416.9 | 1029.2 | 139.1 |
| 1.183 | 9007.8 | 1662.8 | 2477.1 | 1431.3 |
| 1.185 | 4216.6 | 403.6 | 1152.2 | 761.8 |
| 1.186 | 10237.7 | 1867.1 | 1612.5 | 982.8 |
| 1.195 | 21975.8 | 379.4 | 2731.0 | 1192.9 |
| 1.197 | 64051.2 | 47694.4 | 8688.8 | 366.2 |
| 1.200 | 10608.5 | 131.2 | 3903.1 | 3979.1 |
| 1.204 | 1908.2 | 34.3 | 926.8 | 122.9 |
| 1.206 | 529.1 | 22.0 | 314.4 | 209.6 |
| 1.208 | 345.7 | 19.4 | 720.6 | 705.8 |
| 1.212 | 390.2 | 3.8 | 894.0 | 580.3 |
| 1.213 | 3207.8 | 140.6 | 2097.2 | 112.7 |
| 1.215 | 14753.0 | 1613.1 | 1285.8 | 108.5 |
| 1.217 | 10301.1 | 93.6 | 3501.9 | 3691.2 |
| 1.219 | 38297.7 | 11679.7 | 4969.9 | 1893.5 |
| 1.223 | 11139.0 | 1467.2 | 3101.9 | 1629.9 |
| 1.226 | 531.0 | 1.1 | 1348.5 | 1389.6 |
| 1.227 | 3476.0 | 196.6 | 1580.9 | 623.5 |
| 1.229 | 24557.8 | 17008.1 | 3128.5 | 322.4 |
| 1.233 | 2628.6 | 182.4 | 2004.9 | 815.1 |
| 1.236 | 3716.5 | 474.9 | 2755.4 | 2914.8 |
| 1.237 | 7910.2 | 217.5 | 9873.2 | 7272.6 |
| 1.238 | 4171.1 | 173.1 | 2609.6 | 1573.2 |
| 1.239 | 17657.7 | 4393.7 | 10026.9 | 8534.5 |
| 1.246 | 1096.1 | 9.5 | 1879.2 | 1883.4 |
| 1.249 | 1599.7 | 63.8 | 937.5 | 226.8 |
| 1.252 | 205.0 | 11.9 | 170.7 | 84.1 |
| 1.253 | 2597.1 | 29.9 | 2515.0 | 1464.8 |
| 1.258 | 315.2 | 94.1 | 531.5 | 229.6 |
| 1.262 | 861.0 | 1.0 | 5436.6 | 49.5 |
| 2.009 | 3725.8 | 198.3 | 1280.8 | 361.0 |
| 2.022 | 4115.1 | 209.4 | 501.1 | 6.9 |
| 2.025 | 966.4 | 103.5 | 498.8 | 74.2 |
| 2.026 | 2076.0 | 196.5 | 536.0 | 4.6 |
| 2.027 | 657.7 | 58.8 | 509.0 | 70.6 |
| 2.031 | 1357.9 | 0.6 | 326.4 | 52.7 |
| 2.038 | 2553.9 | 184.2 | 1397.0 | 345.6 |
| 2.039 | 1988.0 | 66.7 | 1010.3 | 195.5 |
| 2.041 | 3443.4 | 187.8 | 2095.1 | 161.9 |
| 2.046 | 1975.4 | 142.9 | 758.9 | 401.2 |
| 2.047 | 1942.1 | 163.1 | 437.5 | 184.9 |
| 2.054 | 414.8 | 5.7 | 438.9 | 207.3 |
| 2.055 | 977.5 | 72.3 | 311.6 | 180.9 |
| 2.058 | 1936.0 | 136.7 | 212.6 | 44.7 |
| 2.059 | 119.8 | 24.5 | 207.9 | 173.8 |
| 2.060 | 328.8 | 10.3 | 181.3 | 102.7 |
| 2.064 | 382.0 | 6.7 | 178.2 | 103.4 |
| 2.066 | 2510.4 | 30.5 | 368.3 | 133.1 |

### Example 21

### Kinase Panel Screen

### Relevance:

This assay demonstrates a compound's ability to inhibit members of a panel of kinases known to be involved in signaling pathways connected to inflammatory processes.

### Protocol

Compounds of Formulae I and II were examined for activity against a selected panel of kinases using the KinaseProfiler™ enzyme profiling services (Upstate, Millipore Bioscience Division). Percent kinase activity at 10 μM and 1 μM test compound and 10 μM ATP was determined against 40 wild-type recombinant human kinases according to Upstate's standard protocol: ASK1, BTK, CSK, c-RAF, GCK, GSK3β, IKKα, IKKβ, IRAK1, IRAK4, JNK1α1, JNK2α2, JNK3, ERK1, ERK2, MAPKAP-K2, MAPKAP-K3, MEK1, MKK4, MKK6, MKK7β, Mnk2, MSK1, PAK3, PDK1, PRAK, ROCK1, Rsk2, SAPK2a, SAPK2b, SAPK3, SAPK4, SRPK1, SRPK2, Syk, TAK1, TBK1, PI3-Kβ, PI3-Kγ, PI3-Kδ.

### Results:

Percent inhibition results are reported in Table 9 for four compounds against six kinases in the panel. Only compounds in which R₂ is R₂-2 were found to inhibit significantly GCK, ERK1/2, Mnk2 and IRAK1/2. Only ERK1/2 were inhibited by ˜50% at 1 μM by both compounds 2.059 and 2.066.

TABLE 9
Percent inhibition data for six of the tested kinases

| | | | | | | | Compound | |
|---|---|---|---|---|---|---|---|---|
| | Compound | | Compound | | Compound | | 1.162 | |
| | 2.059 | | 2.066 | | 1.161 | | | 10 |
| | 1 μM | 10 μM | 1 μM | 10 μM | 1 μM | 10 μM | 1 μM | μM |
| ERK1 | 37 | 4 | 52 | 15 | 97 | 75 | 84 | 50 |
| ERK2 | 56 | 12 | 50 | 12 | 104 | 92 | 89 | 60 |
| Mnk2 | 49 | 12 | 99 | 54 | 108 | 106 | 111 | 65 |
| IRAK4 | 63 | 22 | 77 | 25 | 96 | 109 | 105 | 88 |
| IRAK1 | 87 | 30 | 74 | 32 | 106 | 99 | 100 | 97 |
| GCK | 75 | 34 | 39 | 7 | 96 | 91 | 93 | 75 |

### Example 22

### Rodent Pharmacokinetic Analyses of ROCK Inhibitors

Plasma (EDTA K2 anticoagulant) was collected from male, cannulated, CD Sprague Dawley rats to determine the pharmacokinetics of formulations containing compound inhibitors of Rho kinase. Each animal was dosed orally with a 4 ml/kg solution or suspension of each test compound in 10 mM acetate buffered saline, pH 4.5 at a final concentration range of 20-30 μmol/kg. Blood was collected at 0.25, 0.5, 1, 2, 4, 6, 8, and 24 hours. Plasma samples were assayed for the concentration of the test compound using an on-line, solid phase extraction LC/MS/MS analysis system.

Samples were analyzed on a QSTAR Elite, hybrid quadrupole time-of-flight mass spectrometer (Applied Biosystems, Framingham, Mass.) coupled with a Symbiosis Pharma integrated, on-line SPE-HPLC system (Spark Holland Inc., Plainsboro, N.J.). Analyst QS 2.0 software was used for instrument control, data acquisition and processing. An aliquot of each sample was injected onto a Luna C18 column (50×2 mm, 4 um, 80A, Phenomenex, Torrance, Calif.), and elution was carried out using a gradient from 2-98% acetonitrile. Mobile Phase A consisted of 0.1% ammonium hydroxide in water and Mobile Phase B consisted of 0.1% formic acid in acetonitrile. Pharmacokinetic analyses were performed using WinNonlin software version 5.2 (Pharsight Corporation, Mountain View, Calif.).

The pharmacokinetic results based on the observed plasma concentrations of the test compounds in rats are shown in Table 10.

TABLE 10
Pharmacokinetic results from rat oral PK studies (mean plasma
values for n = 3 rats)

| | Tmax | Cmax | AUC (0-last) | t½ | Vz_F |
|---|---|---|---|---|---|
| Compound | (hr) | (nM) | (nM * hr) | (hr) | (L/kg) |
| 1.131 | 0.83 | 5610 | 10825 | 1.55 | 6.8 |
| 1.092 | 0.25 | 2101 | 1849 | 1.74 | 19.0 |
| 1.123 | 0.33 | 2044 | 2064 | 0.9 | 14.8 |
| 2.038 | 0.5 | 1037 | 1283 | 0.71 | 22.5 |
| 2.039 | 0.33 | 783 | 905 | 1.13 | 59.4 |
| 1.074 | 0.42 | 735 | 1167 | 0.86 | 45.7 |
| 1.107 | 1.67 | 544 | 1586 | 1.28 | 36.3 |
| 1.124 | 0.5 | 415 | 535 | 1.39 | 93.4 |
| 2.045 | 0.67 | 223 | 456 | 1.59 | 226 |
| 1.108 | 0.83 | 209 | 415 | 1.36 | 116 |
| 1.091 | BLQ | BLQ | BLQ | BLQ | BLQ |
| 2.026 | BLQ | BLQ | BLQ | BLQ | BLQ |
| 1.136 | BLQ | BLQ | BLQ | BLQ | BLQ |

BLQ indicates that the compound was below the limit of quantitation in the assay

As determined from the plasma concentration versus time curves, the time to peak and peak exposure are represented by the values Tmax and Cmax, respectively. The AUC values (nM*hr) shown were calculated as the areas under the plasma concentration versus time curves from time zero through the time of the last observable value and represent the total exposure of the compound over the course of the study. Half-life values or the amount of time required for the plasma levels of the compound to decline to half the initial value are represented as t½. The volume of distribution (Vz_F expressed in L/kg) relates the amount of theoretical volume needed to account for the observed concentration of a given dose of a compound. For rats, the total body water content is approximately 0.15 L/kg. Calculated volumes of distribution below 0.15 L/kg are considered low, whereas values between 5 and 100 L/kg are considered high. The volume of distribution varies depending on the degree of plasma protein binding as well as partitioning of the compound into fat and tissues. Table 10 provides evidence that our ROCK inhibiting compounds have a varying degree of pharmacokinetic properties that would allow them to be optimized for multiple routes of administration. These compounds are quickly absorbed, as indicated by a Tmax of generally less than 1 hour, with varying degrees of peak and total exposure as indicated by Cmax and AUC, with higher values indicating greater exposure. Regardless of exposure, these compounds demonstrate a similar clearance, t½.

Additionally, compound concentrations were determined in the plasma and lungs of male, ovalbumin-sensitized, Balb/c mice from a murine model of asthma. Test compounds were formulated in water or 1% polysorbate 80 and dosed at 15 μmol/kg for intraperitoneal (IP) or oral (PO) administration or formulated for intratracheal (IT) administration and dosed at 5 μmol/kg, which directly targets the lungs. Following completion of the in vivo study, mice were euthanized and blood and plasma collected approximately 2.5-3 hours post administration of test compound for bronchodialator (BD) studies and 24 hours post administration for anti-inflamatory (AI) studies. Lungs were homogenized in Matrix A lysing tubes using a FastPrep 24 tissue and cell homogenizer (MP Biomedicals, Solon, Ohio). Both plasma samples and lung extracts were assayed for compound concentrations using an on-line, solid phase extraction LC/MS/MS system. The actual lung tissue concentrations of each compound in mouse were extrapolated from the lung and plasma concentrations, data are shown in Table 11. The results of a set of experiments using unsensitized mice and collecting only plasma 15 minutes post administration of test compounds are shown in Table 12.

TABLE 11
Compound concentrations in ova-sensitized,
ova-challenged mice lungs post IP, PO and IT administration
(mean plasma corrected lung values for n = 9 or 10 mice)

| Compound | Efficacy Model | Route | Time Point, h | Lung, nM¹ |
|---|---|---|---|---|
| 1.131 | BD | PO | 3 | 7353 |
| 2.038 | BD | PO | 3 | 440 |
| 1.092 | BD | PO | 3 | 152 |
| 1.091 | BD | IP | 3 | 117 |
| 1.091 | BD | IT | 2.5 | 123 |
| 1.131 | AI | PO | 24 | 33 |
| 2.038 | AI | PO | 24 | 11 |

¹for calculation of lung concentrations, it was assumed that 22.6% of the lung mass was plasma (R. H. Storey, Cancer Research, 943-947, 1951)

TABLE 12
Compound concentrations in mice at 15 min post administration
(mean plasma values for n = 3 mice)

| | | Plasma |
|---|---|---|
| | Plasma Mean | Concentration |
| Compound | Concentration, nM | StdDev, nM |
| 1.072 | 1770.9 | 320.9 |
| 1.074 | 506.1 | 407.9 |
| 1.075 | 348.0 | 83.9 |
| 1.076 | 1715.0 | 474.9 |
| 1.077 | 25.9 | 0.2 |
| 1.078 | 1018.8 | 75.8 |
| 1.079 | 2442.5 | 302.9 |
| 1.090 | 5.9 | 5.2 |
| 1.091 | 333.8 | 82.7 |
| 1.092 | 314.3 | 60.4 |
| 1.093 | 362.6 | 148.7 |
| 1.106 | 441.4 | 146.7 |
| 1.107 | 211.1 | 129.5 |
| 1.108 | 394.5 | 9.0 |
| 1.109 | 187.2 | 36.0 |
| 1.110 | 792.0 | 311.9 |
| 1.123 | 71.4 | 11.8 |
| 1.124 | 118.0 | 2.4 |
| 1.126 | 0.0 | 0.0 |
| 1.127 | 980.2 | 757.5 |
| 1.131 | 444.5 | 130.0 |
| 1.132 | 982.4 | 207.7 |
| 1.133 | 1097.9 | 234.3 |
| 1.134 | 1550.8 | 623.9 |
| 1.135 | 656.8 | 115.4 |
| 1.136 | 25.9 | 6.3 |
| 1.137 | 556.9 | 279.8 |
| 1.138 | 1863.8 | 378.7 |
| 1.141 | 1643.1 | 368.6 |
| 1.142 | 329.7 | 171.6 |
| 1.143 | 274.5 | 68.8 |
| 1.145 | 109.0 | 117.9 |
| 1.146 | 1255.7 | 703.5 |
| 1.148 | 767.1 | 63.9 |
| 1.149 | 1559.4 | 789.6 |
| 1.150 | 1392.3 | 1278.3 |
| 1.151 | 478.6 | 173.6 |
| 1.152 | 435.4 | 44.5 |
| 1.153 | 521.5 | 61.3 |
| 1.154 | 1039.5 | 447.9 |
| 1.155 | 32.4 | 36.3 |
| 1.156 | 88.0 | 37.5 |
| 1.157 | 357.2 | 131.9 |
| 1.158 | 101.6 | 54.4 |
| 1.159 | 250.5 | 343.2 |
| 1.161 | 392.5 | 14.9 |
| 1.162 | 76.1 | 12.9 |
| 1.163 | 10.1 | 1.1 |
| 1.164 | 1504.3 | 580.6 |
| 1.165 | 93.5 | 49.6 |
| 1.166 | 342.4 | 118.1 |
| 1.168 | 587.5 | 258.9 |
| 1.170 | 638.6 | 154.7 |
| 1.171 | 368.8 | 208.9 |
| 1.172 | 111.1 | 32.0 |
| 1.173 | 144.4 | 72.6 |
| 1.175 | 1126.5 | 112.5 |
| 1.176 | 89.1 | 69.1 |
| 1.177 | 283.1 | 125.6 |
| 1.182 | 452.5 | 297.7 |
| 1.183 | 708.5 | 359.6 |
| 1.185 | 1023.6 | 492.8 |
| 1.186 | 2169.4 | 1599.1 |
| 1.191 | 260.0 | 58.8 |
| 1.193 | 55.4 | 26.0 |
| 1.194 | 355.0 | 133.5 |
| 1.195 | 107.9 | 23.1 |
| 1.197 | 453.1 | 354.0 |
| 1.198 | 643.2 | 112.1 |
| 1.200 | 0.0 | 0.0 |
| 1.202 | 129.7 | 71.9 |
| 1.203 | 1134.7 | 44.2 |
| 1.204 | 549.1 | 183.6 |
| 1.206 | 671.5 | 80.9 |
| 1.208 | 281.1 | 45.4 |
| 1.210 | 285.8 | 122.9 |
| 1.212 | 863.4 | 104.1 |
| 1.213 | 396.4 | 135.1 |
| 1.215 | 2651.2 | 529.0 |
| 1.217 | 292.5 | 176.0 |
| 1.219 | 1678.9 | 516.3 |
| 1.223 | 12.8 | 0.6 |
| 1.226 | 526.1 | 157.9 |
| 1.227 | 1859.4 | 603.7 |
| 1.229 | 1453.9 | 465.0 |
| 1.233 | 41.1 | 11.6 |
| 1.234 | 239.6 | 79.4 |
| 1.236 | 47.7 | 18.1 |
| 1.237 | 178.4 | 64.6 |
| 1.238 | 48.3 | 29.6 |
| 1.239 | 258.9 | 111.8 |
| 1.241 | 991.4 | 134.5 |
| 1.242 | 579.8 | 314.0 |
| 1.245 | 1524.0 | 127.5 |
| 1.246 | 587.4 | 299.7 |
| 1.249 | 2147.1 | 688.2 |
| 1.252 | 1259.2 | 1210.0 |
| 1.253 | 240.0 | 20.3 |
| 1.258 | 567.5 | 223.5 |
| 1.259 | 264.4 | 39.1 |
| 1.260 | 291.2 | 120.7 |
| 1.262 | 285.2 | 76.2 |
| 2.025 | 73.7 | 21.2 |
| 2.026 | 629.5 | 94.6 |
| 2.027 | 502.6 | 248.5 |
| 2.031 | 1430.4 | 139.2 |
| 2.034 | 664.7 | 649.4 |
| 2.036 | 1343.9 | 1603.3 |
| 2.038 | 728.9 | 222.8 |
| 2.039 | 92.0 | 47.6 |
| 2.041 | 986.5 | 287.0 |
| 2.043 | 60.8 | 24.7 |
| 2.046 | 488.1 | 96.1 |
| 2.047 | 3.0 | 1.7 |
| 2.054 | 765.5 | 214.3 |
| 2.055 | 656.1 | 172.6 |
| 2.056 | 1257.0 | 230.6 |
| 2.057 | 431.2 | 41.5 |
| 2.058 | 193.6 | 167.4 |
| 2.059 | 89.6 | 21.5 |
| 2.060 | 307.6 | 157.6 |
| 2.061 | 73.2 | 21.1 |
| 2.062 | 659.9 | 582.8 |
| 2.063 | 347.9 | 248.5 |
| 2.064 | 201.6 | 78.7 |
| 2.065 | 236.4 | 29.8 |
| 2.066 | 491.6 | |

The results of these quantitative analyses have enabled the selection of compounds for additional studies based on desirable pharmacokinetic profiles and preferential distribution in the target organ (lungs). We have identified compounds which possess high bioavailability and efficacy against airway hyperreactivity when dosed orally, as well as compounds that are efficacious when administered intraperitoneally or intratracheally, but do not reach systemic levels when dosed orally and thus are not efficacious by the oral route. Characterization of the pharmacokinetic properties and distribution of these Rho Kinase inhibitors is an essential part of the selection of compounds for drug development.

### Example 23

### Efficacy of Compounds of Formula I or II to Inhibit Proliferation of Primary Smooth-Muscle Like Cells Derived from Human LAM Patients

### Relevance

This assay measures the ability of a compound to directly inhibit the proliferation of primary smooth-muscle like cells derived from human LAM patients. Activity of compounds in this assay supports the use of these compounds for the treatment of diseases with a proliferative component.

### Protocol

LAM cells were dissociated from LAM nodules from the lung of patients with LAM who have undergone lung transplant. In brief, cells were dissociated by enzymatic digestion in M199 medium containing 0.2 mM CaCl₂, 2 mg/ml collagenase D, 1 mg/ml trypsin inhibitor, and 3 mg/ml elastase. The cell suspension was filtered and then washed with equal volumes of cold DF8 medium, consisting of equal amounts of Ham's F-12 and Dulbecco's modified Eagle's medium supplemented with 1.6×10⁻⁶ M ferrous sulfate, 1.2×10⁻⁵ U/ml vasopressin, 1.0×10⁻⁹ M triiodothyronine, 0.025 mg/ml insulin, 1.0×10⁻⁸ M cholesterol, 2.0×10⁻⁷ M hydrocortisone, 10 pg/ml transferrin, and 10% fetal bovine serum. The cells were cultured in DF8 medium and were passaged twice per week. All LAM cells had a high degree of proliferative activity in the absence of any stimuli. Two separate LAM cell lines were tested and denoted as LAM1 or LAM2 cells. LAM cells in subculture during the 3rd through 12th cell passages were used. DNA synthesis was measured using a [³H]thymidine incorporation assay. In brief, near-confluent cells that were serum-deprived for 48 h were incubated with 10 μM of compound or with vehicle (control). After 18 h of incubation, cells were labeled with [methyl-³H]thymidine for 24 hours. The cells were then scraped and lysed, and DNA was precipitated with 10% trichloroacetic acid. The precipitants were aspirated on glass filters and extensively washed and dried, and [³H]thymidine incorporation was counted (Goncharova et al., Mol Pharmacol 73:778-788, 2008)

### Results

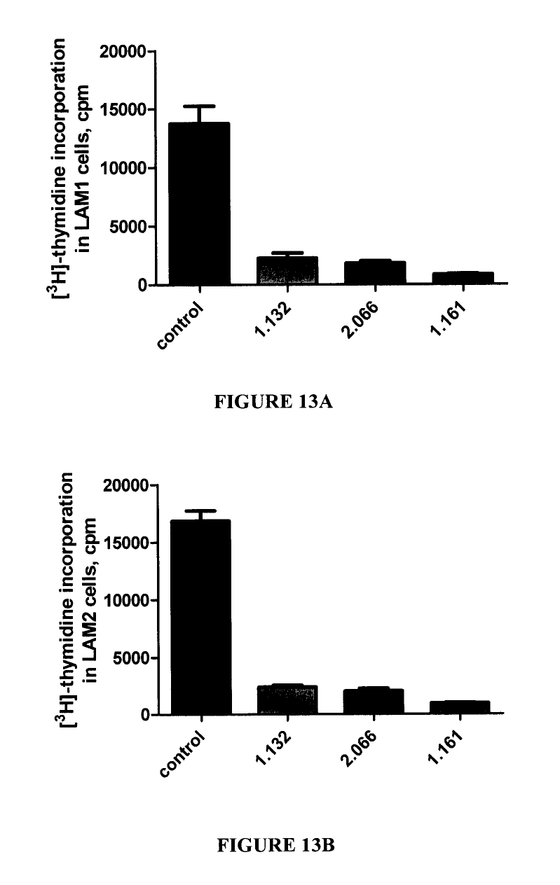

As shown in FIGS. 13A and 13B, compounds of Formula I and II reduced proliferation of LAM1 (FIG. 13A) and LAM2 (FIG. 13B) cells when dosed in vitro at 10 μM. These results demonstrate that Compounds of Formula I and II are efficacious in inhibiting the proliferation of primary cells.

### Example 24

### Summary of Data of Preferred Compounds

Principal biological data describing the preferred compounds of the invention have been collected into Table 13. Displayed in this table are ROCK1 and ROCK2 average Ki values in nM (as detailed in Example 1), Akt3 and p70S6K average Ki values in nM (as detailed in Example 20), average percent of PDGF stimulated proliferation at 10 and 1 μM of test compound (as detailed in Example 18), average percent of stimulated IL-1β, IL-6, and TNF-α secretion from human monocytes at 10 μM of test compound (as detailed in Example 16), average IC₅₀ for inhibition of fMLP-induced neutrophil chemotaxis in μM (as detailed in Example 2), mean compound plasma concentrations in mice at 15 minutes post oral administration (as detailed in Example 22).

TABLE 14
Summary of Data of Preferred Compounds

| | | | | | | | | | | Chemotaxis | Mouse |
|---|---|---|---|---|---|---|---|---|---|---|---|
| Com- | ROCK1 | ROCK2 | Akt3 | p70S6K | Proliferation | Proliferation | IL-1β | IL-6, | TNF-α | IC50, | Oral PK, |
| pound | Ki, nM | Ki, nM | Ki, nM | Ki, nM | at 10 μM, % | at 1 μM, % | % | % | % | μM | nM |
| 1.074 | 40.1 | 4.1 | 437.4 | 548.3 | 46.9 | 79.9 | 43.9 | 96.0 | 87.7 | | 506 |
| 1.075 | 48.7 | 4.4 | 5321.5 | 974.6 | | | 49.7 | 73.9 | 51.6 | | 348 |
| 1.076 | 14.3 | 2.6 | 240.9 | 414.3 | 53.7 | 84.0 | 51.0 | 81.2 | 78.9 | | 1715 |
| 1.077 | 76.1 | 11.1 | 5253.2 | 715.5 | | | 30.3 | 43.3 | 52.3 | | 26 |
| 1.079 | 71.5 | 4.7 | 7191.7 | 3012.8 | | | 59.3 | 31.1 | 56.5 | | 2443 |
| 1.091 | 71.4 | 3.3 | 5388.5 | 1420.4 | 69.3 | 85.7 | 165.5 | 108.2 | 104.6 | 2.3 | 334 |
| 1.093 | 64.5 | 7.7 | 1824.9 | 2025.6 | | | 109.0 | 49.7 | 76.1 | | 363 |
| 1.108 | 25.6 | 6.5 | 205.0 | 510.6 | 43.7 | 83.1 | 131.3 | 89.8 | 116.7 | | 395 |
| 1.109 | 58.8 | 9.6 | 5190.9 | 2495.5 | | | 190.5 | 312.9 | 118.3 | | 187 |
| 1.123 | 82.3 | 9.6 | 2406.9 | 2810.7 | | | 82.6 | 64.7 | 62.7 | 3.1 | 71 |
| 1.124 | 64.5 | 3.3 | 7868.0 | 3325.3 | 61.6 | 68.5 | 99.5 | 101.4 | 61.5 | 3.4 | 118 |
| 1.126 | 76.2 | 17.2 | | | | | | | | | 0 |
| 1.131 | 19.7 | 3.8 | 282.6 | 502.8 | 36.6 | 61.7 | 48.3 | 68.6 | 85.2 | 1.6 | 445 |
| 1.132 | 22.5 | 3.5 | 81.8 | 514.6 | 30.3 | 48.9 | 58.6 | 72.5 | 80.3 | | 982 |
| 1.133 | 25.0 | 4.3 | 148.3 | 531.8 | | | 54.5 | 70.7 | 66.2 | | 1098 |
| 1.134 | 22.4 | 4.4 | 150.7 | 519.7 | | | 43.2 | 74.6 | 69.1 | | 1551 |
| 1.135 | 40.3 | 5.4 | 444.2 | 588.6 | 35.0 | 52.6 | 57.0 | 123.2 | 108.0 | | 657 |
| 1.136 | 25.8 | 5.1 | 289.7 | 1236.7 | 39.8 | 71.4 | 66.3 | 95.0 | 71.5 | 2.6 | 26 |
| 1.137 | 36.3 | 7.2 | 197.9 | 353.6 | | | 40.3 | 46.2 | 58.0 | | 557 |
| 1.138 | 41.1 | 6.3 | 91.3 | 443.5 | 27.0 | 46.3 | 257.4 | 76.6 | 130.9 | 1.9 | 1864 |
| 1.141 | 28.5 | 3.8 | 1263.0 | 387.5 | | | 50.4 | 71.7 | 75.7 | | 1643 |
| 1.148 | 24.3 | 3.6 | 1131.4 | 435.5 | 63.5 | 56.9 | 63.9 | 78.6 | 56.1 | | 767 |
| 1.149 | 46.8 | 4.2 | 7395.9 | 1888.4 | | | 69.8 | 121.5 | 119.9 | | 1559 |
| 1.150 | 33.2 | 3.2 | 3183.1 | 1273.8 | | | 78.2 | 89.2 | 94.4 | | 1392 |
| 1.152 | 19.8 | 3.3 | 1976.2 | 523.5 | | | 74.7 | 94.7 | 120.1 | | 435 |
| 1.153 | 62.8 | 4.2 | 9950.2 | 2376.1 | | | 64.1 | 106.2 | 74.3 | | 522 |
| 1.155 | 45.4 | 7.0 | 5680.5 | 1751.6 | | | 76.7 | 121.8 | 79.7 | | 32 |
| 1.156 | 135.8 | 13.0 | 8772.6 | 3244.6 | | | 60.7 | 92.5 | 70.5 | | 88 |
| 1.157 | 263.8 | 8.8 | 29192.3 | 8693.4 | | | 121.4 | 92.6 | 65.1 | | 357 |
| 1.158 | 64.1 | 5.1 | 5905.2 | 1971.7 | | | 80.8 | 133.1 | 86.6 | | 102 |
| 1.161 | 9.9 | 2.5 | 63.5 | 129.4 | 33.4 | 50.0 | 87.7 | 86.3 | 153.5 | | 392 |
| 1.162 | 15.2 | 2.8 | 92.0 | 387.4 | 42.5 | 55.6 | 95.5 | 99.8 | 158.7 | | 76 |
| 1.163 | 33.6 | 2.9 | 4423.8 | 1875.2 | | | 166.7 | 140.9 | 91.6 | | 10 |
| 1.164 | 42.4 | 6.1 | 4306.8 | 1957.4 | | | 80.1 | 109.5 | 89.0 | | 1504 |
| 1.165 | 50.7 | 3.4 | 4140.0 | 1627.1 | 57.9 | 74.8 | 129.9 | 114.3 | 103.5 | | 94 |
| 1.166 | 95.2 | 8.0 | 18132.9 | 5163.5 | | | 107.0 | 87.2 | 82.2 | | 342 |
| 1.171 | 109.2 | 16.0 | 9326.9 | 3419.0 | | | 78.9 | 91.8 | 72.2 | | 369 |
| 1.173 | 15.1 | 3.6 | 157.0 | 339.7 | 35.8 | 55.4 | 86.1 | 79.5 | 80.1 | | 144 |
| 1.175 | 65.9 | 7.6 | 2820.2 | 853.0 | 49.0 | 58.2 | 29.3 | 38.2 | 47.4 | | 1126 |
| 1.176 | 314.3 | 11.2 | 20941.5 | 8755.7 | | | 95.2 | 112.4 | 72.4 | | 89 |
| 1.186 | 129.3 | 11.9 | 10237.7 | 1612.5 | | | 64.1 | 105.3 | 68.2 | | 2169 |
| 1.193 | 64.9 | 14.8 | | | | | | | | | 55 |
| 1.195 | 196.2 | 10.3 | 21975.8 | 2731.0 | | | 115.4 | 94.4 | 67.7 | | 108 |
| 1.197 | 120.2 | 5.0 | 64051.2 | 8688.8 | 48.9 | 52.5 | 179.1 | 128.8 | 83.3 | | 453 |
| 1.200 | 76.5 | 5.9 | 10608.5 | 3903.1 | | | 0.0 | 0.0 | 0.2 | | 0 |
| 1.206 | 64.4 | 9.1 | 529.1 | 314.4 | 51.1 | 77.5 | 88.7 | 164.0 | 97.3 | | 672 |
| 1.212 | 44.2 | 3.9 | 390.2 | 894.0 | | | 116.3 | 111.0 | 108.1 | | 863 |
| 1.213 | 106.3 | 3.0 | 3207.8 | 2097.2 | 52.3 | 70.1 | 111.1 | 81.7 | 77.4 | | 396 |
| 1.215 | 102.8 | 3.5 | 4753.0 | 1285.8 | 54.0 | 70.8 | 136.7 | 63.2 | 60.4 | | 2651 |
| 1.217 | 70.1 | 12.1 | 10301.1 | 3501.9 | | | 118.6 | 73.8 | 71.3 | | 293 |
| 1.219 | 343.6 | 15.4 | 38297.7 | 4969.9 | | | 138.9 | 127.7 | 82.1 | | 1679 |
| 1.223 | 239.5 | 15.7 | 11139.0 | 3101.9 | | | 117.0 | 88.5 | 60.7 | | 13 |
| 1.233 | 47.2 | 1.3 | 2628.6 | 2004.9 | | | 78.5 | 78.9 | 79.0 | | 41 |
| 1.236 | 49.3 | 2.1 | 3716.5 | 2755.4 | | | 75.2 | 93.0 | 98.0 | | 48 |
| 1.237 | 286.7 | 4.0 | 7910.2 | 9873.2 | 51.4 | 63.5 | 97.1 | 100.9 | 70.6 | | 178 |
| 1.238 | 61.2 | 1.5 | 4171.1 | 2609.6 | 48.6 | 40.7 | 101.1 | 62.9 | 73.2 | | 48 |
| 1.239 | 282.6 | 6.3 | 17657.7 | 10026.9 | 37.8 | 41.7 | 39.4 | 84.7 | 58.5 | | 259 |
| 1.249 | 91.7 | 8.6 | 1599.7 | 937.5 | | | 133.8 | 56.2 | 60.0 | | 2147 |
| 1.252 | 30.5 | 4.5 | 205.0 | 170.7 | | | 139.2 | 68.3 | 101.6 | | 1259 |
| 1.253 | 59.9 | 1.7 | 2597.1 | 2515.0 | 47.9 | 44.8 | 160.6 | 228.6 | 126.8 | | 240 |
| 1.258 | 9.5 | 1.3 | 315.2 | 531.5 | 43.4 | 50.5 | 104.1 | 83.5 | 94.0 | | 567 |
| 1.259 | 19.5 | 2.1 | | | | | | | | | 264 |
| 1.260 | 70.9 | 7.1 | | | | | | | | | 291 |
| 1.261 | 307.4 | 14.8 | | | | | | | | | |
| 1.262 | 54.9 | 4.0 | 861.0 | 5436.6 | | | 145.7 | 156.6 | 135.3 | | 285 |
| 1.270 | 130.5 | 9.9 | | | | | | | | | |
| 1.273 | 31.3 | 8.2 | | | | | | | | | |
| 1.275 | 401.7 | 14.1 | | | | | | | | | |
| 1.277 | 42.3 | 4.6 | | | | | | | | | |
| 1.281 | 71.8 | 7.4 | | | | | | | | | |
| 2.025 | 6.9 | 2.9 | 966.4 | 498.8 | 68.0 | 69.8 | | | | 1.7 | 74 |
| 2.026 | 38.0 | 13.0 | 2076.0 | 536.0 | 52.0 | 74.5 | 166.0 | 180.7 | 109.1 | 3.8 | 629 |
| 2.031 | 14.6 | 5.3 | 1357.9 | 326.4 | 52.6 | 90.3 | 49.0 | 89.3 | 66.4 | | 1430 |
| 2.038 | 28.9 | 6.3 | 2553.9 | 1397.0 | 62.7 | 58.6 | 90.8 | 79.7 | 70.2 | 0.7 | 729 |
| 2.039 | 18.8 | 6.7 | 1988.0 | 1010.3 | | | 49.8 | 70.3 | 47.8 | 1.6 | 92 |
| 2.041 | 30.8 | 9.6 | 3443.4 | 2095.1 | 61.5 | 81.8 | | | | | 987 |
| 2.046 | 16.7 | 5.6 | 1975.4 | 758.9 | 32.1 | 57.4 | | | | | 488 |
| 2.047 | 26.4 | 7.0 | 1942.1 | 437.5 | 53.8 | 65.3 | | | | | 3 |
| 2.054 | 17.1 | 3.7 | 414.8 | 438.9 | 84.6 | 68.2 | 24.0 | 56.8 | 37.9 | | 765 |
| 2.055 | 16.0 | 6.4 | 977.5 | 311.6 | | | | | | | 656 |
| 2.057 | 6.2 | 3.7 | | | | | | | | | 431 |
| 2.058 | 15.3 | 3.3 | 1936.0 | 212.6 | | | 1.2 | 1.3 | 10.6 | | 194 |
| 2.059 | 3.9 | 2.7 | 119.8 | 207.9 | 25.5 | 75.0 | 0.3 | 0.0 | 6.9 | | 90 |
| 2.060 | 4.9 | 3.2 | 328.8 | 181.3 | | | 5.9 | 19.6 | 33.0 | | 308 |
| 2.061 | 10.5 | 1.8 | | | | | | | | | 73 |
| 2.064 | 4.1 | 2.2 | 382.0 | 178.2 | 56.2 | 53.1 | 14.3 | 45.7 | 66.2 | | 202 |
| 2.065 | 4.1 | 1.8 | | | | | | | | | 236 |
| 2.066 | 10.2 | 2.3 | 2510.4 | 368.3 | 19.8 | 20.0 | 0.0 | 0.0 | 25.2 | | 492 |
| 2.067 | 19.6 | 4.2 | | | | | | | | | |
| 2.068 | 8.0 | 5.8 | | | | | | | | | |
| 2.069 | 16.7 | 2.4 | | | | | | | | | |
| 2.072 | 7.5 | 4.4 | | | | | | | | | |
| 2.073 | 12.7 | 4.2 | | | | | | | | | |
| 2.076 | 8.0 | 2.4 | | | | | | | | | |
| 2.077 | 33.7 | 5.0 | | | | | | | | | |
| 2.078 | 18.3 | 2.6 | | | | | | | | | |
| 2.079 | 18.5 | 2.3 | | | | | | | | | |
| 2.082 | 131.7 | 9.0 | | | | | | | | | |
| 2.096 | 70.2 | 9.6 | | | | | | | | | |
| 2.097 | 35.4 | 2.8 | | | | | | | | | |
| 2.099 | 15.0 | 3.8 | | | | | | | | | |

Although the invention has been described with reference to the presently preferred embodiments, it should be understood that various modifications could be made without departing from the scope of the invention.

What is claimed:

1. A method of treating allergic conjunctivitis, corneal hyposensitivity, neurotrophic keratopathy, dry eye disease, proliferative vitreal retinopathy, macular edema, macular degeneration, or blepharitis; comprising the steps of first identifying a subject suffering from allergic conjunctivitis, corneal hyposensitivity, neurotrophic keratopathy, dry eye disease, proliferative vitreal retinopathy, macular edema, macular degeneration, or blepharitis; then administering to the subject an effective amount of a compound of Formula II to treat allergic conjunctivitis, corneal hyposensitivity, neurotrophic keratopathy, dry eye disease, proliferative vitreal retinopathy, macular edema, macular degeneration, or blepharitis; 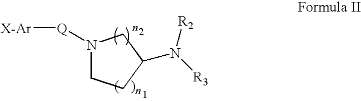
wherein:
Q is C═O, SO₂, or (CR₄R₅)ₙ₃;
n₁ is 1, 2, or 3;
n₂ is 1 or 2;
n₃ is 0, 1, 2, or 3;
wherein the ring represented by 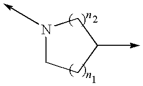
is optionally substituted by alkyl, halo, oxo, OR₆, NR₆R₇, or SR₈;
R₂ is R₂-1 or R₂-2, optionally substituted: 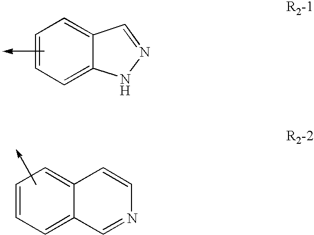
Ar is a monocyclic or bicyclic aryl or heteroaryl ring;
X is from 1 to 3 substituents on Ar, and each is independently selected from the group consisting of OR₈, NR₈R₉, SR₈, SOR₈, SO₂R₈, SO₂NR₈R₉, NR₈SO₂R₉, CONR₈R₉, NR₈C(═O)R₉, NR₈C(═O)OR₉, OC(═O)NR₈R₉, and NR₈C(═O)NR₉R₁₀,
R₃-R₇ are independently H, alkyl, alkenyl, alkynyl, cycloalkyl, cycloalkenyl, cycloalkylalkyl, cycloalkylalkenyl, or cycloalkylalkynyl, optionally substituted;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, and NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅, NR₁₄C(═O)R₁₅, NR₁₄C(═O)OR₁₅, OC(═O)NR₁₄R₁₅, and NR₁₄C(═O)NR₁₅R₁₆;
wherein any two of the groups R₈, R₉ and R₁₀ are optionally joined with a link selected from the group consisting of bond, —O—, —S—, —SO—, —SO₂—, and —NR₁₇— to form a ring;
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle;
with the first proviso that if X is acyclic and is connected to Ar by a carbon atom, then X contains at least one nitrogen or sulfur atom,
with the second proviso that if X is acyclic and is connected to Ar by an oxygen or nitrogen atom, then X contains at least one additional oxygen, nitrogen or sulfur atom, and
with the third proviso that if X is connected to Ar by a —SO₂— linkage, then R₂ is not nitrogen- or oxygen-substituted R₂-2.

2. The method according to claim 1, wherein said compound of Formula II is a compound of Formula Ia, IIb, or IIc: 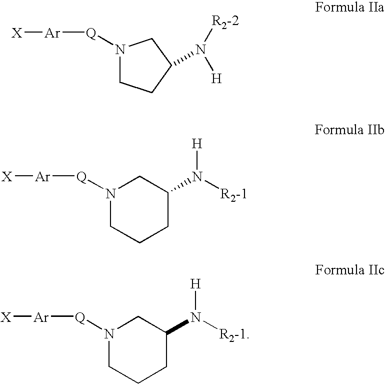
wherein Ar is phenyl, a 6,5-fused bicyclic heteroaryl ring, or a 6,6-fused bicyclic heteroaryl ring; Ar is substituted by 1 or 2 substituents X, and Q is CH₂.

3. The method according to claim 2, wherein Ar is 3-substituted phenyl; 4-substituted phenyl; 3,4-disubstituted phenyl; or 2,3-disubstituted phenyl.

4. The method according to claim 2, wherein Ar is benzofuran, benzothiophene, indole, and benzimidazole.

5. The method according to claim 1, wherein said compound is Compound 1.074, which is (R)—N-(1-(4-(methylthio)benzyl)piperidin-3-yl)-1H-indazol-5-amine; Compound 1.075, which is (S)—N-(1-(4-(methylthio)benzyl)piperidin-3-yl)-1H-indazol-5-amine; Compound 1.091, which is (S)—N-(3-((3-(1H-indazol-5-ylamino)piperidin-1-yl)methyl)phenyl)methanesulfonamide; Compound 1.093, which is (R)—N-(3-((3-(1H-indazol-5-ylamino)piperidin-1-yl)methyl)phenyl)methanesulfonamide; Compound 1.123, which is (R)—N-(3-((3-(1H-indazol-5-ylamino)piperidin-1-yl)methyl)phenyl)ethanesulfonamide; Compound 1.124, which is (S)—N-(3-((3-(1H-indazol-5-ylamino)piperidin-1-yl)methyl)phenyl)ethanesulfonamide; Compound 1.126, which is (R)-2-(3-((3-(1H-indazol-5-ylamino)piperidin-1-yl)methyl)phenoxy)-N-(pyridin-3-yl)acetamide; Compound 1.152, which is (S)-2-(5-((3-(1H-indazol-5-ylamino)piperidin-1-yl)methyl)-2-methylphenoxy)ethanol; Compound 1.157, which is (S)—N-(1-(3-(methylsulfonylmethyl)benzyl)piperidin-3-yl)-1H-indazol-5-amine; Compound 1.158, which is (S)—N-(1-(3-(methylthio)benzyl)piperidin-3-yl)-1H-indazol-5-amine; Compound 1.161, which is (R)-2-(5-((3-(1H-indazol-5-ylamino)piperidin-1-yl)methyl)-2-methylphenoxy)ethanol; Compound 1.195, which is (S)-2-(3-((3-(1H-indazol-5-ylamino)piperidin-1-yl)methyl)phenoxy)acetamide; Compound 1.200, which is (S)-ethyl 2-(3-((3-(1H-indazol-5-ylamino)piperidin-1-yl)methyl)phenoxy)acetate; Compound 1.212, which is (R)—N-(5-((3-(1H-indazol-5-ylamino)piperidin-1-yl)methyl)-2-chlorophenyl)methanesulfonamide; Compound 1.213, which is (S)—N-(5-((3-(1H-indazol-5-ylamino)piperidin-1-yl)methyl)-2-chlorophenyl)methanesulfonamide; Compound 1.215, which is (S)-3-((3-(1H-indazol-5-ylamino)piperidin-1-yl)methyl)benzenesulfonamide; Compound 1.219, which is (S)-3-((3-(1H-indazol-5-ylamino)piperidin-1-yl)methyl)benzamide; Compound 1.233, which is (S)—N-(5-((3-(1H-indazol-5-ylamino)piperidin-1-yl)methyl)-2-methylphenyl)methanesulfonamide; Compound 1.236, which is (S)—N-(5-((3-(1H-indazol-5-ylamino)piperidin-1-yl)methyl)-2-methylphenyl)butane-1-sulfonamide; Compound 1.237, which is (S)—N-(2-((3-(1H-indazol-5-ylamino)piperidin-1-yl)methyl)-5-methylphenyl)-N′,N′dimethylaminosulfamide; Compound 1.238, which is (S)—N-(5-((3-(1H-indazol-5-ylamino)piperidin-1-yl)methyl)-2-methylphenyl)propane-1-sulfonamide; Compound 1.239, which is (S)—N-(5-((3-(1H-indazol-5-ylamino)piperidin-1-yl)methyl)-2-methylphenyl)-4-methylbenzenesulfonamide; Compound 1.249, which is (R)-3-((3-(1H-indazol-5-ylamino)piperidin-1-yl)methyl)benzenesulfonamide; Compound 1.253, which is (S)—N-(5-((3-(1H-indazol-5-ylamino)piperidin-1-yl)methyl)-2-methylphenyl)ethanesulfonamide; Compound 1.258, which is (R)—N-(5-((3-(1H-indazol-5-ylamino)piperidin-1-yl)methyl)-2-methylphenyl)methanesulfonamide; Compound 1.259, which is (R)—N-(5-((3-(1H-indazol-5-ylamino)piperidin-1-yl)methyl)-2-methylphenyl)ethanesulfonamide; Compound 1.260, which is (R)—N-(5-((3-(1H-indazol-5-ylamino)piperidin-1-yl)methyl)-2-methylphenyl)-4-methylbenzenesulfonamide; Compound 1.261, which is (S)—N-(3-((3-(1H-indazol-5-ylamino)piperidin-1-yl)methyl)phenyl)-N′,N′dimethylaminosulfamide; Compound 1.262, which is (R)—N-(2-((3-(1H-indazol-5-ylamino)piperidin-1-yl)methyl)-5-methylphenyl)-N′,N′dimethylaminosulfamide; Compound 1.270, which is (S)—N-(3-((3-(1H-indazol-5-ylamino)piperidin-1-yl)methyl)phenyl)piperidine-1-sulfonamide; Compound 1.275, which is (S)—N-(3-((3-(1H-indazol-5-ylamino)piperidin-1-yl)methyl)-2-methylphenyl)-N′,N′dimethylaminosulfamide; Compound 1.281, which is (R)-2-(5-((3-(1H-indazol-5-ylamino)piperidin-1-yl)methyl)-2-methylphenyl 1H-indazol-5-ylamino)piperidin-1-yl)methyl)-2-methylphenoxy)acetamide; Compound 2.026, which is (R)—N-(1-(4-(methylthio)benzyl)pyrrolidin-3-yl)isoquinolin-5-amine; Compound 2.038, which is (R)—N-(3-((3-(isoquinolin-5-ylamino)pyrrolidin-1-yl)methyl)phenyl)methanesulfonamide; Compound 2.039, which is (R)-2-(3-((3-(isoquinolin-5-ylamino)pyrrolidin-1-yl)methyl)phenoxy)ethanol; Compound 2.041, which is (R)—N-(3-((3-(isoquinolin-5-ylamino)pyrrolidin-1-yl)methyl)phenyl)ethanesulfonamide; Compound 2.054, which is (R)—N-(5-((3-(isoquinolin-5-ylamino)pyrrolidin-1-yl)methyl)-2-methylphenyl)ethanesulfonamide; Compound 2.064, which is (R)-2-(5-((3-(isoquinolin-5-ylamino)pyrrolidin-1-yl)methyl)-2-methylphenoxy)ethanol; Compound 2.067, which is (R)-2-(5-((3-(isoquinolin-5-ylamino)pyrrolidin-1-yl)methyl)-2-methoxyphenoxy)ethanol; Compound 2.068, which is (R)-2-(2-fluoro-5-((3-(isoquinolin-5-ylamino)pyrrolidin-1-yl)methyl)phenoxy)ethanol; Compound 2.069, which is (R)—N-(3-((3-(isoquinolin-5-ylamino)pyrrolidin-1-yl)methyl)phenyl)piperidine-1-sulfonamide; Compound 2.073, which is (R)-2-(5-((3-(isoquinolin-5-ylamino)pyrrolidin-1-yl)methyl)-2-methylphenoxy)acetic acid; Compound 2.076, which is (R)—N-(5-((3-(isoquinolin-5-ylamino)pyrrolidin-1-yl)methyl)-2-methylphenyl)methanesulfonamide; Compound 2.077, which is (R)—N-(5-((3-(isoquinolin-5-ylamino)pyrrolidin-1-yl)methyl)-2-methylphenyl)-N′,N′dimethylaminosulfamide; Compound 2.078, which is (R)—N-(3-((3-(isoquinolin-5-ylamino)pyrrolidin-1-yl)methyl)-2-methylphenyl)methanesulfonamide; Compound 2.079, which is (R)—N-(3-((3-(isoquinolin-5-ylamino)pyrrolidin-1-yl)methyl)-2-methylphenyl)-N′,N′dimethylaminosulfamide; Compound 2.082, which is (R)—N-(1-((2-(methylthio)pyrimidin-4-yl)methyl)pyrrolidin-3-yl)isoquinolin-5-amine; Compound 2.096, which is (R)—N-(3-((3-(isoquinolin-5-ylamino)pyrrolidin-1-yl)methyl)-2-methoxyphenyl)methanesulfonamide; Compound 2.097, which is (R)—N-(3-((3-(isoquinolin-5-ylamino)pyrrolidin-1-yl)methyl)-2-methoxyphenyl)-N′,N′dimethylaminosulfamide; or Compound 2.099, which is (R)-2-(5-((3-(isoquinolin-5-ylamino)pyrrolidin-1-yl)methyl)-2-methylphenoxy)acetamide.

6. A method of treating allergic conjunctivitis, corneal hyposensitivity, neurotrophic keratopathy, dry eye disease, proliferative vitreal retinopathy, macular edema, macular degeneration, or blepharitis; comprising the steps of first identifying a subject suffering from allergic conjunctivitis, corneal hyposensitivity, neurotrophic keratopathy, dry eye disease, proliferative vitreal retinopathy, macular edema, macular degeneration, or blepharitis; then administering to the subject an effective amount of a compound of Formula II to treat allergic conjunctivitis, corneal hyposensitivity, neurotrophic keratopathy, dry eye disease, proliferative vitreal retinopathy, macular edema, macular degeneration, or blepharitis; 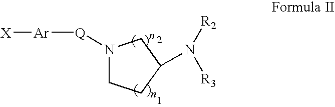
wherein:
Q is C═O, SO₂, or (CR₄R₅)ₙ₃;
n1 is 1, 2, or 3;
n₂ is 1 or 2;
n₃ is 0, 1, 2, or 3;
wherein the ring represented by 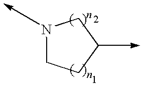
is optionally substituted by alkyl, halo, oxo, OR₆, NR₆R₇, or SR₆;
R₂ is R₂-1 or R₂-2, optionally substituted: 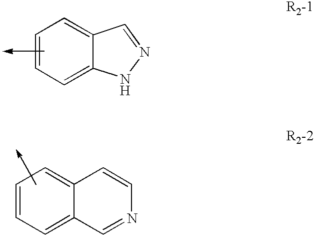
Ar is a monocyclic or bicyclic aryl or heteroaryl ring;
X is from 1 to 3 substituents on Ar, each independently in the form Y-Z, in which Z is attached to Ar;
Y is one or more substituents on Z, and each is independently selected from the group consisting of H, halogen, OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, and NR₈C(═O)NR₉R₁₀;
Z is alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, and (heterocycle)alkynyl;
R₃-R₇ are independently H, alkyl, alkenyl, alkynyl, cycloalkyl, cycloalkenyl, cycloalkylalkyl, cycloalkylalkenyl, or cycloalkylalkynyl, optionally substituted;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, and NR₁₁C(═O)NR₁₂R₁₃;
R₉ and R₁₀ are independently H, allyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)allyl, (heterocycle)alkenyl, (heterocycle)alkynyl, or heterocycle; optionally substituted by one or more halogen or heteroatom-containing substituents selected from the group consisting of OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅, NR₁₄C(═O)R₁₅, NR₁₄C(═O)OR₁₅, OC(═O)NR₁₄R₁₅, and NR₁₄C(═O)NR₁₅R₁₆;
wherein any two of the groups R₈, R₉ and R₁₀ are optionally joined with a link selected from the group consisting of bond, —O—, —S—, —SO—, —SO₂—, and —NR₁₇— to form a ring; and
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle.

7. The method according to claim 6, wherein said compound of Formula II is a compound of Formula IIa, IIb, or IIc: 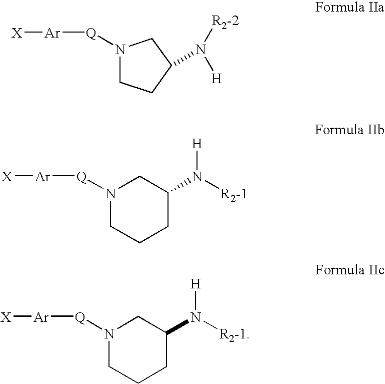
wherein Ar is phenyl, a 6,5-fused bicyclic heteroaryl ring, or a 6,6-fused bicyclic heteroaryl ring; Ar is substituted by 1 or 2 substituents X, and Q is CH₂.

8. The method according to claim 6, wherein said compound is Compound 1.076, which is (R)—N-(1-(4-ethynylbenzyl)piperidin-3-yl)-1H-indazol-5-amine; Compound 1.077, which is (S)—N-(1-(4-ethynylbenzyl)piperidin-3-yl)-1H-indazol-5-amine; Compound 1.153, which is (S)—N-(1-(3-ethynylbenzyl)piperidin-3-yl)-1H-indazol-5-amine; Compound 1.186, which is (S)—N-(1-(3-cyclopropylbenzyl)piperidin-3-yl)-1H-indazol-5-amine; Compound 1.193, which is (R)—N-(1-(3-ethynylbenzyl)piperidin-3-yl)-1H-indazol-5-amine; Compound 1.206, which is (R)—N-(1-(4-cyclopropylbenzyl)piperidin-3-yl)-1H-indazol-5-amine; or Compound 2.031, which is (R)—N-(1-(4-ethynylbenzyl)pyrrolidin-3-yl)isoquinolin-5-amine.

9. A method of treating allergic conjunctivitis, corneal hyposensitivity, neurotrophic keratopathy, dry eye disease, proliferative vitreal retinopathy, macular edema, macular degeneration, or blepharitis; comprising the steps of first identifying a subject suffering from allergic conjunctivitis, corneal hyposensitivity, neurotrophic keratopathy, dry eye disease, proliferative vitreal retinopathy, macular edema, macular degeneration, or blepharitis; then administering to the subject an effective amount of a compound of Formula II to treat allergic conjunctivitis, corneal hyposensitivity, neurotrophic keratopathy, dry eye disease, proliferative vitreal retinopathy, macular edema, macular degeneration, or blepharitis; 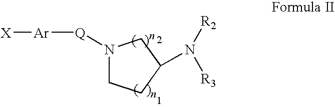
wherein:
Q is C═O, SO₂, or (CR₄R₅)ₙ₃;
n₁ is 1, 2, or 3;
n₂ is 1 or 2;
n₃ is, 1, 2, or 3;
wherein the ring represented by 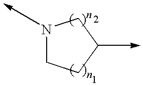
is optionally substituted by alkyl, halo, oxo, OR₆, NR₆R₇, or SR₆;
R₂ is R₂-1 or R₂-2, optionally substituted: 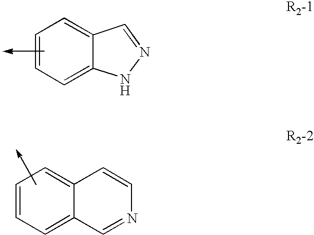
Ar is a monocyclic or bicyclic aryl or heteroaryl ring;
X is from 1 to 3 substituents on Ar, each independently in the form Y-Z, in which Z is attached to Ar;
Y is one or more substituents on Z, and each is independently OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀,
Z is alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl;
R₃-R₇ are independently H, alkyl, alkenyl, alkynyl, cycloalkyl, cycloalkenyl, cycloalkylalkyl, cycloalkylalkenyl, or cycloalkylalkynyl, optionally substituted;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, and NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₄, NR₁₄R₁₅, NO₂, SR₁₄, SOR₁₄, SO₂R14, SO₂NR₁₄R₁₅, NR₁₄SO₂R₁₅, OCF₃, CONR₁₄R₁₅, NR₁₄C(═O)R₁₅, NR₁₄C(═O)OR₁₅, OC(═O)NR₁₄R₁₅, or NR₁₄C(═O)NR₁₅R₁₆;
wherein any two of the groups R₈, R₉ and R₁₀ are optionally joined with a link selected from the group consisting of bond, —O—, —S—, —SO—, —SO₂—, and —NR₁₇— to form a ring; and
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle;
with the proviso that when Z is selected from the group consisting of alkyl, alkenyl, and alkynyl, and Y falls on the carbon by which Z is attached to Ar, then Y contains at least one nitrogen or sulfur atom.

10. The method according to claim 9, wherein Ar is a heteroaryl.

11. The method according to claim 9, wherein said compound of Formula II is a compound of Formula IIa, IIb, or IIc: 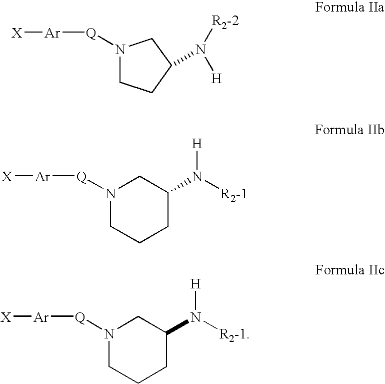
wherein Ar is phenyl, a 6,5-fused bicyclic heteroaryl ring, or a 6,6-fused bicyclic heteroaryl ring; Ar is substituted by 1 or 2 substituents X, and Q is CH₂.

12. The method according to claim 9, wherein said compound is Compound 1.108, which is (R)-2-(6-((3-(1H-indazol-5-ylamino)piperidin-1-yl)methyl)-1H-indol-1-yl)ethanol; Compound 1.109, which is (S)-2-(6-((3-(1H-indazol-5-ylamino)piperidin-1-yl)methyl)-1H-indol-1-yl)ethanol; Compound 1.162, which is (R)-2-(5-((3-(1H-indazol-5-ylamino)piperidin-1-yl)methyl)-1H-indol-1-yl)acetamide; Compound 1.165, which is (S)-2-(5-((3-(1H-indazol-5-ylamino)piperidin-1-yl)methyl)-1H-indol-1-yl)acetamide; Compound 1.176, which is (S)-tert-butyl 3-((4-(1H-indazol-5-ylamino)piperidin-1-yl)methyl)benzylcarbamate; Compound 1.197, which is (S)—N-(1H-indazol-5-ylamino)piperidin-1-yl)methyl)benzyl)acetamide; Compound 1.217, which is (S)-2-(6-((3-(1H-indazol-5-ylamino)piperidin-1-yl)methyl)indolin-1-yl)ethanol; Compound 1.223, which is (S)-(4-((3-(1H-indazol-5-ylamino)piperidin-1-yl)methyl)phenyl)methanol; Compound 1.273, which is (R)-2-(3-((3-(1H-indazol-5-ylamino)piperidin-1-yl)methyl)-1H-indol-1-yl)ethanol; Compound 2.058, which is (R)-2-(6-((3-(isoquinolin-5-ylamino)pyrrolidin-1-yl)methyl)-1H-indol-1-yl)acetamide; Compound 2.059, which is (R)-2-(5-((3-(isoquinolin-5-ylamino)pyrrolidin-1-yl)methyl)-1H-indol-1-yl)acetamide; Compound 2.060, which is (R)-2-(6-((3-(isoquinolin-5-ylamino)pyrrolidin-1-yl)methyl)-1H-indol-1-yl)ethanol; Compound 2.066, which is (R)-2-(5-((3-(isoquinolin-5-ylamino)pyrrolidin-1-yl)methyl)-1H-indol-1-yl)ethanol; or Compound 2.072, which is (R)-2-(3-((3-(isoquinolin-5-ylamino)pyrrolidin-1-yl)methyl)-1H-indol-1-yl)ethanol.

13. A method of treating allergic conjunctivitis, corneal hyposensitivity, neurotrophic keratopathy, dry eye disease, proliferative vitreal retinopathy, macular edema, macular degeneration, or blepharitis; comprising the steps of first identifying a subject suffering from allergic conjunctivitis, corneal hyposensitivity, neurotrophic keratopathy, dry eye disease, proliferative vitreal retinopathy, macular edema, macular degeneration, or blepharitis; then administering to the subject an effective amount of a compound of Formula II to treat allergic conjunctivitis, corneal hyposensitivity, neurotrophic keratopathy, dry eye disease, proliferative vitreal retinopathy, macular edema, macular degeneration, or blepharitis; 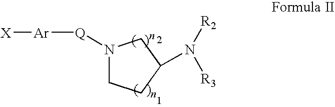
wherein:
Q is C═O, SO₂, or (CR₄R₅)ₙ₃;
n₁ is 1, 2, or 3;
n₂ is 1 or 2;
n₃ is 0, 1, 2, or 3;
wherein the ring represented by 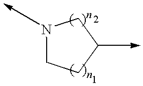
is optionally substituted by alkyl, halo, oxo, OR₆, NR₆R₇, or SR₆;
R₂-5 is 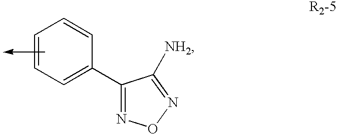
optionally substituted;
Ar is a monocyclic or bicyclic aryl or heteroaryl ring;
X is from 1 to 3 substituents on Ar, each independently in the form Y-Z, in which Z is attached to Ar;
Y is one or more substituents on Z, and each is independently selected from the group consisting of H, halogen, OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, and NR₈C(═O)NR₉R₁₀;
Z is independently selected from the group consisting of absent, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, heterocycle, (heterocycle)alkyl, (heterocycle)alkenyl, and (heterocycle)alkynyl;
R₃-R₇ are independently H, alkyl, alkenyl, alkynyl, cycloalkyl, cycloalkenyl, cycloalkylalkyl, cycloalkylalkenyl, or cycloalkylalkynyl, optionally substituted;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, and NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅, NR₁₄C(═O)R₁₅, NR₁₄C(═O)OR₁₅, OC(═O)NR₁₄R₁₅, and NR₁₄C(═O)NR₁₅R₁₆;
wherein any two of the groups R₈, R₉ and R₁₀ are optionally joined with a link selected from the group consisting of bond, —O—, —S—, —SO—, —SO₂—, and —NR₁₇— to form a ring; and
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle.

14. A method of treating allergic conjunctivitis, corneal hyposensitivity, neurotrophic keratopathy, dry eye disease, proliferative vitreal retinopathy, macular edema, macular degeneration, or blepharitis; comprising the steps of first identifying a subject suffering from allergic conjunctivitis, corneal hyposensitivity, neurotrophic keratopathy, dry eye disease, proliferative vitreal retinopathy, macular edema, macular degeneration, or blepharitis; then administering to the subject an effective amount of a compound of Formula Ia, Ib, or Ic to treat allergic conjunctivitis, corneal hyposensitivity, neurotrophic keratopathy, dry eye disease, proliferative vitreal retinopathy, macular edema, macular degeneration, or blepharitis; 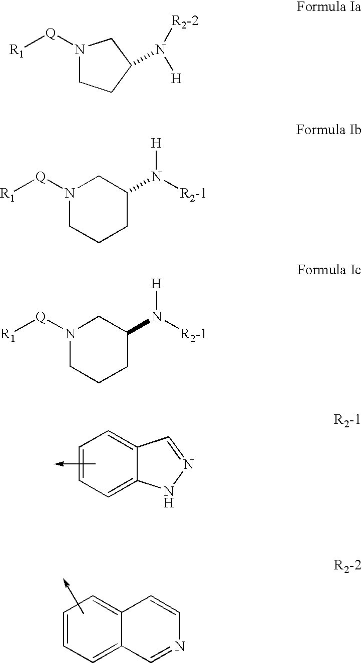
wherein R₁ is phenyl, thiophene, 6,5-fused bicyclic heteroaryl ring, or 6,6-fused bicyclic heteroaryl ring, R1 is either unsubstituted or is optionally substituted with 1, 2 or 3 substituents independently selected from halogen, methyl, ethyl, hydroxyl, methoxy, or ethoxy;
Q is C═O, SO₂, or (CR₄R₅)ₙ₃;
R₂-1 and R₂-2 are optionally substituted;
R₄ and R₅ are independently H, alkyl, cycloalkyl, optionally substituted.

15. The method according to claim 14, wherein R₁ is 3-substituted phenyl, 4-substituted phenyl, 3,4-disubstituted phenyl, or 6,5-fused bicyclic heteroaryl ring.

16. The method according to claim 15, wherein R₁ is benzofuran, benzothiophene, indole, and benzimidazole.

17. The method according to claim 14, wherein R₄ and R₅ are independently H or an unsubstituted alkyl.

18. The method according to claim 14, wherein said compound of Formula Ia is Compound 2.025, which is (R)—N-(1-(4-methylbenzyl)pyrrolidin-3-yl)isoquinolin-5-amine; Compound 2.046, which is (R)—N-(1-benzylpyrrolidin-3-yl)isoquinolin-5-amine; Compound 2.047, which is (R)—N-(1-(4-methoxybenzyl)pyrrolidin-3-yl)isoquinolin-5-amine; Compound 2.055, which is (R)—N-(1-(benzofuran-5-ylmethyl)pyrrolidin-3-yl)isoquinolin-5-amine; Compound 2.057, which is (R)—N-(1-((1H-indol-6-yl)methyl)pyrrolidin-3-yl)isoquinolin-5-amine; Compound 2.061, which is (R)-3-((3-(isoquinolin-5-ylamino)pyrrolidin-1-yl)methyl)phenol; or Compound 2.065, which is (R)—N-(1-((1H-indol-5-yl)methyl)pyrrolidin-3-yl)isoquinolin-5-amine.

19. The method according to claim 14, wherein said compound of Formula Ic is Compound 1.079, which is (S)—N-(1-(4-methoxybenzyl)piperidin-3-yl)-1H-indazol-5-amine; Compound 1.141, which is (S)—N-(1-(4-chlorobenzyl)piperidin-3-yl)-1H-indazol-5-amine; Compound 1.148, which is (S)—N-(1-((1H-indol-6-yl)methyl)piperidin-3-yl)-1H-indazol-5-amine; Compound 1.149, which is (S)—N-(1-((1H-indol-5-yl)methyl)piperidin-3-yl)-1H-indazol-5-amine; Compound 1.150, which is (S)—N-(1-(benzofuran-5-ylmethyl)piperidin-3-yl)-1H-indazol-5-amine; Compound 1.155, which is (S)—N-(1-(2,4-dimethylbenzyl)piperidin-3-yl)-1H-indazol-5-amine; Compound 1.156, which is (S)—N-(1-(2,3-dimethylbenzyl)piperidin-3-yl)-1H-indazol-5-amine; Compound 1.163, which is (S)-3-((3-(1H-indazol-5-ylamino)piperidin-1-yl)methyl)phenol; Compound 1.164, which is (S)—N-(1-(4-fluorobenzyl)piperidin-3-yl)-1H-indazol-5-amine; Compound 1.166, which is (S)—N-(1-((2,3-dihydrobenzo[b][1,4]dioxin-6-yl)methyl)piperidin-3-yl)-1H-indazol-5-amine; Compound 1,171, which is (S)—N-(1-(3-methylbenzyl)piperidin-3-yl)-1H-indazol-5-amine; Compound 1.175, which is (S)—N-(1-(benzo[b]thiophen-5-ylmethyl)piperidin-3-yl)-1H-indazol-5-amine; or Compound 1.277, which is (S)—N-(1-(thiophen-3-ylmethyl)piperidin-3-yl)-1H-indazol-5-amine.

20. The method according to claim 14, wherein said compound of Formula Ib is Compound 1.131, which is (R)—N-(1-(benzofuran-5-ylmethyl)piperidin-3-yl)-1H-indazol-5-amine; Compound 1.132, which is (R)—N-(1-(4-chlorobenzyl)piperidin-3-yl)-1H-indazol-5-amine; Compound 1.133, which is (R)—N-(1-(4-methylbenzyl)piperidin-3-yl)-1H-indazol-5-amine; Compound 1.134, which is (R)—N-(1-(4-bromobenzyl)piperidin-3-yl)-1H-indazol-5-amine; Compound 1.135, which is (R)—N-(1-(4-ethylbenzyl)piperidin-3-yl)-1H-indazol-5-amine; Compound 1.136, which is (R)—N-(1-(2,4-dimethylbenzyl)piperidin-3-yl)-1H-indazol-5-amine; Compound 1.137, which is (R)—N-(1-(benzo[b]thiophen-5-ylmethyl)piperidin-3-yl)-1H-indazol-5-amine; Compound 1.138, which is (R)—N-(1-((1H-indol-6-yl)methyl)piperidin-3-yl)-1H-indazol-5-amine; Compound 1.173, which is (R)-5-((3-(1H-indazol-5-ylamino)piperidin-1-yl)methyl)-2-methylphenol; or Compound 1.252, which is (R)—N-(1-((1H-indol-3-yl)methyl)piperidin-3-yl)-1H-indazol-5-amine.
